(19) United States
(12) Patent Application Publication
(10) Pub. No.: US 2010/0029647 A1
(43) Pub. Date: Feb. 4, 2010

(54) 3,4,(5)-SUBSTITUTED TETRAHYDROPHYRIDINES

(76) Inventors: Keiichi Masuya, Bottmingen (CH); Osamu Irie, Ibaraki (JP); Atsuko Nihonyanagi, Ibaraki (JP); Atsushi Toyao, Ibaraki (JP); Takanori Kanazawa, Ibaraki (JP)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/722,398

(22) PCT Filed: Jan. 12, 2006
(86) PCT No.: PCT/EP06/00216

§ 371 Date: Apr. 10, 2008

(30) Foreign Application Priority Data
Jan. 14, 2005 (GB) 0500784.4

(51) Int. Cl. A61K 31/5377; C07D 401/12; C07D 213/56; C07D 413/12; A61K 31/4439; A61K 31/4406

(52) U.S. Cl. 514/235.5; 546/277.4; 546/316; 544/124; 514/339; 514/355

(57) ABSTRACT

3,4(,5)-substituted tetrahydropyridine compounds, these compounds for use in the diagnostic and therapeutic treatment of a warm-blooded animal, especially for the treatment of a disease that depends on activity of renin; the use of a compound of that class for the preparation of a pharmaceutical formulation for the treatment of a disease that depends on activity of renin; the use of a compound of that class in the treatment of a disease that depends on activity of renin; pharmaceutical formulations comprising a 3,4(,5)-substituted tetrahydropyridine compound, and/or a method of treatment comprising administering a 3,4(,5)-substituted tetrahydropyridine compound, a method for the manufacture of a 3,4(,5)-substituted tetrahydropyridine compound, and novel intermediates and partial steps for its synthesis.

The 3,4(,5)-substituted tetrahydropyridine compounds have the formula I

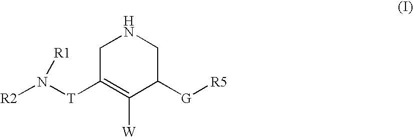

wherein the substituents and symbols are as described in the specification.

The invention relates to 3,4(,5)-substituted tetrahydropyridine compounds, these compounds for use in the diagnostic and therapeutic treatment of a warm-blooded animal, especially for the treatment of a disease (=disorder) that depends on activity of renin; the use of a compound of that class for the preparation of a pharmaceutical formulation for the treatment of a disease that depends on activity of renin; the use of a compound of that class in the treatment of a disease that depends on activity of renin; pharmaceutical formulations comprising a 3,4(,5)-substituted tetrahydropyridine compound; and/or a method of treatment comprising administering a 3,4(,5)-substituted tetrahydropyridine compound, a method for the manufacture of a 3,4(,5)-substituted tetrahydropyridine compound, and novel intermediates and partial steps for its synthesis.

The present invention relates to a compound of the formula I

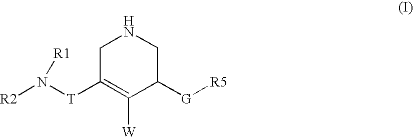

wherein
R1 is unsubstituted or substituted alkyl, unsubstituted or substituted alkenyl, unsubstituted or substituted alkynyl, unsubstituted or substituted aryl, unsubstituted or substituted heterocyclyl or unsubstituted or substituted cycloalkyl;
R2 is hydrogen, unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, or acyl;
W is a moiety selected from those of the formulae IA, IB and IC,

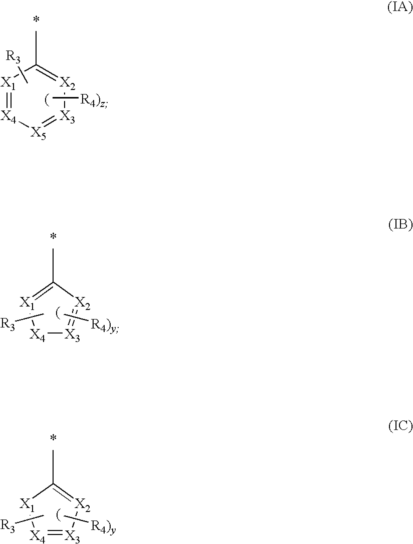

wherein the asterisk (*) denotes the position where the moiety W is bound to the 4-carbon in the piperidine ring in formula I, and wherein
X₁, X₂, X₃, X₄ and X₅ are independently selected from carbon and nitrogen, where X₄ in formula IB and X₁ in formula IC may have one of these meanings or further be selected from S and O, where carbon and nitrogen ring atoms can carry the required number of hydrogen or substituents R₃ or (if present within the limitations given below) R₄ to complete the number of bonds emerging from a ring carbon to four, from a ring nitrogen to three; with the proviso that in formula IA at least 2, preferably at least 3 of X₁ to X₅ are carbon and in formulae IB and IC at least one of X₁ to X₄ is carbon, preferably two of X₁ to X₄ are carbon;
y is 0, 1, 2 or 3;
z is 0, 1, 2, 3 or 4
(the obligatory moiety) R3 which can only be bound to any one of X₁, X₂, X₃ and X₄ (instead of a hydrogen and replacing it) is hydrogen or preferably unsubstituted or substituted C₁-C₇-alkyl, unsubstituted or substituted C₂-C₇-alkenyl, unsubstituted or substituted C₂-C₇-alkynyl, unsubstituted or substituted aryl, unsubstituted or substituted heterocyclyl, unsubstituted or substituted cycloalkyl, halo, hydroxy, etherified or esterified hydroxy, unsubstituted or substituted mercapto, unsubstituted or substituted sulfinyl (—S(═O)—), unsubstituted or substituted sulfonyl (—S(═O)₂—), amino, mono- or di-substituted amino, carboxy, esterified or amidated carboxy, unsubstituted or substituted sulfamoyl, nitro or cyano, with the proviso that if R3 is hydrogen then y and z are 0 (zero);
R4 (which is preferably bound to a ring atom other than that to which R₃ is bound) is—if y or z is 2 or more, independently—selected from a group of substituents consisting of unsubstituted or substituted C₁-C₇-alkyl, unsubstituted or substituted C₂-C₇-alkenyl, unsubstituted or substituted C₂-C₇-alkynyl, halo, hydroxy, etherified or esterified hydroxy, unsubstituted or substituted mercapto, unsubstituted or substituted sulfinyl (—S(═O)—), unsubstituted or substituted sulfonyl (—S(═O)₂—), amino, mono- or di-substituted amino, carboxy, esterified or amidated carboxy, unsubstituted or substituted sulfamoyl, nitro and cyano;
T is carbonyl (—C(═O)—; and
G is methylene, oxy (—O—), thio (—S—), imino (—NH—) or substituted imino (—NR6-) wherein R6 is unsubstituted or substituted alkyl; and
R5 is hydrogen, unsubstituted or substituted alkyl, unsubstituted or substituted alkyloxy (then
G is preferably methylene) or acyl;
or -G-R5 is hydrogen;
or a (preferably pharmaceutically acceptable) salt thereof.

The compounds of the present invention exhibit inhibitory activity on the natural enzyme renin. Thus, compounds of formula I may be employed for the treatment (this term also including prophylaxis) of one or more disorders or diseases selected from, inter alia, hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders.

Listed below are definitions of various terms used to describe the compounds of the present invention as well as their use and synthesis, starting materials and intermediates and the like. These definitions, either by replacing one, more than one or all general expressions or symbols used in the present disclosure and thus yielding preferred embodiments of the invention, preferably apply to the terms as they are used throughout the specification unless they are otherwise limited in specific instances either individually or as part of a larger group.

The term “lower” or “C₁-C₇-” defines a moiety with up to and including maximally 7, especially up to and including maximally 4, carbon atoms, said moiety being branched (one or more times) or straight-chained and bound via a terminal or a non-terminal carbon. Lower or C₁-C₇-alkyl, for example, is n-pentyl, n-hexyl or n-heptyl or preferably C₁-C₄-alkyl, especially as methyl, ethyl, n-propyl, sec-propyl, n-butyl, isobutyl, sec-butyl, tert-butyl.

Halo or halogen is preferably fluoro, chloro, bromo or iodo, most preferably fluoro, chloro or bromo; where halo is mentioned, this can mean that one or more (e.g. up to three) halogen atoms are present, e.g. in halo-C₁-C₇-alkyl, such as trifluoromethyl, 2,2-difluoroethyl or 2,2,2-trifluoroethyl.

Unsubstituted or substituted alkyl is preferably C₁-C₂₀-alkyl, more preferably C₁-C₇-alkyl, that is straight-chained or branched (one or, if desired and possible, more times), and which is unsubstituted or substituted by one or more, e.g. up to three moieties selected from unsubstituted or substituted aryl as described below, especially phenyl or naphthyl each of which is unsubstituted or substituted as described below for unsubstituted or substituted aryl, unsubstituted or substituted heterocyclyl as described below, especially pyrrolyl, furanyl, thienyl, pyrazolyl, triazolyl, tetrazolyl, oxetidinyl, 3-(C₁-C₇-alkyl)-oxetidinyl, pyridyl, pyrimidinyl, morpholino, thiomorpholino, piperidinyl, piperazinyl, pyrrolidinyl, tetrahydrofuran-onyl, tetrahydro-pyranyl, indolyl, 1H-indazanyl, benzofuranyl, benzothiophenyl, quinolinyl, isoquinolinyl, 1,2,3,4-tetrahydro-1,4-benzoxazinyl, 2H-1,4-benzoxazin-3(4H)-onyl, 2H,3H-1,4-benzodioxinyl or benzo[1,2,5]oxadiazolyl each of which is unsubstituted or substituted as described below for unsubstituted or substituted heterocyclyl, unsubstituted or substituted cycloalkyl as described below, especially cyclopropyl, cyclobutyl, cyclopentyl or cyclohexyl each of which is unsubstituted or substituted as described below for unsubstituted or substituted cycloalkyl, halo, hydroxy, C₁-C₇-alkoxy, halo-C₁-C₇-alkoxy, such as trifluoromethoxy, hydroxy-C₁-C₇-alkoxy, C₁-C₇-alkoxy-C₁-C₇-alkoxy, phenyl or naphthyloxy, phenyl- or naphthyl-C₁-C₇-alkyloxy, C₁-C₇-alkanoyloxy, benzoyl- or naphthoyloxy, C₁-C₇-alkylthio, halo-C₁-C₇-alkylthio, such as trifluoromethylthio, C₁-C₇-alkoxy-C₁-C₇-alkylthio, phenyl- or naphthylthio, phenyl or naphthyl-C₁-C₇-alkylthio, C₁-C₇-alkanoylthio, benzoyl- or naphthoylthio, nitro, amino, mono- or di-(C₁-C₇-alkyl and/or C₁-C₇-alkoxy-C₁-C₇-alkyl)-amino, mono- or di-(naphthyl- or phenyl-C₁-C₇-alkyl)-amino, C₁-C₇-alkanoylamino, benzoyl- or naphthoylamino, C₁-C₇-alkylsulfonylamino, phenyl- or naphthylsulfonylamino wherein phenyl or naphthyl is unsubstituted or substituted by one or more, especially one to three, C₁-C₇-alkyl moieties, phenyl- or naphthyl-C₁-C₇-alkylsulfonylamino, carboxyl, C₁-C₇-alkyl-carbonyl, C₁-C₇-alkoxy-carbonyl, phenyl- or naphthyloxycarbonyl, phenyl- or naphthyl-C₁-C₇-alkoxycarbonyl, carbamoyl, N-mono- or N,N-di-(C₁-C₇-alkyl)-aminocarbonyl, N-mono- or N,N-di-(naphthyl- or phenyl-C₁-C₇-alkyl)-aminocarbonyl, cyano, C₁-C₇-alkenylene or alkynylene, C₁-C₇-alkylenedioxy, sulfenyl (—S—OH), sulfinyl (—S(═O)—OH), C₁-C₇-alkylsulfinyl (C₁-C₇-alkyl-S(═O)—), phenyl- or naphthylsulfinyl wherein phenyl or naphthyl is unsubstituted or substituted by one or more, especially one to three, C₁-C₇-alkyl moieties, phenyl- or naphthyl-C₁-C₇-alkylsulfinyl, sulfonyl (—S(O)₂OH), C₁-C₇-alkylsulfonyl (C₁-C₇-alkyl-SO₂—), phenyl- or naphthylsulfonyl wherein phenyl or naphthyl is unsubstituted or substituted by one or more, especially one to three, C₁-C₇-alkyl moieties, phenyl- or naphthyl-C₁-C₇-alkylsulfonyl, sulfamoyl and N-mono or N,N-di-(C₁-C₇-alkyl, phenyl, naphthyl, phenyl-C₁-C₇-alkyl or naphthyl-C₁-C₇-alkyl)-aminosulfonyl.

Unsubstituted or substituted alkenyl preferably has 2 to 20 carbon atoms and includes one or more double bonds, and is more preferably C₂-C₇-alkenyl that is unsubstituted or substituted as described above for unsubstituted or substituted alkyl. Examples are vinyl or allyl.

Unsubstituted or substituted alkynyl preferably has 2 to 20 carbon atoms and includes one or more triple bonds, and is more preferably C₂-C₇-alkynyl that is unsubstituted or substituted as described above for unsubstituted or substituted alkyl. An example is prop-2-ynyl.

Unsubstituted or substituted aryl preferably is a mono- or polycyclic, especially monocyclic, bicyclic or tricyclic aryl moiety with 6 to 22 carbon atoms, especially phenyl (very preferred), naphthyl (very preferred), indenyl, fluorenyl, acenaphthylenyl, phenylenyl or phenanthryl, and is unsubstituted or substituted by one or more, especially one to three, moieties, preferably independently selected from the group consisting of

a substituent of the formula —(C₀-C₇-alkylene)-(X)ᵣ—(C₁-C₇-alkylene)-(Y)ₛ—(C₀-C₇-alkylene)-H where C₀-alkylene means that a bond is present instead of bound alkylene, r and s, each independently of the other, are 0 or 1 and each of X and Y, if present and independently of the others, is —O—, —NV—, —S—, —C(═O)—, —C(═S), —O—CO—, —CO—O—, —NV—CO—; —CO—NV—; —NV— SO₂—, —SO₂—NV; —NV—CO—NV—, —NV—CO—O—, —O—CO—NV—, —NV—SO₂—NV— wherein V is hydrogen or unsubstituted or substituted alkyl as defined below, especially selected from C₁-C₇-alkyl, phenyl, naphthyl, phenyl- or naphthyl-C₁-C₇-alkyl and halo-C₁-C₇-alkyl; e.g. C₁-C₇-alkyl, such as methyl, ethyl, n-propyl, isopropyl, n-butyl, isobutyl, sec-butyl or tert-butyl, hydroxy-C₁-C₇-alkyl, C₁-C₇-alkoxy-C₁-C₇-alkyl, such as 3-methoxypropyl or 2-methoxyethyl, C₁-C₇-alkoxy-C₁-C₇-alkoxy-C₁-C₇-alkyl, C₁-C₇-alkanoyloxy-C₁-C₇-alkyl, C₁-C₇-alkyloxycarbonyl-C₁-C₇-alkyl, amino-C₁-C₇-alkyl, such as aminomethyl, (N-) mono- or (N,N-) di-(C₁-C₇-alkyl)-amino-C₁-C₇-alkyl, C₁-C₇-alkoxy-C₁-C₇-alkylamino-C₁-C₇-alkyl, mono-(naphthyl or phenyl)-amino-C₁-C₇-alkyl, mono-(naphthyl- or phenyl-C₁-C₇-alkyl)-amino-C₁-C₇-alkyl, C₁-C₇-alkanoylamino-C₁-C₇-alkyl, C₁-C₇-alkyl-O—CO—NH—C₁-C₇-alkyl, C₁-C₇-alkylsulfonylamino-C₁-C₇-alkyl, C₁-C₇-alkyl-NH—CO—NH—C₁-C₇-alkyl, C₁-C₇-alkyl-NH—SO, —NH—C₁-C₇-alkyl, C₁-C₇-alkoxy, hydroxy-C₁-C₇-alkoxy, C₁-C₇-alkoxy-C₁-C₇-alkoxy, C₁-C₇-alkanoylamino-C₁-C₇-alkyloxy, carboxy-C₁-C₇-alkyloxy, C₁-C₇-alkyloxycarbonyl-C₁-C₇-alkoxy, mono- or di-(C₁-C₇-alkyl)-aminocarbonyl-C₁-C₇-alkyloxy, C₁-C₇-alkanoyloxy, mono- or di(C₁-C₇-alkyl)-amino, mono-di-(naphthyl- or phenyl-C₁-C₇-alkyl)-amino, N-mono-C₁-C₇-alkoxy-C₁-C₇-alkylamino, C₁-C₇-alkanoylamino, C₁-C₇-alkylsulfonylamino, C₁-C₇-alkyl-carbonyl, halo-C₁-C₇-alkylcarbonyl, hydroxy-C₁-C₇-alkylcarbonyl, C₁-C₇-alkoxy-C₁-C₇-alkylcarbonyl, amino-C₁-C₇-alkylcarbonyl, (N-) mono- or (N,N-) di-(C₁-C₇-alkyl)-amino-C₁-C₇-alkylcarbonyl, C₁-C₇-alkanoylamino-C₁-C₇-alkylcarbonyl, C₁-C₇-alkoxy-carbonyl, hydroxy-C₁-C₇-alkoxycarbonyl, C₁-C₇-alkoxy-C₁-C₇-alkoxycarbonyl, amino-C₁-C₇-alkoxycarbonyl, (N—) mono-(C₁-C₇-alkyl)-amino-C₁-C₇-alkoxycarbonyl, C₁-C₇-alkanoylamino-C₁-C₇-alkoxycarbonyl, N-mono- or N,N-di-(C₁-C₇-alkyl)-aminocarbonyl, N—C₁-C₇-alkoxy-C₁-C₇-alkylcarbamoyl or N-mono- or N,N-di-C₁-C₇-alkyl)-aminosulfonyl;
from C₂-C₇-alkenyl, C₂-C₇-alkynyl, phenyl, naphtyl, heterocyclyl, especially as defined below for heterocyclyl, preferably selected from pyrrolyl, furanyl, thienyl, pyrimidinyl, pyrazolyl, pyrazolidinonyl, N—(C₁-C₇-alkyl, phenyl, naphthyl, phenyl-C₁-C₇-alkyl or naphthyl-C₁-C₇-alkyl)-pyrazolidinonyl, triazolyl, tetrazolyl, oxetidinyl, 3-C₁-C₇-alkyl-oxetidinyl, pyridyl, pyrimidinyl, morpholino, piperidinyl, piperazinyl, pyrrolidinyl, tetrahydrofuran-onyl, tetrahydropyranyl, indolyl, indazolyl, 1H-indazolyl, benzofuranyl, benzothiophenyl, quinolinyl, isoquinolinyl, 1,2,3,4-tetrahydro-1,4-benzoxazinyl, 2H-1,4-benzoxazin-3(4H)-onyl, benzo[1,2,5]oxadiazolyl or 2H,3H-1,4-benzodioxinyl, phenyl- or naphthyl- or heterocycyl-C₁-C₇-alkyl or —C₁-C₇-alkyloxy wherein heterocyclyl is as defined below, preferably selected from pyrrolyl, furanyl, thienyl, pyrimidinyl, pyrazolyl, pyrazolidinonyl, N—(C₁-C₇-alkyl, phenyl, naphthyl, phenylC₁-C₇-alkyl or naphthyl-C₁-C₇-alkyl)-pyrazolidinonyl, triazolyl, tetrazolyl, oxetidinyl, pyridyl, pyrimidinyl, morpholino, piperidinyl, piperazinyl, tetrahydrofuran-onyl, indolyl, indazolyl, 1H-indazanyl, benzofuranyl, benzothiophenyl, quinolinyl, isoquinolinyl, 1,2,3,4-tetrahydro-1,4-benzoxazinyl, 2H-1,4-benzoxazin-3(4H)-onyl- or benzo[1,2,5]oxadiazolyl; such as benzyl or naphthylmethyl, halo-C₁-C₇-alkyl, such as trifluoromethyl, phenyloxy- or naphthyloxy-C₁-C₇-alkyl, phenyl-C₁-C₇-alkoxy- or naphthyl-C₁-C₇-alkoxy-C₁-C₇-alkyl, di-(naphthyl- or phenyl)-amino-C₁-C₇-alkyl, di-(naphthyl- or phenyl-C₁-C₇-alkyl)-amino-C₁-C₇-alkyl, benzoyl or naphthoylamino-C₁-C₇-alkyl, phenyl- or naphthylsulfonylamino-C₁-C₇-alkyl wherein phenyl or naphthyl is unsubstituted or substituted by one or more, especially one to three, C₁-C₇-alkyl moieties, phenyl- or naphthyl-C₁-C₇-alkylsulfonylamino-C₁-C₇-alkyl, carboxyl-C₁-C₇-alkyl, halo, especially fluoro or chloro, hydroxy, phenyl-C₁-C₇-alkoxy wherein phenyl is unsubstituted or substituted by C₁-C₇-alkoxy and/or halo, halo-C₁-C₇-alkoxy, such as trifluoromethoxy, phenyl- or naphthyloxy, phenyl- or naphthyl-C₁-C₇-alkyloxy, phenyl- or naphthyl-oxy-C₁-C₇-alkyloxy, benzoyl- or naphthoyloxy, halo-C₁-C₇-alkylthio, such as trifluoromethylthio, phenyl- or naphthylthio, phenyl- or naphthyl-C₁-C₇-alkylthio, benzoyl or naphthoylthio, nitro, amino, di-(naphthyl- or phenyl-C₁-C₇-alkyl)-amino, benzoyl- or naphthoylamino, phenyl or naphthylsulfonylamino wherein phenyl or naphthyl is unsubstituted or substituted by one or more, especially one to three, C₁-C₇-alkoxy-C₁-C₇-alkyl or C₁-C₇-alkyl moieties, phenyl- or naphthyl-C₁-C₇-alkylsulfonylamino, carboxyl, (N,N-) di-(C₁-C₇-alkyl)-amino-C₁-C₇-alkoxycarbonyl, halo-C₁-C₇-alkoxycarbonyl, phenyl- or naphthyloxycarbonyl, phenyl- or naphthyl-C₁-C₇-alkoxycarbonyl, (N,N-) di-(C₁-C₇-alkyl)-amino-C₁-C₇-alkoxycarbonyl, carbamoyl, N-mono or N,N-di-(naphthyl-, phenyl-, C₁-C₇-alkyloxyphenyl and/or C₁-C₇-alkyloxynapthtyl-)aminocarbonyl, N-mono- or N,N-di-(naphthyl- or phenyl-C₁-C₇-alkyl)-aminocarbonyl, cyano, C₁-C₇-alkylene which is unsubstituted or substituted by up to four C₁-C₇-alkyl substituents and bound to two adjacent ring atoms of the aryl moiety, C₂-C₇-alkenylene or -alkynylene which are bound to two adjacent ring atoms of the aryl moiety, sulfenyl, sulfinyl, C₁-C₇-alkylsulfinyl, phenyl- or naphthylsulfinyl wherein phenyl or naphthyl is unsubstituted or substituted by one or more, especially one to three, C₁-C₇-alkoxy-C₁-C₇-alkyl or C₁-C₇-alkyl moieties, phenyl- or naphthyl-C₁-C₇-alkylsulfinyl, sulfonyl, C₁-C₇-alkylsulfonyl, halo-C₁-C₇-alkylsulfonyl, hydroxy-C₁-C₇-alkylsulfonyl, C₁-C₇-alkoxy-C₁-C₇-alkylsulfonyl, amino-C₁-C₇-alkylsulfonyl, (N,N-) di-(C₁-C₇-alkyl)-amino-C₁-C₇-alkylsulfonyl, C₁-C₇-alkanoylamino-C₁-C₇-alkylsulfonyl, phenyl- or naphthylsulfonyl wherein phenyl or naphthyl is unsubstituted or substituted by one or more, especially one to three, C₁-C₇-alkoxy-C₁-C₇-alkyl or C₁-C₇-alkyl moieties, phenyl- or naphthyl-C₁-C₇-alkylsulfonyl, sulfamoyl and N-mono or N,N-di-(C₁-C₇-alkyl, phenyl-, naphthyl, phenyl-C₁-C₇-alkyl and/or naphthyl-C₁-C₇-alkyl)-aminosulfonyl. Especially preferably aryl is phenyl or naphthyl, each of which is unsubstituted or substituted by one or more, e.g. up to three, substituents independently selected from the group consisting of C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, amino-C₁-C₇-alkyl, C₁-C₇-alkoxy-C₁-C₇-alkylamino-C₁-C₇-alkyl, carboxy-C₁-C₇-alkyl, C₁-C₇-alkoxycarbonyl-C₁-C₇-alkyl, halo, especially fluoro, chloro or bromo, hydroxy, C₁-C₇-alkoxy, hydroxy-C₁-C₇-alkoxy C₁-C₇-alkoxy-C₁-C₇-alkoxy, amino-C₁-C₇-alkoxy, N—C₁-C₇-alkanoylamino-C₁-C₇-alkoxy, carboxyl-C₁-C₇-alkyloxy, C₁-C₇-alkoxycarbonyl-C₁-C₇-alkyloxy, carbamoyl-C₁-C₇-alkoxy, N-mono- or N,N-di-(C₁-C₇-alkyl)-carbamoyl-C₁-C₇-alkoxy, morpholino-C₁-C₇-alkoxy, pyridyl-C₁-C₇-alkoxy, amino, C₁-C₇-alkanoylamino, C₁-C₇-alkanoyl, C₁-C₇-alkoxy-C₁-C₇-alkanoyl, carboxy, carbamoyl, N—(C₁-C₇-alkoxy-C₁-C₇-alkyl)-carbamoyl, pyrazolyl, pyrazolyl-C₁-C₇-alkoxy, 4-C₁-C₇-alkylpiperidin-1-yl, nitro and cyano.

Unsubstituted or substituted heterocyclyl is preferably a mono- or polycyclic, preferably a mono-, or bi- or (less preferably) tricyclic-, unsaturated, partially saturated or saturated ring system with preferably 3 to 22 (more preferably 3 to 14) ring atoms and with one or more, preferably one to four, heteroatoms independently selected from nitrogen (═N—, —NH— or substituted —NH—), oxygen, sulfur (—S—, —S(═O)— or —S—(═O)₂—), and is unsubstituted or substituted by one or more, e.g. up to three, substitutents preferably independently selected from the substitutents mentioned above for aryl and from oxo. Preferably, heterocyclyl (which is unsubstituted or substituted as just mentioned) is selected from the following moieties (the asterisk marks the point of binding to the rest of the molecule of formula I):

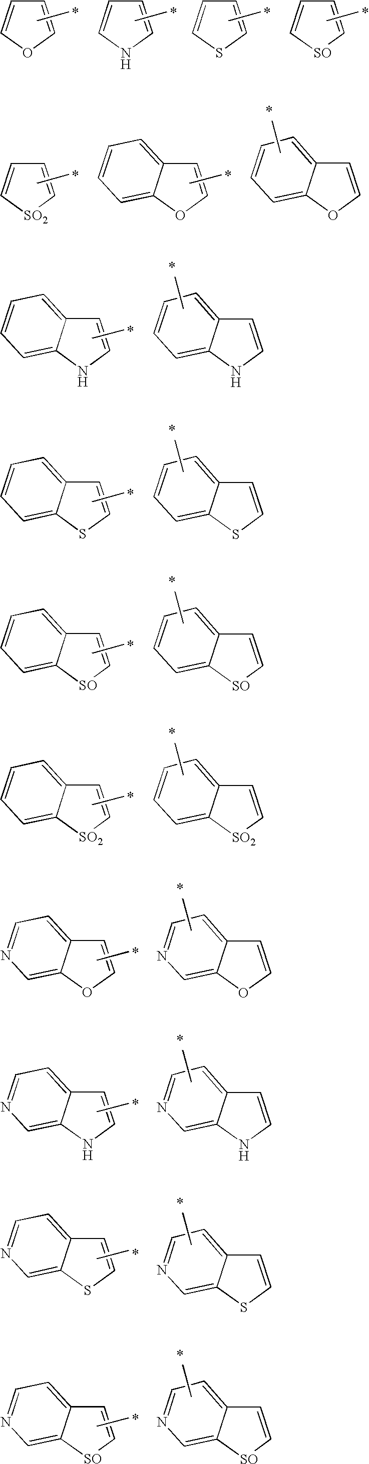

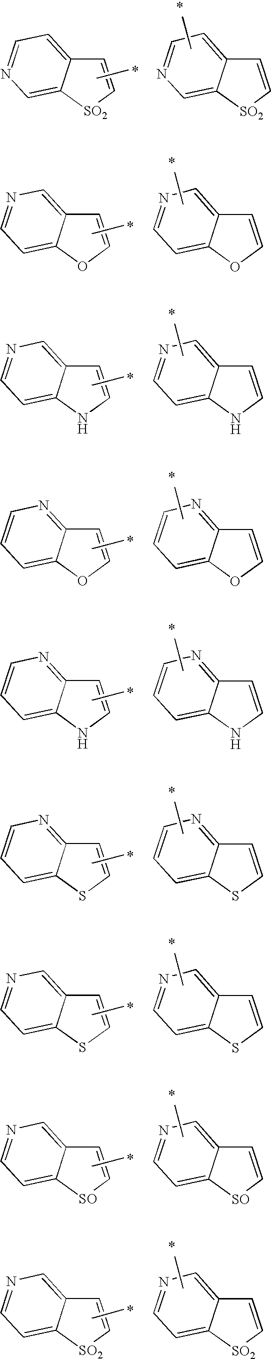

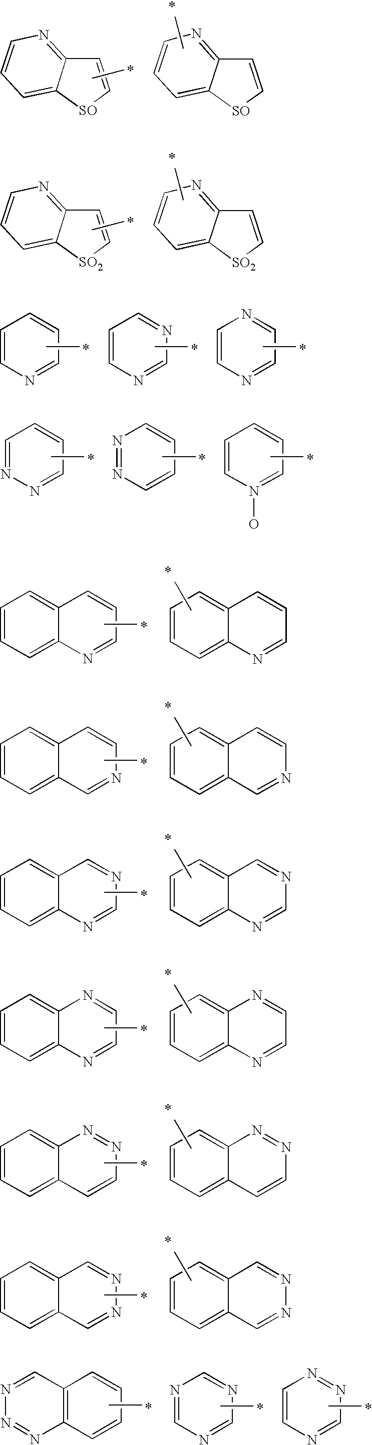

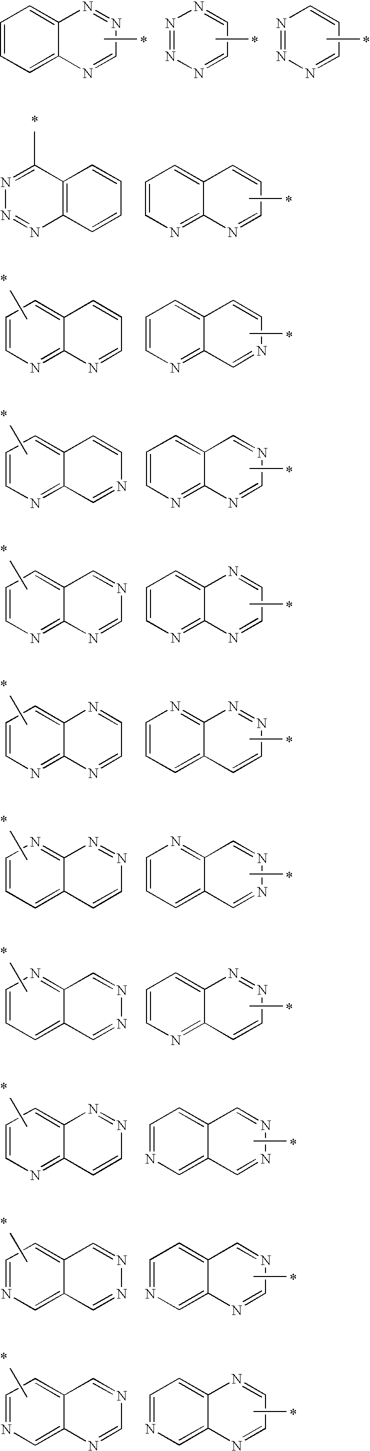

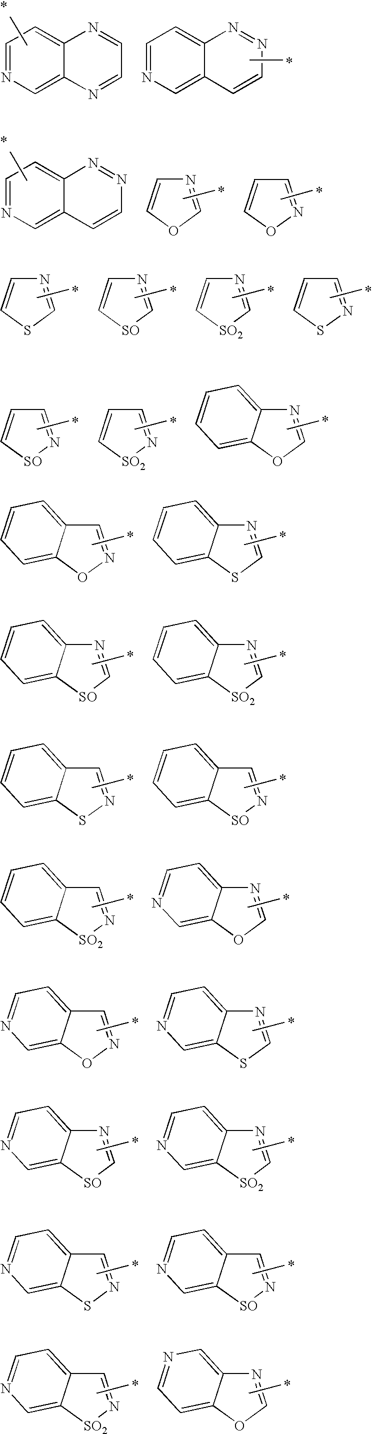

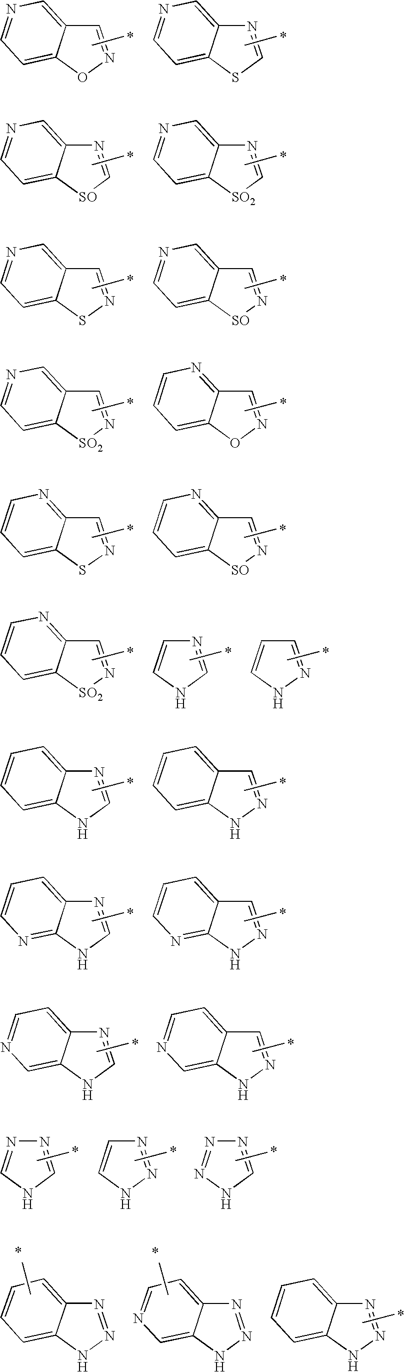

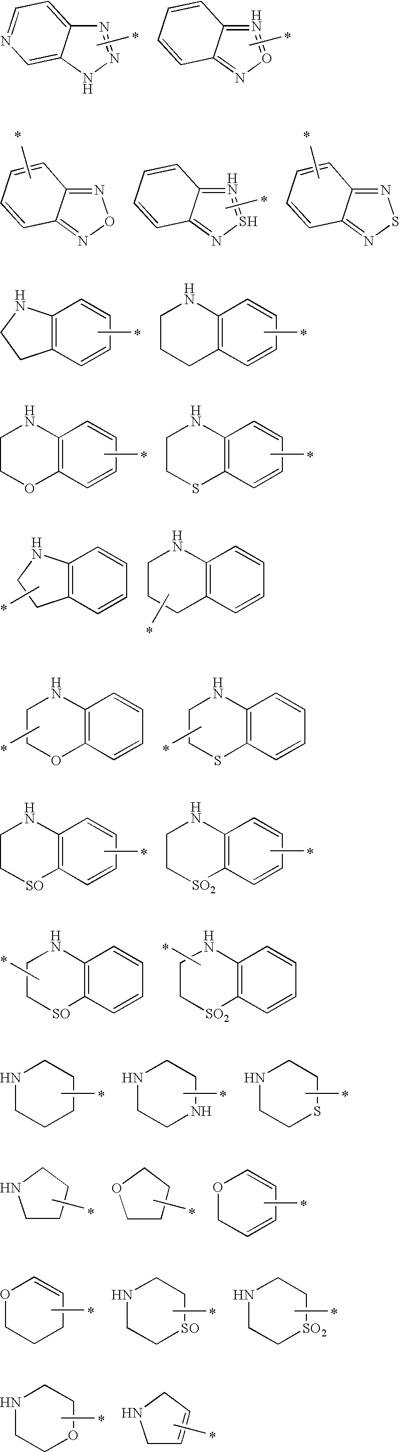

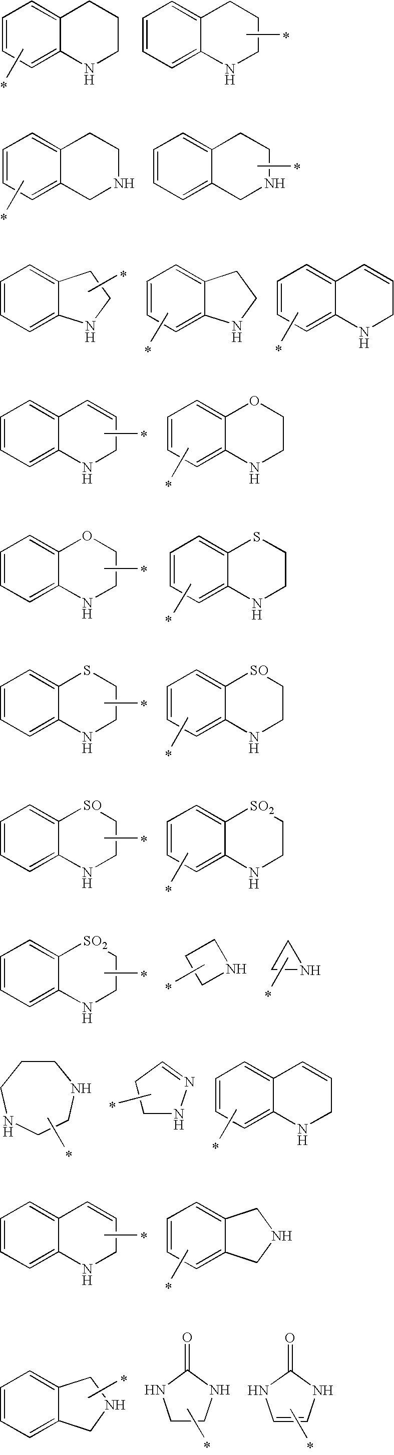

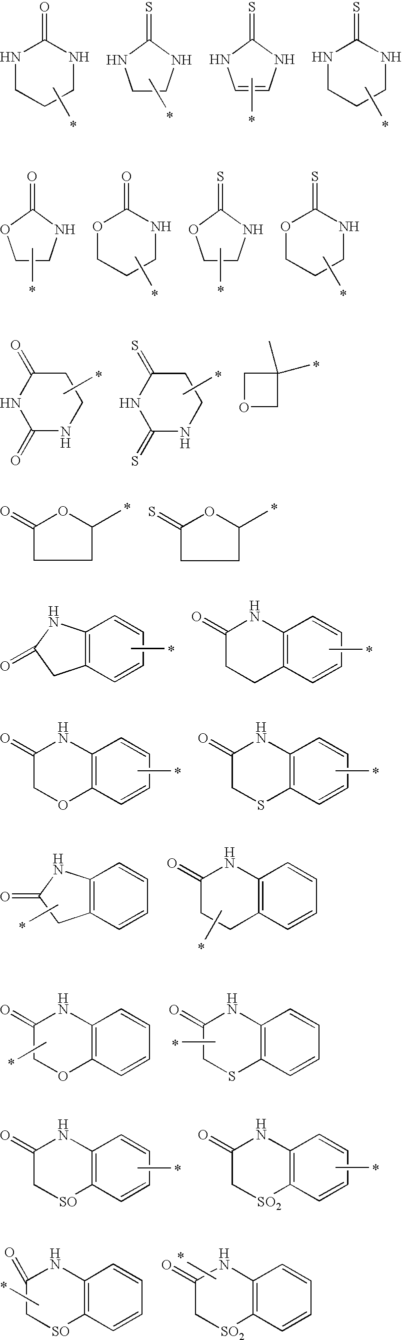

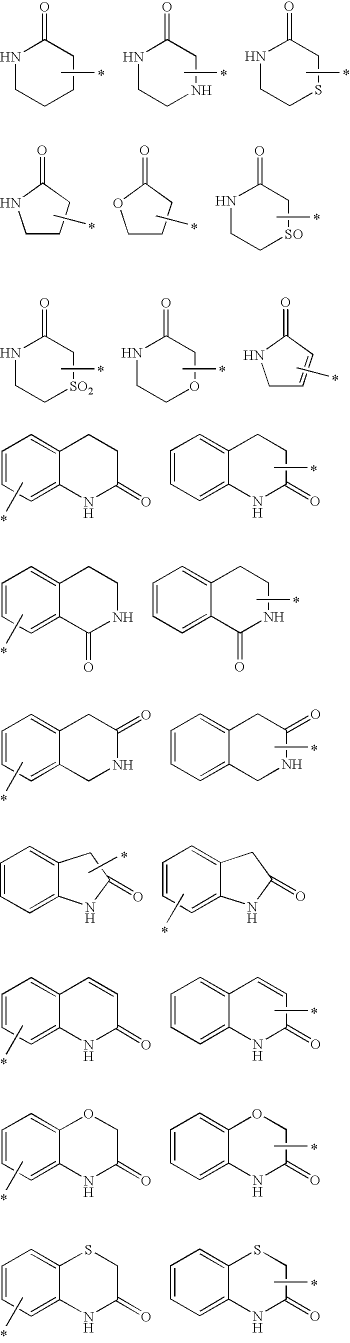

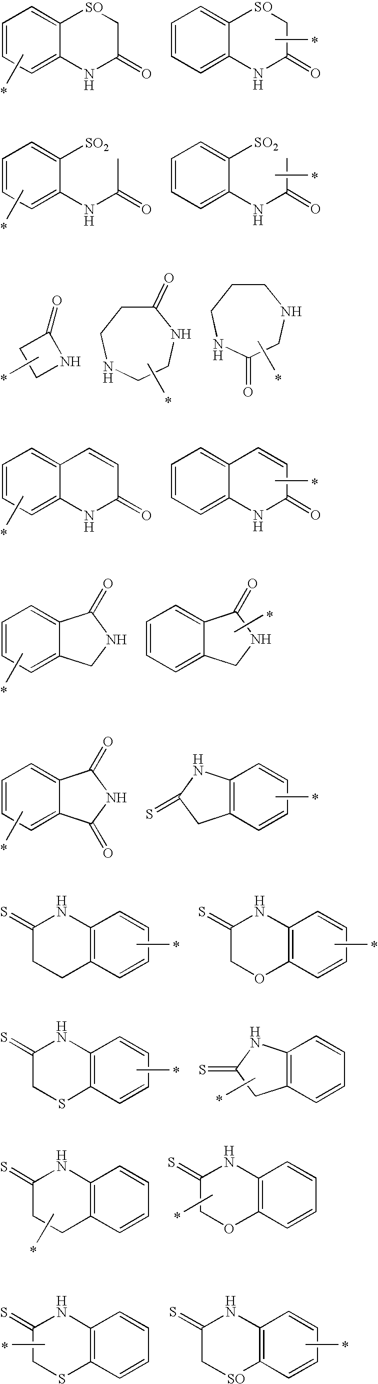

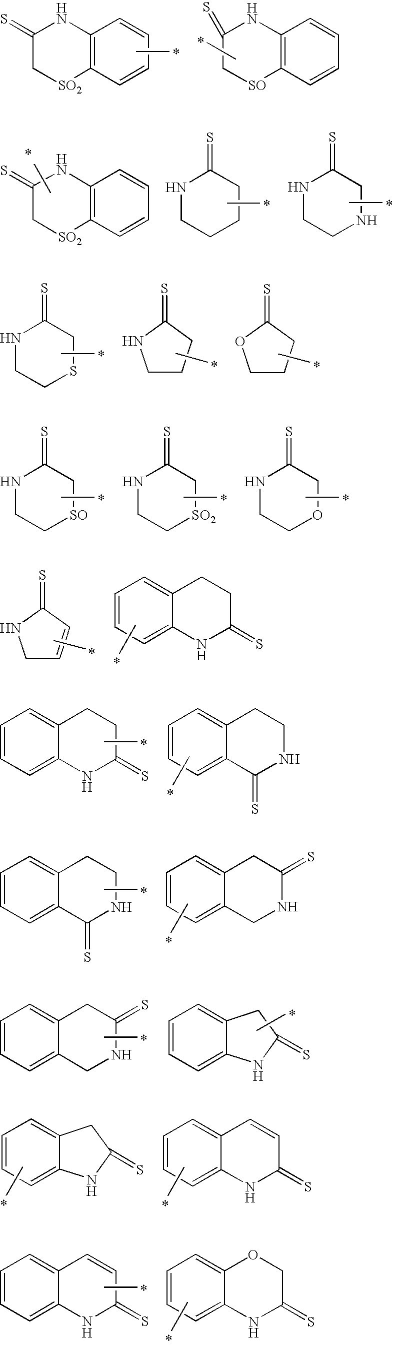

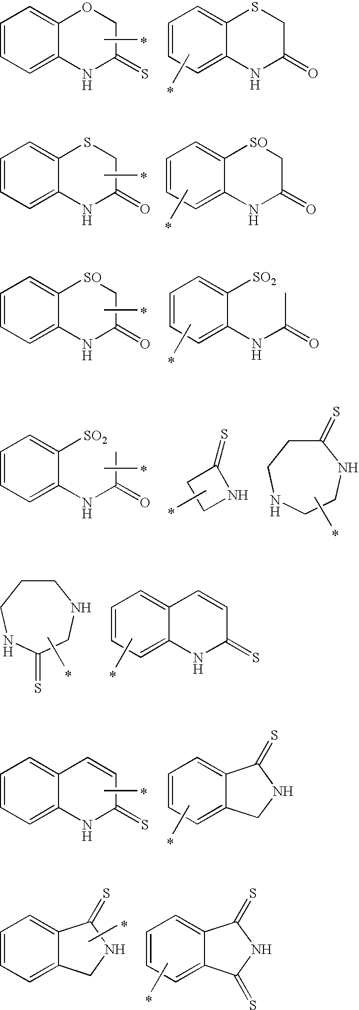

where in each case where an NH is present the bond with the asterisk connecting the respective heterocyclyl moiety to the rest of the molecule the H may be replaced with said bond and/or the H may be replaced by a substituent, preferably as defined above. Especially preferred as heterocyclyl is pyrrolyl, furanyl, thienyl, pyrimidinyl, pyrazolyl, pyrazolidinonyl (=oxo-pyrazolidinyl), triazolyl, tetrazolyl, oxetidinyl, pyridyl, pyrimidinyl, morpholino, piperidinyl, piperazinyl, pyrrolidinyl, tetrahydrofuran-onyl (=oxo-tetrahydrofuranyl), tetrahydro-pyranyl, indolyl, indazolyl, 1H-indazanyl, benzofuranyl, benzothiophenyl, quinolinyl, isoquinolinyl, 1,2,3,4-tetrahydro-1,4-benzoxazinyl, 2H-1,4-benzoxazin-3(4H)-onyl, 2H,3H-1,4-benzodioxinyl, benzo[1,2,5]oxadiazolyl, thiophenyl, pyridyl, indolyl, 1H-indazolyl, quinolyl, isoquinolyl or 1-benzothiophenyl; each of which is unsubstituted or substituted by one or more, e.g. up to three, substituents as mentioned above for substituted aryl, preferably independently selected from the group consisting of C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, amino-C₁-C₇-alkyl, C₁-C₇-alkoxy-C₁-C₇-alkylamino-C₁-C₇-alkyl, carboxy-C₁-C₇-alkyl, C₁-C₇-alkoxy-C₁-C₇-alkyl, halo, hydroxy, C₁-C₇-alkoxy, C₁-C₇-alkoxy-C₁-C₇-alkoxy, amino-C₁-C₇-alkoxy, N—C₁-C₇-alkanoylamino-C₁-C₇-alkoxy, carbamoyl-C₁-C₇-alkoxy, N—C₁-C₇-alkylcarbamoyl-C₁-C₇-alkoxy, C₁-C₇-alkanoyl, C₁-C₇-alkoxy-C₁-C₇-alkanoyl, carboxy, carbamoyl and N—C₁-C₇-alkoxy-C₁-C₇-alkylcarbamoyl. In the case of heterocycles including an NH ring member, the substitutents, as far as bound via a carbon or oxygen atom, are preferably bound at the nitrogen instead of the H.

Unsubstituted or substituted cycloalkyl is preferably mono- or polycyclic, more preferably monocyclic, C₃-C₁₀-cycloalkyl which may include one or more double (e.g. in cycloalkenyl) and/or triple bonds (e.g. in cycloalkynyl), and is unsubstituted or substituted by one or more, e.g. one to three substitutents preferably independently selected from those mentioned above as substituents for aryl. Preferred is cyclopropyl, cyclobutyl, cyclopentyl, cyclohexyl or cycloheptyl.

Acyl is preferably unsubstituted or substituted aryl-carbonyl or -sulfonyl, unsubstituted or substituted heterocyclylcarbonyl or -sulfonyl, unsubstituted or substituted cycloalkylcarbonyl or -sulfonyl, formyl or unsubstituted, substituted alkylcarbonyl or -sulfonyl, or (especially in if G is oxy or preferably imino) as acyl R5) substituted aryloxycarbonyl or -oxysulfonyl, unsubstituted or substituted heterocyclyloxycarbonyl or -oxysulfonyl, unsubstituted or substituted cycloalkyloxycarbonyl or -oxysulfonyl, unsubstituted or substituted alkyloxycarbonyl or -oxysulfonyl or N-mono- or N,N-di-(substituted aryl-, unsubstituted or substituted heterocyclyl, unsubstituted or substituted cycloalkyl or unsubstituted or substituted alkyl)-aminocarbonyl, wherein unsubstituted or substituted aryl, unsubstituted or substituted heterocyclyl, unsubstituted or substituted cycloalkyl and unsubstituted or substituted alkyl are preferably as described above. Preferred is C₁-C₇-alkanoyl, unsubstituted or mono-, di- or tri-(halo)-substituted benzoyl or naphthoyl, unsubstituted or phenyl-substituted pyrrolidinylcarbonyl, especially phenyl-pyrrolidinocarbonyl, C₁-C₇-alkylsulfonyl or (unsubstituted or C₁-C₇-alkyl-substituted) phenylsulfonyl.

“-Oxycarbonyl-” means —O—C(═O), “aminocarbonyl” means in the case of mono-substitution —NH—C(═O)—, in the case of double substitution also the second hydrogen is replaced by the corresponding moiety.

Etherified or esterified hydroxy is especially hydroxy that is esterified with acyl as defined above, especially in C₁-C₇-alkanoyloxy, or preferably etherified with alkyl, alkenyl, alkynyl, aryl, heterocyclyl or cycloalkyl each of which is unsubstituted or substituted and is preferably as described above for the corresponding unsubstituted or substituted moieties. Especially preferred is

unsubstituted or especially substituted C₁-C₇-alkyloxy, especially with a substituent selected from C₁-C₇-alkoxy; phenyl, tetrazolyl, tetrahydrofuran-onyl, oxetidinyl, 3-(C₁-C₇-alkyl)-oxetidinyl, pyridyl or 2H,3H-1,4-benzodioxinyl, each of which is unsubstituted or substituted by one or more, preferably up to three, e.g. 1 or two substituents independently selected from C₁-C₇-alkyl, hydroxy, C₁-C₇-alkoxy, phenyloxy wherein phenyl is unsubstituted or substituted by C₁-C₇-alkoxy and/or halo, phenyl-C₁-C₇-alkoxy wherein phenyl is unsubstituted or substituted by C₁-C₇-alkoxy and/or halo; halo, amino, N-mono- or N,N-di(C₁-C₇-alkyl, phenyl, naphthyl, phenyl-C₁-C₇-alkyl or naphthyl-C₁-C₇-alkyl)amino, C₁-C₇-alkanoylamino, carboxy, N-mono- or N,N-di(C₁-C₇-alkyl, phenyl, naphthyl, phenyl-C₁-C₇-alkyl or naphthyl-C₁-C₇-alkyl)-aminocarbonyl, morpholino, morpholino-C₁-C₇-alkoxy, pyridyl-C₁-C₇-alkoxy, pyrazolyl, 4-C₁-C₇-alkylpiperidin-1-yl and cyano; or selected from morpholino;
or unsubstituted or substituted aryloxy with unsubstituted or substituted aryl as described above, especially phenyloxy with phenyl that is unsubstituted or substituted as just described; or
unsubstituted or substituted heterocyclyloxy with unsubstituted or substituted heterocyclyl as described above, preferably tetrahydropyranyloxy.

Substituted mercapto can be mercapto that is thioesterified with acyl as defined above, especially with lower alkanoyloxy; or preferably thioetherified with alkyl, alkenyl, alkynyl, aryl, heterocyclyl or cycloalkyl each of which is unsubstituted or substituted and is preferably as described above for the corresponding unsubstituted or substituted moieties. Especially preferred is unsubstituted or especially substituted C₁-C₇-alkylthio or unsubstituted or substituted arylthio with unsubstituted or substituted C₁-C₇-alkyl or aryl as just described for the corresponding moieties under etherified hydroxy.

Substituted sulfinyl or sulfonyl can be substituted with alkyl, alkenyl, alkynyl, aryl, heterocyclyl or cycloalkyl each of which is unsubstituted or substituted and is preferably as described above for the corresponding unsubstituted or substituted moieties. Especially preferred is unsubstituted or especially substituted C₁-C₇-alkylsulfinyl or -sulfonyl or unsubstituted or substituted arylsulfinyl or -sulfonyl with unsubstituted or substituted C₁-C₇-alkyl or aryl as just described for the corresponding moieties under etherified hydroxy.

In mono- or di-substituted amino, amino is preferably substituted by one or more substituents selected from one acyl, especially C₁-C₇-alkanoyl, phenylcarbonyl (=benzoyl), C₁-C₇-alkylsulfonyl or phenylsulfonyl wherein phenyl is unsubstituted or substituted by one to 3 C₁-C₇-alkyl groups, and one or two moieties selected from alkyl, alkenyl, alkynyl, aryl, heterocyclyl and cycloalkyl each of which is unsubstituted or substituted and is preferably as described above for the corresponding unsubstituted or substituted moieties. Preferred is C₁-C₇-alkanoylamino, mono- or di-(phenyl, naphthyl, C₁-C₇-alkoxy-phenyl, C₁-C₇-alkoxynaphthyl, naphthyl-C₁-C₇-alkyl or phenyl-C₁-C₇-alkyl)-carbonylamino (e.g. 4-methoxybenzoylamino), mono- or di-(C₁-C₇-alkyl and/or C₁-C₇-alkoxy-C₁-C₇-alkyl)-amino or mono- or di-(phenyl, naphthyl, C₁-C₇-alkoxy-phenyl, C₁-C₇-alkoxynaphthyl, phenyl-C₁-C₇-alkyl, naphthyl-C₁-C₇-alkyl, C₁-C₇-alkoxy-naphthyl-C₁-C₇-alkyl or C₁-C₇-alkoxy-phenyl-C₁-C₇-alkyl)-amino.

Esterified carboxy is preferably alkyloxycarbonyl, aryloxycarbonyl, heterocyclyloxycarbonyl or cycloalkyloxycarbonyl, wherein alkyl, aryl, heterocyclyl and cycloalkyl are unsubstituted or substituted and the corresponding moieties and their substituents are preferably as described above. Preferred is C₁-C₇-alkoxycarbonyl, phenyl-C₁-C₇-alkyloxycarbonyl, phenoxycarbonyl or naphthoxycarbonyl.

In amidated carboxy, the amino part bound to the carbonyl in the amido function (D₂N—C(═O)—) wherein each D is independently of the other hydrogen or an amino substituent) is unsubstituted or substituted as described for substituted amino, but preferably without acyl as amino substituent. Preferred is mono- or di-(C₁-C₇-alkyl and/or C₁-C₇-alkoxy-C₁-C₇-alkyl)-aminocarbonyl or mono- or di-(C₁-C₇-alkyloxyphenyl, C₁-C₇-alkyloxynaphthyl, naphthyl-C₁-C₇-alkyl or phenyl-C₁-C₇-alkyl)-aminocarbonyl.

In substituted sulfamoyl, the amino part bound to the sulfonyl in the sulfamoyl function (D₂N—S(═O)₂—) wherein each D is independently of the other hydrogen or an amino substituent) is unsubstituted or substituted as described for substituted amino, but preferably without acyl as amino substituent. Preferred is mono- or di-(C₁-C₇-alkyl and/or C₁-C₇-alkoxy-C₁-C₇-alkyl)-aminosulfonyl or mono- or di-(C₁-C₇-alkyloxyphenyl, C₁-C₇-alkyloxynaphthyl, naphthyl-C₁-C₇-alkyl or phenyl-C₁-C₇-alkyl)-aminosulfonyl.

Unsubstituted or substituted C₁-C₇-alkyl, unsubstituted or substituted C₁-C₇-alkenyl and unsubstituted or substituted C₂-C₇-alkynyl and their substituents are defined as above under the corresponding (un)substituted alkyl, (un)substituted alkynyl and (un)substituted alkynyl moieties but with the given number of carbon atoms in the alkyl, alkenyl or alkynyl moieties.

The following preferred embodiments of the moieties and symbols in formula I can be employed independently of each other to replace more general definitions and thus to define specially preferred embodiments of the invention, where the remaining definitions can be kept broad as defined in embodiments of the inventions defined above of below.

As G, methylene, oxy and imino are preferred, as R5 hydrogen, C₁-C₇-alkyl, C₁-C₇-alkoxy-C₁-C₇-alkyl, C₁-C₇-alkoxy, C₁-C₇-alkanoyl, C₁-C₇-alkylsulfonyl or (unsubstituted or C₁-C₇-alkyl-substituted phenyl)-sulfonyl, or also (especially if G is imino) N-mono- or N,N-di-(C₁-C₇-alkyl, phenyl, naphthyl, phenyl-C₁-C₇-alkyl and/or napthyl-C₁-C₇-alkyl)-aminocarbonyl or (C₁-C₇-alkyl, phenyl, naphthyl, phenyl-C₁-C₇-alkyl and/or napthyl-C₁-C₇-alkyl)-oxycarbonyl; or G-R5 is preferably hydrogen.

R2 preferably has one of the meanings given for R2 herein other than acyl or is unsubstituted or phenyl-substituted pyrrolidinylcarbonyl, especially phenyl-pyrrolidinocarbonyl.

As R1, C₁-C₇-alkyl, halo-C₁-C₇-alkyl, di-(phenyl)-C₁-C₇-alkyl, C₃-C₈-cyclopropyl, (unsubstituted or C₁-C₇-alkoxy-substituted naphthyl)-C₁-C₇-alkyl, (halo-phenyl)-C₁-C₇-alkyl, or phenyl substituted by C₁-C₇-alkyl, halo, C₁-C₇-alkyloxy and/or C₁-C₇-alkoxy-C₁-C₇-alkyloxy is especially preferred.

As R2, these or the other mentioned moieties mentioned herein are preferred, especially unsubstituted or substituted alkyl, unsubstituted or substituted aryl or unsubstituted or substituted heterocyclyl. R2 preferably has one of the meanings given for R2 herein other than acyl or is unsubstituted or substituted benzoyl (=phenylcarbonyl) or naphthoyl (=naphthylcarbonyl), or unsubstituted or phenyl-substituted pyrrolidinylcarbonyl, especially phenyl-pyrrolidinocarbonyl.

In a moiety W of the formula IA, preferably one of X₁ and X₂ is nitrogen or CH, while the other and X₃, X₄ and X₅ are CH.

In a moiety W of the formula IB, preferably X₄ is CH₂, NH, S or O and one of X₁, X₂ and (preferably if X₄ is CH₂ or N) X₃, more preferably X₂, is N, while the others are each CH, with the proviso that at least one ring nitrogen (N or in the case or X₄ NH) is present R3 is then preferably bound to X₃ instead of a hydrogen.

In a moiety W if the formula IC, preferably X₁ is CH₂, NH, S or O and one of X₂, X₃ and X₄ is N, while the others are CH, with the proviso that at least one ring nitrogen (N or in the case or X₁ NH) is present. R3 is then preferably bound to X₂ or more preferably to X₃ or to X₄ instead of a hydrogen.

The skilled person will understand that a substituent R3 (and, where present, R4) can only be present at the position of and instead of a hydrogen bound to a ring member X₁ to X₄ selected from CH, CH₂ or NH so that only four-bonded carbon or three-bonded nitrogen (which, in the case of salt formation, may however be protonated to become four-bonded and then positively charged) is present.

y is 0, 1, 2 or 3, preferably 0 or 1, most preferably 0, and z is 0, 1, 2, 3 or 4, preferably 0 or 1.

As R₃, phenyl, pyridyl, hydroxyphenyl, halophenyl, mono- or di-C₁-C₇-alkyloxy)-phenyl, C₁-C₇-alkanoylaminophenyl, mono- or di-(C₁-C₇-alkyloxy)-pyridyl, phenyl substituted by halo and C₁-C₇-alkyloxy, pyridyl substituted by halo and/or C₁-C₇-alkyloxy, N-mono- or N,N-di-(C₁-C₇-alkyl)-aminopyridyl, morpholino- or thiomorpholino-C₁-C₇-alkyloxyphenyl, phenyloxy, phenyl-C₁-C₇-alkyloxy, pyridyl-C₁-C₇-alkyloxy, mono- or di-(halo)phenyl-C₁-C₇-alkyloxy, mono- or di-(C₁-C₇-alkyloxy)-phenyl-C₁-C₇-alkyloxy, mono- or di-(C₁-C₇-alkyloxy)-pyridyl-C₁-C₇-alkyloxy, phenyl-C₁-C₇-alkyloxy with phenyl substituted by halo and C₁-C₇-alkyloxy, pyridyl-C₁-C₇-alkyloxy with pyridyl substituted by halo and C₁-C₇-alkyloxy, N-mono- or N,N-di-(C₁-C₇-alkyl)-aminopyridyl-C₁-C₇-alkyloxy, morpholino-C₁-C₇-alkoxy, thiomorpholino-C₁-C₇-alkoxy, C₁-C₇-alkyloxy-C₁-C₇-alkyloxy, cyanophenyl-C₁-C₇-alkyloxy, pyrazolylphenyl-C₁-C₇-alkyloxy, N—C₁-C₇-alkylpiperazinophenyl-C₁-C₇-alkyloxy, phenoxy-C₁-C₇-alkyloxy, tetrahydropyranyloxy, 2H,3H-1,4-benzodioxinyl-C₁-C₇-alkyloxy, N—(C₁-C₇-alkyloxyphenyl)-aminocarbonyl or C₁-C₇-alkyloxybenzoyl-amino are especially preferred. Other preferred substituents are carboxyphenyl, C₁-C₇-alkylaminocarbonylphenyl, carboxy-C₁-C₇-alkyloxyphenyl, C₁-C₇-alkylaminocarbonyl-C₁-C₇-alkyloxyphenyl, tetrazolyl, 2-oxo-3-phenyl-tetrahydropyrazolidin-1-yl, oxetidin-3-yl-C₁-C₇-alkyloxy, 3-C₁-C₇-alkyl-oxetidin-3-yl-C₁-C₇-alkyloxy, 2-oxo-tetrahydrofuran-4-yl-C₁-C₇-alkyloxy or C₁-C₇-alkyoxyphenylaminocarbonyl. Most preferably, these moieties are bound to X₃ or to X₄. More generally, R₃ is hydrogen or more preferably a moiety different from hydrogen selected from the definitions for R₃ herein.

As R₄, hydroxy, halo or C₁-C₇-alkoxy are especially preferred or R₄ is absent.

In all definitions above the person having skill in the art will, without undue experimentation or considerations, be able to recognize which are relevant (e.g. those that are sufficiently stable for the manufacture of pharmaceuticals, e.g. having a half-life of more than 30 seconds) and thus are preferably encompassed by the present claims and that only chemically feasible bonds and substitutions (e.g. in the case of double or triple bonds, hydrogen carrying amino or hydroxy groups and the like) are encompassed, as well as tautomeric forms where present. For example, preferably, for reasons of stability or chemical feasability, in -G-R5 G and the atom binding as part of R5 are not simultaneously oxy plus oxy, thio plus oxy, oxy plus thio or thio plus thio. Substitutents binding via an O or S that is part of them are preferably not bound to nitrogen e.g. in rings.

Salts are especially the pharmaceutically acceptable salts of compounds of formula I. They can be formed where salt forming groups, such as basic or acidic groups, are present that can exist in dissociated form at least partially, e.g. in a pH range from 4 to 10 in aqueous solutions, or can be isolated especially in solid form.

Such salts are formed, for example, as acid addition salts, preferably with organic or inorganic acids, from compounds of formula I with a basic nitrogen atom (e.g. imino or amino), especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, lactic acid, fumaric acid, succinic acid, citric acid, amino acids, such as glutamic acid or aspartic acid, maleic acid, hydroxymaleic acid, methylmaleic acid, benzoic acid, methane- or ethane-sulfonic acid, ethane-1,2-disulfonic acid, benzenesulfonic acid, 2-naphthalenesulfonic acid, 1,5-naphthalene-disulfonic acid, N-cyclohexylsulfamic acid, N-methyl-, N-ethyl- or N-propyl-sulfamic acid, or other organic protonic adds, such as ascorbic acid.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

When a basic group and an acid group are present in the same molecule, a compound of formula I may also form internal salts.

For isolation or purification purposes it is also possible to use pharmaceutically unacceptable salts, for example picrates or perchlorates. For therapeutic use, only pharmaceutically acceptable salts or free compounds are employed (where applicable comprised in pharmaceutical preparations), and these are therefore preferred.

In view of the close relationship between the compounds in free form and in the form of their salts, including those salts that can be used as intermediates, for example in the purification or Identification of the compounds or salts thereof, any reference to “compounds” and “Intermediates” hereinbefore and hereinafter, especially to the compound(s) of the formula I, is to be understood as referring also to one or more salts thereof or a mixture of a free compound and one or more salts thereof, each of which is intended to include also any solvate, metabolic precursor such as ester or amide of the compound of formula I, or salt of any one or more of these, as appropriate and expedient and if not explicitly mentioned otherwise. Different crystal forms may be obtainable and then are also included.

Where the plural form is used for compounds, salts, pharmaceutical preparations, diseases, disorders and the like, this is intended to mean one (preferred) or more single compound(s), salt(s), pharmaceutical preparation(s), disease(s), disorder(s) or the like, where the singular or the indefinite article (“a” “an”) is used, this is intended to include the plural or preferably the singular.

The compounds of the present invention possess two or more asymmetric centers depending on the choice of the substituents. The preferred absolute configuration at the carbon carrying the G-R5 moiety in the central piperidine moiety is as indicated herein specifically. However, any possible isolated or pure diastereoisomers, enantiomers and geometric enantiomers, and mixtures thereof, e.g., racemates, are encompassed by the present invention.

As described herein above, the present invention provides 3,4(,5)-substituted piperidine derivatives of formula I, these compounds for use in the (prophylactic and/or therapeutic) treatment of a disease (=condition, disorder) in a warm-blooded animal, especially a human, preferably of a disease dependent on (especially inappropriate) renin activity, a pharmaceutical composition comprising a compound of the formula I, methods for preparing said compound or pharmaceutical preparation, and methods of treating conditions dependent on (especially inappropriate) renin activity by administration of a therapeutically effective amount of a compound of the formula I, or a pharmaceutical composition thereof.

“Inappropriate” renin activity preferably relates to a state of a warm-blooded animal, especially a human, where renin shows a renin activity that is too high in the given situation (e.g. due to one or more of misregulation, overexpression e.g. due to gene amplification or chromosome rearrangement or infection by microorganisms such as virus that express an aberrant gene, abnormal activity e.g. leading to an erroneous substrate specificity or a hyperactive renin e.g. produced in normal amounts, too low activity of renin activity product removing pathways, high substrate concentration and/or the like) and/or leads to or supports a renin dependent disease or disorder as mentioned above and below, e.g. by too high renin activity. Such inappropriate renin activity may, for example, comprise a higher than normal activity, or further an activity in the normal or even below the normal range which, however, due to preceding, parallel and or subsequent processes, e.g. signaling, regulatory effect on other processes, higher substrate or product concentration and the like, leads to direct or indirect support or maintenance of a disease or disorder, and/or an activity that supports the outbreak and/or presence of a disease or disorder in any other way. The inappropriate activity of renin may or may not be dependent on parallel other mechanisms supporting the disorder or disease, and/or the prophylactic or therapeutic effect may or may include other mechanisms in addition to inhibition of renin. Therefore ‘dependent’ has to be read as “dependent inter alia”, (especially in cases where a disease or disorder is really exclusively dependent only on renin) preferably as “dependent mainly”, more preferably as “dependent essentially only”. A disease dependent on (especially inappropriate) activity of renin may also be one that simply responds to modulation of renin activity, especially responding in a beneficial way in case of renin inhibition.

Where a disease or disorder dependent on inappropriate activity of a renin is mentioned (such in the definition of “use” in the following paragraph and also especially where a compound of the formula I is mentioned for use in the diagnostic or therapeutic treatment which is preferably the treatment of a disease or disorder dependent on inappropriate renin activity, this refers preferably to any one or more diseases or disorders that depend on inappropriate activity of natural renin and/or one or more altered or mutated forms thereof.

Where subsequently or above the term “use” is mentioned (as verb or noun) (relating to the use of a compound of the formula I or of a pharmaceutically acceptable salt thereof, or a method of use thereof), this (if not indicated differently or to be read differently in the context) includes any one or more of the following embodiments of the invention, respectively (if not stated otherwise): the use in the treatment of a disease or disorder that depends on (especially inappropriate) activity of renin, the use for the manufacture of pharmaceutical compositions for use in the treatment of a disease or disorder that depends on (especially inappropriate) activity of renin; a method of use of one or more compounds of the formula I in the treatment of a disease or disorder that depends on (especially inappropriate) activity of renin; a pharmaceutical preparation comprising one or more compounds of the formula I for the treatment of a disease or disorder that depends on (especially inappropriate) activity of renin; and one or more compounds of the formula I for use in the treatment of a disease or disorder in a warm-blooded animal, especially a human, preferably a disease that depends on (especially inappropriate) activity of renin; as appropriate and expedient, if not stated otherwise.

The terms “treat”, “treatment” or “therapy” refer to the prophylactic (e.g. delaying or preventing the onset of a disease or disorder) or preferably therapeutic (including but not limited to preventive, delay of onset and/or progression, palliative, curing, symptom-alleviating, symptom-reducing, patient condition ameliorating, renin-modulating and/or renin-inhibiting) treatment of said disease(s) or disorder(s), especially of the one or more disease or disorder mentioned above or below.

### PREFERRED EMBODIMENTS ACCORDING TO THE INVENTION

The groups of preferred embodiments of the invention mentioned below are not to be regarded as exclusive, rather, e.g., in order to replace general expressions or symbols with more specific definitions, parts of those groups of compounds can be interchanged or exchanged using the definitions given above, or omitted, as appropriate, and each of the more specific definitions, independent of any others, may be introduced independently of or together with one or more other more specific definitions for other more general expressions or symbols.

Preferred is a compound of the formula I with the following configuration

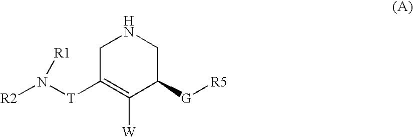

wherein R1, R2, R5, T, G and W are as defined for a compound of the formula I, or a pharmaceutically acceptable salt thereof.

Preferred is also a compound of the formula I with the following configuration

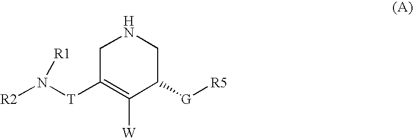

wherein R1, R2, R5, T, G and W are as defined for a compound of the formula I, or a pharmaceutically acceptable salt thereof.

Preferred is a compound of the formula I, wherein

R1 is C₁-C₇-alkyl, halo-C₁-C₇-alkyl, di-(phenyl)-C₁-C₇-alkyl, C₃-C₈-cyclopropyl, (unsubstituted or C₁-C₇-alkoxy-substituted naphthyl)-C₁-C₇-alkyl, (halo-phenyl)-C₁-C₇-alkyl or phenyl substituted by C₁-C₇-alkyl, halo, C₁-C₇-alkyloxy and/or C₁-C₇-alkoxy-C₁-C₇-alkyloxy,
R2 is hydrogen, phenyl-C₁-C₇-alkyl, di-(phenyl)-C₁-C₇-alkyl, naphthyl-C₁-C₇-alkyl, phenyl, naphthyl, pyridyl-C₁-C₇-alkyl, indolyl-C₁-C₇-alkyl, 1H-indazolyl-C₁-C₇-alkyl, quinolyl-C₁-C₇-alkyl, isoquinolyl-C₁-C₇-alkyl, 1,2,3,4-tetrahydro-1,4-benzoxazinyl-C₁-C₇-alkyl, 2H-1,4-benzoxazin-3(4H)-onyl-C₁-C₇-alkyl, 1-benzothiophenyl-C₁-C₇-alkyl, pyridyl, indolyl, 1H-indazolyl, quinolyl, isoquinolyl, 1,2,3,4-tetrahydro-1,4-benzoxazinyl, 2H-1,4-benzoxazin-3(4H)-onyl, 1-benzothiophenyl, phenylcarbonyl (benzoyl) or naphthylcarbonyl (naphthoyl), where each phenyl, naphthyl, pyridyl, indolyl, 1H-indazolyl, quinolyl, isoquinolyl, 1,2,3,4-tetrahydro-1,4-benzoxazinyl, 2H-1,4-benzoxazin-3(4H)-onyl or 1-benzothiophenyl is unsubstituted or substituted by one or more, e.g. up to three, substituents independently selected from the group consisting of C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkanoyloxy-C₁-C₇-alkyl, amino-C₁-C₇-alkyl, C₁-C₇-alkoxy-C₁-C₇-alkylamino-C₁-C₇-alkyl, C₁-C₇-alkanoylamino-C₁-C₇-alkyl, C₁-C₇-alkylsulfonylamino-C₁-C₇-alkyl, carboxy-C₁-C₇-alkyl, C₁-C₇-alkoxycarbonyl-C₁-C₇-alkyl, halo, hydroxy, C₁-C₇-alkoxy, hydroxy-C₁-C₇-alkyloxy, C₁-C₇-alkoxy-C₁-C₇-alkoxy, amino-C₁-C₇-alkoxy, N—C₁-C₇-alkanoylamino, —C₁-C₇-alkoxy, carboxy-C₁-C₇-alkyloxy, C₁-C₇-alkyloxycarbonyl-C₁-C₇-alkoxy, carbamoyl-C₁-C₇-alkoxy, N-mono- or N,N-di-C₁-C₇-alkyl)-carbamoyl-C₁-C₇-alkoxy, morpholino-C₁-C₇-alkoxy, pyridyl-C₁-C₇-alkoxy, amino, C₁-C₇-alkanoylamino, C₁-C₇-alkanoyl, C₁-C₇-alkoxy-C₁-C₇-alkanoyl, C₁-C₇-alkanoyl, C₁-C₇-alkyloxy-C₁-C₇-alkanoyl, C₁-C₇-alkoxy-C₁-C₇-alkanoyl, carboxyl, carbamoyl, N—C₁-C₇-alkoxy-C₁-C₇-alkylcarbamoyl, pyrazolyl, pyrazolyl-C₁-C₇-alkoxy, 4-C₁-C₇-alkylpiperidin-1-yl, nitro and cyano;
W is a moiety of the formula IA,

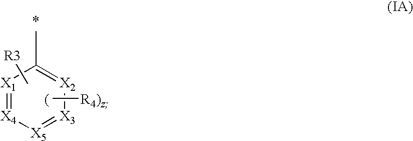

wherein the asterisk (*) denotes the position where the moiety W is bound to the 4-carbon in the piperidine ring in formula I, and wherein one of X₁ and X₂ is nitrogen or CH, while the other and X₃, X₄ and X₅ are CH; preferably with the proviso that R3 is bound to X₁ or X₂ or preferably to X₃ or X₄; or a moiety of the formula IB,

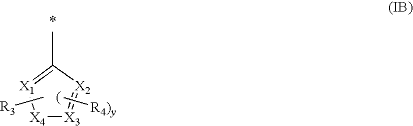

wherein the asterisk (*) denotes the position where the moiety W is bound to the 4-carbon in the piperidine ring in formula I, and wherein X₄ is CH₂, NH, S or O and one of X₁, X₂ and (preferably if X₄ is CH₂ or N) X₃, more preferably X₂, is N, while the others are each CH, with the proviso that at least one ring nitrogen (N or in the case or X₄ NH) is present and that R3 is then preferably bound to X₃; preferably, X₁ is CH or N, X₂ is CH or N, X₃ is CH or N and X₄ is NH, O or S, with the proviso that not more than one of X₁, X₂ and X₃ is N; and preferably with the proviso that R3 is bound to X₁ or X₂ or preferably to X₃ or X₄;
or a moiety of the formula IC,

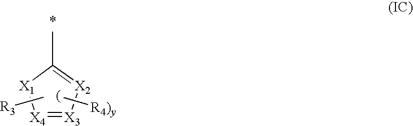

wherein the asterisk (*) denotes the position where the moiety W is bound to the 4-carbon in the piperidine ring in formula I, and wherein
X₁ is CH₂, NH, S or O and one of X₂, X₃ and X₄ is N, while the others are CH, with the proviso that at least one ring nitrogen (N or in the case or X₁ NH) is present; preferably, X₁ is S or O, X₂ is CH or N, X₃ is CH or N, and X₄ is CH or N, with the proviso that not more than one of X₂, X₃ and X₄ is N; and preferably with the proviso that R3 is bound to X₂ or preferably to X₃ or X₄;
where in each case where R3 is bond to a moiety of the formula IA, IB or IC, instead of a hydrogen atom at a ring member NH, CH₂ or CH mentioned so far where R3 is bound a moiety R3 is present;
y is 0 or 1, preferably 0, and z is 0, 1 or 2, preferably 0 or 1;
R3 is hydrogen or preferably C₁-C₇-alkyloxy-C₁-C₇-alkyloxy, phenyloxy-C₁-C₇-alkyl, phenyl, phenyl-C₁-C₇-alkoxy, naphthyl, naphthyl-C₁-C₇-alkoxy, pyridyl, pyridyl-C₁-C₇-alkoxy, phenyloxy, napthyloxy, phenyloxy-C₁-C₇-alkoxy, morpholino-C₁-C₇-alkoxy, tetrahydropyranyloxy, 2H,3H-1,4-benzodioxinyl-C₁-C₇-alkoxy, phenylaminocarbonyl or phenylcarbonylamino, wherein in each case where present under R3 phenyl, naphthyl or pyridyl is unsubstituted or substituted by one or more, preferably up to three, moieties independently selected from the group consisting of C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, amino-C₁-C₇-alkyl, C₁-C₇-alkoxy-C₁-C₇-alkylamino-C₁-C₇-alkyl, carboxy-C₁-C₇-alkyl, halo, especially fluoro, chloro or bromo, hydroxy, C₁-C₇-alkoxy, C₁-C₇-alkoxy-C₁-C₇-alkoxy, amino-C₁-C₇-alkoxy, N—C₁-C₇-alkanoylamino-C₁-C₇-alkoxy, carbamoyl-C₁-C₇-alkoxy, N-mono- or N,N-di-(C₁-C₇-alkyl)-carbamoyl-C₁-C₇-alkoxy, morpholino-C₁-C₇-alkoxy, pyridyl-C₁-C₇-alkoxy, amino, C₁-C₇-alkanoylamino, C₁-C₇-alkanoyl, C₁-C₇-alkoxy-C₁-C₇-alkanoyl, carboxy, carbamoyl, N—(C₁-C₇-alkoxy-C₁-C₇-alkyl)-carbamoyl, pyrazolyl, pyrazolyl-C₁-C₇-alkoxy, 4-C₁-C₇-alkylpiperidin-1-yl, nitro and cyano
R4 if present (which is the case if y or z is other than zero) is hydroxy, halo or C₁-C₇-alkoxy;
T is carbonyl (—C(═O)—);
G is methylene, oxy or imino; and R5 is hydrogen, C₁-C₇-alkyl, C₁-C₇-alkoxy-C₁-C₇-alkyl, C₁-C₇-alkoxy, C₁-C₇-alkanoyl, C₁-C₇-alkylsulfonyl or (unsubstituted or C₁-C₇-alkyl-substituted phenyl)-sulfonyl or
-G-R5 is hydrogen;
or a pharmaceutically acceptable salt thereof.

More preferably, the invention relates to a compound of the formula I, wherein

R1 is C₁-C₇-alkyl, halo-C₁-C₇-alkyl, di-(phenyl)-C₁-C₇-alkyl, C₃-C₈-cyclopropyl, (unsubstituted or C₁-C₇-alkoxy-substituted naphthyl)-C₁-C₇-alkyl, (halo-phenyl)-C₁-C₇-alkyl or phenyl substituted by C₁-C₇-alkyl, halo, C₁-C₇-alkyloxy and/or C₁-C₇-alkoxy-C₁-C₇-alkyloxy,
R2 is hydrogen, phenyl-C₁-C₇-alkyl, di-(phenyl)-C₁-C₇-alkyl, naphthyl-C₁-C₇-alkyl, phenyl, naphthyl, pyridyl-C₁-C₇-alkyl, indolyl-C₁-C₇-alkyl, 1H-indazolyl-C₁-C₇-alkyl, quinolyl-C₁-C₇-alkyl, isoquinolyl-C₁-C₇-alkyl, 1-benzothiophenyl-C₁-C₇-alkyl or phenylcarbonyl (benzoyl), where each phenyl, naphthyl, pyridyl, indolyl, 1H-indazolyl, quinolyl, isoquinolyl or 1-benzothiophenyl is unsubstituted or substituted by one or more, e.g. up to three, substituents independently selected from the group consisting of C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, amino-C₁-C₇-alkyl, C₁-C₇-alkoxy-C₁-C₇-alkylamino-C₁-C₇-alkyl, C₁-C₇-alkanoylamino-C₁-C₇-alkyl, C₁-C₇-alkoxycarbonyl-C₁-C₇-alkyl, halo, C₁-C₇-alkoxy, hydroxy-C₁-C₇-alkyloxy, C₁-C₇-alkoxy-C₁-C₇-alkoxy, amino-C₁-C₇-alkoxy, N—C₁-C₇-alkanoylamino-C₁-C₇-alkoxy, carboxy-C₁-C₇-alkyloxy, C₁-C₇-alkyloxycarbonyl-C₁-C₇-alkoxy, carbamoyl-C₁-C₇-alkoxy, N-mono- or N,N-di-(C₁-C₇-alkyl)-carbamoyl-C₁-C₇-alkoxy, C₁-C₇-alkanoyl, C₁-C₇-alkyloxy-C₁-C₇-alkanoyl, carbamoyl and N—C₁-C₇-alkoxy-C₁-C₇-alkylcarbamoyl;
W is a moiety of the formula IA,

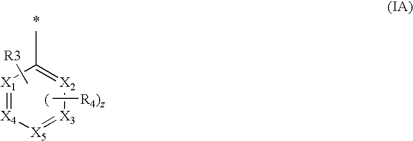

wherein the asterisk (*) denotes the position where the moiety W is bound to the 4-carbon in the piperidine ring in formula I, and wherein
X₁ is N or CH and each of X₂, X₃, X₄ and X₅ is CH;
or a moiety of the formula IC,

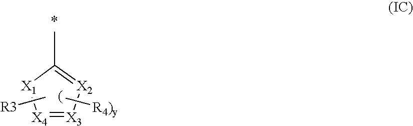

wherein the asterisk (*) denotes the position where the moiety W is bound to the 4-carbon in the piperidine ring in formula I, and wherein
X₁ is CH₂ or O, X₄ is N and X₂ and X₃ each are CH, with the proviso that R3 is bound to X₃ instead of the hydrogen;
z is 0 or 1; y is 0;
R3 is phenyl, phenyl-C₁-C₇-alkoxy, pyridyl, pyridyl-C₁-C₇-alkoxy, phenyloxy, phenyloxy-C₁-C₇-alkoxy or morpholino-C₁-C₇-alkoxy, wherein in each case where present under R3 phenyl or pyridyl is unsubstituted or substituted by one or more, preferably up to three, moieties independently selected from the group consisting of halo, especially fluoro, chloro or bromo, hydroxy, C₁-C₇-alkoxy, morpholino-C₁-C₇-alkoxy, C₁-C₇-alkanoylamino, pyrazolyl, 4-C₁-C₇-alkylpiperidin-1-yl and cyano;
R4 (present if z is 1) is a moiety independently selected from hydroxy and C₁-C₇-alkoxy;
T is carbonyl; and
G-R5 is hydrogen, hydroxy, C₁-C₇-alkyloxy, C₁-C₇-alkoxy-C₁-C₇-alkyloxy, amino, C₁-C₇-alkanoylamino, C₁-C₇-alkylsulfonylamino or (unsubstituted or C₁-C₇-alkyl-substituted phenyl)-sulfonylamino;
or a pharmaceutically acceptable salt thereof.

Particular embodiments of the invention, especially of compounds of the formula I and/or salts thereof, are provided in the Examples—the invention thus, in a very preferred embodiment, relates to a compound of the formula I, or a salt thereof, selected from the compounds given in the Examples, as well as the use thereof.

### Process of Manufacture

A compound of formula I, or a salt thereof, is prepared analogously to methods that, for other compounds, are in principle known in the art, so that for the novel compounds of the formula I the process is novel at least as analogy process, especially as described or in analogy to methods described herein in the illustrative Examples, or modifications thereof, preferably in general by

(a) for the synthesis of a compound of the formula I wherein the moieties are as defined for a compound of the formula I, reacting a carbonic acid compound of the formula II

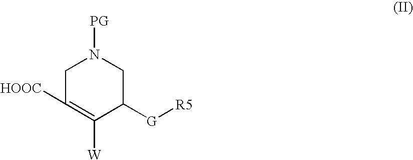

wherein W, G and R5 or -G- are as defined for a compound of the formula I and PG is a protecting group, or an active derivative thereof, with an amine of the formula III,

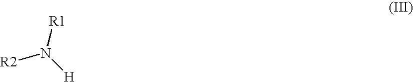

wherein R1 and R2 are as defined for a compound of the formula I, and removing protecting groups to give the corresponding compound of the formula I, or
(b) for the preparation of a compound of the formula I wherein R3 is unsubstituted or substituted aryl or unsubstituted or substituted alkyoxy and W is a moiety of the formula IA given above, by reacting a compound of the formula IV,

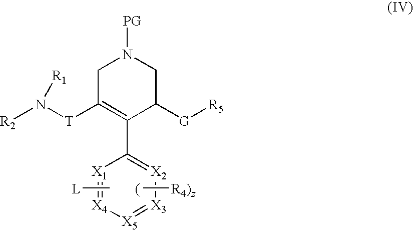

wherein R1, R2, T, G, R5, X₁, X₂, X₃, X₄, X₅, z and R₄ are as defined for a compound of the formula I, PG is a protecting group and L is a leaving group or hydroxy, with a compound of the formula V,

R3-Q (V)

wherein R3 is as just defined and Q is —B(OH)₂ or a leaving group, and removing protecting groups to give the corresponding compound of the formula I,
and, if desired, subsequent to any one or more of the processes mentioned above converting an obtainable compound of the formula I or a protected form thereof into a different compound of the formula I, converting a salt of an obtainable compound of formula I into the free compound or a different salt, converting an obtainable free compound of formula I into a salt thereof, and/or separating an obtainable mixture of isomers of a compound of formula I into individual isomers;
where in any of the starting materials (especially of the formulae II to IV), in addition to specific protecting groups mentioned, further protecting groups may be present, and any protecting groups are removed at an appropriate stage in order to obtain a corresponding compound of the formula I, or a salt thereof.

### Preferred Reaction Conditions

The preferred reaction conditions for the reactions mentioned above, as well as for the transformations and conversions, are as follows (or analogous to methods used in the Examples or as described there)

The reaction under (a) between an add of the formula II, or a reactive derivative thereof, and an amino compound of the formula III preferably takes place under customary condensation conditions, where among the possible reactive derivatives of an acid of the formula II reactive esters (such as the hydroxybenzotriazole (HOBT), pentafluorophenyl, 4-nitrophenyl or N-hydroxysuccinimide ester), acid halogenides (such as the add chloride or bromide) or reactive anhydrides (such as mixed anhydrides with lower alkanoic acids or symmetric anhydrides) are preferred. Reactive carbonic acid derivatives can also be formed in situ. The reaction is carried out by dissolving the compounds of formulae II and III in a suitable solvent, for example a halogenated hydrocarbon, such as methylene chloride, N,N-dimethylformamide, N,N-dimethylacetamide, N-methyl-2-pyrrolidone, methylene chloride, or a mixture of two or more such solvents, and by the addition of a suitable base, for example triethylamine, diisopropylethylamine (DIEA) or N-methylmorpholine and, if the reactive derivative of the acid of the formula II is formed in situ, a suitable coupling agent that forms a preferred reactive derivative of the carbonic acid of formula III in situ, for example dicyclohexylcarbodiimide/1-hydroxybenzotriazole (DCC/HOBT); bis(2-oxo-3-oxazolidinyl)phosphinic chloride (BOPCl); O-(1,2-dihydro-2-oxo-1-pyridyl)-N,N,N′,N′-tetramethyluronium tetrafluoroborate (TPTU); O-benzotriazol-1-yl)-N,N,N′,N′-tetramethyluronium tetrafluoroborate (TBTU); (benzotriazol-1-yloxy)-tripyrrolidinophosphonium-hexafluorophosphate (PyBOP), 1-(3-dimethylaminopropyl)-3-ethylcarbodiimide hydrochloride/hydroxybenzotriazole or/1-hydroxy-7-azabenzotriazole (EDC/HOBT or EDC/HOAt) or HOAt alone, or with (1-chloro-2-methyl-propenyl-dimethylamine. For review of some other possible coupling agents, see e.g. Klauser; Bodansky, Synthesis 1972, 453-463. The reaction mixture is preferably stirred at a temperature of between approximately −20 and 50° C., especially between 0° C. and 30° C., e.g. at room temperature. The reaction is preferably carried out under an inert gas, e.g. nitrogen or argon.

The subsequent removal of a protecting group, e.g. PG, such as tert-butoxycarbonyl, benzyl or 2-(trimethylsilyl)-ethoxycarbonyl, takes place under standard conditions, see also the literature mentioned below under General Process Conditions. For example, tert-butoxycarbonyl is removed in the presence of an acid, e.g. a hydrohalic add, such as HCl, in an appropriate solvent, e.g. an ether, such as dioxane, or an alcohol, e.g. isopropanol, at customary temperatures, e.g. at room temperature, the removal of benzyl can be achieved e.g. by reaction with ethylchloroformate in an appropriate solvent, e.g. toluene, at elevated temperatures, e.g. from 80 to 110° C., and subsequent removal of the resulting ethoxycarbonyl group by hydrolysis in the presence of a base, e.g. an alkali metal hydroxide, such as potassium hydroxide, in an appropriate solvent, e.g. in an alcohol, such as ethanol, at elevated temperatures, e.g. from 80 to 120° C., or by removal by means of trimethylsilyl trifluoroacetate in a tertiary nitrogen base, such as 2,6-lutidine, in the presence of an appropriate solvent, such as a halogenated hydrocarbon, e.g. methylene chloride, and the removal of 2-(trimethylsilyl)-ethoxycarbonyl can be achieved, for example, by reaction with a tetra-lower alkylammonium fluoride, such as tetraethylammoniumfluoride, in an appropriate solvent or solvent mixture, e.g. a halogenated hydrocarbon, such as methylene chloride, and/or a nitrile, such as acetonitrile, preferably at elevated temperatures, e.g. under reflux conditions.

Where the reaction under (b) takes place with a compound of the formula IV wherein L is a leaving group and with a compound of the formula V wherein Q is —B(OH)₂, L is preferably halo, such as bromo or iodo, or trifluoromethylsulfonyloxy, and the reaction preferably takes place in an appropriate solvent, such as dioxane in the presence or absence of water, a basic buffering substance, e.g. potassium phosphate or potassium carbonate, and catalyst, e.g. Pd(PPh₃)₄, at preferably elevated temperatures, e.g. between 60° C. and the reflux temperature of the mixture. Where the reaction under (b) takes place with a compound of the formula IV wherein L is hydroxy and with a compound of the formula V wherein Q is a leaving group, the leaving group is preferably halo, e.g. bromo or Iodo, and the coupling reaction preferably takes place in the presence of a base, such as potassium carbonate, in an appropriate solvent, e.g. N,N-dimethylformamide, at preferably elevated temperatures, e.g. from 30 to 80° C. Removal of protecting groups can take place as described above under (a) and below in the general process conditions. Note that wherever —B(OH)₂ is mentioned, alternatively a moiety —B(OR)₂ is possible wherein the moieties OR together form a linear of branched alkylene bridge.

Where desired, R₂ other than hydrogen can subsequently be introduced by reaction with a compound of the formula VII wherein preferably D is—the reaction preferably takes place under customary substitution conditions, e.g. in the case where an aryl moiety R2 is to be coupled and Z is halo, e.g. iodo, in the presence of copper (e.g. Venus copper), sodium iodide and a base, such as potassium carbonate, in the presence or preferably absence of an appropriate solvent, e.g. at elevated temperatures in the range from, for example, 150 to 250° C., or (especially if Z in formula VIII is bromo) in the presence of a strong base, such as an alkali metal alkoholate, e.g. sodium tert-butylate, in the presence of an appropriate catalyst, such as [Pd(μ-Br)(t-Bu₃P)]₂, and of an appropriate solvent, e.g. an aromatic solvent, such as toluene, at preferred temperatures between room temperature and the reflux temperature of the mixture, or (e.g. where the moiety R2 is unsubstituted or substituted alkyl) in the presence of a base, such as an alkali metal carbonate, such as potassium carbonate, if useful in the presence of an alkali metal halogenide, e.g. sodium iodide, in an appropriate solvent, such as dimethyl formamide, at preferably elevated temperatures, e.g. between 50° C. and the reflux temperature of the mixture, or in presence of NaN(TMS)₂ in an appropriate solvent such as tetrahydrofurane at preferred temperatures from −20 to 30° C., e.g. at about 0° C., or, where R¹ is to be bound via a carbonyl or sulfonyl group, under condensation conditions e.g. as described above for reaction (a). The removal of protecting groups, both with or without preceding reaction with a compound of the formula VII, takes place e.g. as described above under the preferred conditions for reaction (a).

### Optional Reactions and Conversions

Compounds of the formula I, or protected forms thereof directly obtained according to any one of the preceding procedures or after introducing protecting groups anew, which are included subsequently as starting materials for conversions as well even if not mentioned specifically, can be converted into different compounds of the formula I according to known procedures, where required after removal of protecting groups.

Where R2 is hydrogen in a compound of the formula I, this can be converted into the corresponding compound wherein R2 has a meaning other than hydrogen given for compounds of the formula I by reaction with a compound of the formula VII,

R2*-D (VII)

wherein R2* is defined as R2 in a compound of the formula I other than hydrogen and D is a leaving group, or wherein D is —CHO and then R2* is the complementary moiety for a moiety R2 that includes a methylene group (resulting in a group R2*-CH₂—) e.g. under reaction conditions as follows: The reductive amination preferably takes place under customary conditions for reductive amination, e.g. in the presence of an appropriate hydrogenation agent, such as hydrogen in the presence of a catalyst or a complex hydride, e.g. sodium triacetoxyborohydride or sodium cyanoborhydride, in an appropriate solvent, such as a halogenated hydrocarbon, e.g. methylene chloride or 1,2-dichloroethane, and optionally a carbonic acid, e.g. acetic acid, at preferred temperatures between −10° C. and 50° C., e.g. from 0° C. to room temperature.

Hydroxy substituents, e.g. as substitutents of aryl in alkyl substituted by aryl R1, R2 or in other aryl substituents, can be transformed into unsubstituted or substituted alkoxy, e.g. by alkylation reaction with the corresponding unsubstituted or substituted alkylhalogenide, e.g. iodide, in the presence of a base, e.g. potassium carbonate, in an appropriate solvent, e.g. N,N-dimethylformamide, e.g. at preferred temperatures between 0 and 50° C.

Carboxy substitutents can be converted into esterified carboxy by reaction with corresponding alcohols, e.g. C₁-C₇-alkanols, or into amidated carboxy by reaction with corresponding amines, e.g. under condensation conditions analogous to those described above under reaction (a).

Esterified carboxy substituents can be converted into free carboxy by hydrolysis, e.g. in the presence of a base, such as potassium hydroxide, in an appropriate solvent, e.g. tetrahydrofurane, preferably at elevated temperatures, e.g. from 50° C. to the reflux temperature of the reaction mixture.

A moiety -G-R5 wherein G is O and R5 is hydrogen can be converted into amino by first converting the —OH into a leaving group, e.g. by halogenation or preferably by reaction with an organic sulfonylhalogenide, such as methylsulfonylchloride, in the presence of a tertiary nitrogen base, such as triethylamine, and in the presence of an appropriate solvent, e.g. dichloromethane, preferably at lower temperatures, e.g. in the range from −30 to 20° C., followed by reaction with an alkali metal azide, e.g. sodium azide, in an appropriate solvent, such as dichloromethane, in the presence of a tertiary nitrogen base, e.g. triethylamine, and preferably at lower temperatures, e.g. in the range from −30 to 20° C. to give the corresponding azido group, which is then converted into the amino group e.g. by reaction with triphenylphosphine in an appropriate solvent, e.g. tetrahydrofurane in the presence of water, at preferably lower temperatures, e.g. in the range from −30 to 20° C.

A group -G-R5 wherein G is NH and R5 is H (thus being amino) can be converted into the corresponding group wherein G is NH and R5 is unsubstituted or substituted alkyl or acyl by alkylation or acylation. For example, acylation may take place using the corresponding acid halogenide (e.g. the chloride) in the presence of a tertiary nitrogen base, such as triethylamine, in an appropriate solvent, such as dichloromethane, preferably at lower temperatures, e.g. in the range from −30 to 20° C.

In some cases, the conversions preferably take place with compounds of the formula I in protected form; the subsequent removal of protecting group can be achieved as above for reaction (a) and below under “General Process Conditions”, yielding a corresponding compound of the formula I.

Salts of compounds of formula I having at least one salt-forming group may be prepared in a manner known per se. For example, salts of compounds of formula I having acid groups may be formed, for example, by treating the compounds with metal compounds, such as alkali metal salts of suitable organic carboxylic acids, e.g. the sodium salt of 2-ethylhexanoic add, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Add addition salts of compounds of formula I are obtained in customary manner, e.g. by treating the compounds with an add or a suitable anion exchange reagent, internal salts of compounds of formula I containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers.

A salt of a compound of the formula I can be converted in customary manner into the free compound; metal and ammonium salts can be converted, for example, by treatment with suitable acids, and acid addition salts, for example, by treatment with a suitable basic agent. In both cases, suitable ion exchangers may be used.

Stereoisomeric mixtures, e.g. mixtures of diastereomers, can be separated into their corresponding isomers in a manner known per se by means of appropriate separation methods. Diastereomeric mixtures for example may be separated into their individual diastereomers by means of fractionated crystallization, chromatography, solvent distribution, and similar procedures. This separation may take place either at the level of one of the starting compounds or in a compound of formula I itself. Enantiomers may be separated through the formation of diastereomeric salts, for example by salt formation with an enantiomer-pure chiral add, or by means of chromatography, for example by HPLC, using chromatographic substrates with chiral ligands.

Intermediates and final products can be worked up and/or purified according to standard methods, e.g. using chromatographic methods, distribution methods, (re-) crystallization, and the like.

### Starting Materials

Starting Materials, including intermediates, for compounds of the formula I, such as the compounds of the formulae II, III, IV, V and VII, can be prepared, for example, according to methods that are known in the art, according to methods described in the examples or methods analogous to those described in the examples, and/or they are known or commercially available.

In the subsequent description of starting materials and intermediates and their synthesis, R1, R2, R2*, R3, R4, R5, R6, T, G, W, X₁, X₂, X₃, X₄, X₅, y, z and PG have the meanings given above or in the Examples for the respective starting materials or intermediates, if not indicated otherwise directly or by the context. Protecting groups, if not specifically mentioned, can be introduced and removed at appropriate steps in order to prevent functional groups, the reaction of which is not desired in the corresponding reaction step or steps, employing protecting groups, methods for their introduction and their removal are as described above or below, e.g. in the references mentioned under “General Process Conditions”. The person skilled in the art will readily be able to decide whether and which protecting groups are useful or required.

A compound of the formula II can, for example, be prepared by reacting a compound of the formula VIII,

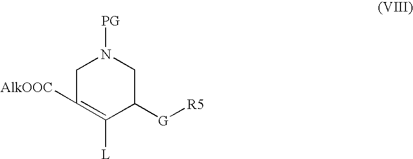

wherein L is as described above for a compound of the formula IV, Alk is unsubstituted or substituted alkyl, especially C₁-C₇-alkyl, and the other moieties have the meanings described for a compound of the formula II, with a compound of the formula IX,

.W-Q (IX)

wherein W is as described for a compound of the formula I and Q is —B(OH)₂ or a leaving group as defined for a compound of the formula V, under reaction conditions analogous to those described under reaction (b) above. Removal of the Alk moiety according to standard hydrolysis conditions, e.g. with a base, such as potassium hydroxide, in an appropriate solvent, e.g. tetrahydrofurane and water, at elevated temperatures, e.g. from 50° C. to the reflux temperature of the reaction mixture, yields the corresponding compound of the formula II.

A compound of the formula VIII wherein W is a moiety of the formula IC wherein X₁ is O, X₂ is CH, X₃ is CH and X₄ is N and R3 is bound instead of the H at position X₃ can be prepared from a compound of the formula VIII given above by reaction with trimethylsilyl-acetylene (Me₃-Si—C≡CH) in the presence e.g. of CuI and a tertiary nitrogen base, such as triethylamine, and a catalyst, e.g. Pd(PPh₃)₄, in an appropriate solvent, such as dimethylformamide, and at appropriate temperatures, e.g. from 30 to 70° C., to give the corresponding compound of the formula VI,

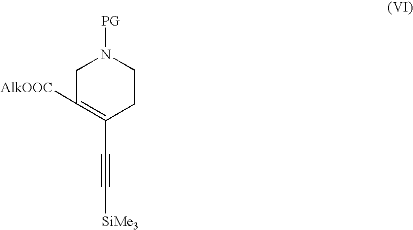

which is then reacted under desilylation, e.g. with cesium fluoride in an appropriate solvent, such as methanol and/or water, at an appropriate temperature, e.g. from 0 to 50° C., followed by reaction of the free acetylene compound (where in formula VI instead of the SiMe₃ group a hydrogen is present) with an carboximidoylhalogenide of the formula VA,

R3-C(═NH—OH)-Hal (VA)

wherein Hal is halogen, especially chloro, in the presence of a nitrogen base, e.g. triethylamine, in an appropriate solvent, e.g. methylene chloride, and at appropriate temperatures, e.g. from 0 to 50° C.; thus obtaining the corresponding compound of the formula VIII with the ring IC as described.

A compound of the formula IV can, for example, be prepared analogously to a compound of the formula I but using starting materials (e.g. corresponding to those of the formula II) wherein instead of W the moiety

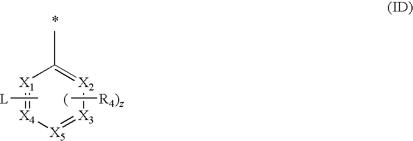

is present wherein the symbols have the meanings given under a compound of the formula IV and the asterisk denotes the point of binding to the rest of the molecule. The processes can then be analogous to those described under (a) used for the synthesis of compounds of the formula I, the starting materials can be analogous to those mentioned there as starting materials, e.g. analogues of the compounds of the formula II wherein instead of the moiety W one analogous to a moiety of the formula IA wherein instead of R3 L is present can be used. The reaction conditions can be as described for the other starting materials given hereinbefore.

Starting materials of the formula IV wherein L is hydroxy and the other symbols have the meanings given under formula IV can, for example, be prepared from the precursors wherein instead of hydroxy L a protected hydroxy is present by removal of the protecting group, e.g. in case of methoxymethyl by reaction with an acid, such as TFA, in an appropriate solvent, e.g. dichloromethane, for example at temperatures between 0 and 50° C. These precursors can be prepared in analogy to an analogue of a compound of the formula VIII and II or I wherein instead of the group W the moiety of the formula IA with protected hydroxy instead of L is present, e.g. from analogues of compounds of the formula IX wherein instead of W the moiety of the formula IC with protected hydroxy instead of L is present, in each case under conditions analogous to those for the corresponding compounds as given above.

Compounds of the formula III, wherein R2 is bound via methylene (as part of R2), can, for example, be prepared by reacting a compound of the formula X,

R2a-CHO (X)

(obtainable e.g. from the corresponding acids or their esters by reduction to a hydroxymethyl group and then oxidation to the —CHO group, e.g. under comparable conditions as described for the reductive amination under the conversion reactions described above) wherein R2a is a moiety that together with —CH₂— by which it is bound in formula III forms a corresponding moiety R2 in a compound of the formula I, under conditions of reductive amination, e.g. analogous to those described under the conversion reactions above, with an amine of the formula XI,

R1-NH₂ (XI)

wherein R1 is as defined for a compound of the formula I.

Alternatively, compounds of the formula III as described under reaction (b) above can be prepared by reaction of a compound of the formula XII,

R2-LG (XII)

wherein R2 is as defined for compounds of the formula I and LG is a leaving group, e.g. halo, under customary substitution reaction conditions with a compound of the formula XI as described above. Compounds of the formula XII can be obtained from precursors wherein instead of LG hydroxy is present by introducing LG, e.g. by halogenation with halosuccinimides or with thionylhalogenides, such as thionylchloride, in the presence of an appropriate solvent, e.g. dichloromethane, at elevated temperatures, e.g. from 30° C. to the reflux temperature of the reaction mixture, or by reaction with CBr₄ in the presence of PPh₃ in an appropriate solvent, e.g. diethylether, at preferred temperatures from −10 to 50° C.

Where the compound of the formula XII comprises a moiety R2 bound via a methylene group that is part of said R2, that is a group R2a as defined above for a compound of the formula X, that is, a compound of the formula XIIIa

R2a-CHO (XIIa)

is used as starting material of the formula XII, this can be obtained from the corresponding carboxylic acid or carboxylic acid precursor by reduction to the hydroxymethylene compound under customary conditions, e.g. by first reducing the carboxy function in the presence of an appropriate complex hydride, e.g. borane dimethylsulfide, in an appropriate solvent, e.g. tetrahydrofurane, at preferred temperatures between −20 and 40° C., or an alkylated carboxy function with LiAlH₄ with or without an appropriate solvent at lower temperatures, e.g. from −30 to 20° C., to the corresponding hydroxymethylene group which can then be oxidized to the aldehyde group, for example in the presence of Dess Martin periodinane e.g. in methylene chloride and/or water or of 2,2,6,6,-tetramethyl-1-piperidinyloxy free radical e.g. in toluene and/or ethyl acetate in the presence of potassium bromide, water and potassium hydrogencarbonate, at preferred temperatures in the range from 0 to 50° C., or using MnO₂ in an appropriate solvent, e.g. toluene, at preferred temperatures from 0 to 50° C., to obtain the corresponding hydroxymethylene precursors and subsequent replacement of the hydroxy group by LG as described for the synthesis of a compound of the formula XII.

Starting materials of the formula VIII, can be prepared from the corresponding oxo compounds of the formula XIII,

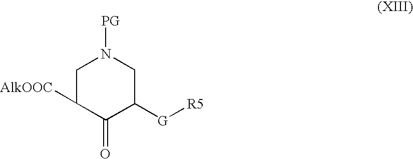

by reaction with a strong base, e.g. lithium diisopropylamide, in an appropriate solvent, e.g. tetrahydrofurane, at lower temperatures, e.g. from −30 to 20° C., followed by protection of the resulting hydroxy group, e.g. by reaction with methoxymethylchloride e.g. in the same reaction mixture at preferred temperatures from 0 to 50° C., and subsequent transformation of the hydroxy group into a group L, e.g. by reaction with trifluoroacetic acid anhydride in the presence of an appropriate base, e.g. diisopropylethylamine, in an appropriate solvent, such as dichloromethane, at preferred temperatures from −100 to −50° C.

Starting materials of the formula II wherein G-R5 is hydroxy or unsubstituted or substituted alkyloxy can be prepared, for example, starting from a compound of the formula XIV,

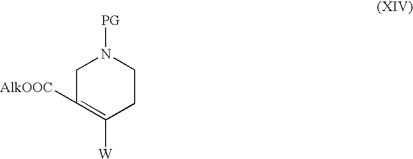

wherein W is as defined for a compound of the formula I, PG is a protecting group and Alk is unsubstituted or substituted alkyl, e.g. methyl, by reaction with a strong base, e.g. lithium diisopropylamide, in an appropriate solvent, e.g. hexamethylphosphoramide and/or tetrahydrofurane, at lower temperatures, e.g. from −100 to −50° C., followed by addition of an ammonium salt, e.g. aqueous ammonium chloride, at a preferred temperature from 30 to 40° C., to give a corresponding compound of the formula XV;

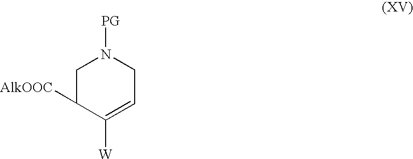

with substituents as defined under formula XIV; this compound can then be converted into an epoxy compound of the formula XVI;

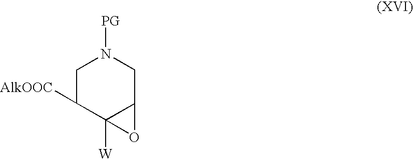

with the moieties as defined under formula XIV, preferably by reaction with an organic peroxide, e.g. m-chloroperbenzoic acid, in an appropriate solvent, e.g. dichloromethane, at temperatures e.g. from −30 to 50° C.; to introduce a hydroxy group and the double bond, this compound can then be reacted with an alkalimetal alkoholate, e.g. sodium methoxide, in an appropriate solvent, e.g. the corresponding alcohol, such as methanol, at elevated temperatures, e.g. from 50° C. to the reflux temperature of the mixture.

The result is a corresponding compound of the formula XVII,

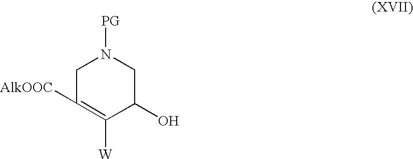

wherein the moieties are as defined for a compound of the formula XIV which can then be converted directly into a compound of the formula II wherein G-R5 is OH and the other moieties are as defined by hydrolysis of the —COOAlk group, or alkylated to a compound of the formula IXVIII

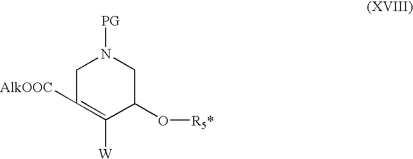

wherein R5* is unsubstituted or substituted alkyl or acyl, by reaction with a compound of the formula XIX,

R₅*—V (XIX)

wherein R₅* is as just defined and V is a leaving group, e.g. halo, such as chloro, (unsubstituted or halo-substituted-C₁-C₇-alkyl)sulfonyl or (unsubstituted or C₁-C₇-alkyl-substituted-phenyl)sulfonyl; the reaction preferably takes place in the presence of a nitrogen base, such as diisopropylethylamine, in an appropriate solvent e.g. dichloromethane, preferably at lower temperatures, e.g. from −30 to 30° C. Hydrolysis of the —COOAlk group yields the corresponding compound of the formula II.

The OH group in formula can also be converted into corresponding groups -G-R5 wherein G is thio, imino or substituted imino (—NR6-) as defined above according to reactions that are well known in the art (e.g. by nucleophilic substitution with a precursor of R5 carrying an SH or NH₂ or NHR6 group after e.g. transformation of the OH group in formula XVII to a halo or toluolsulfonyl or methysulfonyl group).

A halo, e.g. bromo, group in place of Q in a compound of the formula V or in place of L in a compound of the formula IV or in place of L in a compound of the formula VIII can also be converted into the corresponding —B(OH)₂ group e.g. by reaction with a solution of an alkylalkalimetal, such as n-butyllithium, in an appropriate solvent, e.g. hydrocarbons, such as hexane, and/or tetrahydrofurane, first at lower temperatures, e.g. from −100 to −50° C., with subsequent addition of tri-lower alkylborane, e.g. (iPrO)₃B, and reaction at preferred temperatures from 0 to 50° C., thus yielding the corresponding starting materials.

Other starting materials, their synthesis or analogous methods for their synthesis are known in the art, commercially available, and/or they can be found in or derived from the Examples.

### General Process Conditions

The following applies in general to all processes mentioned hereinbefore and hereinafter, while reaction conditions specifically mentioned above or below are preferred:

In any of the reactions mentioned hereinbefore and hereinafter, protecting groups may be used where appropriate or desired, even if this is not mentioned specifically, to protect functional groups that are not intended to take part in a given reaction, and they can be introduced and/or removed at appropriate or desired stages. Reactions comprising the use of protecting groups are therefore included as possible wherever reactions without specific mentioning of protection and/or deprotection are described in this specification.

Within the scope of this disclosure only a readily removable group that is not a constituent of the particular desired end product of formula I is designated a “protecting group”, unless the context indicates otherwise. The protection of functional groups by such protecting groups, the protecting groups themselves, and the reactions appropriate for their introduction and removal are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

All the above-mentioned process steps can be carried out under reaction conditions that are known per se, preferably those mentioned specifically, in the absence or, customarily, in the presence of solvents or diluents, preferably solvents or diluents that are inert towards the reagents used and dissolve them, in the absence or presence of catalysts, condensation or neutralizing agents, for example ion exchangers, such as cation exchangers, e.g. in the H⁺ form, depending on the nature of the reaction and/or of the reactants at reduced, normal or elevated temperature, for example in a temperature range of from about −100° C. to about 190° C., preferably from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofurane or dioxane, liquid aromatic hydrocarbons, such as benzene or toluene, alcohols, such as methanol, ethanol or 1- or 2-propanol, nitrites, such as acetonitrile, halogenated hydrocarbons, e.g. as methylene chloride or chloroform, acid amides, such as dimethylformamide or dimethyl acetamide, bases, such as heterocyclic nitrogen bases, for example pyridine or N-methylpyrrolidin-2-one, carbocylic acid anhydrides, such as lower alkanoic acid anhydrides, for example acetic anhydride, cyclic, linear or branched hydrocarbons, such as cyclohexane, hexane or isopentane, or mixtures of these, for example aqueous solutions, unless otherwise indicated in the description of the processes. Such solvent mixtures may also be used in working up, for example by chromatography or partitioning.

The invention relates also to those forms of the process in which a compound obtainable as Intermediate at any stage of the process is used as starting material and the remaining process steps are carried out, or in which a starting material is formed under the reaction conditions or is used in the form of a derivative, for example in protected form or in the form of a salt, or a compound obtainable by the process according to the invention is produced under the process conditions and processed further in situ. In the process of the present invention those starting materials are preferably used which result in compounds of formula I described as being preferred. Special preference is given to reaction conditions that are identical or analogous to those mentioned in the Examples.

### Pharmaceutical Use, Pharmaceutical Preparations and Methods

As described above, the compounds of the present invention are inhibitors of renin activity and, thus, may be employed for the treatment of hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders, and the like.

The present invention further provides pharmaceutical compositions comprising a therapeutically effective amount of a pharmacologically active compound of the instant invention, alone or in combination with one or more pharmaceutically acceptable carriers.

The pharmaceutical compositions according to the present invention are those suitable for enteral, such as oral or rectal, transdermal and parenteral administration to mammals, including man, to inhibit renin activity, and for the treatment of conditions associated with (especially inappropriate) renin activity. Such conditions include hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intraocular pressure, glaucoma, abnormal vascular growth and/or hyperaldosteronism, and/or further cognitive impairment, alzheimers, dementia, anxiety states and cognitive disorders and the like.

Thus, the pharmacologically active compounds of the invention may be employed in the manufacture of pharmaceutical compositions comprising an effective amount thereof in conjunction or admixture with excipients or carriers suitable for either enteral or parenteral application. Preferred are tablets and gelatin capsules comprising the active ingredient together with:

a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and or polyvinylpyrrolidone; if desired
d) disintegrants, e.g., starches, agar, alginic acid or its sodium salt, or effervescent mixtures; and/or
e) absorbants, colorants, flavors and sweeteners.

Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, preferably about 1-50%, of the active ingredient.

Suitable formulations for transdermal application include a therapeutically effective amount of a compound of the invention with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the compound of the skin of the host at a controlled and pre-determined rate over a prolonged period of time, and means to secure the device to the skin.

Accordingly, the present invention provides pharmaceutical compositions as described above for the treatment of conditions mediated by renin activity, preferably, hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders, as well as methods of their use.

The pharmaceutical compositions may contain a therapeutically effective amount of a compound of the formula I as defined herein, either alone or in a combination with another therapeutic agent, e.g., each at an effective therapeutic dose as reported in the art. Such therapeutic agents include:

a) antidiabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; peroxisome proliferator-activated receptor (PPAR) ligands; protein tyrosine phosphatase-1B (PTP-1B) inhibitors such as PTP-112; GSK3 (glycogen synthase kinase-3) inhibitors such as SB-517955, SB-4195052, SB-216763, NN-57-05441 and NN-57-05445; RXR ligands such as GW-0791 and AGN-194204; sodium-dependent glucose cotransporter inhibitors such as T-1095; glycogen phosphorylase A inhibitors such as BAY R3401; biguanides such as met-formin; alpha-glucosidase inhibitors such as acarbose; GLP-1 (glucagon like peptide-1), GLP-1 analogs such as Exendin-4 and GLP-1 mimetics; and DPPIV (dipeptidyl peptidase IV) inhibitors such as LAF237;
b) hypolipidemic agents such as 3-hydroxy-3-methylglutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (famesoid X receptor) and LXR (liver X receptor) ligands; cholestyramine; fibrates; nicotinic acid and aspirin;
c) anti-obesity agents such as orlistat; and
d) anti-hypertensive agents, e.g., loop diuretics such as ethacrynic acid, furosemide and torsemide; angiotensin converting enzyme (ACE) inhibitors such as benazepril, captopril, enalapril, fosinopril, lisinopril, moexipril, perinodopril, quinapril, ramipril and trandolapril; inhibitors of the Na—K-ATPase membrane pump such as digoxin; neutralendopeptidase (NEP) inhibitors; ACE/NEP inhibitors such as omapatrilat, sampatrilat and fasidotril; anglotensin II antagonists such as candesartan, eprosartan, irbesartan, losartan, telmisartan and valsartan, in particular valsartan; β-adrenergic receptor blockers such as acebutolol, atenolol, betaxolol, bisoprolol, metoprolol, nadolot, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; and aldosterone synthase inhibitors.

Other specific anti-diabetic compounds are described by Patel Mona in Expert Opin Investig Drugs, 2003, 12(4), 623-633, in the FIGS. 1 to 7, which are herein incorporated by reference. A compound of the present invention may be administered either simultaneously, before or after the other active ingredient, either separately by the same or different route of administration or together in the same pharmaceutical formulation.

The structure of the therapeutic agents Identified by code numbers, generic or trade names may be taken from the actual edition of the standard compendium “The Merck Index” or from databases, e.g., Patents International (e.g. IMS World Publications). The corresponding content thereof is hereby incorporated by reference.

Accordingly, the present invention provides pharmaceutical compositions comprising a therapeutically effective amount of a compound of the invention alone or in combination with a therapeutically effective amount of another therapeutic agent, preferably selected from anti-diabetics, hypolipidemic agents, anti-obesity agents or anti-hypertensive agents, most preferably from antidiabetics, anti-hypertensive agents or hypolipidemic agents as described above.

The present invention further relates to pharmaceutical compositions as described above for use as a medicament.

The present invention further relates to use of pharmaceutical compositions or combinations as described above for the preparation of a medicament for the treatment of conditions mediated by (especially inappropriate) renin activity, preferably, hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders, and the like.

Thus, the present invention also relates to a compound of formula I for use as a medicament, to the use of a compound of formula I for the preparation of a pharmaceutical composition for the prevention and/or treatment of conditions mediated by (especially inappropriate) renin activity, and to a pharmaceutical composition for use in conditions mediated by (especially inappropriate) renin activity comprising a compound of formula I, or a pharmaceutically acceptable salt thereof, in association with a pharmaceutically acceptable diluent or carrier material therefore.

The present invention further provides a method for the prevention and/or treatment of conditions mediated by (especially inappropriate) renin activity, which comprises administering a therapeutically effective amount of a compound of the present invention to a warm-blooded animal, especially a human, in need of such treatment.

A unit dosage for a mammal of about 50-70 kg may contain between about 1 mg and 1000 mg, advantageously between about 5-600 mg of the active ingredient. The therapeutically effective dosage of active compound is dependent on the species of warm-blooded animal (especially mammal, more especially human), the body weight, age and individual condition, on the form of administration, and on the compound involved.

In accordance with the foregoing the present invention also provides a therapeutic combination, e.g., a kit, kit of parts, e.g., for use in any method as defined herein, comprising a compound of formula I, or a pharmaceutically acceptable salt thereof, to be used concomitantly or in sequence with at least one pharmaceutical composition comprising at least another therapeutic agent, preferably selected from anti-diabetic agents, hypolipidemic agents, anti-obesity agents or anti-hypertensive agents. The kit may comprise instructions for its administration.

Similarly, the present invention provides a kit of parts comprising: (i) a pharmaceutical composition comprising a compound of the formula I according to the invention; and (ii) a pharmaceutical composition comprising a compound selected from an anti-diabetic, a hypolipidemic agent, an anti-obesity agent, an anti-hypertensive agent, or a pharmaceutically acceptable salt thereof, in the form of two separate units of the components (i) to (ii).

Likewise, the present invention provides a method as defined above comprising co-administration, e.g., concomitantly or in sequence, of a therapeutically effective amount of a compound of formula I, or a pharmaceutically acceptable salt thereof, and at least a second drug substance, said second drug substance preferably being an anti-diabetic, a hypolipidemic agent, an anti-obesity agent or an anti-hypertensive agent, e.g., as indicated above.

Preferably, a compound of the invention is administered to a mammal in need thereof.

Preferably, a compound of the invention is used for the treatment of a disease which responds to a modulation of (especially inappropriate) renin activity.

Preferably, the condition associated with (especially inappropriate) renin activity is selected from hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders.

Finally, the present invention provides a method or use which comprises administering a compound of formula I in combination with a therapeutically effective amount of an anti-diabetic agent, a hypolipidemic agent, an anti-obesity agent or an anti-hypertensive agent.

Ultimately, the present invention provides a method or use which comprises administering a compound of formula I in the form of a pharmaceutical composition as described herein.

The above-cited properties are demonstrable in vitro and in vivo tests using advantageously mammals, e.g., mice, rats, rabbits, dogs, monkeys or isolated organs, tissues and preparations thereof. Said compounds can be applied in vitro in the form of solutions, e.g., preferably aqueous solutions, and in vivo either enterally, parenterally, advantageously intravenously, e.g., as a suspension or in aqueous solution. The concentration level in vitro may range between about 10⁻³ molar and 10⁻¹⁰ molar concentrations. A therapeutically effective amount in vivo may range depending on the route of administration, between about 0.001 and 500 mg/kg, preferably between about 0.1 and 100 mg/kg.

As described above, the compounds of the present invention have enzyme-inhibiting properties. In particular, they inhibit the action of the natural enzyme renin. Renin passes from the kidneys into the blood where it effects the cleavage of angiotensinogen, releasing the decapeptide angiotensin I which is then cleaved in the lungs, the kidneys and other organs to form the octapeptide angiotensin II. The octapeptide increases blood pressure both directly by arterial vasoconstriction and indirectly by liberating from the adrenal glands the sodium-ion-retaining hormone aldosterone, accompanied by an increase in extracellular fluid volume which increase can be attributed to the action of angiotensin II. Inhibitors of the enzymatic activity of renin lead to a reduction in the formation of angiotensin I, and consequently a smaller amount of angiotensin II is produced. The reduced concentration of that active peptide hormone is a direct cause of the hypotensive effect of renin inhibitors.

The action of renin inhibitors may be demonstrated inter alia experimentally by means of in vitro tests, the reduction in the formation of angiotensin I being measured in various systems (human plasma, purified human renin together with synthetic or natural renin substrate).

Inter alia the following in vitro tests may be used:

Recombinant human renin (expressed in Chinese Hamster Ovary cells and purified using standard methods) at 7.5 nM concentration is incubated with test compound at various concentrations for 1 h at RT in 0.1 M Tris-HCl buffer, pH 7.4, containing 0.05 M NaCl, 0.5 mM EDTA and 0.05% CHAPS. Synthetic peptide substrate Arg-Glu(EDANS)-Ile-His-Pro-Phe-His-Leu-Val-Ile-His-Thr-Lys(DABCYL)-Arg9 is added to a final concentration of 2 μM and increase in fluorescence is recorded at an excitation wave-length of 350 nm and at an emission wave-length of 500 nm in a microplate spectro-fluorimeter. IC₅₀ values are calculated from percentage of inhibition of renin activity as a function of test compound concentration (Fluorescence Resonance Energy Transfer, FRET, assay). Compounds of the formula I, in this assay, preferably can show IC₅₀ values in the range from 1 nM to 15 μM

Alternatively, recombinant human renin (expressed in Chinese Hamster Ovary cells and purified using standard methods) at 0.5 nM concentration is incubated with test compound at various concentrations for 2 h at 37° C. in 0.1 M Tris-HCl buffer, pH 7.4, containing 0.05 M NaCl, 0.5 mM EDTA and 0.05% CHAPS. Synthetic peptide substrate Arg-Glu(EDANS)Ile-His-Pro-Phe-His-Leu-Val-Ile-His-Thr-Lys(DABCYL)-Arg9 is added to a final concentration of 4 μM and increase in fluorescence is recorded at an excitation wave-length of 340 nm and at an emission wave-length of 485 nm in a microplate spectro-fluorimeter. IC₅₀ values are calculated from percentage of inhibition of renin activity as a function of test compound concentration (Fluorescence Resonance Energy Transfer, FRET, assay). Compounds of the formula I, in this assay, preferably can show IC₅₀ values in the range from 1 nM to 15 μM.

In another assay, human plasma spiked with recombinant human renin (expressed in Chinese Hamster Ovary cells and purified using standard methods) at 0.8 nM concentration is incubated with test compound at various concentrations for 2 h at 37° C. in 0.1 M Tris/HCl pH 7.4 containing 0.05 M NaCl, 0.5 mM EDTA and 0.025% (w/v) CHAPS. Synthetic peptide substrate Ac-Ile-His-Pro-Phe-His-Leu-Val-Ile-His-Asn-Lys-[DY-505-X5] is added to a final concentration of 2.5 μM. The enzyme reaction is stopped by adding an excess of a blocking inhibitor. The product of the reaction is separated by capillary electrophoresis and quantified by spectrophotometric measurement at 505 nM wave-length. IC₅₀ values are calculated from percentage of inhibition of renin activity as a function of test compound concentration. Compounds of the formula I, in this assay, preferably can show IC₅₀ values in the range from 1 nM to 15 μM.

In another assay, recombinant human renin (expressed in Chinese Hamster Ovary cells and purified using standard methods) at 0.8 nM concentration is incubated with test compound at various concentrations for 2 h at 37° C. in 0.1 M Tris/HCl pH 7.4 containing 0.05 M NaCl, 0.5 mM EDTA and 0.025% (w/v) CHAPS. Synthetic peptide substrate Ac-Ile-His-Pro-Phe-His-Leu-Val-Ile-His-Asn-Lys-[DY-505-X5] is added to a final concentration of 2.5 μM. The enzyme reaction is stopped by adding an excess of a blocking inhibitor. The product of the reaction is separated by capillary electrophoresis and quantified by spectrophotometric measurement at 505 nM wave-length. IC₅₀ values are calculated from percentage of inhibition of renin activity as a function of test compound concentration. Compounds of the formula I, in this assay, preferably show IC₅₀ values in the range from 1 nM to 15 μM.

In animals deficient in salt, renin inhibitors bring about a reduction in blood pressure. Human renin may differ from the renin of other species. In order to test inhibitors of human renin, primates, e.g., marmosets (Callithrix jacchus) may be used, because human renin and primate renin are substantially homologous in the enzymatically active region. Inter alia the following in vivo tests may be used:

Compounds can be tested in vivo in primates as described in the literature (see for example by Schnell C R et al. Measurement of blood pressure and heart rate by telemetry in conscious, unrestrained marmosets. Am J Physiol 264 (Heart Circ Physiol 33). 1993: 1509-1516; or Schnell C R et al. Measurement of blood pressure, heart rate, body temperature, ECG and activity by telemetry in conscious, unrestrained marmosets. Proceedings of the fifth FELASA symposium: Welfare and Science. Eds BRIGHTON. 1993.

### EXAMPLES

The following examples serve to illustrate the invention without limiting the scope thereof:

Abbreviations

| | |
|---|---|
| Ac | acetyl |
| aq. | aqueous |
| Boc | tert-butoxycarbonyl |
| Brine | saturated sodium chloride solution |
| Celite | trademark of Celite Corp. for filtering aid based on |
| | kieselguhr |
| conc. | concentrated |
| DCM | dichloromethane |
| DEAD | diethyl azodicarboxylate |
| DIEA | N,N-diisopropylethylamine |
| DMF | N,N-dimethylformamide |
| DMSO | dimethylsulfoxide |
| EDC | 1-(3-Dimethylaminopropyl)-3-ethylcarbodiimide |
| | hydrochloride |
| ES-MS | electrospray mass spectrometry |
| Et | ethyl |
| EtOAc | ethyl acetate |
| h | hour(s) |
| HMPA | hexamethylphosphoramide |
| HOAt | 1-hydroxy-7-azabenzotriazole |
| HPLC | high-pressure liquid chromatography |
| IPr | isopropyl |
| LAH | lithium aluminium hydride |
| LDA | lithium diisopropylamide |
| mCPBA | 3-chloroperbenzoic acid |
| NaOMe | sodium methoxide |
| Me | methyl |
| min | minute(s) |
| mL | milliliter(s) |
| MOMCl | methoxymethyl chloride |
| MS | Mass Spectrometry |
| MsCl | Methylsulfonylchlorid |
| nBuLi | n-butyllithium |
| n-Hex | n-hexyl |
| NMR | nuclear magnetic resonance |
| Ph | phenyl |
| RT | room temperature |
| t_{RET} | HPLC retention time in min |
| TBTU | O-(benzotriazol-1-yl)-N,N,N′,N′-tetramethylammonium |
| | tetrafluoroborate |
| TFA | trifluoroacetic acid |
| Tf₂O | trifluoromethanesulfonic anhydride |
| THF | tetrahydrofurane |
| TMS | trimethylsilyl |
| TMSOTf | trifluoromethanesulfonic acid trimethylsilyl ester |
| WSCD = | EDC |

### Synthesis

Flash chromatography is performed by using silica gel (Merck; 40-63 μm). For thin layer chromatography, pre-coated silica gel (Merck 60 F254; Merck KgaA, Darmstadt, Germany)) plates are used. ¹NMR measurements are performed on a Bruker DXR 400 spectrometer using tetraethylsilane as internal standard. Chemical shifts (δ) are expressed in ppm downfield from tetramethylsilane. Electrospray mass spectra are obtained with a Fisons Instruments VG Platform II. Commercially available solvents and chemicals are used for syntheses.

### HPLC Condition:

Column: Nucleosil 100-3 C18 HD, 125×4.0 mm (Macherey & Nagel, Düren, Germany).

Flow rate: 1.0 ml/min

Mobile phase: A) TFA/water (0.1/100, v/v), B) TFA/acetonitrile (0.1/100, v/v)

Gradient: linear gradient from 20% B to 100% B in 7 min

Detection: UV at 254 nm

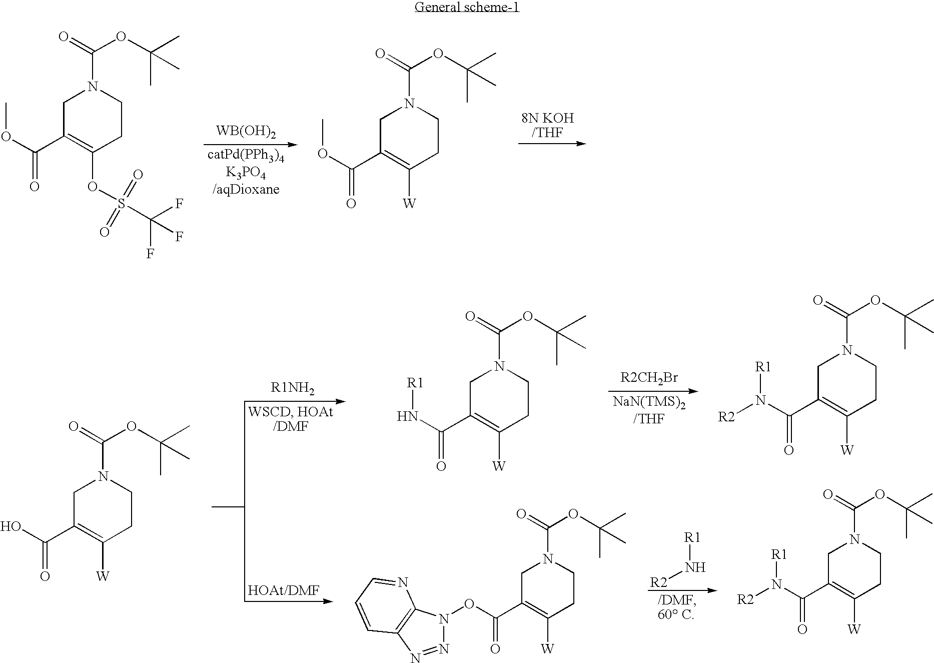

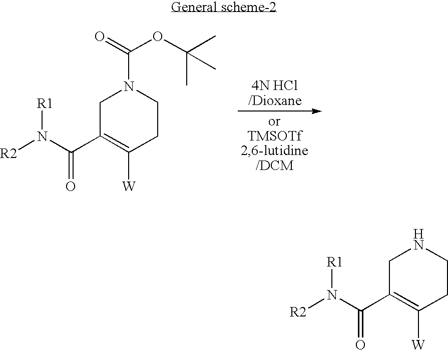

### Example 1

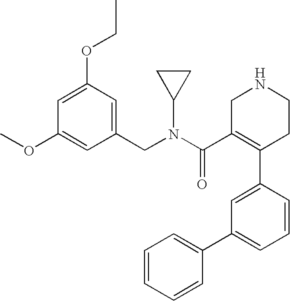

A mixture of Intermediate 1.1 (81 mg, 0.134 mmol) and 4N dioxane solution of HCl (3 mL) is stirred under N₂ at RT. After stirring for 20 min, the reaction mixture is concentrated under reduced pressure to give Example 1 as white solid; ES-MS: M+H=483; HPLC: t_{Ret}=3.49 min

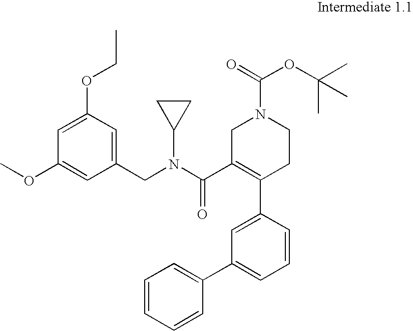

To a mixture of Intermediate 1.2 (200 mg, 0.48 mmol) and Intermediate 1.5 (141 mg, 0.57 mmol) in THF (5 mL), 1 M THF solution of NaN(TMS)₂ (1.0 mL, 1.0 mmol) is added under N₂ at 0° C. After stirring at RT for 3 h and adding H₂O, the reaction mixture is extracted with EtOAc. The combined organic phases are washed with H₂O, brine and dried (Na₂SO₄). Concentration under reduced pressure and silica gel flash chromatography give Intermediate 1.1 as white amorphous material; ES-MS: M+H=583; HPLC: t_{Ret}=5.27 min.

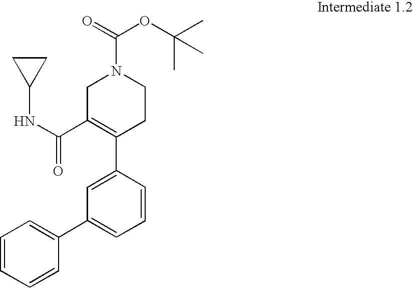

A mixture of Intermediate 1.3 (4.9 g, 13 mmol), cyclopropylamine (1.1 mL, 15.6 mmol), WSCD (3.74 g, 19.5 mmol) and HOAt (2.65 g, 19.5 mmol) in DMF (15 mL) is stirred under N₂ at RT for 3 h. After adding H₂O, the reaction mixture is extracted with EtOAc. The combined organic phases are washed with H₂O, brine and dried (Na₂SO₄). Concentration under reduced pressure and silica gel flash chromatography give Intermediate 1.2 as white amorphous material; ES-MS: M+H=419; HPLC: t_{Ret}=4.43 min.

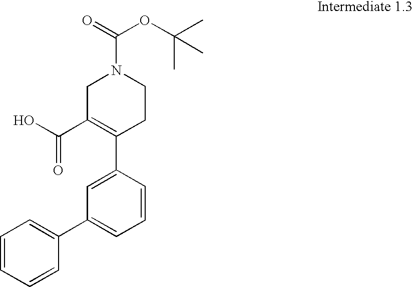

A solution of Intermediate 1.4 (15 g, 38 mmol) in THF (40 mL) and 8N KOH (40 mL) is refluxed under N₂ for 15 h. After cooling down to RT, the reaction mixture is adjusted to weakly acidic pH by slowly adding aqueous saturated critic acid, and the mixture is extracted with EtOAc (30 mL, 3×). The combined organic phases are washed with H₂O, brine and dried (Na₂SO₄). Concentration under reduced pressure and silica gel flash chromatography give Intermediate 1.3 as white amorphous material; ES-MS: M+H=306; HPLC: t_{Ret}=4.45 min.

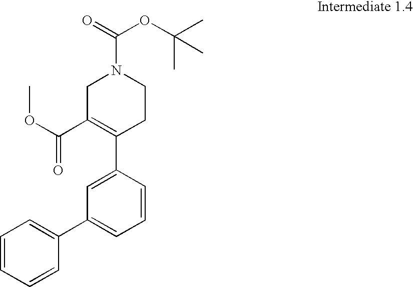

A mixture of 4-trifluoromethanesulfonyloxy-5,6-dihydro-2H-pyridine-1,3-dicarboxylic acid 1-tert-butyl ester 3-methyl ester (14 g, 36 mmol) (see e.g. WO 2004/002957 or US 2003/216441), 3-biphenylboronic acid (11.9 g, 43 mmol), K₃PO₄ (15.3 g, 72 mmol) and Pd(PPh₃)₄ (1.25 g, 1.1 mmol) in dioxane (150 mL) is stirred under N₂ at 80° C. for 5 h. After adding H₂O, the reaction mixture is extracted with EtOAc. The combined organic phases are washed with H₂O, brine and dried (Na₂SO₄). Concentration under reduced pressure and silica gel flash chromatography give Intermediate 1.4 as white amorphous material; ES-MS: M+H=394; HPLC: t_{Ret}=5.12 min.

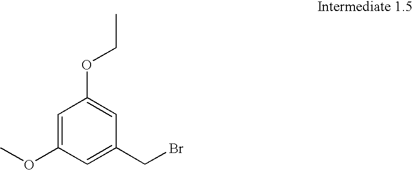

A mixture of Intermediate 1.6 (783 mg, 4.3 mmol), PPh₃ (1.2 g, 4.7 mmol) and CBr₄ (1.6 g, 4.7 mmol) in Et₂O (10 mL) is stirred under N₂ at RT for 1 h. After adding H₂O (10 mL), the reaction mixture is extracted with Et₂O. The combined organic phases are washed with H₂O, brine and dried (Na₂SO₄). Concentration under reduced pressure and silica gel flash chromatography give Intermediate 1.5 as colorless oil; ES-MS: M+=245; HPLC: t_{Ret}=4.03 min.

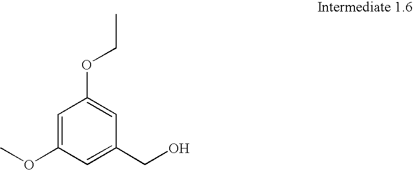

A mixture of 3-ethoxy-5-methoxybenzoic acid ethyl ester (2.4 g, 10.7 mmol) (see Taiwan Kexue, 1996, 49, 1) and LiAlH₄ (610 mg, 16.0 mmol) in THF (20 mL) is stirred under N₂ at OOC for 1.5 h. After adding H₂O, the reaction mixture is extracted with EtOAc. The combined organic phases are washed with H₂O, brine and dried (Na₂SO₄). Concentration under reduced pressure and silica gel flash chromatography give Intermediate 1.6 as colorless oil; ES-MS: M+H=183; HPLC: t_{Ret}=2.89 min.

### Example 2

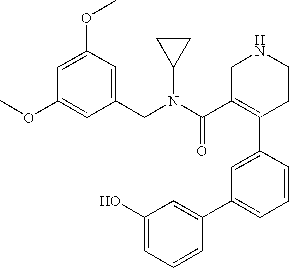

Example 2 is synthesized by deprotection of Intermediate 2.1 (103 mg, 0.18 mmol) analogously to the preparation of compound of Example 1. White solid; ES-MS: M+H=485; HPLC: t_{Ret}=3.12 min.

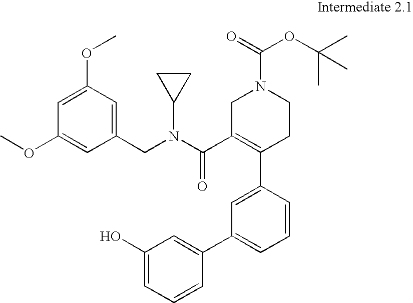

A mixture of Intermediate 2.2 (150 mg, 0.26 mmol), 3-hydroxyphenylboronic acid (47 mg, 0.34 mmol), K₃PO₄ (83 mg, 0.39 mmol) and Pd(PPh₃)₄ (30 mg, 0.026 mmol) in dioxane (4 mL) is refluxed under N₂ for 2 h. After adding H₂O, the reaction mixture is extracted with EtOAc. The combined organic phases are washed with H₂O, brine and dried (Na₂SO₄). Concentration under reduced pressure and silica gel flash chromatography give Intermediate 2.1 as white amorphous material; ES-MS: M+H=585; Rf=0.40 (EtOAc:n-Hex=2:1)

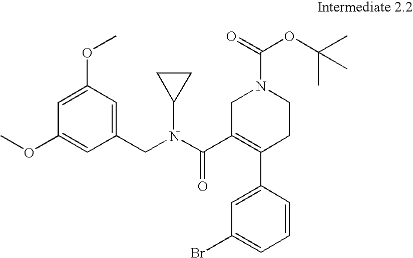

Intermediate 2.2 is synthesized by condensation of Intermediate 2.3 (250 mg, 0.6 mmol) and 1-bromomethyl-3,5-dimethoxy-benzene (246 mg, 1.2 mmol) analogously to the preparation of Intermediate 1.1. White amorphous material; ES-MS: M+H=571; Rf=0.75 (EtOAc-n-Hex=1:1)

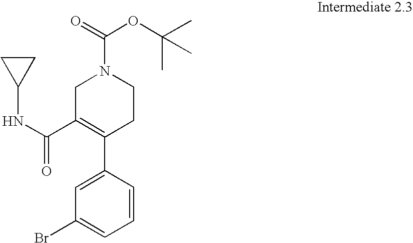

Intermediate 2.3 is synthesized by condensation of Intermediate 2.4 (3.0 g, 7.9 mmol) and cyclopropylamine (5.1 mL, 10.2 mmol) analogously to the preparation of Intermediate 1.2. White amorphous material; ES-MS: M+H=423; HPLC: t_{Ret}=3.95 min.

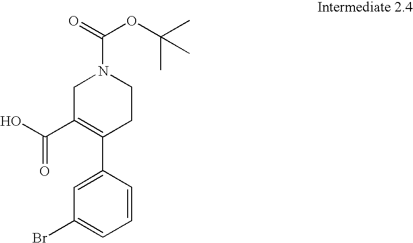

Intermediate 2.4 is synthesized by hydrolysis of Intermediate 2.5 (3.0 g, 7.6 mmol) analogously to the preparation of Intermediate 1.3. White amorphous material; ES-MS: M-^{t}Bu=326; HPLC: t_{Ret}=4.18 min.

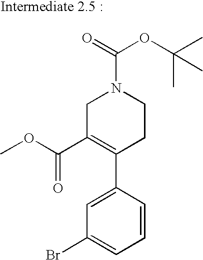

Intermediate 2.5 is synthesized by condensation of 4-trifluoromethanesulfonyloxy-5,6-dihydro-2H-pyridine-1,3-dicarboxylic acid 1-tert-butyl ester 3-methyl ester (see under Intermediate 1.4) 34.4 g, 88.2 mmol) and 3-bromophenylboronic acid (21.3 g, 105.9 mmol) analogously to the preparation of Intermediate 1.4. White amorphous material; ES-MS: M-^{t}Bu=340; HPLC: t_{Ret}=4.89 min.

### Example 3

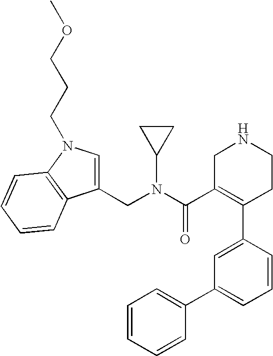

Example 3 is synthesized by deprotection of compound of Intermediate 3.1 (340 mg, 0.55 mmol) analogously to the preparation of Example 1. White solid; ES-MS: M+H=520; HPLC: t_{Ret}=3.68 min.

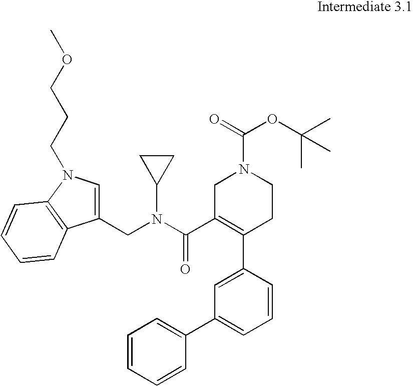

A mixture of Intermediate 3.2 (300 mg, 0.6 mmol) and Intermediate 3.3 (310 mg, 1.2 mmol) in DMF (3 mL) is stirred under N₂ at 60° C. for 1 hour. After adding H₂O, the reaction mixture is extracted with EtOAc. The combined organic phases are washed with H₂O, brine and dried (Na₂SO₄). Concentration under reduced pressure and silica gel flash chromatography give Intermediate 3.1 as white amorphous material; ES-MS: M+H=620; HPLC: t_{Ret}=5.62 min.

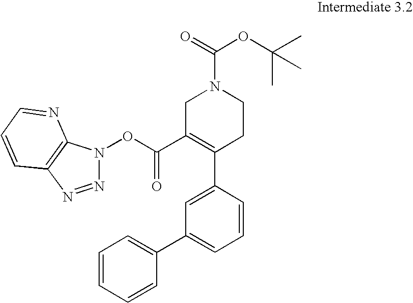

A mixture of Intermediate 1.3 (3.0 g, 7.8 mmol), EDC (1.5 g, 10.2 mmol) and HOAt (1.4 g, 10.2 mmol) in DMF (20 mL) is stirred under N₂ at RT for 30 min. After adding H₂O (20 mL), the reaction mixture is extracted with Et₂O (20 mL, 2×). The combined organic phases are washed with H₂O, brine and dried (Na₂SO₄). Concentration under reduced pressure and silica gel flash chromatography give Intermediate 3.2 as white solid; ES-MS: M+H=498; HPLC: t_{Ret}=5.10 min.

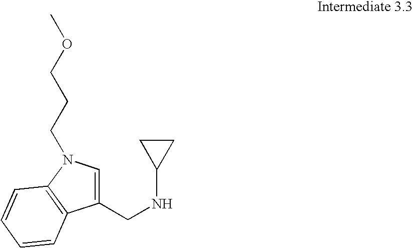

A mixture of Intermediate 3.4 (780 mg, 3.6 mmol), cyclopropylamine (410 mg, 7.2 mmol), AcOH (0.5 mL) and NaBH(OAc)₃ (1.1 g, 5.4 mmol) in DCM (3 mL) and MeOH (1 mL) is stirred under N₂ at 0° C. After stirring at RT for 1 hour, the reaction mixture is quenched with saturated aqueous NaHCO₃ and extracted with DCM. The combined organic phases are washed with H₂O, brine and dried (Na₂SO₄). Concentration under reduced pressure and silica gel flash chromatography give Intermediate 3.3 as yellow oil; ES-MS: M+H=202; HPLC: t_{Ret}=2.67 min

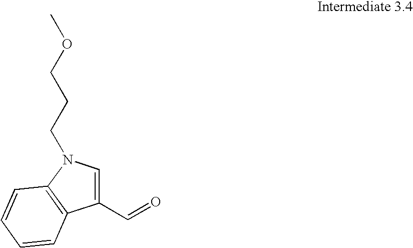

To a mixture of indole-3-carboxaldehyde (1.0 g, 6.9 mmol), toluene-4-sulfonic acid 3-methoxy-propyl ester (2.1 g, 9.0 mmol) and KI (1.1 g, 7.0 mmol) in DMF (15 mL), NaH (320 mg, 7.5 mmol) is added under N₂ at 0° C. After stirring at 50° C. for 4 h, the H₂O is added to the reaction mixture which is then extracted with EtOAc. The combined organic phases are washed with H₂O, brine and dried (Na₂SO₄). Concentration under reduced pressure and silica gel flash chromatography give Intermediate 3.4 as colorless oil; ES-MS: M+H=218, HPLC: t_{Ret}=3.18 min.

The following Examples enlisted in Table 1 are synthesized analogously to the preparation of Example 1-3. As far as not being commercially available, the synthesis of Intermediates for the preparation of compounds of Example 4-112 is described below Table 1 (an asterisk (*) indicates the end of the bond and the end thereof with which the moiety is bound to the rest of the molecule).

TABLE 1

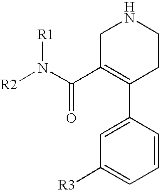

| Example | | | | |
|---|---|---|---|---|
| No. | R1 | R2 | R3 | Analytical data |
| 4 | 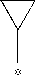 | 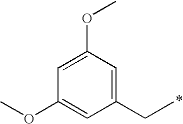 | 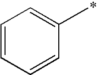 | MS: [M + 1]⁺ = 469 HPLC t_{Ret} = 3.50 min. |
| 5 | 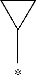 | 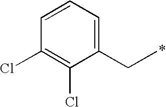 | 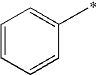 | MS: [M + 1]⁺ = 477 HPLC t_{Ret} = 3.87 min. |
| 6 | 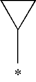 | 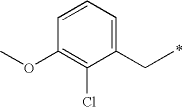 | 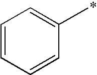 | MS: [M + 1]⁺ = 473 HPLC t_{Ret} = 3.30 min. |
| 7 | 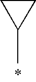 | 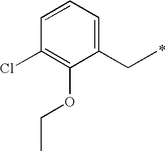 | 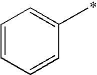 | MS: [M + 1]⁺ = 487 HPLC t_{Ret} = 3.67 min. |
| 8 | 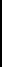 | 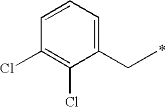 | 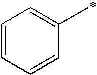 | MS: [M + 1]⁺ = 451 HPLC t_{Ret} = 3.45 min. |
| 9 | 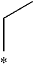 | 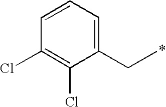 | 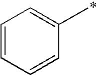 | MS: [M + 1]⁺ = 465 HPLC t_{Ret} = 3.63 min. |
| 10 | 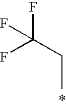 | 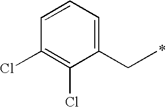 | 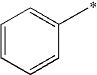 | MS: [M + 1]⁺ = 519 HPLC t_{Ret} = 3.75 min. |
| 11 | 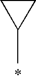 | 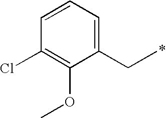 | 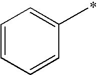 | MS: [M + 1]⁺ = 473 HPLC t_{Ret} = 3.59 min. |
| 12 | 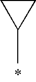 | 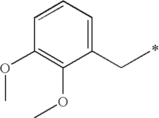 | 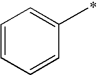 | MS: [M + 1]⁺ = 469 HPLC t_{Ret} = 3.34 min. |
| 13 | 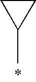 | 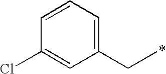 | 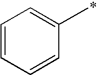 | MS: [M + 1]⁺ = 443 HPLC t_{Ret} = 3.55 min. |
| 14 | H | 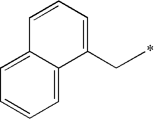 | 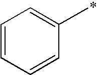 | MS: [M + 1]⁺ = 419 HPLC t_{Ret} = 3.82 min. |
| 15 | 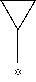 | 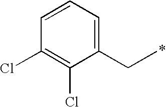 | 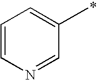 | MS: [M]⁺ = 478 HPLC t_{Ret} = 2.48 min. |
| 16 | 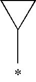 | 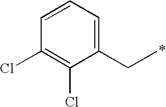 | 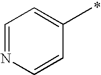 | MS: [M]⁺ = 478 HPLC t_{Ret} = 2.45 min. |
| 17 | 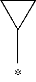 | 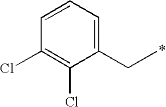 | 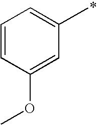 | MS: [M]⁺ = 507 HPLC t_{Ret} = 3.70 min. |
| 18 | 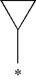 | 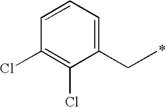 | 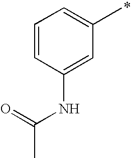 | MS: [M]⁺ = 534 HPLC t_{Ret} = 3.20 min. |
| 19 | 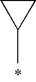 | 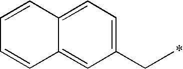 | 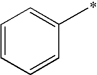 | MS: [M + 1]⁺ = 459 HPLC t_{Ret} = 3.65 min. |
| 20 | 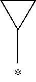 | 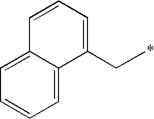 | 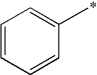 | MS: [M + 1]⁺ = 459 HPLC t_{Ret} = 3.67 min. |
| 21 | 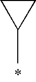 | 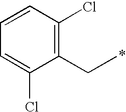 | 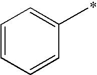 | MS: [M + 1]⁺ = 477 HPLC t_{Ret} = 3.55 min. |
| 22 | 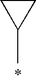 | 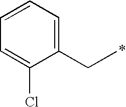 | 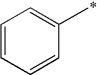 | MS: [M + 1]⁺ = 443 HPLC t_{Ret} = 3.54 min. |
| 23 | 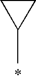 | 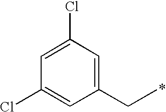 | 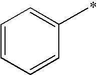 | MS: [M + 1]⁺ = 477 HPLC t_{Ret} = 3.80 min. |
| 24 | 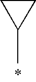 | 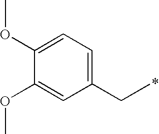 | 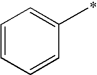 | MS: [M + 1]⁺ = 469 HPLC t_{Ret} = 3.09 min. |
| 25 | 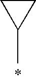 | 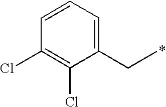 | 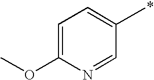 | MS: [M]⁺ = 508 HPLC t_{Ret} = 3.38 min. |
| 26 | 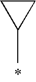 | 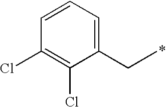 | 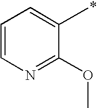 | MS: [M]⁺ = 508 HPLC t_{Ret} = 3.45 min. |
| 27 | 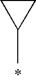 | 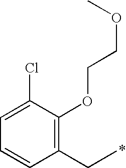 | 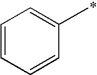 | MS: [M + 1]⁺ = 517 HPLC t_{Ret} = 3.55 min. |
| 28 | 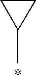 | 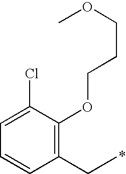 | 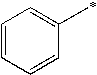 | MS: [M + 1]⁺ = 531 HPLC t_{Ret} = 3.68 min. |
| 29 | 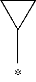 | 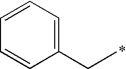 | 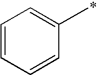 | MS: [M + 1]⁺ = 409 HPLC t_{Ret} = 3.32 min. |
| 30 | 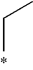 | 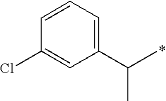 | 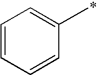 | MS: [M]⁺ = 445 HPLC t_{Ret} = 3.60 min. |
| 31 | H | 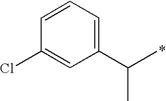 | 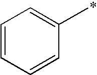 | MS: [M]⁺ = 417 HPLC t_{Ret} = 3.37 min. |
| 32 | 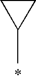 | 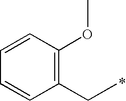 | 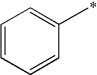 | MS: [M + 1]⁺ = 439 HPLC t_{Ret} = 3.40 min. |
| 33 | 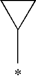 | 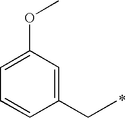 | 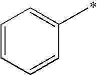 | MS: [M + 1]⁺ = 439 HPLC t_{Ret} = 3.35 min. |
| 34 | 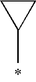 | 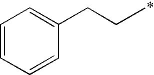 | 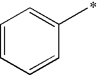 | MS: [M + 1]⁺ = 423 HPLC t_{Ret} = 3.54 min. |
| 35 | 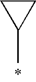 | 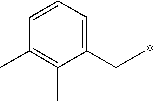 | 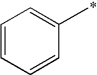 | MS: [M + 1]⁺ = 437 HPLC t_{Ret} = 3.62 min. |
| 36 | 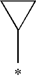 | 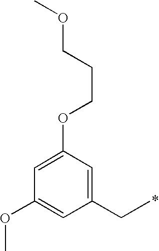 | 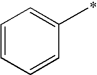 | MS: [M + 1]⁺ = 527 HPLC t_{Ret} = 3.45 min. |
| 37 | 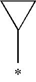 | 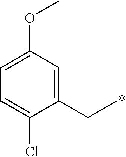 | 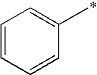 | MS: [M]⁺ = 473 HPLC t_{Ret} = 3.57 min. |
| 38 | 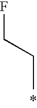 | 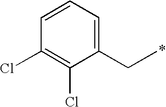 | 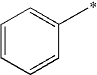 | MS: [M]⁺ = 483 HPLC t_{Ret} = 3.60 min. |
| 39 | 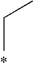 | 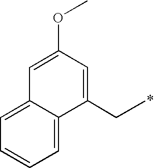 | 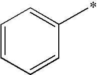 | MS: [M + 1]⁺ = 477 HPLC t_{Ret} = 3.61 min. |
| 40 | 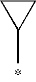 | 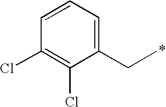 | 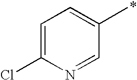 | MS: [M]⁺ = 512 HPLC t_{Ret} = 3.48 min. |
| 41 | 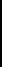 | 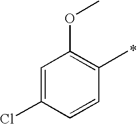 | 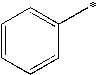 | MS: [M + 1]⁺ = 433 HPLC t_{Ret} = 3.57 min. |
| 42 | 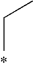 | 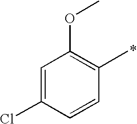 | 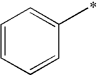 | MS: [M + 1]⁺ = 447 HPLC t_{Ret} = 3.75 min. |
| 43 | 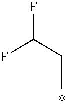 | 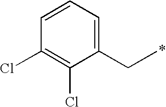 | 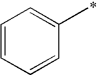 | MS: [M]⁺ = 501 HPLC t_{Ret} = 3.70 min. |
| 44 | 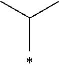 | 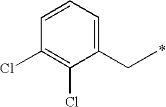 | 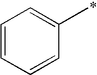 | MS: [M]⁺ = 479 HPLC t_{Ret} = 3.84 min. |
| 45 | 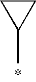 | 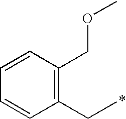 | 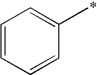 | MS: [M + 1]⁺ = 453 HPLC t_{Ret} = 3.30 min. |
| 46 | 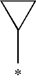 | 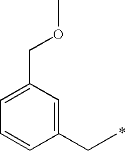 | 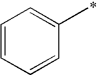 | MS: [M + 1]⁺ = 453 HPLC t_{Ret} = 3.27 min. |
| 47 | 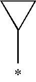 | 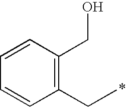 | 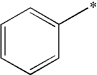 | MS: [M + 1]⁺ = 439 HPLC t_{Ret} = 2.72 min. |
| 48 | 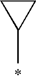 | 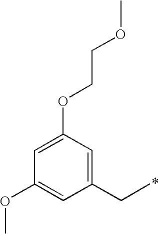 | 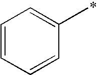 | MS: [M + 1]⁺ = 513 HPLC t_{Ret} = 3.30 min. |
| 49 | 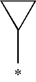 | 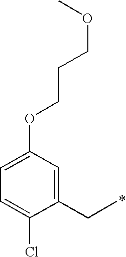 | 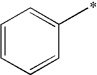 | MS: [M]⁺ = 531 HPLC t_{Ret} = 3.70 min. |
| 50 | 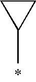 | 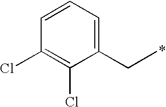 | 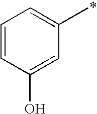 | MS: [M]⁺ = 493 HPLC t_{Ret} = 3.32 min. |
| 51 | 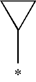 | 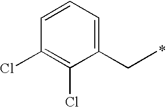 | 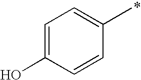 | MS: [M]⁺ = 493 HPLC t_{Ret} = 3.27 min. |
| 52 | 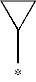 | 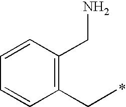 | 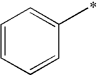 | MS: [M + 1]⁺ = 438 HPLC t_{Ret} = 2.63 min. |
| 53 | 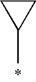 | 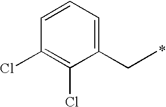 | 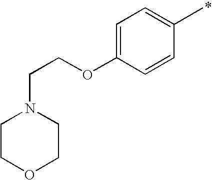 | MS: [M]⁺ = 606 HPLC t_{Ret} = 2.84 min. |
| 54 | 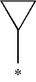 | 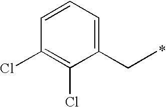 | 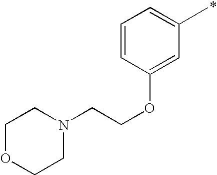 | MS: [M]⁺ = 606 HPLC t_{Ret} = 2.88 min. |
| 55 | 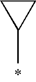 | 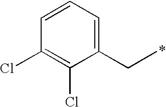 | 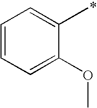 | MS: [M]⁺ = 507 HPLC t_{Ret} = 3.79 min. |
| 56 | 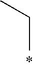 | 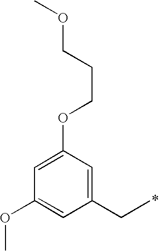 | 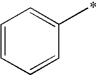 | MS: [M + 1]⁺ = 515 HPLC t_{Ret} = 3.48 min. |
| 57 | 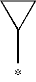 | 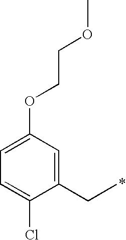 | 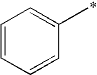 | MS: [M + 1]⁺ = 517 HPLC t_{Ret} = 3.55 min. |
| 58 | 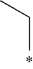 | 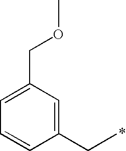 | 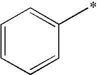 | MS: [M + 1]⁺ = 441 HPLC t_{Ret} = 3.29 min. |
| 59 | 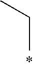 | 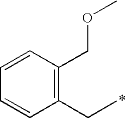 | 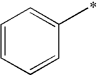 | MS: [M + 1]⁺ = 441 HPLC t_{Ret} = 3.34 min. |
| 60 | 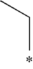 | 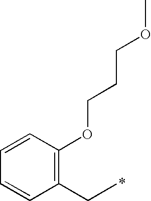 | 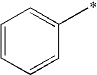 | MS: [M + 1]⁺ = 485 HPLC t_{Ret} = 3.47 min. |
| 61 | 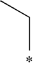 | 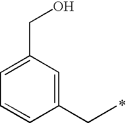 | 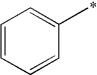 | MS: [M + 1]⁺ = 427 HPLC t_{Ret} = 2.96 min. |
| 62 | 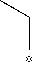 | 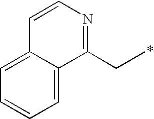 | 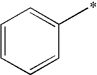 | MS: [M + 1]⁺ = 448 HPLC t_{Ret} = 2.68 min. |
| 63 | 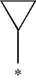 | 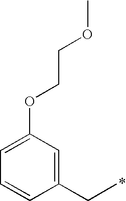 | 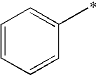 | MS: [M + 1]⁺ = 483 HPLC t_{Ret} = 3.25 min. |
| 64 | 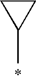 | 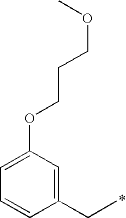 | 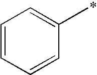 | MS: [M + 1]⁺ = 497 HPLC t_{Ret} = 3.38 min. |
| 65 | 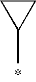 | 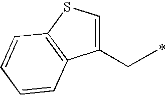 | 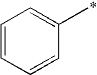 | MS: [M + 1]⁺ = 465 HPLC t_{Ret} = 3.65 min. |
| 66 | 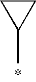 | 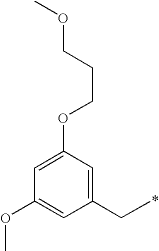 | 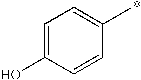 | MS: [M + 1]⁺ = 543 HPLC t_{Ret} = 3.10 min. |
| 67 | 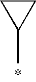 | 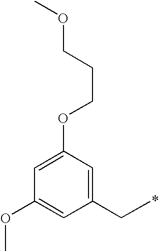 | 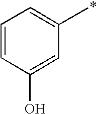 | MS: [M + 1]⁺ = 543 HPLC t_{Ret} = 3.17 min. |
| 68 | 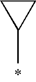 | 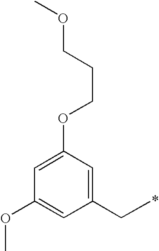 | 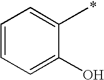 | MS: [M + 1]⁺ = 543 HPLC t_{Ret} = 3.29 min. |
| 69 | 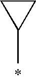 | 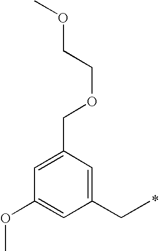 | 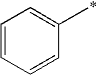 | MS: [M + 1]⁺ = 527 HPLC t_{Ret} = 3.32 min. |
| 70 | 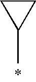 | 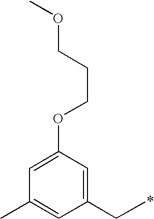 | 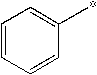 | MS: [M + H]⁺ = 511 HPLC t_{Ret} = 3.55 min. |
| 71 | 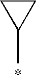 | 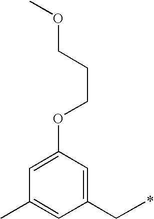 | 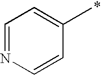 | MS: [M + 1]⁺ = 512 HPLC t_{Ret} = 2.62 min. |
| 72 | 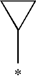 | 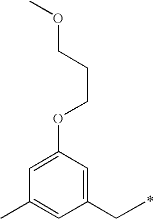 | 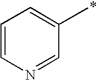 | MS: [M + 1]⁺ = 512 HPLC t_{Ret} = 2.62 min. |
| 73 | 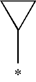 | 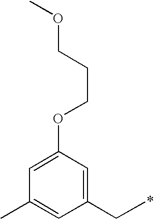 | 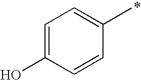 | MS: [M + 1]⁺ = 527 HPLC t_{Ret} = 3.25 min. |
| 74 | 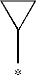 | 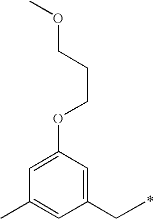 | 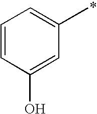 | MS: [M + 1]⁺ = 527 HPLC t_{Ret} = 3.32 min. |
| 75 | 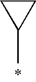 | 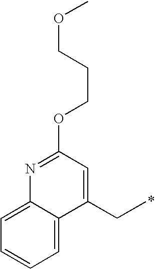 | 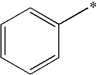 | MS: [M + H]⁺ = 548 HPLC t_{Ret} = 3.52 min. |
| 76 | 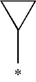 | 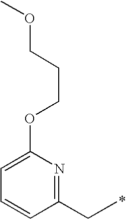 | 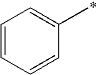 | MS: [M]⁺ = 498 HPLC t_{Ret} = 3.43 min. |
| 77 | 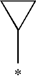 | 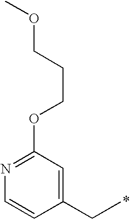 | 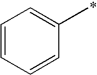 | MS: [M]⁺ = 498 HPLC t_{Ret} = 3.00 min. |
| 78 | 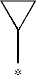 | 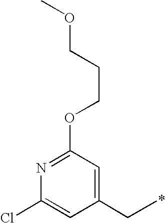 | 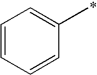 | MS: [M]⁺ = 532 HPLC t_{Ret} = 3.62 min. |
| 79 | 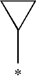 | 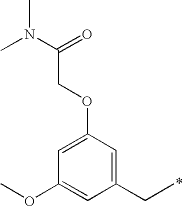 | 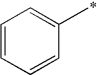 | MS: [M + 1]⁺ = 540 HPLC t_{Ret} = 3.05 min. |
| 80 | 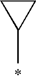 | 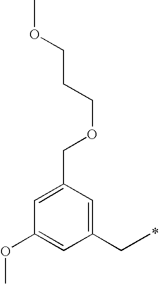 | 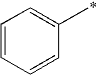 | MS: [M + 1]⁺ = 541 HPLC t_{Ret} = 3.51 min. |
| 81 | 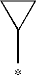 | 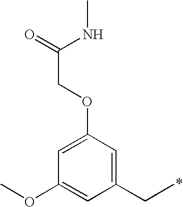 | 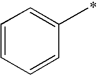 | MS: [M + 1]⁺ = 526 HPLC t_{Ret} = 3.23 min. |
| 82 | 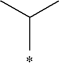 | 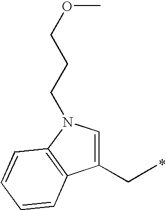 | 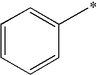 | MS: [M + 1]⁺ = 522 HPLC t_{Ret} = 3.85 min. |
| 83 | 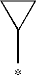 | 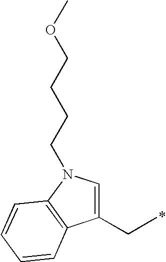 | 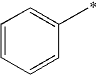 | MS: [M + 1]⁺ = 534 HPLC t_{Ret} = 3.80 min. |
| 84 | 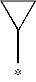 | 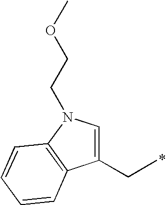 | 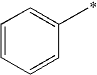 | MS: [M]⁺ = 506 HPLC t_{Ret} = 3.65 min. |
| 85 | 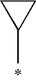 | 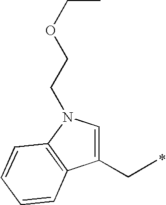 | 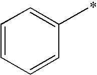 | MS: [M]⁺ = 520 HPLC t_{Ret} = 3.78 min. |
| 86 | 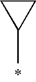 | 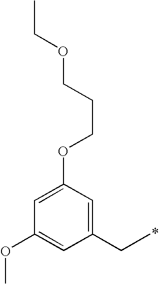 | 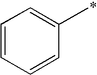 | MS: [M + 1]⁺ = 541 HPLC t_{Ret} = 3.73 min. |
| 87 | 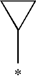 | 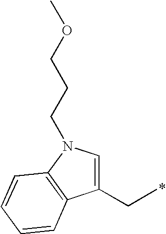 | 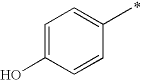 | MS: [M + H]⁺ = 536 HPLC t_{Ret} = 3.27 min. |
| 88 | 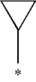 | 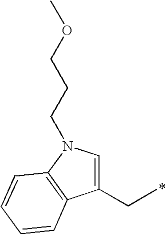 | 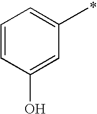 | MS: [M + H]⁺ = 536 HPLC t_{Ret} = 3.27 min. |
| 89 | 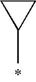 | 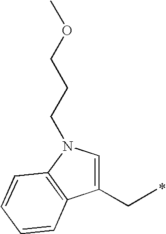 | 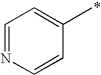 | MS: [M + H]⁺ = 521 HPLC t_{Ret} = 2.62 min. |
| 90 | 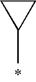 | 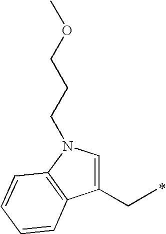 | 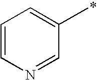 | MS: [M + H]⁺ = 521 HPLC t_{Ret} = 2.65 min. |
| 91 | 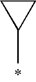 | 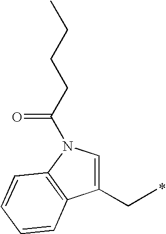 | 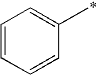 | MS: [M + H]⁺ = 532 HPLC t_{Ret} = 4.02 min. |
| 92 | 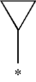 | 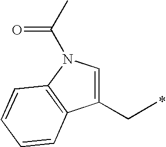 | 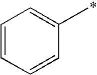 | MS: [M + H]⁺ = 490 HPLC t_{Ret} = 3.48 min. |
| 93 | 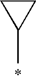 | 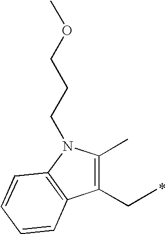 | 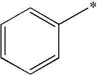 | MS: [M + H]⁺ = 534 HPLC t_{Ret} = 3.77 min. |
| 94 | 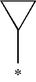 | 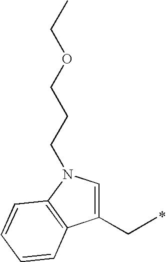 | 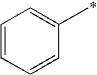 | MS: [M + 1]⁺ = 534 HPLC t_{Ret} = 3.88 min. |
| 95 | 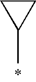 | 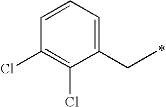 | 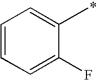 | MS: [M + 1]⁺ = 495 HPLC t_{Ret} = 3.86 min. |
| 96 | 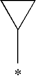 | 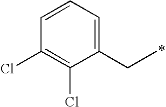 | 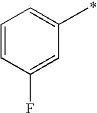 | MS: [M + 1]⁺ = 495 HPLC t_{Ret} = 3.88 min. |
| 97 | 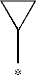 | 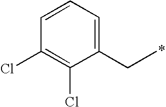 | 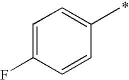 | MS: [M + 1]⁺ = 495 HPLC t_{Ret} = 3.92 min. |
| 98 | 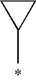 | 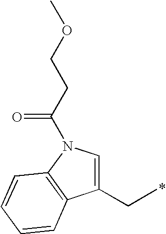 | 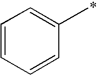 | MS: [M + H]⁺ = 534 HPLC t_{Ret} = 3.52 min. |
| 99 | 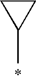 | 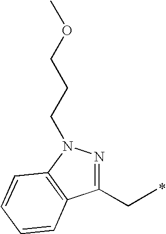 | 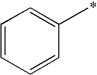 | MS: [M]⁺ = 521 HPLC t_{Ret} = 3.43 min. |
| 100 | 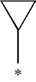 | 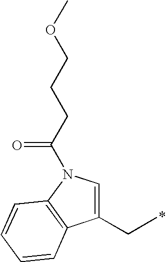 | 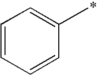 | MS: [M + H]⁺ = 548 HPLC t_{Ret} = 3.43 min. |
| 101 | 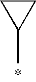 | 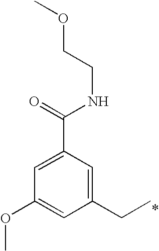 | 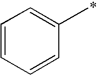 | MS: [M]⁺ = 540 HPLC t_{Ret} = 3.09 min. |
| 102 | 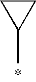 | 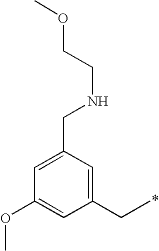 | 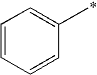 | MS: [M]⁺ = 526 HPLC t_{Ret} = 2.80 min. |
| 103 | 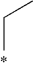 | 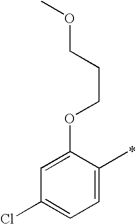 | 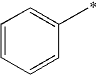 | MS: [M + 1]⁺ = 505 HPLC t_{Ret} = 3.85 min. |
| 104 | 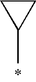 | 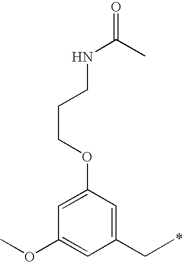 | 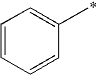 | MS: [M + 1]⁺ = 554 HPLC t_{Ret} = 2.98 min. |
| 105 | 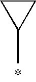 | 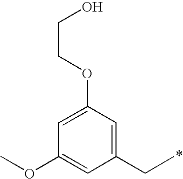 | 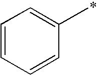 | MS: [M]⁺ = 499 HPLC t_{Ret} = 2.95 min. |
| 106 | 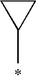 | 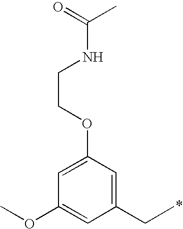 | 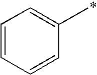 | MS: [M]⁺ = 540 HPLC t_{Ret} = 2.93 min. |
| 107 | 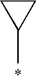 | 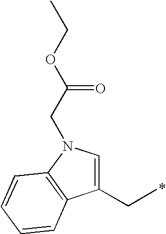 | 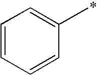 | MS: [M + 1]⁺ = 534 HPLC t_{Ret} = 3.54 min. |
| 108 | 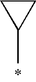 | 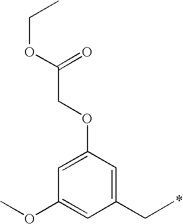 | 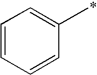 | MS: [M + 1]⁺ = 541 HPLC t_{Ret} = 3.37 min. |
| 109 | 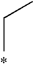 | 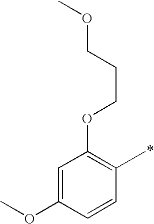 | 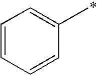 | MS: [M + 1]⁺ = 501 HPLC t_{Ret} = 3.50 min. |
| 110 | 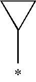 | 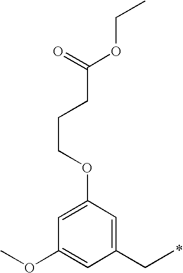 | 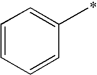 | MS: [M + 1]⁺ = 569 HPLC t_{Ret} = 3.59 min. |
| 111 | 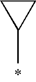 | 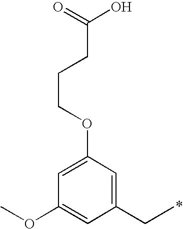 | 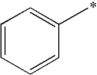 | MS: [M + 1]⁺ = 541 HPLC t_{Ret} = 3.12 min. |
| 112 | 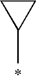 | 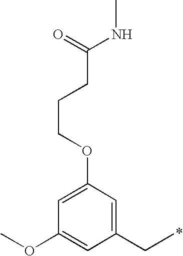 | 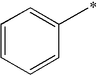 | MS: [M + 1]⁺ = 554 HPLC t_{Ret} = 3.02 min. |

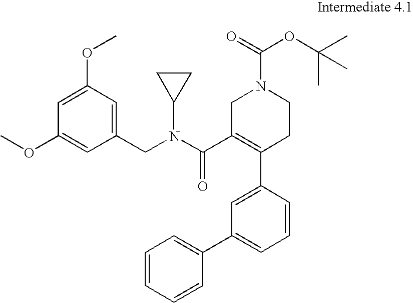

Intermediate 4.1 is synthesized by condensation of Intermediate 1.2 (278 mg, 0.66 mmol) analogously to the preparation of Intermediate 1.1. White amorphous material; Rf=0.21 (AcOEt/Hexane=1/2).

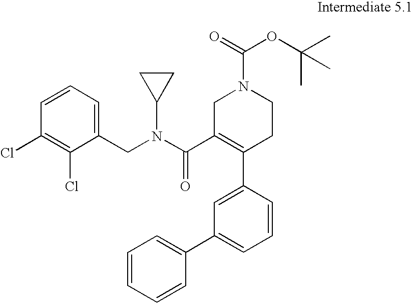

Intermediate 5.1 is synthesized by condensation of Intermediate 1.2 (292 mg, 0.7 mmol) analogously to the preparation of Intermediate 1.1. White amorphous material; ES-MS: M+H=577; HPLC: t_{Ret}=5.60 min.

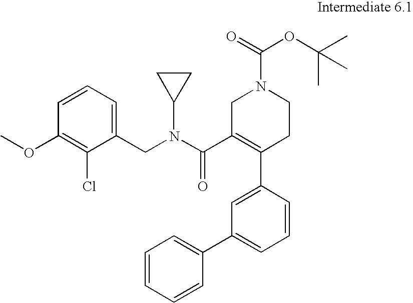

Intermediate 6.1 is synthesized by condensation of Intermediate 1.2 (241 mg, 0.58 mmol) analogously to the preparation of Intermediate 1.1. White amorphous material; Rf=0.53 (EtOAc/Hexane=1/1).

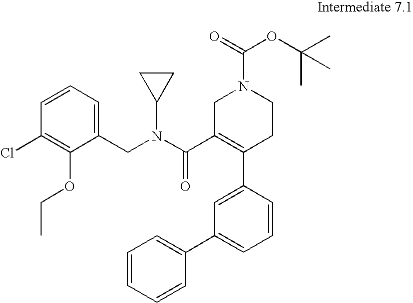

Intermediate 7.1 is synthesized by condensation of Intermediate 12 (320 mg, 0.77 mmol) and Intermediate 7.2 (230 mg, 0.92 mmol) analogously to the preparation of Intermediate 1.1. White amorphous material; ES-MS: M+H=587; HPLC: t_{Ret}=5.65 min.

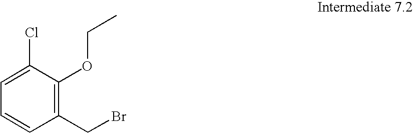

Intermediate 7.2 is synthesized by bromination of the corresponding alcohol which is made by the reduction of Intermediate 7.3 analogously to the preparation of Intermediate 1.5. Colorless oil; Rf=0.44 (Et₂O:Hex=1:4); ¹H NMR (CDCl₃) δ 1.49 (t, 3H), 3.92 (s, 3H), 4.19 (q, 2H), 4.56 (s, 2H), 7.03 (t, 1H), 7.29-7.34 (m, 2H).

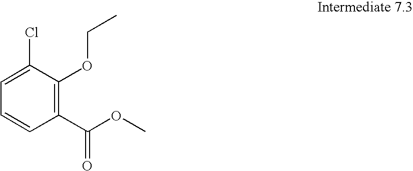

A mixture of compound of 3-chloro-2-hydroxybenzoic add methyl ester (475 mg, 2.55 mmol) (see Organic and Biomolecular Chemistry, 2004, 2, 7, 963-964 and U.S. Pat. No. 4,895,860), EtI (0.22 mL, 2.81 mmol) and K₂CO₃ (422 mg, 3.05 mmol) in DMF (5 mL) is stirred under N₂ at RT for 30 min. After adding H₂O (20 mL), the reaction mixture is extracted with Et₂O (20 mL, 2×). The combined organic phases are washed with H₂O, brine and dried (Na₂SO₄). Concentration under reduced pressure and silica gel flash chromatography give Intermediate 7.3 as colorless oil; Rf=0.57 (EtOAc:Hex=1:2); ¹H NMR (CDCl₃) δ 1.45 (t, 3H), 3.92 (s, 3H), 4.09-4.15 (q, 2H), 7.09 (t, 1H), 7.53-7.55 (dd, 1H), 7.68-7.70 (dd, 1H).

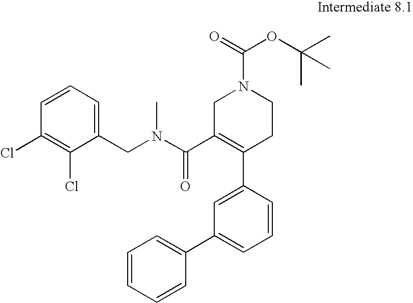

Intermediate 8.1 is synthesized by condensation of Intermediate 8.2 (236 mg, 0.6 mmol) analogously to the preparation of Intermediate 1.1. White amorphous material; ES-MS: M+H=551; HPLC: t_{Ret}=5.43 min.

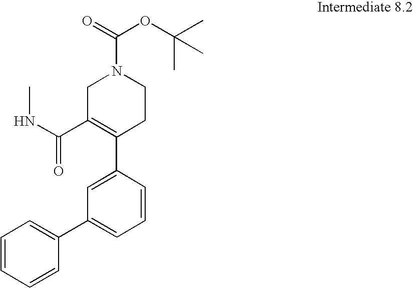

Intermediate 8.2 is synthesized by condensation of Intermediate 1.3 (950 mg, 2.5 mmol) and 2M THF solution of methylamine (1.38 mL, 2.75 mmol) analogously to the preparation of Intermediate 1.2. White amorphous material; ES-MS: M+H=392; HPLC: t_{Ret}=4.15 min.

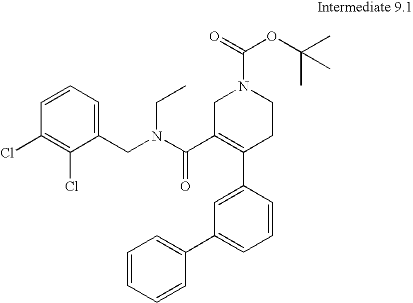

Intermediate 9.1 is synthesized by condensation of Intermediate 9.2 (203 mg, 0.5 mmol) analogously to the preparation of Intermediate 1.1. White amorphous material; ES-MS: M+H=565; HPLC: t_{Ret}=5.62 min.

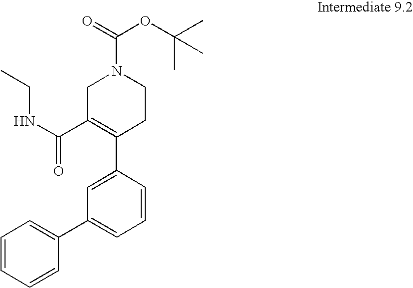

Intermediate 9.2 is synthesized by condensation of Intermediate 1.3 (4.0 g, 10.5 mmol) and 2M THF solution of ethylamine (6.3 mL, 12.6 mmol) analogously to the preparation of Intermediate 1.2. White amorphous material; ES-MS: M+H=407; HPLC: t_{Ret}=4.15 min.

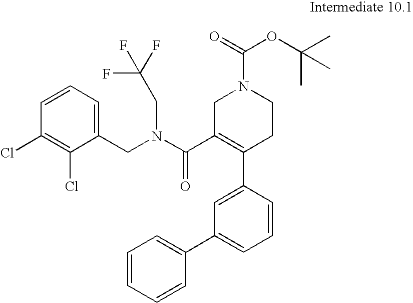

Intermediate 10.1 is synthesized by condensation of Intermediate 10.2 (156 mg, 0.34 mmol) analogously to the preparation of Intermediate 1.1. White amorphous material; ES-MS: M+H=619; HPLC: t_{Ret}=5.70 min.

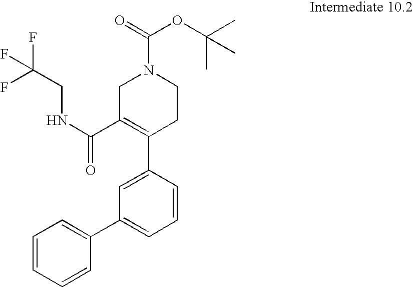

Intermediate 10.2 is synthesized by condensation of Intermediate 1.3 (152 mg, 0.4 mmol) and 2,2,2-trifluoroethylamine hydrochloride (65 mg, 0.48 mmol) analogously to the preparation of Intermediate 1.2. White amorphous material; ES-MS: M+H=461; HPLC: t_{Ret}=4.42 min.

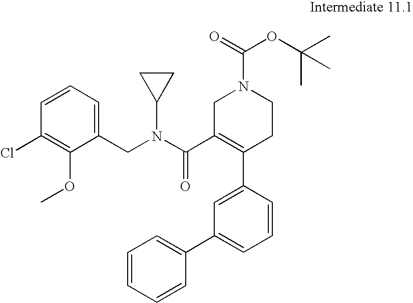

Intermediate 11.1 is synthesized by condensation of Intermediate 1.2 (281 mg, 0.67 mmol) analogously to the preparation of Intermediate 1.1. White amorphous material; ES-MS: M+H=573; HPLC: t_{Ret}=5.45 min.

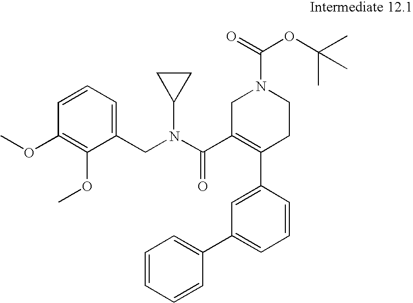

Intermediate 12.1 is synthesized by condensation of Intermediate 1.2 (1.32 g, 3.28 mmol) analogously to the preparation of Intermediate 1.1. White amorphous material; ES-MS: M+H=569; HPLC: t_{Ret}=5.25 min.

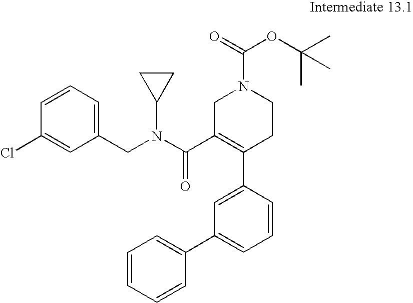

Intermediate 13.1 is synthesized by condensation of Intermediate 1.2 (190 mg, 0.45 mmol) analogously to the preparation of Intermediate 1.1. White amorphous material; ES-MS: M+H=543; HPLC: t_{Ret}=5.43 min.

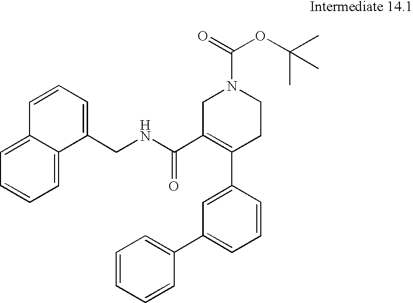

Intermediate 14.1 is synthesized by condensation of Intermediate 1.3 (100 mg, 0.26 mmol) analogously to the preparation of Intermediate 1.2. White amorphous material; ES-MS: M+H=519; HPLC: t_{Ret}=4.64 min.

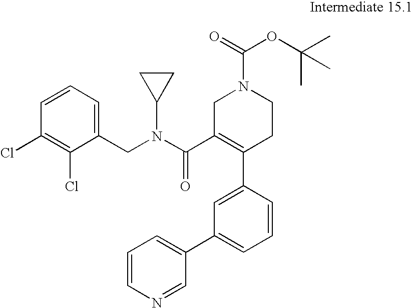

Intermediate 15.1 is synthesized by coupling of Intermediate 152 (174.9 mg, 0.30 mmol) and 3-pyridyl boronic acid (55.6 mg, 0.45 mmol) analogously to the preparation of Intermediate 2.1. White amorphous material; ES-MS: M+=578; HPLC: t_{Ret}=3.73 min.

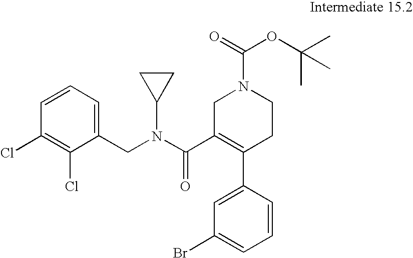

Intermediate 15.2 is synthesized by condensation of Intermediate 2.3 (1.01 mg, 2.40 mmol) analogously to the preparation of Intermediate 1.1. White amorphous material; ES-MS: M+H=581; HPLC: t_{Ret}=5.64 min.

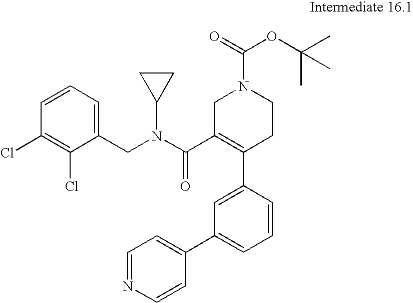

Intermediate 16.1 is synthesized by coupling of Intermediate 15.2 (175.9 mg, 0.3 mmol) and 4-pyridyl boronic acid (55.9 mg, 0.45 mmol) analogously to the preparation of Intermediate 2.1. White amorphous material; ES-MS: M+=587; HPLC: t_{Ret}=3.68 min.

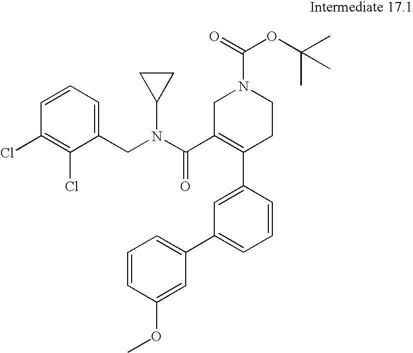

Intermediate 17.1 is synthesized by coupling of Intermediate 15.2 (125 mg, 0.22 mmol) and 3-methoxyphenyl boronic acid (49 mg, 0.32 mmol) analogously to the preparation of Intermediate 2.1. White amorphous material; Rf=0.35 (EtOAc:n-Hex=1:2).

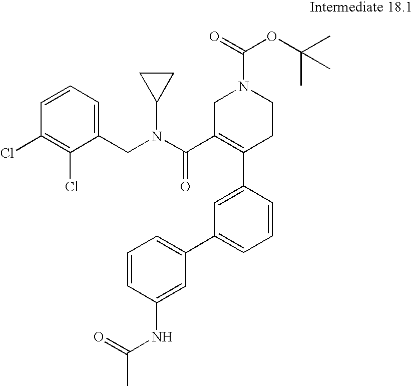

Intermediate 18.1 is synthesized by coupling of Intermediate 15.2 (130 mg, 0.22 mmol) and 3-acetylamidephenyl boronic acid (60 mg, 0.33 mmol) analogously to the preparation of Intermediate 2.1. White amorphous material; Rf=0.28 (EtOAc:n-Hex=1:2)

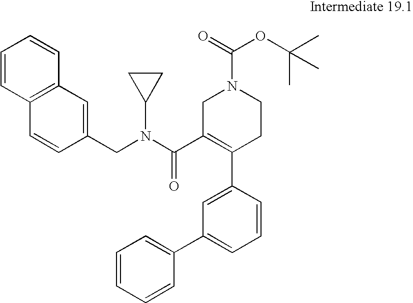

Intermediate 19.1 is synthesized by condensation of Intermediate 1.2 (100 mg, 0.24 mmol) analogously to the preparation of Intermediate 1.1. White amorphous material; Rf=0.29 (EtOAc:n-Hex=1:2).

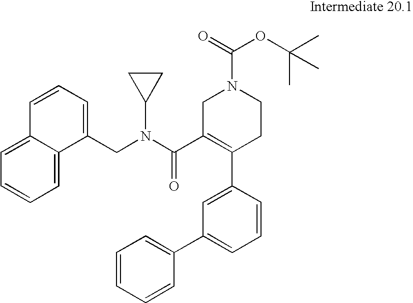

Intermediate 20.1 is synthesized by condensation of Intermediate 1.2 (100 mg, 0.24 mmol) analogously to the preparation of Intermediate 1.1. White amorphous material; ES-MS: M+H=559; HPLC: t_{Ret}=5.43 min.

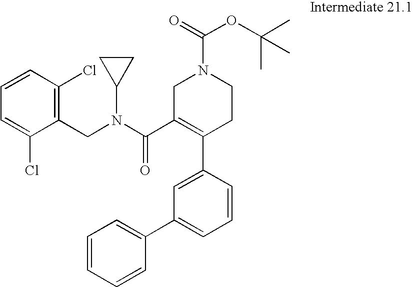

Intermediate 21.1 is synthesized by condensation of Intermediate 1.2 (100 mg, 0.24 mmol) analogously to the preparation of Intermediate 1.1. White amorphous material; ES-MS: M+=577; HPLC: t_{Ret}=5.02 min.

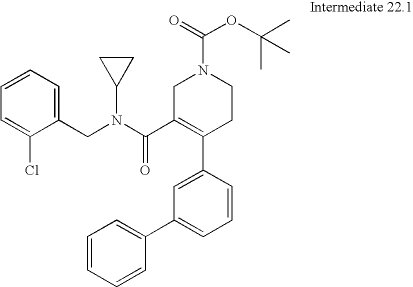

Intermediate 22.1 is synthesized by condensation of Intermediate 1.2 (100 mg, 0.24 mmol) analogously to the preparation of Intermediate 1.1. White amorphous material; ES-MS: M+H=543; HPLC: t_{Ret}=5.02 min.

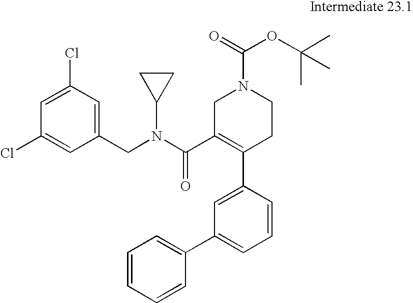

Intermediate 23.1 is synthesized by condensation of Intermediate 1.2 (100 mg, 0.24 mmol) analogously to the preparation of Intermediate 1.1. White amorphous material; Rf=0.33 (EtOAc:n-Hex=1:2).

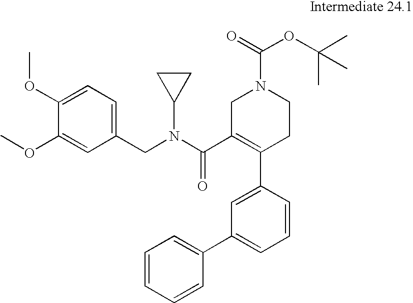

Intermediate 24.1 is synthesized by condensation of Intermediate 1.2 (169 mg, 0.40 mmol) analogously to the preparation of Intermediate 1.1. White amorphous material; ES-MS: M+H=569; HPLC: t_{Ret}=4.97 min.

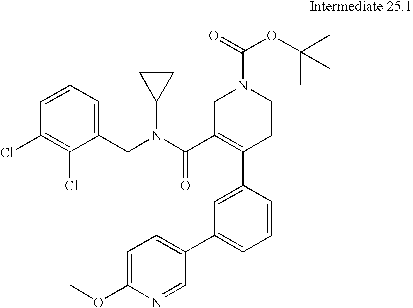

Intermediate 25.1 is synthesized by coupling of Intermediate 15.2 (164.4 mg, 028 mmol) and 2-methoxypyridine-5-boronic add (65.0 mg, 0.42 mmol) analogously to the preparation of Intermediate 2.1. White amorphous material; ES-MS: M+=608; HPLC: t_{Ret}=5.43 min.

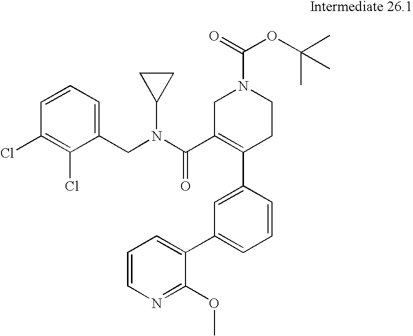

Intermediate 26.1 is synthesized by coupling of Intermediate 15.2 (160.0 mg, 0.28 mmol) and 2-methoxypyridine-3-boronic acid (63.3 mg, 0.41 mmol) analogously to the preparation of Intermediate 2.1. White amorphous material; ES-MS: M+=608; HPLC: t_{Ref}=5.65 min.

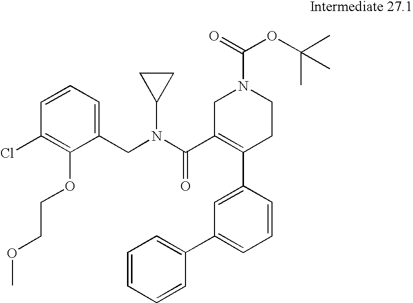

Intermediate 27.1 is synthesized by condensation of Intermediate 12 (293 mg, 0.7 mmol) and Intermediate 27.2 (234 mg, 0.84 mmol) analogously to the preparation of Intermediate 1.1. White amorphous material; ES-MS: M+H=617; HPLC: t_{Ret}=5.59 min.

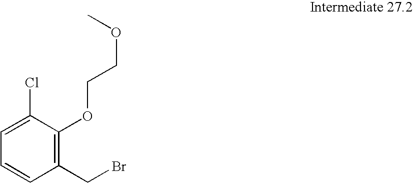

Intermediate 27.2 is synthesized by bromination of the corresponding alcohol which is made by the reduction of corresponding ester. This ester is synthesized by alkylation of 3-chloro-2-hydroxy-benzoic add methyl ester (493 mg, 2.64 mmol) (see e.g. Organic and Biomolecular Chemistry, 2004, 2, 963-964 and U.S. Pat. No. 4,895,860) analogously to the preparation of Intermediate 1.5. Colorless oil; Rf=0.48 (EtOAc:n-Hex=1:2); HPLC: t_{Ret}=4.24 min.

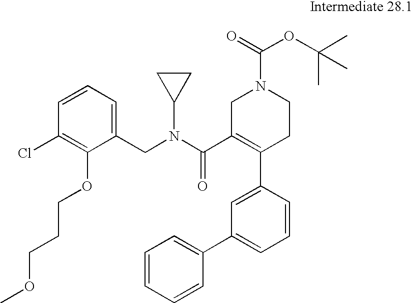

Intermediate 28.1 is synthesized by condensation of Intermediate 1.2 (324 mg, 0.77 mmol) and Intermediate 28.2 (272 mg, 0.93 mmol) analogously to the preparation of Intermediate 1.1. White amorphous material; ES-MS: M+H=631; HPLC: t_{Ret}=5.75 min.

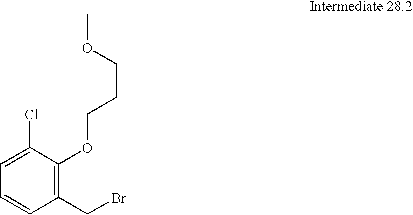

Intermediate 28.2 is synthesized by bromination of the corresponding alcohol which is made by the reduction of corresponding ester. This ester is synthesized by alkylation of 3-chloro-2-hydroxy-benzoic acid methyl ester (625 mg, 3.35 mmol) (see Organic and Biomolecular Chemistry, 2004, 2, 7, 963-964 and U.S. Pat. No. 4,895,860) analogously to the preparation of Intermediate 1.5. Colorless oil; Rf=0.43 (EtOAc:n-Hex=1:2); HPLC: t_{Ret}=4.50 min.

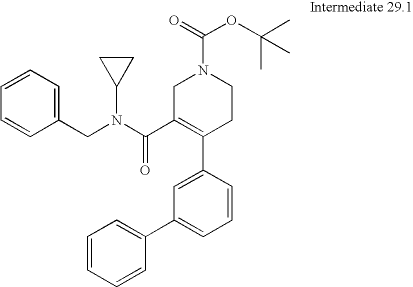

Intermediate 29.1 is synthesized by condensation of Intermediate 1.2 (176 mg, 0.42 mmol) analogously to the preparation of Intermediate 1.1. White amorphous material; ES-MS: M+H=509; HPLC: t_{Ret}=5.15 min.

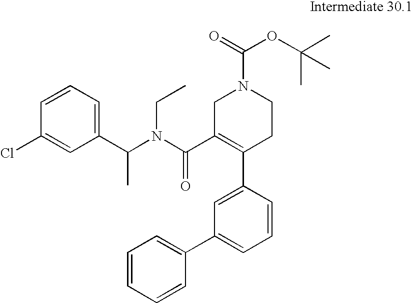

Intermediate 30.1 is synthesized by condensation of Intermediate 31.1 (200 mg, 0.39 mmol) analogously to the preparation of Intermediate 1.1. White amorphous material; ES-MS: M+=545; HPLC: t_{Ret}=5.67 min.

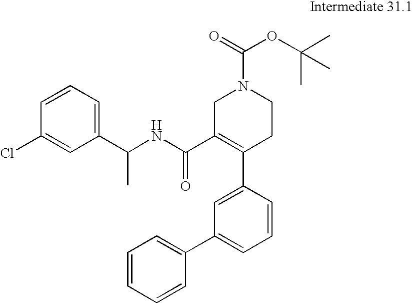

Intermediate 31.1 is synthesized by condensation of Intermediate 1.3 (400 mg, 1.05 mmol) analogously to the preparation of Intermediate 12. White amorphous material; ES-MS: M+=517; HPLC: t_{Ret}=5.22 min.

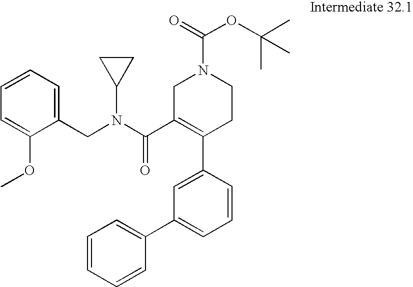

Intermediate 32.1 is synthesized by condensation of Intermediate 1.2 (100 mg, 0.24 mmol) analogously to the preparation of Intermediate 1.1. White amorphous material; Rf=0.70 (n-Hex:AcOEt=2:1).

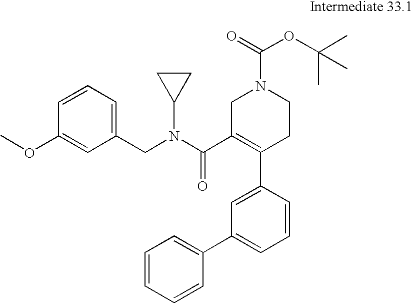

Intermediate 33.1 is synthesized by condensation of Intermediate 1.2 (100 mg, 0.24 mmol) analogously to the preparation of Intermediate 1.1. White amorphous material; ES-MS: M+H=539; HPLC: t_{Ret}=4.74 min.

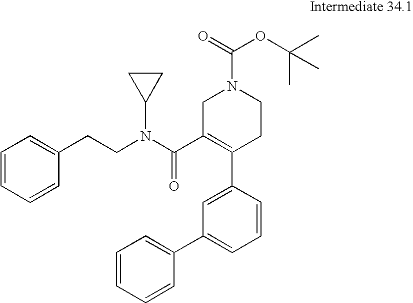

Intermediate 34.1 is synthesized by condensation of Intermediate 1.2 (100 mg, 0.24 mmol) analogously to the preparation of Intermediate 1.1. White amorphous material; ES-MS: M+H=523; HPLC: t_{Ret}=5.64 min.

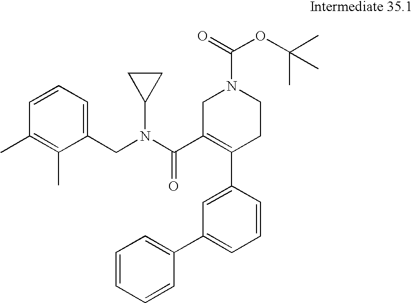

Intermediate 35.1 is synthesized by condensation of Intermediate 1.2 (100 mg, 0.24 mmol) analogously to the preparation of Intermediate 1.1. White amorphous material; ES-MS: M+H=537; HPLC: t_{Ret}=5.42 min.

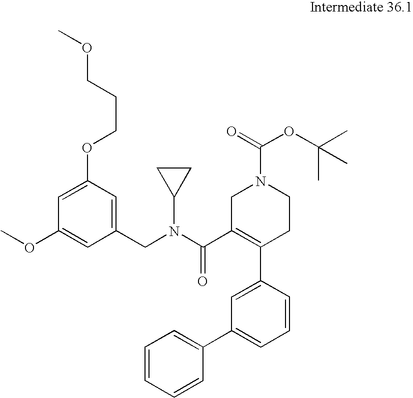

Intermediate 36.1 is synthesized by condensation of Intermediate 1.2 (200 mg, 0.48 mmol) and Intermediate 36.2 (208 mg, 0.72 mmol) analogously to the preparation of Intermediate 1.1. White amorphous material; ES-MS: M+H=627; HPLC: t_{Ret}=5.39 min.

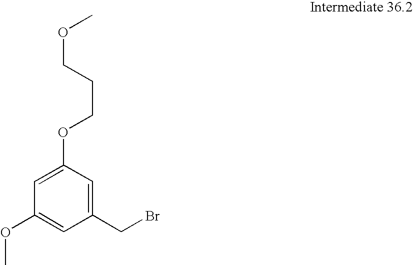

Intermediate 36.2 is synthesized by bromination of Intermediate 36.3 (1.1 g, 4.7 mmol) analogously to the preparation of Intermediate 1.5. Colorless oil; ES-MS: M+H=291; HPLC: t_{Ret}=4.09 min

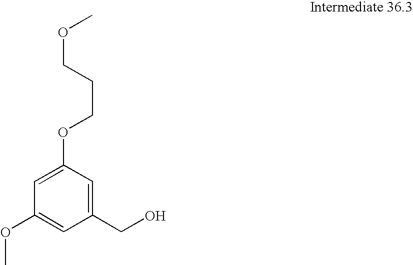

Intermediate 36.3 is synthesized by reduction of Intermediate 36.4 (5 g, 19.7 mmol) analogously to the preparation of Intermediate 1.6. Colorless oil; ES-MS: M+H=227; HPLC: t_{Ret}=2.85 min

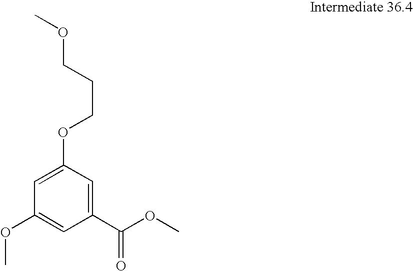

Intermediate 36.4 is synthesized by alkylation of 3-methoxy-5-hydroxybenzoic acid methyl ester (1.09 g, 6.44 mmol) analogously to the preparation of Intermediate 7.3. Amorphous material; ES-MS: M+H=255; HPLC: t_{Ret}=3.80 min

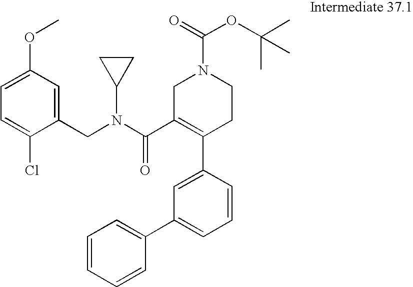

Intermediate 37.1 is synthesized by condensation of Intermediate 1.2 (200 mg, 0.48 mmol) analogously to the preparation of Intermediate 1.1. White amorphous material; ES-MS: M+=573; HPLC: t_{Ret}=5.65 min.

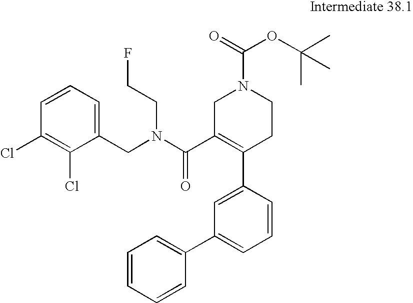

Intermediate 38.1 is synthesized by condensation of Intermediate 382 (140 mg, 0.33 mmol) analogously to the preparation of Intermediate 1.1. White amorphous material; ES-MS: M+H=586; HPLC: t_{Ret}=5.59 min.

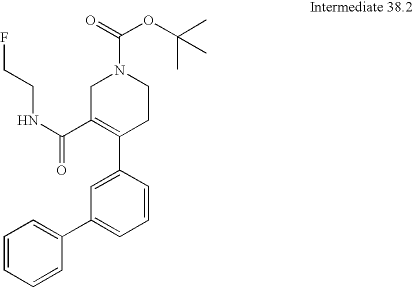

Intermediate 38.2 is synthesized by condensation of Intermediate 1.3 (200 mg, 0.53 mmol) and 2-fluoroethylamine (79 mg, 0.74 mmol) analogously to the preparation of Intermediate 1.2. White amorphous material; ES-MS: M+H=425; HPLC: t_{Ret}=4.32 min.

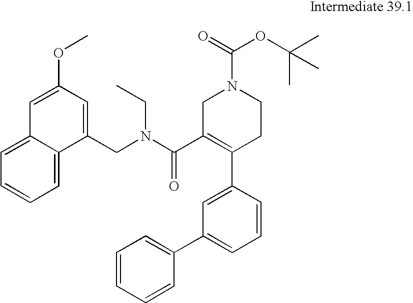

Intermediate 39.1 is synthesized by alkylation of Intermediate 39.2 (198.5 mg, 0.36 mmol) analogously to the preparation of Intermediate 1.1. White amorphous material; ES-MS: M+H=577; HPLC: t_{Ret}=5.60 min.

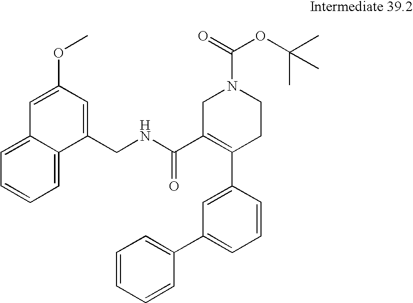

Intermediate 392 is synthesized by condensation of Intermediate 1.3 (289.0 mg, 0.76 mmol) analogously to the preparation of Intermediate 1.2. White amorphous material; ES-MS: M+H=549; HPLC: t_{Ret}=5.20 min.

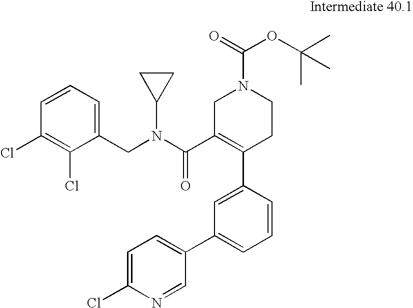

Intermediate 40.1 is synthesized by coupling of Intermediate 15.2 (156.3 mg, 0.27 mmol) and 2-chloropyridine-5-boronic acid (63.5 mg, 0.40 mmol) analogously to the preparation of Intermediate 2.1. White amorphous material; ES-MS: M+H=614; HPLC: t_{Ret}=5.55 min.

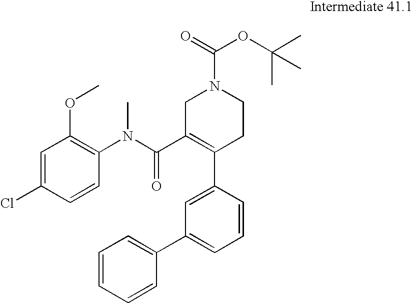

Intermediate 41.1 is synthesized by alkylation of Intermediate 41.2 (207 mg, 0.4 mmol) analogously to the preparation of Intermediate 1.1. White amorphous material; ES-MS: M+H=533; HPLC: t_{Ret}=5.49 min.

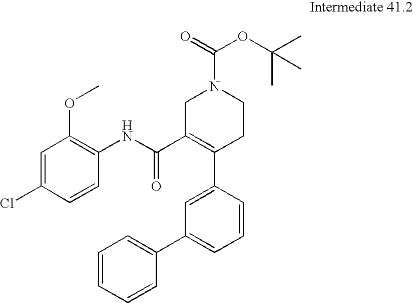

Intermediate 412 is synthesized by condensation of Intermediate 1.3 (680 mg, 1.8 mmol) analogously to the preparation of Intermediate 12. White amorphous material; M+H=519; HPLC: t_{Ret}=5.55 min.

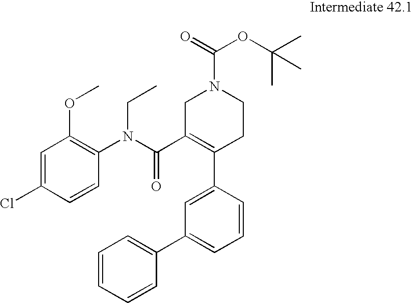

Intermediate 42.1 is synthesized by alkylation of Intermediate 41.2 (207 mg, 0.4 mmol) analogously to the preparation of Intermediate 1.1. White amorphous material; ES-MS: M+H=547; HPLC: t_{Ret}=5.70 min.

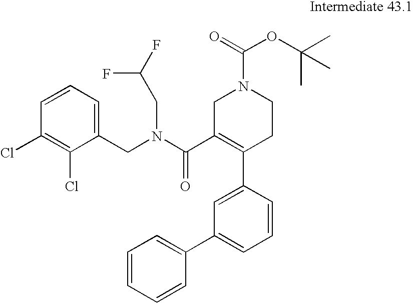

Intermediate 43.1 is synthesized by condensation of Intermediate 43.2 (170 mg, 0.38 mmol) analogously to the preparation of Intermediate 1.1. White amorphous material; ES-MS: M+H=601; HPLC: t_{Ret}=5.70 min.

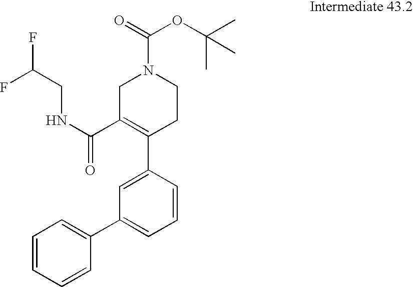

Intermediate 43.2 is synthesized by condensation of Intermediate 1.3 (200 mg, 0.53 mmol) and 2,2-difluoroethylamine (64 mg, 0.74 mmol) analogously to the preparation of Intermediate 1.2. White amorphous material; ES-MS: M+H=443; HPLC: t_{Ret}=4.49 min.

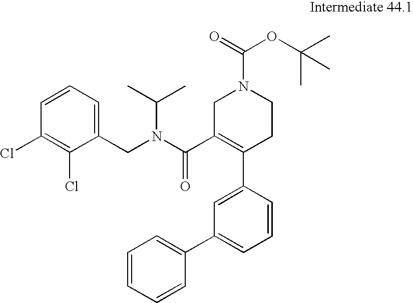

Intermediate 44.1 is synthesized by condensation of Intermediate 44.2 (230 mg, 0.55 mmol) analogously to the preparation of Intermediate 1.1. White amorphous material; ES-MS: M+H=579; HPLC: t_{Ret}=5.92 min.

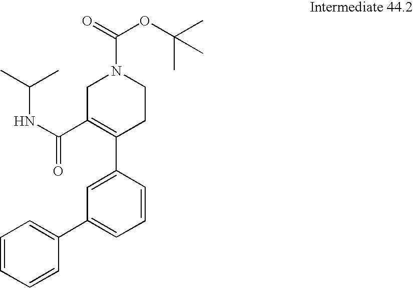

Intermediate 44.2 is synthesized by condensation of Intermediate 1.3 (300 mg, 0.79 mmol) and isopropylamine (0.1 g, 1.2 mmol) analogously to the preparation of Intermediate 12. White amorphous material; ES-MS: M+H=421; HPLC: t_{Ret}=4.57 min.

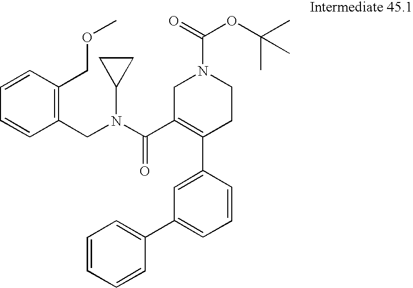

Intermediate 45.1 is synthesized by condensation of Intermediate 1.2 (261 mg, 0.62 mmol) analogously to the preparation of Intermediate 1.1. White amorphous material; ES-MS: M+H=553; HPLC: t_{Ret}=5.15 min.

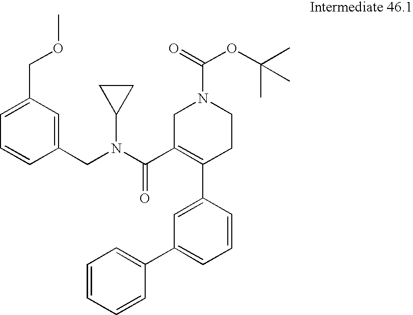

Intermediate 46.1 is synthesized by condensation of Intermediate 1.2 (280 mg, 0.67 mmol) analogously to the preparation of Intermediate 1.1. White amorphous material; ES-MS: M+H=553; HPLC: t_{Ret}=5.07 min.

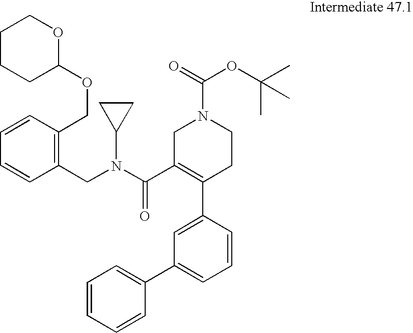

Intermediate 47.1 is synthesized by condensation of Intermediate 1.2 (57.7 mg, 0.14 mmol) and Intermediate 47.2 (39.4 mg, 0.14 mmol) analogously to the preparation of Intermediate 1.1. White amorphous material; Rf=0.40 (n-Hex:AcOEt=2:1).

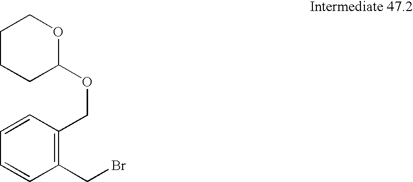

To a mixture of (2-bromomethyl-phenyl)-methanol (27 mg, 0.13 mmol) and 3,4-dihydro-2H-pyrane (16.9 mg, 0.20 mmol) in dichloromethane, cat. PPTS is added under N₂ at RT. After stirring at RT for 30 min, aqueous NaHCO₃ is added to the reaction mixture, and the mixture is extracted with dichloromethane. The combined organic phases are dried over Na₂SO₄. Concentration under reduced pressure and purified by silica gel flash chromatography to give Intermediate 47.2 as colorless oil; Rf=0.80 (n-Hex:AcOEt=3:1); ¹H NMR (CDCl₃), δ: 1.27 (3H, m), 1.57 (2H, m), 1.67 (1H, m), 3.3.8 (1H, m), 3.78 (1H, td), 4.31 (1H, d), 4.39 (1H, d), 4.57 (1H, d), 4.62 (1H, t), 4.95 (1H, d), 6.98 (m, 3H), 7.32 (d, 1H).

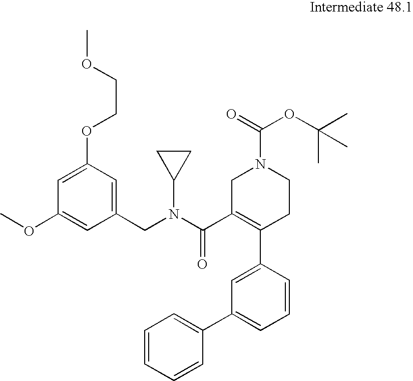

Intermediate 48.1 is synthesized by condensation of Intermediate 1.2 (200 mg, 0.48 mmol) and Intermediate 48.2 (107 mg, 0.72 mmol) analogously to the preparation of Intermediate 1.1. White amorphous material; ES-MS: M+H=613; HPLC: t_{Ret}=5.17 min.

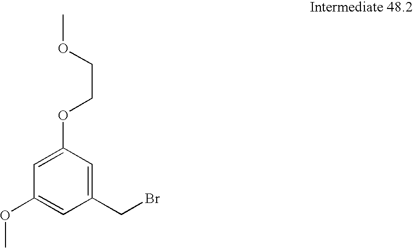

Intermediate 48.2 is synthesized by bromination of Intermediate 48.3 (1.4 g, 6.6 mmol) analogously to the preparation of Intermediate 1.5. Colorless oil; ES-MS: M+H=277; HPLC: t_{Ret}=3.77 min

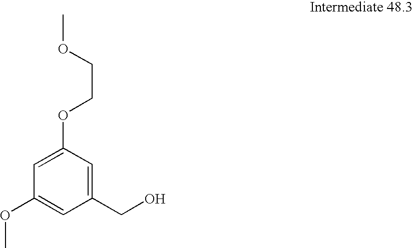

Intermediate 48.3 is synthesized by reduction of Intermediate 48.4 (1.3 g, 5.4 mmol) analogously to the preparation of Intermediate 1.6. Colorless oil; ES-MS: M+H=213; HPLC: t_{Ret}=2.85 min

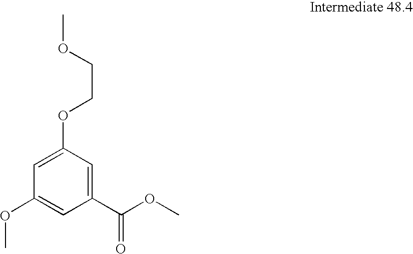

Intermediate 48.4 is synthesized by alkylation of 3-methoxy-5-hydroxybenzoic acid methyl ester (1.1 g, 6.44 mmol) analogously to the preparation of Intermediate 7.3. White powder; ES-MS: M+H=241; HPLC: t_{Ret}=3.42 min

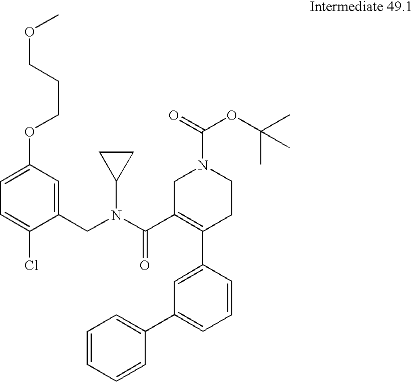

Intermediate 49.1 is synthesized by condensation of Intermediate 12 (200 mg, 0.48 mmol) and Intermediate 49.2 (211 mg, 0.72 mmol) analogously to the preparation of Intermediate 1.1. White amorphous material; ES-MS: M+H=631; HPLC: t_{Ret}=5.63 min.

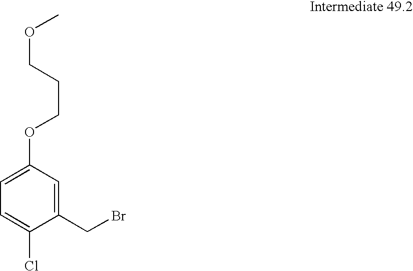

Intermediate 49.2 is synthesized by bromination of Intermediate 19.2 (1.14 g, 4.94 mmol) starting from Intermediate 49.3 analogously to the preparation of Intermediate 1.5. Colorless oil; ES-MS: M+=291; HPLC: t_{Ret}=2.67 min

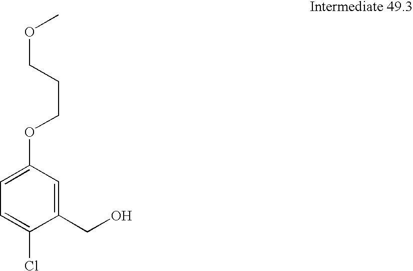

Intermediate 49.3 is synthesized by reduction of Intermediate 49.4 (1.27 g, 4.91 mmol) analogously to the preparation of Intermediate 1.6. Colorless oil; ES-MS: M+H=231; HPLC: t_{Ret}=3.17 min

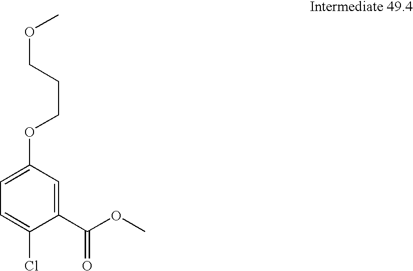

Intermediate 49.4 is synthesized by alkylation of 2-chloro-5-hydroxybenzoic acid methyl ester (1.00 g, 5.36 mmol) (see e.g. WO 99/52907 or WO 04/004632) analogously to the preparation of Intermediate 7.3. White solid; ES-MS: M+H=259; HPLC: t_{Ret}=3.73 min

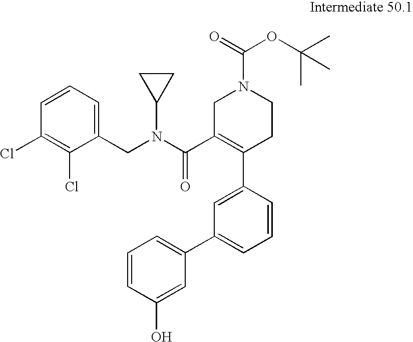

Intermediate 50.1 is synthesized by coupling of Intermediate 152 (605.2 mg, 1.04 mmol) and 3-hydroxyphenylboronic acid (215.8 mg, 1.56 mmol) analogously to the preparation of Intermediate 2.1. White amorphous material; ES-MS: M+=593; HPLC: t_{Ret}=5.15 min.

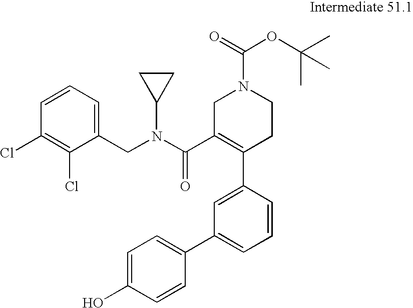

Intermediate 51.1 is synthesized by coupling of Intermediate 15.2 (599.7 mg, 1.03 mmol) and 4-hydroxyphenylboronic acid (213.8 mg, 1.55 mmol) analogously to the preparation of Intermediate 2.1. White amorphous material; ES-MS: M+=593; HPLC: t_{Ret}=5.05 min.

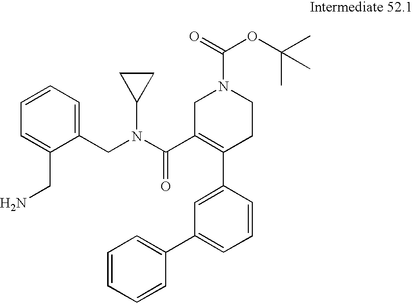

To a solution of Intermediate 52.2 (420 mg, 0.63 mmol) in EtOH (5 mL) was added Hydrazine hydrate (95 mg, 1.90 mmol) under N₂. After stirring at 60° C. for 1 h, the reaction mixture is quenched by the addition of Iced H₂O. The resulting mixture is extracted with EtOAc, and the organic extracts are washed with brine. The organic layer is dried (MgSO₄), filtered, and concentrated in vacuo. After concentration, the residue is purified by silica gel flash chromatography to give Intermediate 52.1 as colorless oil; ES-MS: M+H=538; HPLC: _{A}t_{Ret}=3.82 min.

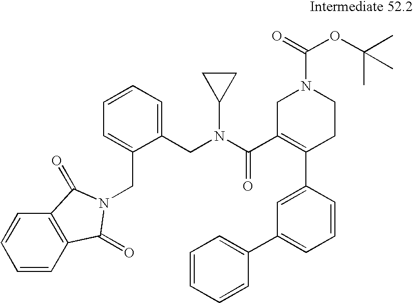

Intermediate 52.2 is synthesized by condensation of Intermediate 1.2 (250 mg, 0.60 mmol) and 2-(2-bromomethyl-benzyl)-isoindole-1,3-dione (270 mg, 0.81 mmol) (see e.g. Journal of the Chemical Society, Chemical Communications. 1989, 9, 602-3) analogously to the preparation of Intermediate 1.1. White amorphous material; ES-MS: M+H=668; HPLC: t_{Ret}=5.47 min.

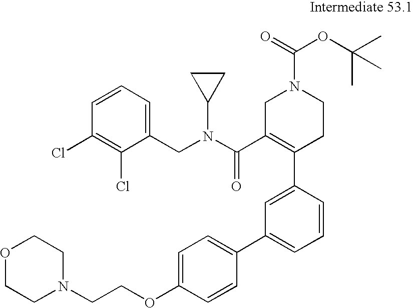

Intermediate 53.1 is synthesized by alkylation of Intermediate 51.1 (150.7 mg, 0.25 mmol) analogously to the preparation of Intermediate 7.3. White amorphous material; ES-MS: M+=706; HPLC: t_{Ret}=4.15 min.

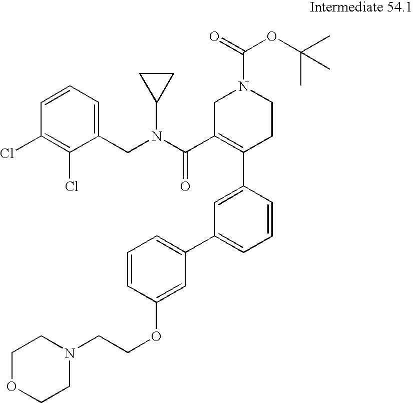

Intermediate 54.1 is synthesized by alkylation of Intermediate 50.1 (149.2 mg, 0.25 mmol) analogously to the preparation of Intermediate 7.3. White amorphous material; ES-MS: M+=706; HPLC: t_{Ret}=4.20 min.

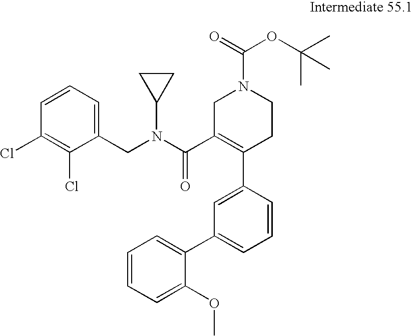

Intermediate 55.1 is synthesized by coupling of Intermediate 15.2 (300 mg, 0.52 mmol) and 2-methoxyphenylboronic acid (102 mg, 0.67 mmol) analogously to the preparation of Intermediate 2.1. White amorphous material; ES-MS: M+=607; HPLC: t_{Ret}=5.87 min.

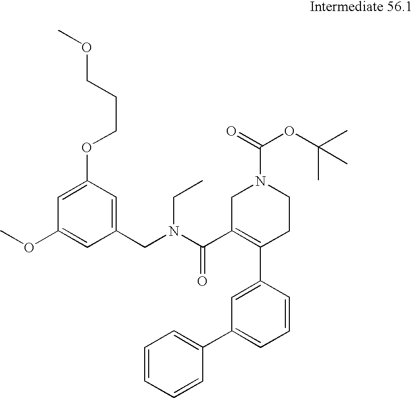

Intermediate 56.1 is synthesized by condensation of Intermediate 9.2 (200 mg, 0.49 mmol) and Intermediate 36.2 (210 mg, 0.74 mmol) analogously to the preparation of Intermediate 1.1. White amorphous material; ES-MS: M+H=615; HPLC: t_{Ret}=4.78 min.

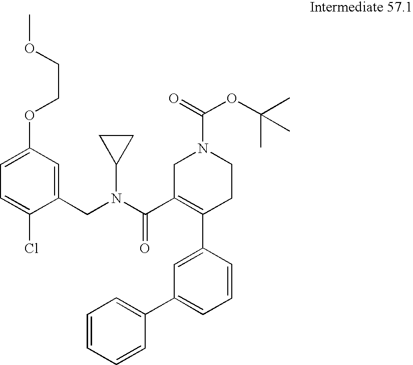

Intermediate 57.1 is synthesized by condensation of Intermediate 1.2 (200 mg, 0.48 mmol) and Intermediate 57.2 (201 mg, 0.72 mmol) analogously to the preparation of Intermediate 1.1. White amorphous material; Rf=0.48 (EtOAc:n-Hex=1:1) ES-MS: M+Na=639.

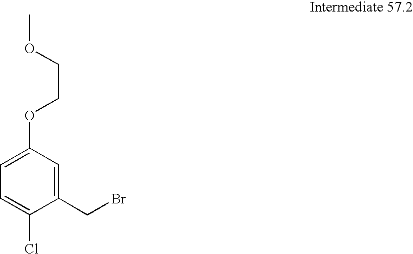

Intermediate 57.2 is synthesized by bromination of Intermediate 57.3 (1.0 g, 4.61 mmol) analogously to the preparation of Intermediate 1.5. Colorless oil; ES-MS: M+=279; HPLC: t_{Ret}=2.38 min

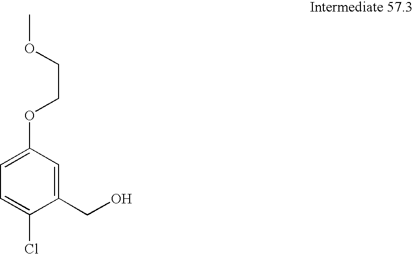

Intermediate 57.3 is synthesized by reduction of Intermediate 57.4 (1.31 g, 5.36 mmol) analogously to the preparation of Intermediate 1.6. Colorless oil; Rf=0.49 (EtOAc:n-Hex=1:1); ES-MS: M+H=217.

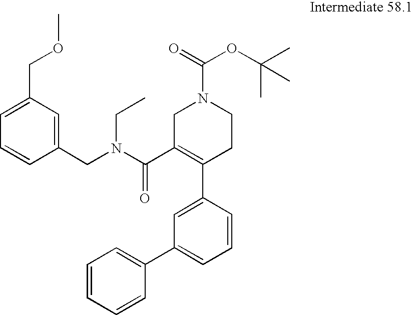

Intermediate 58.1 is synthesized by condensation of Intermediate 92 (200 mg, 0.49 mmol) analogously to the preparation of Intermediate 1.1. White amorphous material; ES-MS: M+H=541; HPLC: t_{Ret}=5.20 min.

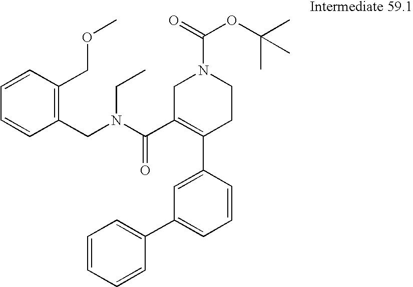

Intermediate 59.1 is synthesized by condensation of Intermediate 92 (243 mg, 0.6 mmol) analogously to the preparation of Intermediate 1.1. White amorphous material; ES-MS: M+H=541; HPLC: t_{Ret}=5.25 min.

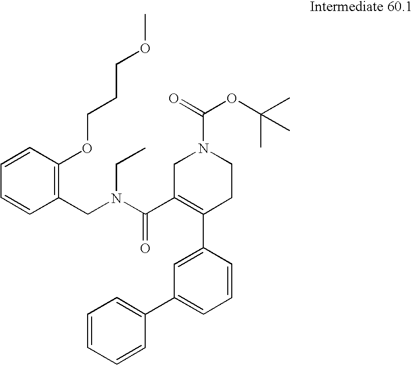

Intermediate 60.1 is synthesized by condensation of Intermediate 9.2 (100 mg, 0.24 mmol) analogously to the preparation of Intermediate 1.1. White amorphous material; ES-MS: M+H=585; HPLC: t_{Ret}=5.21 min.

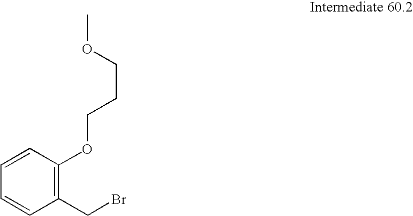

Intermediate 60.2 is synthesized by bromination of Intermediate 60.3 (367 mg, 1.87 mmol) analogously to the preparation of Intermediate 1.5. Solid powder; Rf=0.75 (n-Hex:EtOAc=2:1), ¹H NMR (CDCl₃), δ: 2.11 (2H, m), 3.48 (3H, s), 3.63 (2H, t), 4.13 (2H, d), 4.57 (s, 2H), 6.88 (2H, d), 6.91 (1H, t), 7.26 (1H, t), 7.32 (1H, t).

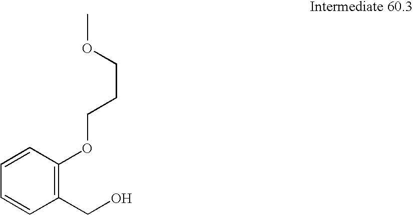

To a solution of 2-hydroxy-benzoic acid methyl ester (500 mg, 3.29 mmol) and 3-methoxy-propan-1-ol (355 mg, 3.94 mmol) in dry THF, PPh₃ (1.03 g, 3.94 mmol) and DEAD (1.79 ml, 3.94 mmol) are added under N₂ at room temperature. After stirring at 66° C. for 12 h, the mixture is concentrated under reduced pressure, and the residue is purified by silica gel flash chromatography to give the alkylation product as colorless oil. Subsequently, to a solution of this alkylation product (420 mg, 1.87 mmol) in dry THF, LAH (142 mg, 3.74 mmol) is added under N₂ at 0° C. After stirring at room temperature for one hour, Na₂SO₄.10H₂O is added to the reaction mixture, and it is then diluted with hexane, followed by addition of Na₂SO₄. After filtration over Celite, the mixture is concentrated under reduced pressure, and the residue is purified by silica gel flash chromatography to give Intermediate 60.3 as colorless oil; Rf=0.28 (n-Hex:AcOEt=2:1); ¹H NMR (CDCl₃), δ: 2.09 (2H, m), 3.45 (3H, s), 3.56 (2H, t), 4.1.3 (2H, t), 4.67 (2H, s), 6.88 (1H, d), 6.93 (1H, t), 7.26 (1H, t), 7.27 (1H, d).

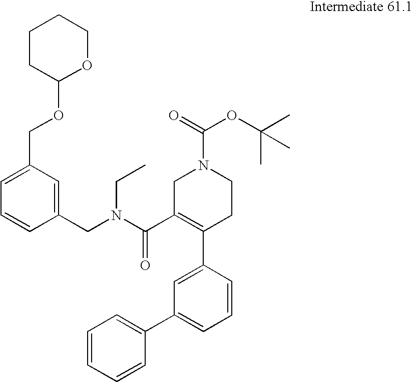

Intermediate 61.1 is synthesized by condensation of Intermediate 9.2 (100 mg, 0.24 mmol) and Intermediate 47.2 (81.7 mg, 0.29 mmol) analogously to the preparation of Intermediate 1.1. White amorphous material; ES-MS: M+H=611; HPLC: t_{Ret}=5.45 min.

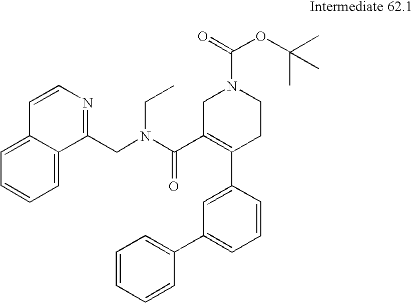

Intermediate 62.1 is synthesized by condensation of Intermediate 92 (100 mg, 0.24 mmol) analogously to the preparation of Intermediate 1.1. White amorphous material; ES-MS: M+H=548; HPLC: t_{Ret}=3.93 min.

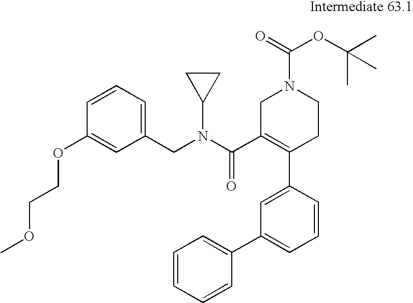

Intermediate 63.1 is synthesized by alkylation of Intermediate 63.2 (79 mg, 0.2 mmol) analogously to the preparation of Intermediate 7.3. White amorphous material; ES-MS: M+H=583; HPLC: t_{Ret}=5.15 min.

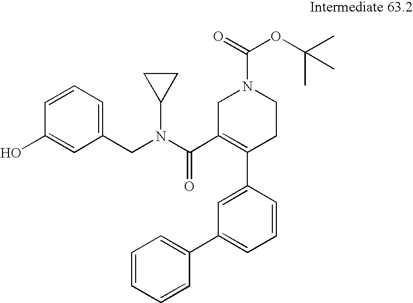

A mixture of Intermediate 63.3 (1.1 g, 2.0 mmol) and 4N dioxane solution of HCl (10 mL) is stirred under N₂ at RT. After stirring for 0.5 h, the reaction mixture is concentrated under reduced pressure to give crude compound. Then a mixture of crude compound, DIEA (377 mL, 2.2 mmol) and (Boc)₂O (0.46 mL, 2.0 mmol) in DCM (20 mL) is stirred under N₂ at RT for 1 h. After adding aqueous KHSO₄, the reaction mixture is extracted with EtOAc. The combined organic phases are washed with H₂O, brine and dried (Na₂SO₄). Concentration under reduced pressure and purified by silica gel flash chromatography to give Intermediate 63.2 as white amorphous material; ES-MS: M+H=525; HPLC: t_{Ret}=4.67 min.

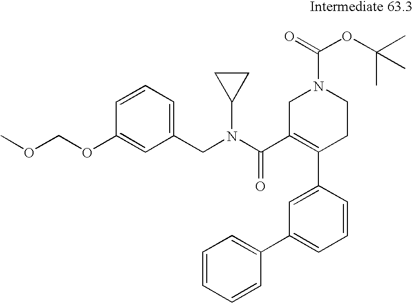

Intermediate 63.3 is synthesized by condensation of Intermediate 1.2 (1.7 g, 3.0 mmol) and 2-(methoxymethoxy)benzyl bromide (774 mg, 3.4 mmol) (see e.g. J. Org. Chem. 2000, 65, 5644-5646) analogously to the preparation of Intermediate 1.1. White amorphous material; ES-MS: M+H=569; HPLC: t_{Ret}=5.20 min

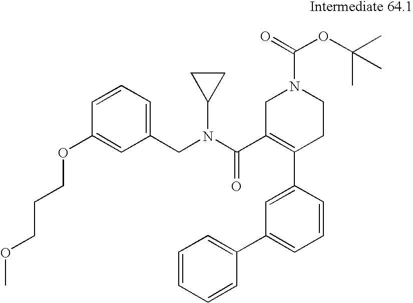

Intermediate 64.1 is synthesized by alkylation of Intermediate 63.2 (105 mg, 0.2 mmol) analogously to the preparation of Intermediate 7.3. White amorphous material; ES-MS: M+H=597; HPLC: t_{Ret}=5.24 min.

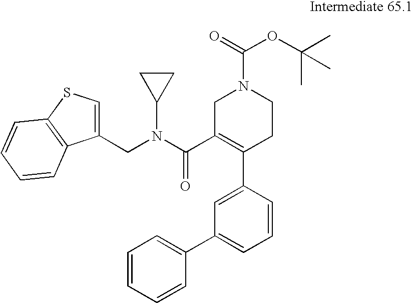

Intermediate 65.1 is synthesized by condensation of Intermediate 12 (100 mg, 0.24 mmol) and 3-bromomethylbenzothiophene (81 mg, 0.36 mmol) analogously to the preparation of Intermediate 1.1. White amorphous material; ES-MS: M+H=565; HPLC: t_{Ret}=5.70 min.

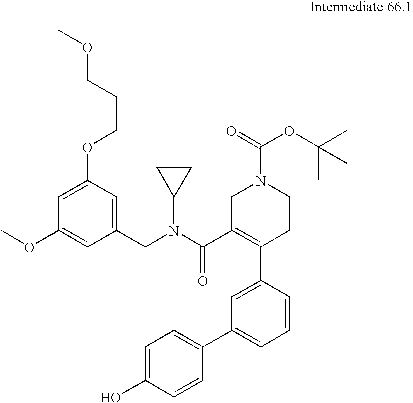

Intermediate 66.1 is synthesized by coupling of Intermediate 662 (250 mg, 0.4 mmol) and 4-hydroxyphenyl boronic acid (82 mg, 0.6 mmol) analogously to the preparation of Intermediate 2.1. White amorphous material; ES-MS: M+H=544; HPLC: t_{Ret}=4.68 min.

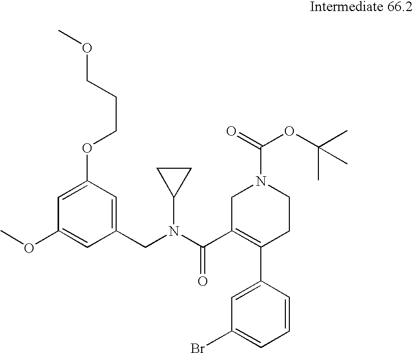

Intermediate 66.2 is synthesized by condensation of Intermediate 2.3 (2.0 g, 4.75 mmol) and Intermediate 36.2 (1.65 g, 5.7 mmol) analogously to the preparation of Intermediate 1.1. White amorphous material; ES-MS: M+=629; HPLC: t_{Ret}=5.20 min.

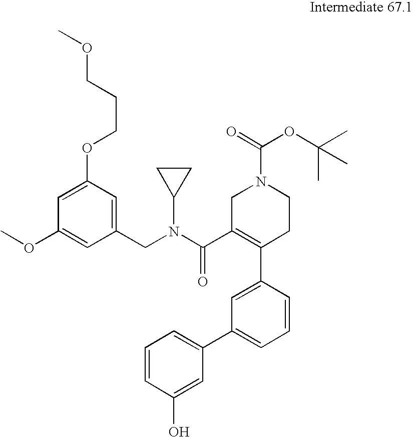

Intermediate 67.1 is synthesized by coupling of Intermediate 662 (250 mg, 0.4 mmol) and 3-hydroxyphenyl boronic acid (82 mg, 0.6 mmol) analogously to the preparation of Intermediate 2.1. White amorphous material; ES-MS: M+H=643; HPLC: t_{Ret}=4.84 min.

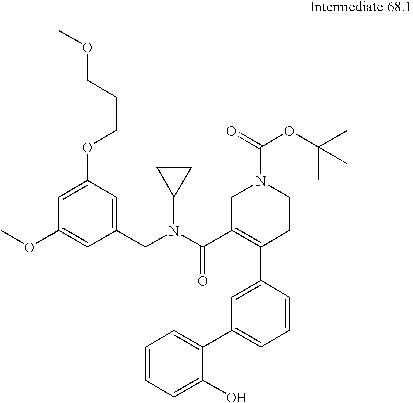

Intermediate 68.1 is synthesized by coupling of Intermediate 66.2 (337 mg, 0.53 mmol) and 2-hydroxyphenyl boronic acid (110 mg, 0.80 mmol) analogously to the preparation of Intermediate 2.1. White amorphous material; ES-MS: M+H=644; HPLC: t_{Ret}=4.92 min.

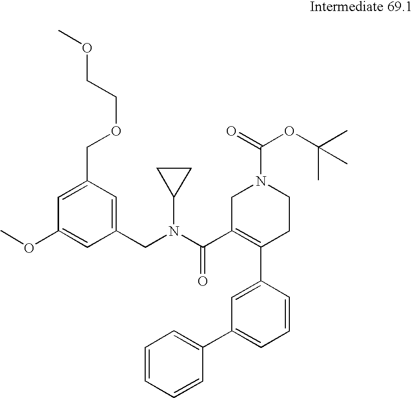

Intermediate 69.1 is synthesized by condensation of Intermediate 12 (200 mg, 0.48 mmol) and Intermediate 69.2 (140 mg, 0.48 mmol) analogously to the preparation of Intermediate 1.1. White amorphous material; ES-MS: M+H=627; HPLC: t_{Ret}=5.12 min.

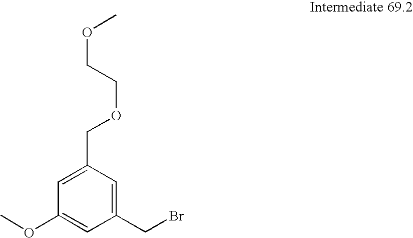

Intermediate 692 is synthesized by bromination of Intermediate 69.3 (740 mg, 3.27 mmol) analogously to the preparation of Intermediate 1.5. White powder, ES-MS: M+H=288; HPLC: t_{Ret}=3.79 min

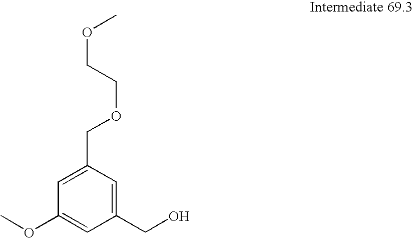

Intermediate 69.3 is synthesized by reduction of Intermediate 69.4 (824 mg, 3.3 mmol) analogously to the preparation of Intermediate 1.6. White powder; HPLC: t_{Ret}=2.52 min; Rf=0.21 (EtOAc:n-Hex=1:1)

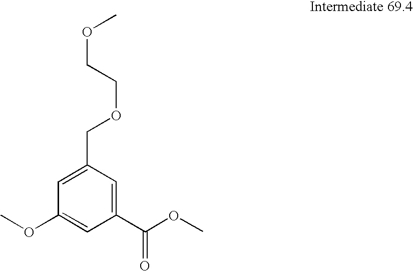

Intermediate 69.4 is synthesized by alkylation of 3-hydroxymethyl)-5-methoxy-benzoic acid methylester (1.859, 9.4 mmol) (see e.g. Synth. Commun. 2001, 31, 1921-1926) analogously to the preparation of Intermediate 7.3.: Amorphous material; ES-MS: M+H=255; HPLC: t_{Ret}=3.44 min

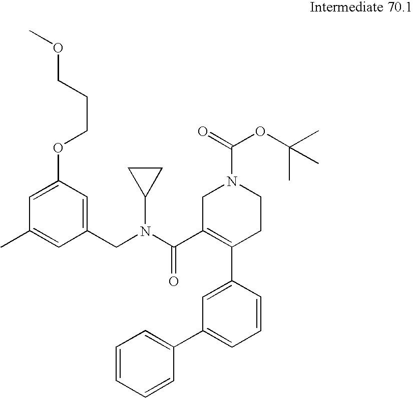

Intermediate 70.1 is synthesized by condensation of Intermediate 1.2 (280 mg, 0.55 mmol) and Intermediate 702 (180 mg, 0.66 mmol) analogously to the preparation of Intermediate 1.1. White amorphous material; ES-MS: M+H=611; HPLC: t_{Ret}=5.62 min.

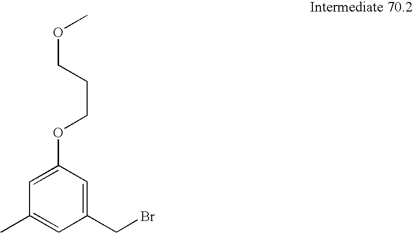

Intermediate 70.2 is synthesized by bromination of Intermediate 70.3 (2.1 g, 10.0 mmol) analogously to the preparation of Intermediate 1.5. Colorless oil; ES-MS: M+H=273; HPLC: t_{Ret}=4.43 min

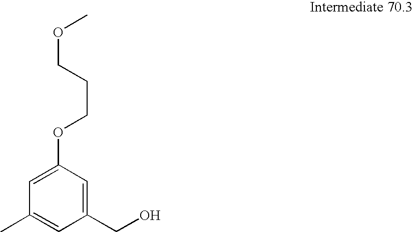

To a mixture of Intermediate 70.4 (5.18 g, 20.4 mmol), trimethylammonium chloride (50 mg) and Et₃N (3.4 mL, 24.4 mmol) in DCM (100 mL), p-toluenesulfonyl chloride (4.27 g, 22.4 mmol) is added at 0° C. After stirring for 50 min, H₂O is added to the reaction mixture, and the mixture is then extracted with EtOAc. The combined organic phases are washed with H₂O, brine and dried (Na₂SO₄), followed by concentrating under reduced pressure to give crude product. Then a solution of this crude product in THF (100 mL) is treated with LiAlH₄ (2.27 g, 59.8) at 0° C. for 2 h. After adding H₂O, the reaction mixture is extracted with EtOAc. The combined organic phases are washed with H₂O, brine and dried (Na₂SO₄). Concentration under reduced pressure and silica gel flash chromatography give Intermediate 70.3 as white amorphous material; ES-MS: M+=211; HPLC: t_{Ret}=3.04 min.

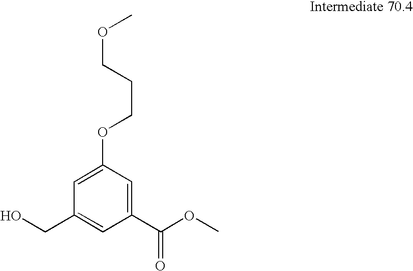

Intermediate 70.5 (5.75 g, 20.4 mmol) and Et₃N (3.7 mL, 26.5 mmol) in THF (100 mL), chloroformic acid ethylester (2.5 mL, 26.5 mmol) is added at 0° C. After surring for 20 min, the reaction mixture is filtered for removing inorganic salt, and the filtrate is concentrated under reduced pressure. A solution of this crude product in MeOH (50 mL) is treated with NaBH₄ (excess) at 0° C. for 20 min. After adding H₂O, the reaction mixture is extracted with EtOAc. The combined organic phases are washed with H₂O, brine and dried (Na₂SO₄). Concentration under reduced pressure and silica gel flash chromatography give Intermediate 70.4 as white amorphous material; ES-MS: M+Na=283; HPLC: t_{Ret}=3.92 min.

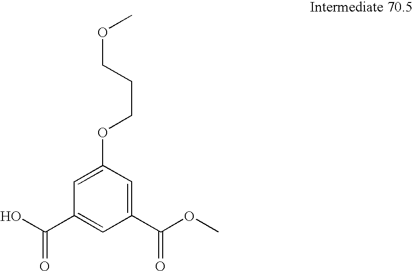

A mixture of Intermediate 70.6 (9.0 g, 31.9 mmol) and KOH (1.61 g, 28.7 mmol) in THF (100 mL) and MeOH (30 mL) is refluxed under N₂ for 3.5 h. After cooling down to RT, the reaction mixture is adjusted to weakly acidic pH by slowly adding conc HCl, and mixture is extracted with Et₂O. The combined organic phases are washed with H₂O, brine and dried (Na₂SO₄). Concentration under reduced pressure and silica gel flash chromatography give Intermediate 70.5 as white amorphous material; ES-MS: M+H=269; HPLC: t_{Ret}=3.15 min.

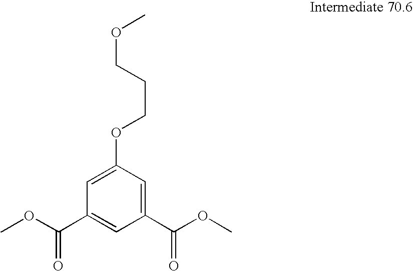

A mixture of 5-hydroxy-isophthalic acid dimethyl ester (7.02 g, 33.4 mmol), toluene-4-sulfonic acid 3-methoxy-propyl ester (8.16 g, 33.4 mmol), KI (6.1 g, 36.7 mmol) and K₂CO₃ (5.1 g, 36.7 mmol) in DMF (100 mL) is stirred under N₂ at 70° C. for 5 h. After adding H₂O, the reaction mixture is extracted with EtOAc. The combined organic phases are washed with H₂O, brine and dried (Na₂SO₄). Concentration under reduced pressure and silica gel flash chromatography give Intermediate 70.6 as white solid; ES-MS: M+H=283; HPLC: t=3.90 min

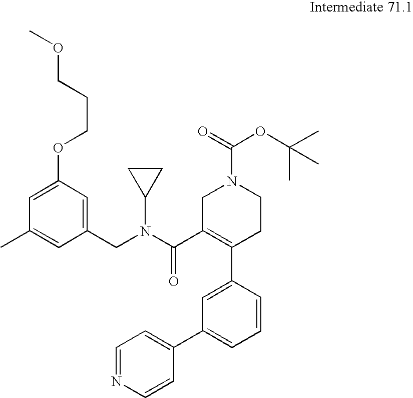

Intermediate 71.1 is synthesized by coupling of Intermediate 71.2 (300 mg, 0.48 mmol) and 4-pyridylboronic acid (294 mg, 2.4 mmol) analogously to the preparation of Intermediate 2.1. White amorphous material; ES-MS: M+H=612; HPLC: t_{Ret}=3.68 min.

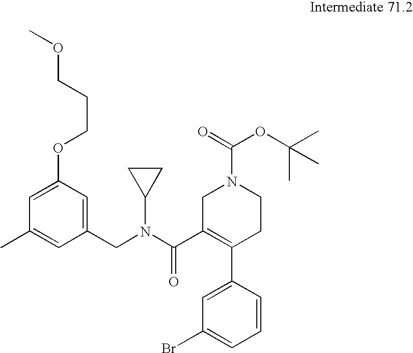

Intermediate 71.2 is synthesized by condensation of Intermediate 2.3 (1.0 g, 2.3 mmol) and Intermediate 70.2 840 mg, 3.1 mmol) analogously to the preparation of Intermediate 1.1. White amorphous material; ES-MS: M=613, M+2H=615; HPLC: t_{Ret}=5.40 min.

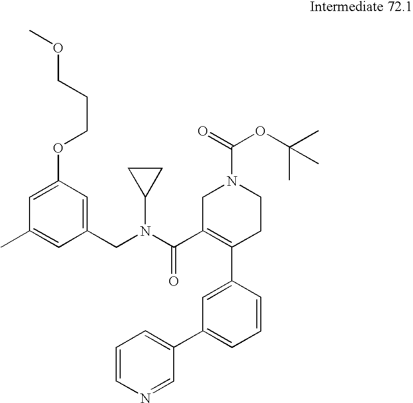

Intermediate 72.1 is synthesized by coupling of Intermediate 71.2 (300 mg, 0.48 mmol) and 3-pyridylboronic acid (294 mg, 2.4 mmol) analogously to the preparation of Intermediate 2.1. White amorphous material; ES-MS: M+H=612; HPLC: t_{Ret}=3.72 min.

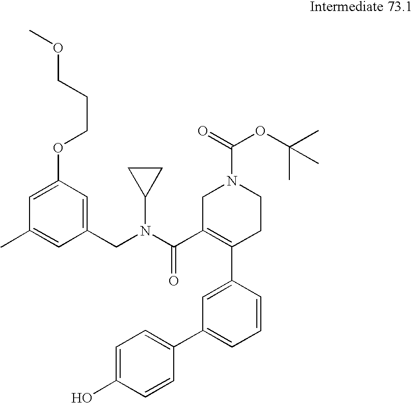

Intermediate 73.1 is synthesized by coupling of Intermediate 712 (100 mg, 0.16 mmol) and 4-hydroxyphenylboronic acid (32 mg, 0.24 mmol) analogously to the preparation of Intermediate 2.1. White amorphous material; ES-MS: M+H=627; HPLC: t_{Ret}=4.84 min.

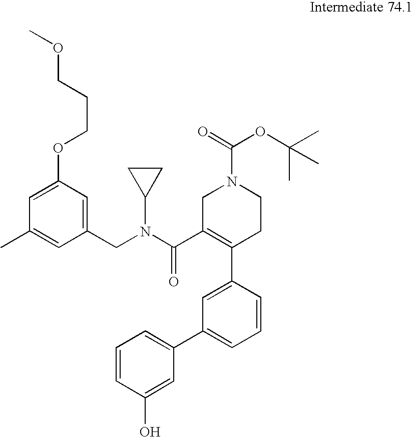

Intermediate 74.1 is synthesized by coupling of Intermediate 71.2 (100 mg, 0.16 mmol) and 3-hydroxyphenylboronic acid (32 mg, 0.24 mmol) analogously to the preparation of Intermediate 2.1. White amorphous material; ES-MS: M+H=627; HPLC: t_{Ret}=4.93 min.

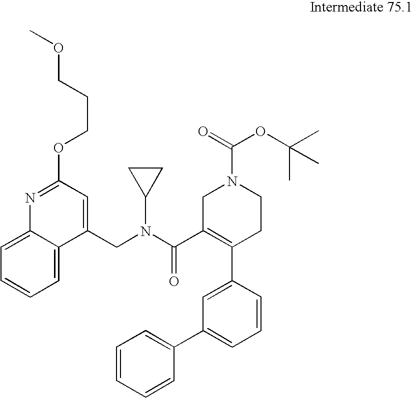

Intermediate 75.1 is synthesized by alkylation of Intermediate 1.2 (100 mg, 0.24 mmol) and Intermediate 75.2 (111 mg, 0.36 mmol) analogously to the preparation of Intermediate 1.1. White amorphous material; ES-MS: M+H=649; HPLC: t_{Ret}=5.42 min.

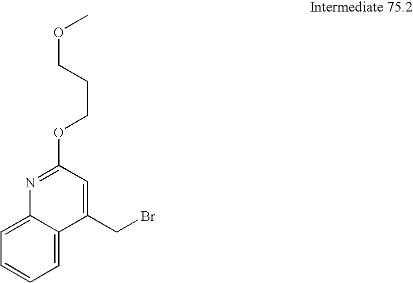

Intermediate 75.2 is synthesized by bromination of Intermediate 75.3 (2.0 g, 8.1 mmol) analogously to the preparation of Intermediate 1.5. Colorless oil; Rf=0.42 (EtOAc:n-Hex=1:5), ¹H NMR (CDCl₃), δ: 2.11 (2H, m), 3.37 (3H, s), 3.58 (2H, t), 4.55 (2H, t), 4.76 (2H, s), 7.26 (1H, s), 7.46 (1H, dd), 7.63 (1H, dd), 7.87 (1H, d), 7.99 (1H, d).

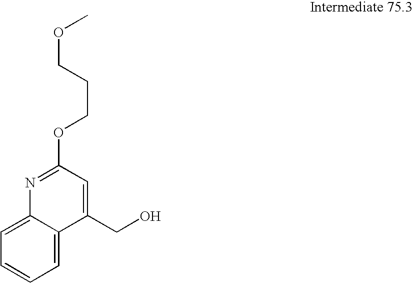

Intermediate 75.3 is synthesized by reduction of Intermediate 75.4 (2.5 g, 9.6 mmol) analogously to the preparation of Intermediate 1.6. Colorless oil, Rf=0.29 (EtOAc n-Hex=1:1), ¹H NMR (CDCl₃), δ: 2.11 (2H, m), 3.37 (3H, s), 3.59 (2H, t), 4.55 (2H, t), 5.11 (2H, s), 7.03 (1H, s), 7.38 (1H, dd), 7.61 (1H, dd), 7.79 (1H, d), 7.85 (1H, d).

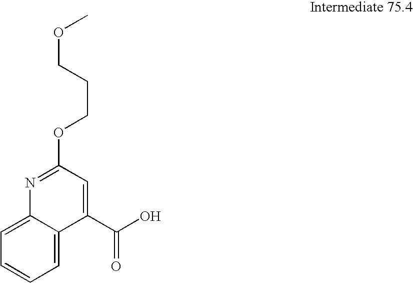

A mixture of 2-chloro-quinoline-4-carboxylic acid (2.0 g, 9.6 mmol), 3-methoxy-propanol (2.1 g, 24 mmol), and NaH (1.0 g, 26 mmol) in DMF (10 mL) is stirred under N₂ at 80° C. After stirring for 4.5 h, the reaction mixture is adjusted to weakly acidic pH by slowly adding conc. HCl, and the mixture is extracted with EtOAc. The combined organic phases are washed with H₂O, brine and dried (Na₂SO₄). Concentration under reduced pressure and silica gel flash chromatography give Intermediate 75.4 as white amorphous material; ES-MS: M+H=262; HPLC: t_{Ret}=3.30 min

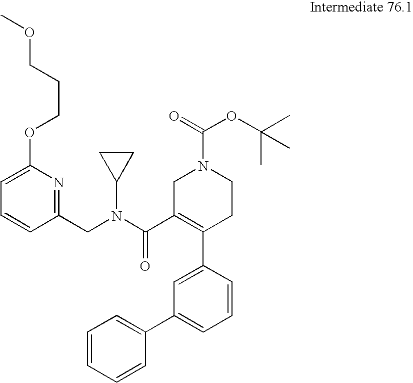

Intermediate 76.1 is synthesized by condensation of Intermediate 3.2 (90 mg, 0.18 mmol) and Intermediate 76.2 (51 mg, 0.22 mmol) analogously to the preparation of Intermediate 3.1. White amorphous material; ES-MS: M+H=598; HPLC: t_{Ret}=5.30 min.

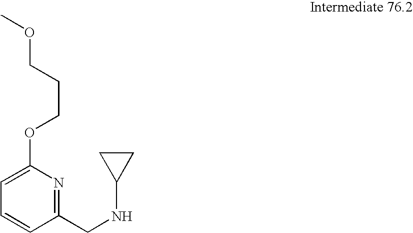

Intermediate 76.2 is synthesized by reductive amination of Intermediate 76.3 (400 mg, 2 mmol) analogously to the preparation of Intermediate 3.3. ES-MS: M+H=237; HPLC: t_{Ret}=2.32 min

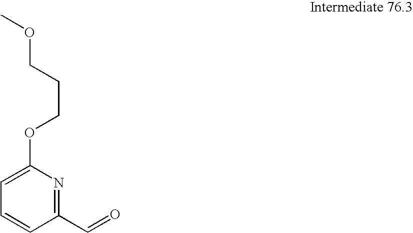

A mixture of Intermediate 76.4 (400 mg, 2.0 mmol) and MnO₂ (2.0 g, excess) in toluene (30 mL) is stirred under N₂ at RT for 1 day. After filtration for removing MnO₂, the filtrate is concentrated under reduced pressure and purified by silica gel flash chromatography to give Intermediate 76.3 as colorless oil; ES-MS: M+H=196; HPLC: t_{Ret}=3.20 min

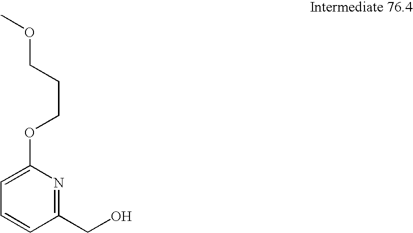

Intermediate 76.4 is synthesized by reduction of Intermediate 76.5 (650 mg, 3.08 mmol) analogously to the preparation of Intermediate 75.3. ES-MS: M+H=198; HPLC: t_{Ret}=1.87 min

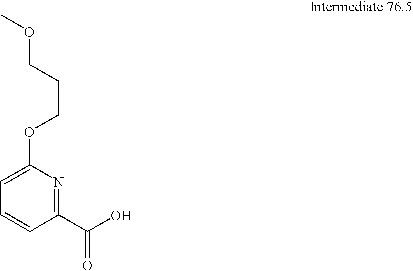

Intermediate 76.5 is synthesized by alkylation of 2-chloro-pyridine-6-carboxylic acid (650 mg, 3.08 mmol) analogously to the preparation of Intermediate 75.4. White powder; HPLC: t_{Ret}=2.05 min; ¹H NMR (CDCl₃) δ 2.04-2.11 (m, 2H), 3.39 (s, 3H), 3.56 (t, 2H), 4.48 (t, 2H), 7.00-7.02 (m, 1H), 7.77-8.02 (m, 2H).

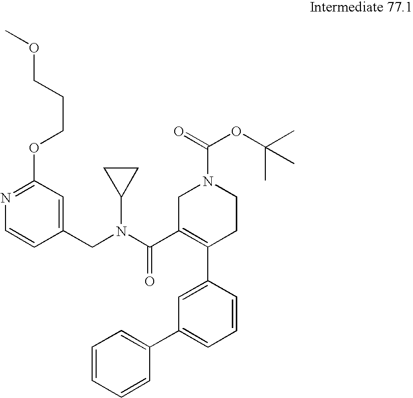

Intermediate 77.1 is synthesized by condensation of compound of Intermediate 32 (150 mg, 0.3 mmol) and Intermediate 77.2 (142 mg, 0.6 mmol) analogously to the preparation of Intermediate 3.1. White amorphous material; ES-MS: M+H=598; HPLC: t_{Ret}=4.55 min.

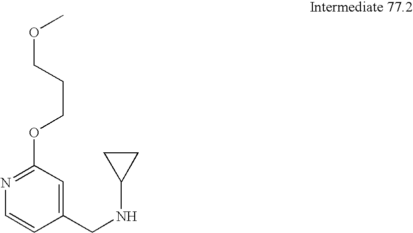

A mixture of Intermediate 77.3 (400 mg, 2.03 mmol) and SOCl₂ (1 mL, 11.4 mmol) in DCM (1 mL) is stirred under N₂ at 60° C. After stirring for 1 hour, the reaction mixture is concentrated under reduced pressure. This crude product is used without purification. A mixture of this crude material and an excess amount of cyclopropylamine in DMF (4 mL) is stirred under N₂ at RT. After stirring for 7 h, the reaction mixture is concentrated under reduced pressure and purified by silica gel flash chromatography to give Intermediate 77.2 as colorless oil (420 mg, 1.78 mmol; 88%); ES-MS: M+H=237; HPLC: t_{Ret}=1.93 min

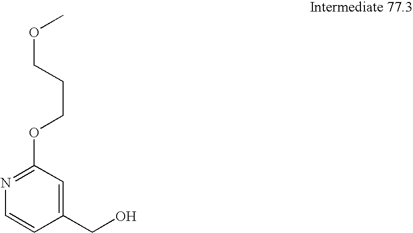

Intermediate 77.3 is synthesized by reduction of Intermediate 77.4 (500 mg, 2.37 mmol) analogously to the preparation of Intermediate 1.6. Colorless oil; ES-MS: M+H=198; HPLC: t_{Ret}=1.75 min

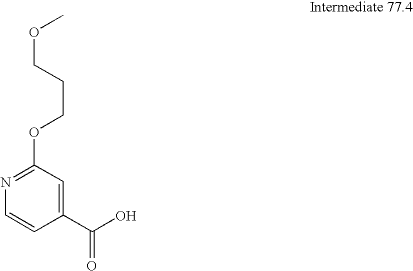

Intermediate 77.4 is synthesized by alkylation of 2-chloro-isonicotinic acid (1.1 g, 6.9 mmol) analogously to the preparation of Intermediate 75.4. Colorless oil; ES-MS: M+H=212; HPLC: t_{Ret}=2.52 min

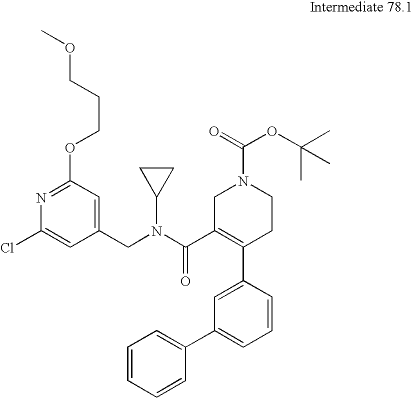

Intermediate 78.1 is synthesized by condensation of Intermediate 3.2 (375 mg, 0.75 mmol) and Intermediate 78.2 (224 mg, 0.83 mmol) analogously to the preparation of Intermediate 3.1. White amorphous material; ES-MS: M+=632; HPLC: t_{Ret}=5.60 min.

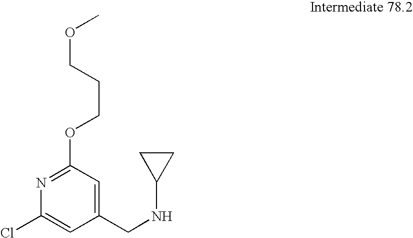

Intermediate 78.2 is synthesized by reductive amination of Intermediate 78.3 (350 mg, 1.52 mmol) analogously to the preparation of Intermediate 3.3. ES-MS: M+H=271; HPLC: t_{Rrt}=2.45 min

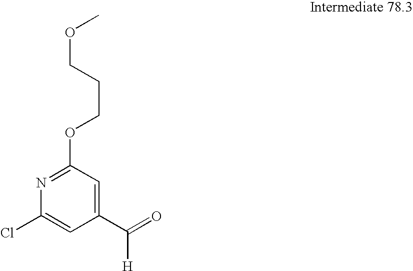

Intermediate 78.3 is synthesized by oxidation of Intermediate 78.4 (2.3 g, 10 mmol) analogously to the preparation of Intermediate 76.3. Colorless oil; Rf=0.66 (EtOAc:n-Hex=1:3); ¹H NMR (CDCl₃); δ 2.08 (dt, 2H), 3.32 (s, 3H), 3.55 (t, 2H), 4.45 (s, 2H), 7.02 (s, 1H), 7.32 (s, 1H), 9.90 (s, 1H).

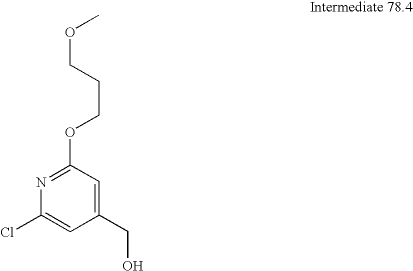

To a solution of 3-methoxy-1-propanol (5.16, 57.3 mmol) and NaH (2.3 g 57.3 mmol) in dry THF, 2-6-dichloroisonicotinic acid (5 g 26 mmol) is added at 0° C. The reaction mixture is stirred at 80° C. for 1.5 h, and then the reaction is quenched by the addition of H₂O. The reaction mixture is extracted with AcOEt, dried over MgSO₄ and filtered, and the filtrate is concentrated under reduced pressure to give 2-chloro-6-(3-methoxy-propoxy)-isonicotinic acid, which is directly used for the next reaction. To a solution of 2-chloro-6-(3-methoxy-propoxy)-isonicotinic acid, ClCO₂Et (3.7 ml, 39 mmol) and Et₃N (5.4 ml, 39 mmol are added at 0° C. After stirring at RT for 30 min, the reaction mixture is filtrated through Celite and concentrated under reduced pressure. The residue is treated with NaBH₄ in EtOH (50 ml) to give Intermediate 78.4 as colorless oil; Rf=0.43 (EtOAc:n-Hex=1:3). ¹H NMR (CDCl₃); δ 2.12 (dt, 2H), 3.36 (s, 3H), 3.55 (t, 2H), 4.45 (s, 2H), 7.05 (s, 1H), 7.32 (s, 1H).

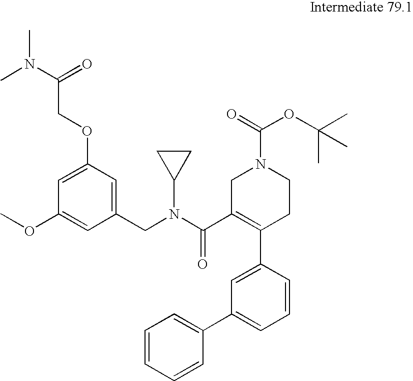

Intermediate 79.1 is synthesized by condensation of Intermediate 792 (117 mg, 0.19 mmol) analogously to the preparation of Intermediate 1.2. White amorphous material; ES-MS: M+H=640; HPLC: t_{Ret}=4.50 min.

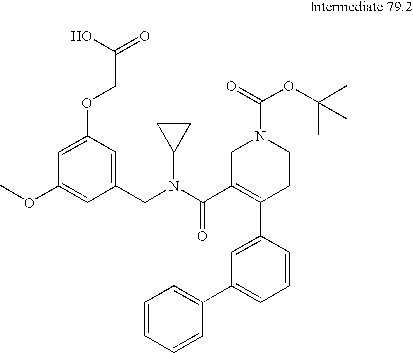

Intermediate 79.2 is synthesized by hydrolysis of Intermediate 79.3 (280 mg, 0.5 mmol) analogously to the preparation of Intermediate 1.3. White amorphous material; ES-MS: M+H=613; HPLC: t_{Ret}=4.59 min.

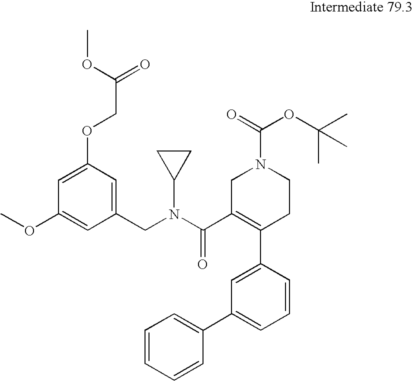

Intermediate 79.3 is synthesized by alkylation of Intermediate 79.4 (385 mg, 0.7 mmol) analogously to the preparation of Intermediate 7.3. White amorphous material; ES-MS: M+H=627; HPLC: t_{Ret}=5.05 min.

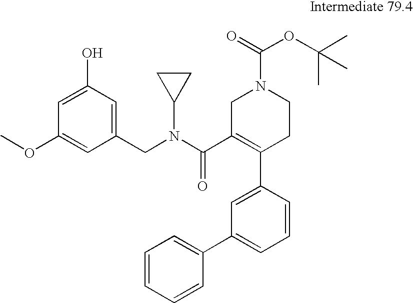

Intermediate 79.4 is synthesized by deprotection and protection of Intermediate 79.5 (2.09 g, 4.7 mmol) analogously to the preparation of Intermediate 632. White amorphous material; ES-MS: M+H=555; HPLC: t_{Ret}=4.82 min.

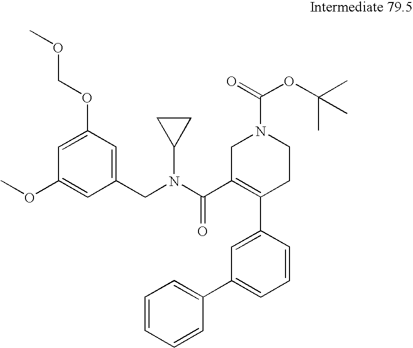

Intermediate 79.5 is synthesized by condensation of Intermediate 12 (3.12 g, 7.5 mmol) analogously to the preparation of Intermediate 1.1. White amorphous material; ES-MS: M+H=599; HPLC: t_{Ret}=5.27 min.

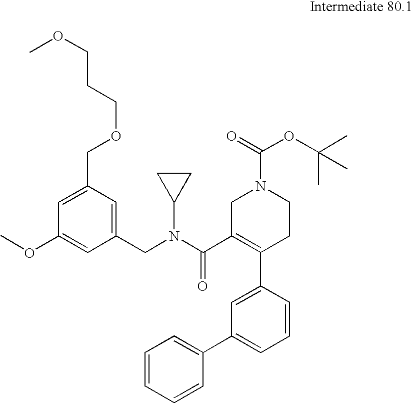

Intermediate 80.1 is synthesized by condensation of Intermediate 1.2 (55 mg, 0.13 mmol) and Intermediate 80.2 (41.3 mg, 0.14 mmol) analogously to the preparation of Intermediate 1.1. White amorphous material; ES-MS: M+H=642; HPLC: t_{Ret}=5.37 min.

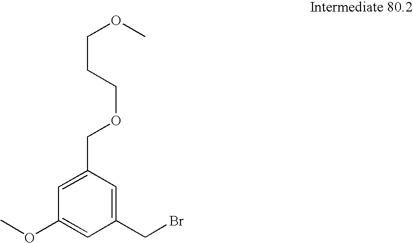

Intermediate 80.2 is synthesized by bromination of Intermediate 80.3 (45 mg, 0.19 mmol) analogously to the preparation of Intermediate 1.5. White powder, Rf=0.72 (EtOAc:n-Hex=1:1). ¹H NMR (CDCl₃); δ 1.85-1.95 (dt, 2H), 3.37 (s, 3H), 3.50 (t, 2H), 3.55 (t, 2H), 3.80 (s, 3H), 4.45 (s, 2H), 4.46 (s, 2H), 6.80-6.85 (m, 2H), 6.94 (brs, 1H).

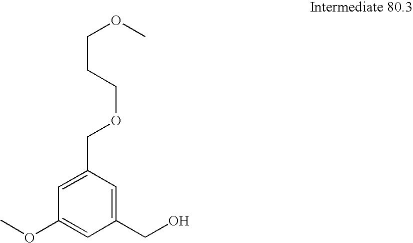

Intermediate 80.3 is synthesized by reduction of Intermediate 80.4 analogously to the preparation of Intermediate 1.6. Colorless oil; ES-MS: M+H=241; HPLC: t_{Ret}=2.74 min

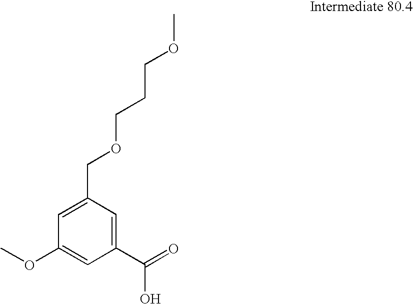

Intermediate 80.4 is synthesized by alkylation of 3-(bromomethyl-5-methoxy-benzoic acid methylester (see e.g. Tetrahedron Lett. 1990, 31, 6313-16) analogously to the preparation of Intermediate 73. White amorphous material; Rf=0.32 (DMC:MeOH=20:1). ¹H NMR (CDCl₃); δ 1.78 (dq, 2H), 3.35 (s, 3H), 3.45 (t, 2H), 3.55 (t, 2H), 3.85 (s, 3H), 4.50 (s, 2H), 6.82-6.85 (m, 1H), 6.93-6.94 (m, 2H).

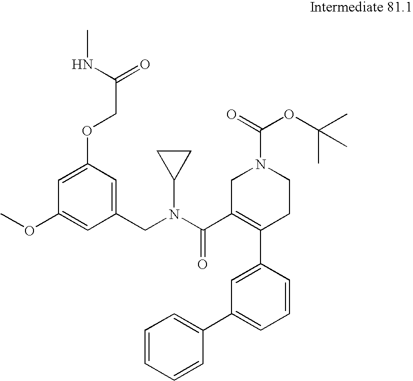

Intermediate 81.1 is synthesized by condensation of Intermediate 79.2 (360 mg, 0.6 mmol) analogously to the preparation of Intermediate 1.2. White amorphous material; ES-MS: M+H=626; HPLC: t_{Ret}=4.59 min.

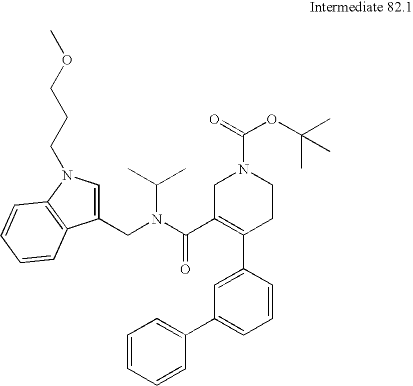

Intermediate 82.1 is synthesized by condensation of Intermediate 3.2 (300 mg, 0.6 mmol) and Intermediate 82.2 (235 mg, 0.9 mmol) analogously to the preparation of Intermediate 3.1. White amorphous material; ES-MS: M+H=622; HPLC: t_{Ret}=5.70 min.

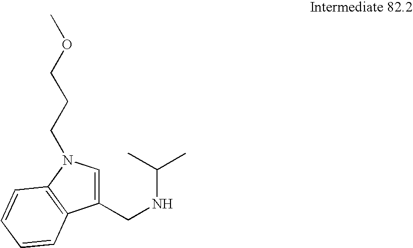

Intermediate 82.2 is synthesized by reductive amination of Intermediate 3.4 (3.3 g, 15 mmol) analogously to the preparation of Intermediate 3.3. Colorless oil; ES-MS: M+H=261; HPLC: t_{Ret}=2.74 min

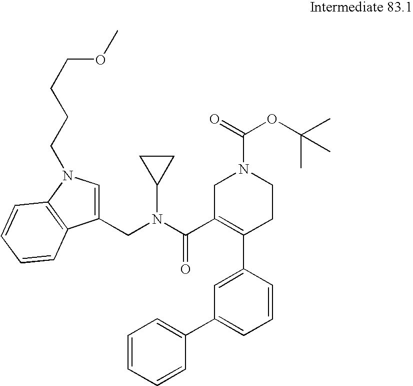

Intermediate 83.1 is synthesized by condensation of Intermediate 3.2 (300 mg, 0.6 mmol) and Intermediate 83.2 (330 mg, 0.9 mmol) analogously to the preparation of Intermediate 3.1. White amorphous material; ES-MS: M+H=634; HPLC: t_{Ret}=5.46 min.

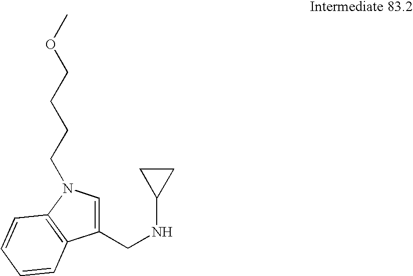

Intermediate 83.2 is synthesized by reductive amination of Intermediate 83.3 (3.3 g, 14.2 mmol) analogously to the preparation of Intermediate 3.3. Colorless oil; HPLC t_{Ret}=2.72 min; Rf=0.19 (CH₂Cl₂:MeOH=5:1)

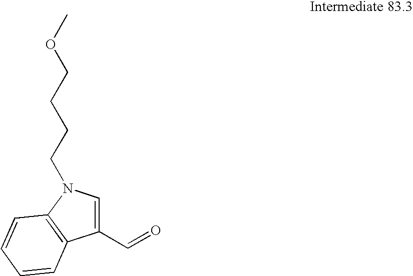

Intermediate 83.3 is synthesized by alkylation reaction of indole-3-carboxaldehyde (1.5 g, mmol) with toluene-4-sulfonic acid 4-methoxy-butyl ester (3.2 g, 12.4 mmol) analogously to the preparation of Intermediate 3.4. Colorless oil; Rf=0.61 (EtOAc:n-Hex=1:1); ES-MS: M+H=232.

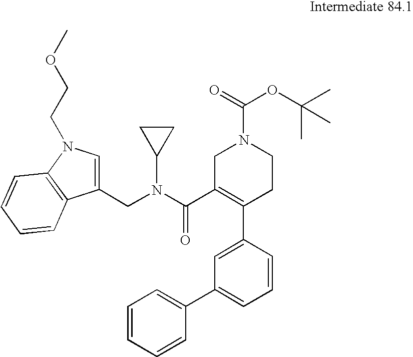

Intermediate 84.1 is synthesized by condensation of Intermediate 3.2 (150 mg, 0.4 mmol) and Intermediate 84.2 (147 mg, 0.6 mmol) analogously to the preparation of Intermediate 3.1. White amorphous material; ES-MS: M+H=606; HPLC: t_{Ret}=5.47 min.

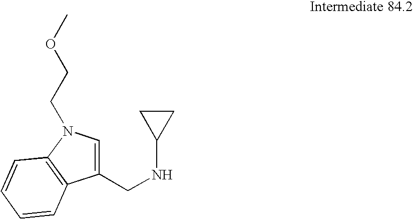

Intermediate 84.2 is synthesized by reductive amination of Intermediate 84.3 (2.0 g, 9.8 mmol) analogously to the preparation of Intermediate 3.3. Colorless oil; ES-MS: M+H=245; HPLC: t_{Ret}=2.47 min

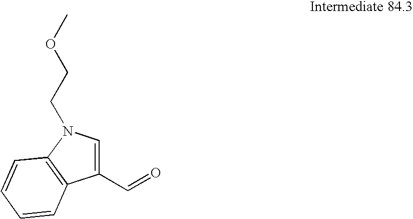

Intermediate 84.3 is synthesized by alkylation of indole-3-carboxaldehyde (2.0 g, 13.8 mmol) with 1-bromo-2-methoxy-ethane (2.3 g, 16.5 mmol) analogously to the preparation of Intermediate 3.4. Colorless oil; ES-MS: M+H=204; HPLC: t_{Ret}=3.04 min

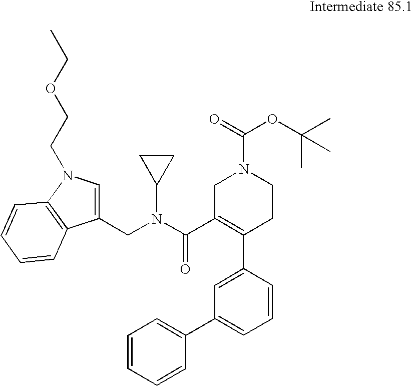

Intermediate 85.1 is synthesized by condensation of Intermediate 3.2 (150 mg, 0.4 mmol) and Intermediate 852 (155 mg, 0.6 mmol) analogously to the preparation of Intermediate 3.1. White amorphous material; ES-MS: M+H=620; HPLC: t_{Ret}=5.64 min.

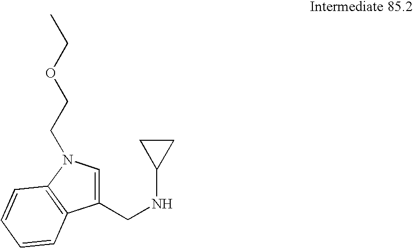

Intermediate 85.2 is synthesized by reductive amination of Intermediate 85.3 (2.6 g, 11.9 mmol) analogously to the preparation of Intermediate 3.3. Brown oil; ES-MS: M+H=259; HPLC: t_{Ret}=2.70 min

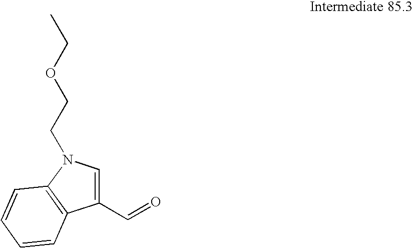

Intermediate 85.3 is synthesized by alkylation of indole-3-carboxaldehyde (2.0 g, 13.8 mmol) with 2-bromo-1-ethoxyethane (2.07 mL, 18.3 mmol) analogously to the preparation of Intermediate 3.4. Colorless oil; ES-MS: M+H=218; HPLC: t_{Ret}=3.34 min

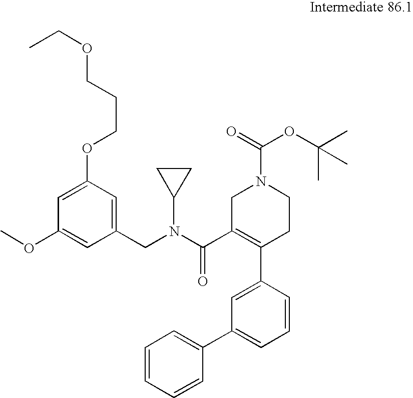

Intermediate 86.1 is synthesized by condensation of Intermediate 12 (150 mg, 0.36 mmol) and Intermediate 862 (130 mg, 0.43 mmol) analogously to the preparation of Intermediate 1.1. White amorphous material; ES-MS: M+H=641; HPLC: t_{Ret}=5.59 min.

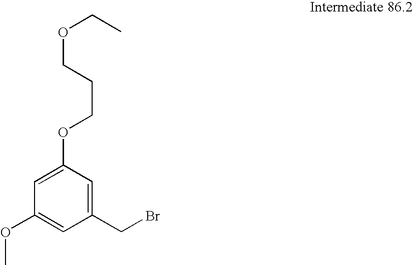

Intermediate 86.2 is synthesized by bromination of Intermediate 86.3 (200 mg, 0.82 mmol) analogously to the preparation of Intermediate 1.5. Colorless oil; ES-MS: M+H=303; HPLC: t=4.45 min

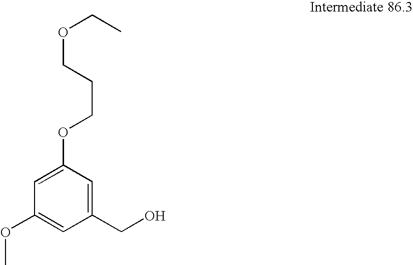

Intermediate 86.3 is synthesized by reduction of Intermediate 86.4 (2.1 g, 7.8 mmol) analogously to the preparation of Intermediate 1.6. Colorless oil; ES-MS: M+H=241; HPLC: t_{Ret}=3.15 min

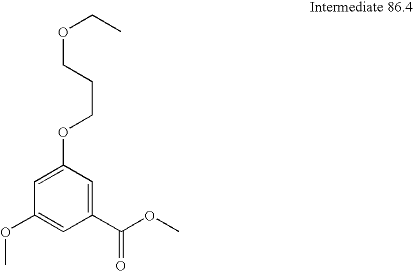

A mixture of 3-methoxy-5-hydroxybenzoic acid methyl ester (1.5 g, 8.2 mmol), 3-ethoxypropanol (1.42 mL, 12 mmol), PPh₃ (4.2 g, 16 mmol) and DEAD (2.53 mL, 16 mmol) in THF (30 mL) is stirred under N₂ at RT for 2 h. The reaction mixture is concentrated under reduced pressure and purified by silica gel flash chromatography to give Intermediate 86.4 as white powder, ES-MS: M+H=269; HPLC: t_{Ret}=4.17 min

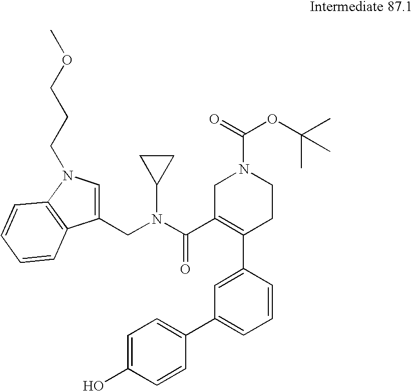

Intermediate 87.1 is synthesized by coupling of Intermediate 87.2 (150 mg, 0.24 mmol) and 4-hydroxyphenylboronic acid (49 mg, 0.36 mmol) analogously to the preparation of Intermediate 2.1. White amorphous material; ES-MS: M+H=636; HPLC: t_{Ret}=4.82 min.

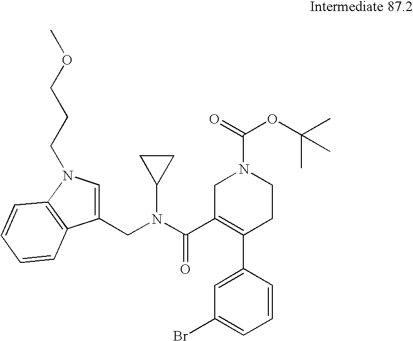

Intermediate 87.2 is synthesized by condensation of Intermediate 2.4 (1.0 g, 2.6 mmol) and Intermediate 3.3 (1.0 g, 3.9 mmol) analogously to the preparation of Intermediate 3.1. White amorphous material; ES-MS: M=622, M+2H=624; HPLC: t_{Ret}=5.42 min.

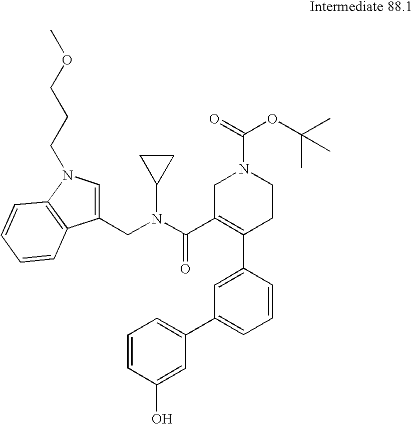

Intermediate 88.1 is synthesized by coupling of Intermediate 87.2 (150 mg, 0.24 mmol) and 3-hydroxyphenylboronic add (49 mg, 0.36 mmol) analogously to the preparation of Intermediate 2.1. White amorphous material; ES-MS: M+H=636; HPLC: t_{Ret}=4.92 min.

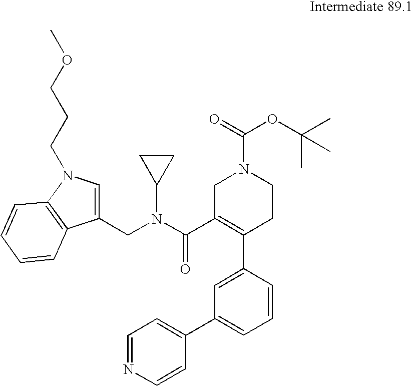

Intermediate 89.1 is synthesized by coupling of Intermediate 87.2 (200 mg, 0.32 mmol) and 4-pyridylboronic acid (196 mg, 1.6 mmol) analogously to the preparation of Intermediate 2.1. White amorphous material; ES-MS: M+H=621; HPLC: t_{Ret}=3.73 min.

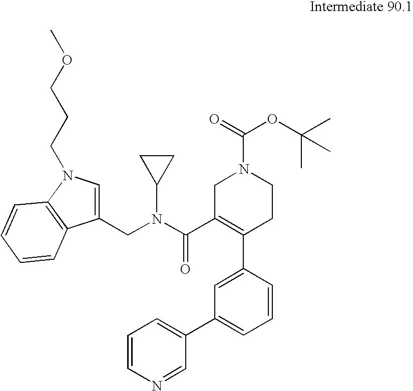

Intermediate 90.1 is synthesized by coupling of Intermediate 87.2 (200 mg, 0.32 mmol) and 3-pyridylboronic add (196 mg, 1.6 mmol) analogously to the preparation of Intermediate 2.1. White amorphous material; ES-MS: M+H=621; HPLC: t_{Ret}=3.70 min.

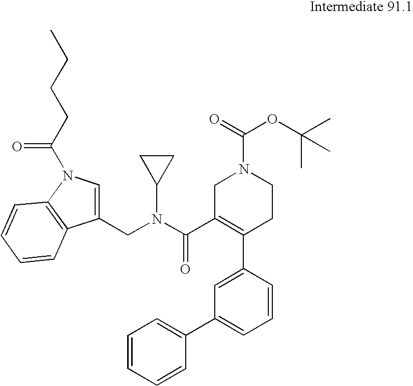

Intermediate 91.1 is synthesized by condensation of Intermediate 3.2 (150 mg, 0.30 mmol) and Intermediate 91.2 (114 mg, 0.45 mmol) analogously to the preparation of Intermediate 3.1. White amorphous material; ES-MS: M+H=632; HPLC: t_{Ret}=5.92 min.

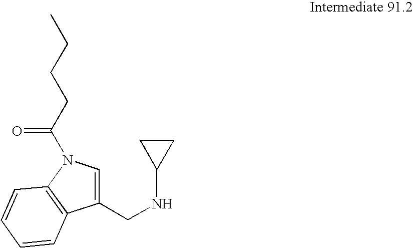

Intermediate 912 is synthesized by reductive amination of Intermediate 91.3 (1.5 g, 6.6 mmol) analogously to the preparation of Intermediate 3.3. Pale yellow solid; ES-MS: M+H=271; HPLC: t_{Ret}=3.09 min

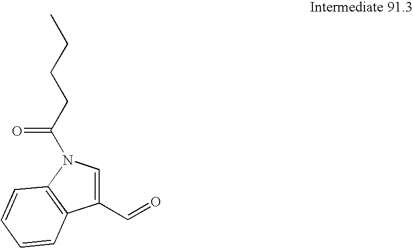

A mixture of indole-3-carboxaldehyde (1.0 g, 7.0 mmol), Et₃N (1.8 mL, 12 mmol), and pentanoyl chloride (1.2 g, 10 mmol) in THF (10 mL) is stirred under N₂ at 0° C. After stirring for 3 h, H₂O is added to the reaction mixture and it is then extracted with EtOAc. The combined organic phases are washed with H₂O, brine and dried (Na₂SO₄). Concentration under reduced pressure gives Intermediate 91.3 as pale yellow solids; Rf=0.88 (EtOAc:n-Hex=1:1), ¹H NMR (CDCl₃), δ: 1.02 (3H, t), 1.52 (2H, dt), 1.87 (2H, m), 3.02 (2H, t), 7.01 (1H, dd), 7.45 (1H, dd), 8.12 (1H, s), 8.27 (1H, d), 8.44 (1H, d).

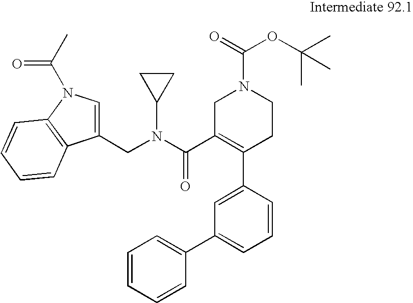

Intermediate 92.1 is synthesized by condensation of Intermediate 32 (150 mg, 0.30 mmol) and Intermediate 922 (102 mg, 0.45 mmol) analogously to the preparation of Intermediate 3.1. White amorphous material; ES-MS: M+H=590; HPLC: t_{Ret}=5.30 min.

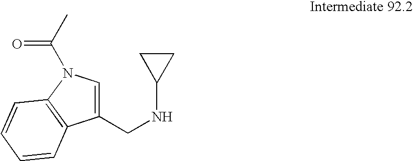

Intermediate 92.2 is synthesized by reductive amination of 1-acetyl-1H-indole-3-carbaldehyde (1.0 g, 5.3 mmol) analogously to the preparation of Intermediate 3.3. Colorless oil; ES-MS: M+H=229; HPLC: t_{Ret}=2.45 min

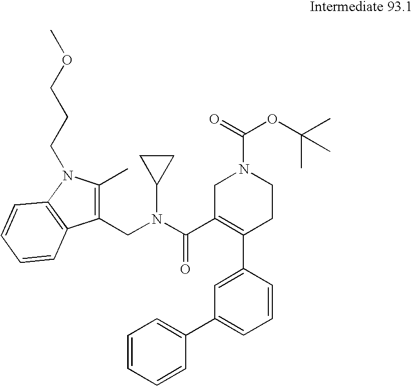

Intermediate 93.1 is synthesized by condensation of Intermediate 32 (150 mg, 0.30 mmol) and Intermediate 93.2 (122 mg, 0.45 mmol) analogously to the preparation of Intermediate 3.1. White amorphous material; ES-MS: M+H=634; HPLC: t_{Ret}=5.70 min.

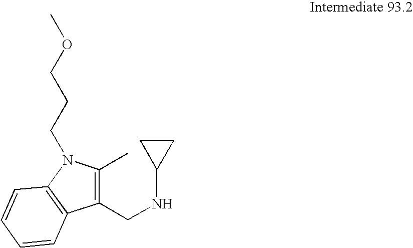

Intermediate 93.2 is synthesized by reductive amination of Intermediate 93.3 (2.3 g, 9.9 mmol) analogously to the preparation of Intermediate 3.3. Colorless oil; ES-MS: M+=272; HPLC: t_{Ret}=2.79 min

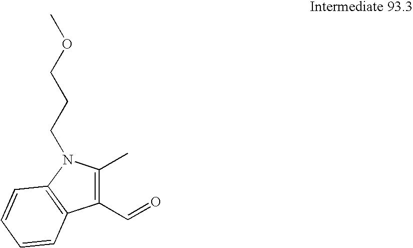

Intermediate 93.3 is synthesized by alkylation of 2-methyl-1H-indole-3-carbaldehyde (2.0 g, 12.5 mmol) with toluene-4-sulfonic acid 3-methoxy-propyl ester (3.7 g, 15.0 mmol) analogously to the preparation of Intermediate 3.4. Colorless oil; ES-MS: M+H=232; HPLC: t_{Ret}=3.38 min

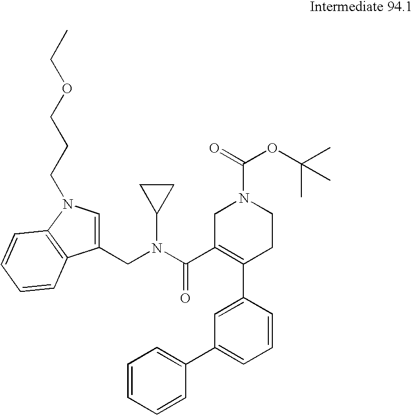

Intermediate 94.1 is synthesized by condensation of Intermediate 3.2 (300 mg, 0.6 mmol) and Intermediate 94.2 (330 mg, 1.2 mmol) analogously to the preparation of Intermediate 3.1. White amorphous material; ES-MS: M+H=634; HPLC: t_{Ret}=5.67 min.

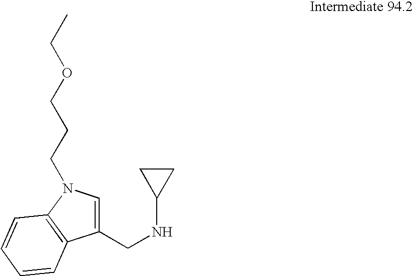

Intermediate 94.2 is synthesized by reductive amination of Intermediate 94.3 (3.2 g, 13.8 mmol) analogously to the preparation of Intermediate 3.3. Colorless oil; ES-MS: M+=273; HPLC: t_{Ret}=2.56 min

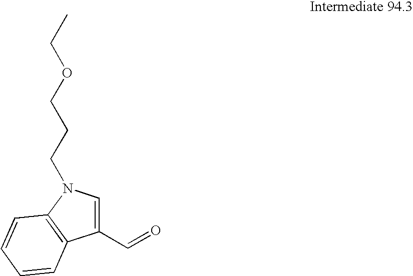

Intermediate 94.3 is synthesized by alkylation of 1H-indole-3-carbaldehyde (2.0 g, 13.8 mmol) with toluene-4-sulfonic acid 3-ethoxy-propyl ester (4.3 g, 16.5 mmol) analogously to the preparation of Intermediate 3.4. Colorless oil; Rf=0.67 (EtOAc:n-Hex=1:1), ¹H NMR (CDCl₃), δ: 1.32 (3H, t), 2.15 (2H, dt), 3.33 (2H, t), 3.45 (2H, q), 4.35 (2H, t), 7.22-7.45 (3H, m), 7.72 (1H, s), 8.30-8.40 (1H, m), 9.95 (1H, s).

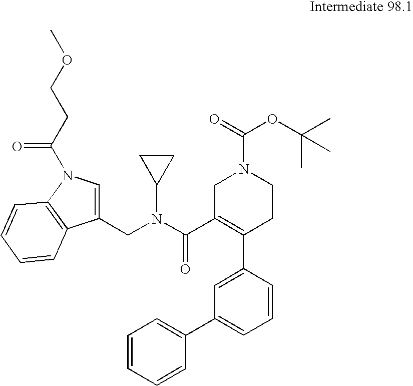

Intermediate 98.1 is synthesized by condensation of Intermediate 3.2 (150 mg, 0.40 mmol) and Intermediate 98.2 (131 mg, 0.48 mmol) analogously to the preparation of Intermediate 3.1. White amorphous material; ES-MS: M+H=634; HPLC: t_{Ret}=5.39 min.

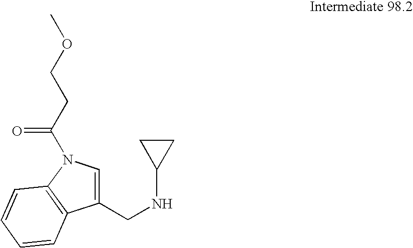

Intermediate 98.2 is synthesized by reductive amination of Intermediate 98.3 (820 mg, 3.3 mmol) analogously to the preparation of Intermediate 3.3. Pale yellow solid; ES-MS: M+H=246; HPLC: t_{Ret}=2.42 min

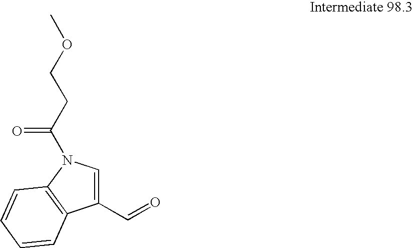

A mixture of indole-3-carboxaldehyde (1.09, 7.0 mmol), Et₃N (3.1 mL, 20 mmol), and 3-methoxy propanoyl chloride (1.0 g, 8.0 mmol) in THF-CH₂Cl₂ (13 mL, 10:3) is stirred under N₂ at 0° C. After stirring at room temperature for 12 h, H₂O is added and the resulting mixture is extracted with EtOAc. The combined organic phases are washed with H₂O, brine and dried (Na₂SO₄). Concentration under reduced pressure gives Intermediate 98.3 as white solids; Rf=0.32 (EtOAc:n-Hex=1:1), ¹H NMR (CDCl₃), δ: 3.25 (2H, t), 3.41 (3H, s), 3.90 (2H, t), 7.39-7.47 (2H, m), 8.16 (1H, s), 8.28 (1H, d), 8.44 (1H, d), 10.13 (1H, s).

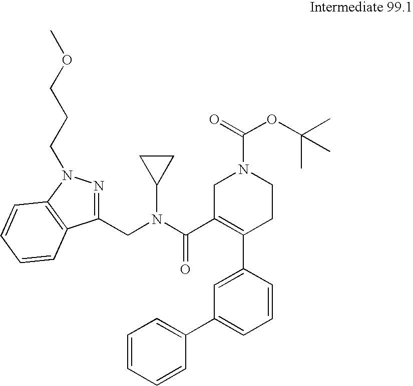

Intermediate 99.1 is synthesized by condensation of Intermediate 3.2 (154 mg, 0.31 mmol) and Intermediate 99.2 (100 mg, 0.39 mmol) analogously to the preparation of Intermediate 3.1. White amorphous material; ES-MS: M+H=621; HPLC: t_{Ret}=5.42 min.

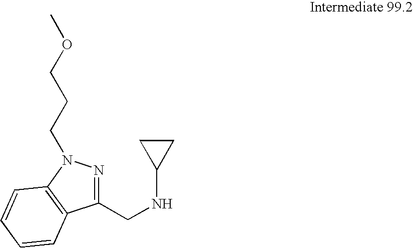

Intermediate 99.2 is synthesized by reductive amination of Intermediate 99.3 (500 mg, 2.3 mmol) analogously to the preparation of Intermediate 3.3. Colorless oil; ES-MS: M+H=260; HPLC: t_{Ret}=2.38 min

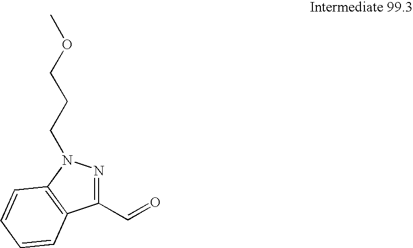

Intermediate 99.3 is synthesized by oxidation of Intermediate 99.4 (700 mg, 3.18 mmol) analogously to the preparation of Intermediate 76.3. Colorless oil; ES-MS: M+H=219; HPLC: t_{Ret}=3.52 min

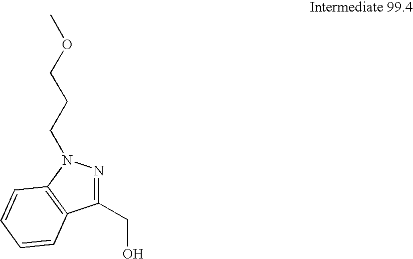

Intermediate 99.4 is synthesized by reduction of Intermediate 99.5 (1.0 g, 3.4 mmol) analogously to the preparation of Intermediate 1.6. Colorless oil; ES-MS: M+H 221; HPLC: t_{Ret}=2.73 min

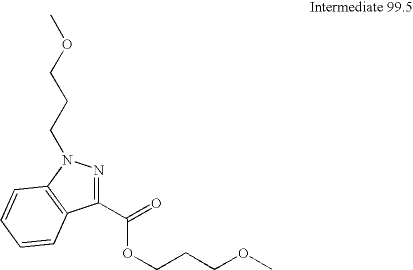

To a mixture of indazole-3-carboxylic acid (2 g, 13.7 mmol) and toluene-4-sulfonic acid 3-methoxy-propyl ester (5 g, 20.6 mmol) in DMF (15 mL), NaH (1.12 g, 28 mmol) is added under N₂ at 0° C. After stirring at 50° C. for 12 h, H₂O is added to the reaction mixture, then conc. HCl aq., and the mixture is extracted with EtOAc. The combined organic phases are washed with H₂O, brine and dried (Na₂SO₄). Concentration under reduced pressure and silica gel flash chromatography give Intermediate 99.5 as colorless oil; ES-MS: M+H=307; HPLC: t_{Ret}=3.65 min

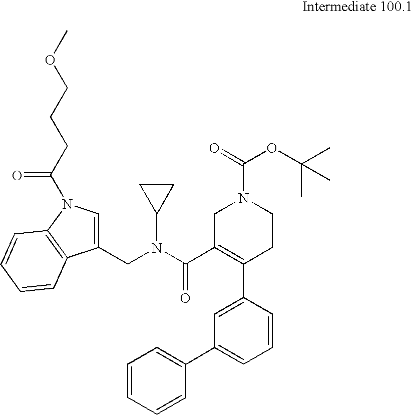

Intermediate 100.1 is synthesized by condensation of Intermediate 1.3 (150 mg, 0.40 mmol) and Intermediate 1002 (137 mg, 0.48 mmol) analogously to the preparation of Intermediate 1.2. White amorphous material; ES-MS: M+H=648; HPLC: _{A}t_{Ret}=5.47 min.

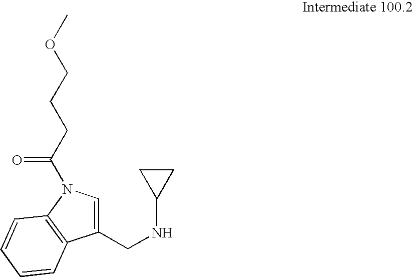

Intermediate 100.2 is synthesized by condensation of Intermediate 100.3 (820 mg, 3.34 mmol) and cyclopropylamine (387 mg, 6.80 mmol) analogously to the preparation of Intermediate 3.3. Colorless oil; ES-MS: M+H=246; HPLC: _{A}t_{Ret}=2.42 min.

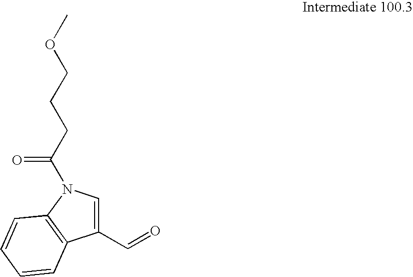

Intermediate 100.3 is synthesized by condensation of indole-3-carbaldehyde (650 mg, 4.5 mmol) and 4-Methoxybutanoyl chloride (929 mg, 6.80 mmol) (see e.g. Canadian Journal of Chemistry 1982, 60, 2295-312, or U.S. Pat. No. 4,559,337.) analogously to the preparation of Intermediate 3.4. Colorless oil; Rf=0.30 (EtOAc:n-Hex=1:1), ¹H NMR (CDCl₃), δ: 2.10-2.18 (2H, m), 3.13 (2H, t), 3.36 (3H, s), 3.53 (2H, t), 7.39-7.47 (2H, m), 8.12 (1H, t), 8.28 (1H, d), 8.44 (1H, d), 10.13 (1H, s).

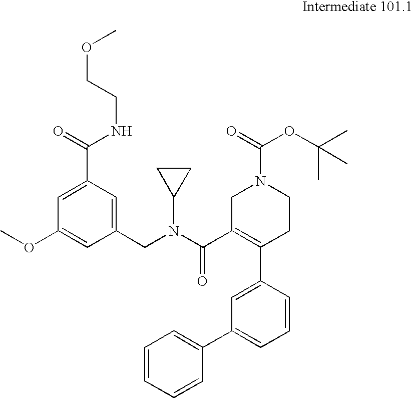

Intermediate 101.1 is synthesized by condensation of Intermediate 101.2 (201 mg, 0.52 mmol) and Intermediate 1.3 (173 mg, 0.35 mmol) analogously to the preparation of Intermediate 3.1. White amorphous material; ES-MS: M+H 640; HPLC: _{A}t_{Ret}=4.60 min.

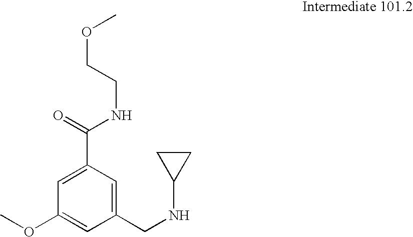

A mixture of Intermediate 101.3 (1.08 g, 3.60 mmol) cyclopropylamine (0.75 mL, 10.8 mmol) and K₂CO₃ (1.0 g, 7.20 mmol) in CH₃CN (5 mL) are stirred at RT for over night. After adding H₂O (20 mL), the reaction mixture is extracted with DCM (20 mL, 2×). The combined organic phases are washed with H₂O, brine and dried (Na₂SO₄), concentrated under reduce pressure to give Intermediate 101.2 as colorless oil; ES-MS: M+H=279; HPLC: t_{Ret}=2.02 minutes

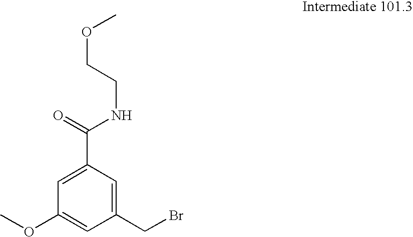

Intermediate 101.3 is synthesized by bromination of Intermediate 101.4 (750 mg, 3.13 mmol) analogously to the preparation of compound of Intermediate 1.5. Colorless oil; ES MS: M+2H=304; HPLC: t_{Ret}=3.09 minutes.

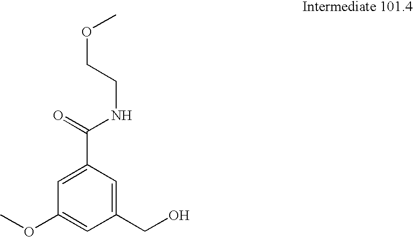

Intermediate 101.4 is synthesized by reduction of Intermediate 101.5 (880 mg, 3.29 mmol) analogously to the preparation of compound of Intermediate 1.6. Colorless oil; Rf=0.43 (AcOEt:n-Hex=2:1); ¹H NMR (400 MHz, CDCl₃); δ 2.91 (s, 3H), 3.55-3.57 (m, 2H), 3.60-3.65 (m, 2H), 3.83 (s, 3H), 4.68 (s, 2H), 6.67 (brs, 1H), 7.02 (s, 1H), 7.23-7.27 (m, 2H).

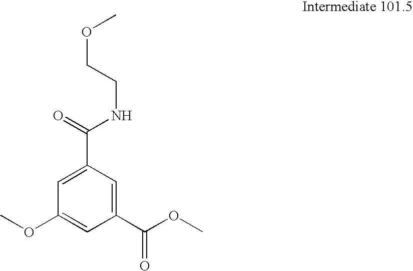

Intermediate 101.5 is synthesized by condensation of 5-Methoxy-isophthalic acid mono methyl ester (1.01 g, 4.24 mmol) and 2-Methoxyethylamine (0.95 g, 12.7 mmol) analogously to the preparation of compound of Intermediate 1.2. White amorphous material; Rf=0.47 (AcOEt:n-Hex=2:1); ¹H NMR (400 MHz, CDCl₃); δ 3.40 (s, 3H), 3.52-3.60 (m, 2H), 3.65-3.75 (m, 2H), 3.88 (s, 3H), 3.95 (s, 3H), 6.51 (brs, 1H), 7.60-7.61 (m, 1H), 7.67-7.69 (m, 1H), 8.14 (brs, 1H).

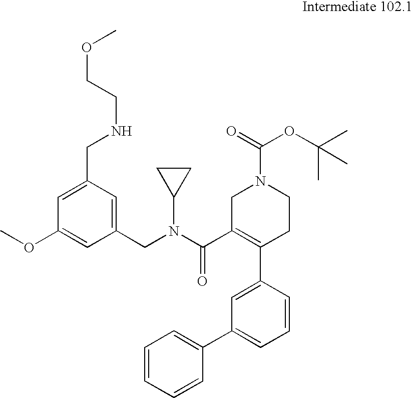

Intermediate 102.1 is synthesized by condensation of Intermediate 102.1 (125 mg, 0.34 mmol) and Intermediate 3.2 (114 mg, 0.23 mmol) analogously to the preparation of Intermediate 3.1. White amorphous material; ES-MS: M+H=726; HPLC: _{A}t_{Ret}=5.65 min.

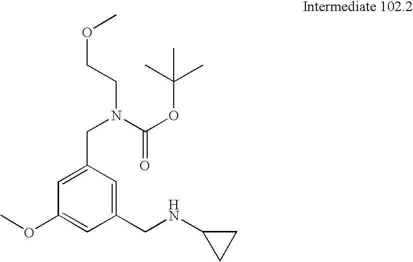

Intermediate 102.2 is synthesized by amination of Intermediate 102.3 (200 mg, 0.52 mmol) analogously to the preparation of Intermediate 3.3. Colorless oil; ES-MS: M+H=365; HPLC: t_{Ret}=2.97 minutes.

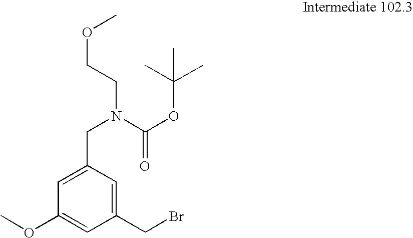

Intermediate 102.3 is synthesized by bromination of Intermediate 102.4 (285 mg, 0.88 mmol) analogously to the preparation of compound of Intermediate 1.5. Colorless oil; Rf=0.80 (AcOEt:n-Hex=1:1); ¹H NMR (400 MHz, CDCl₃); δ 1.40-1.50 (brs, 9H), 3.30 (s, 3H), 3.38-3.55 (m, 4H), 3.80 (s, 3H), 4.40 (s, 2H), 4.45 (brs, 2H), 6.65-6.85 (m, 3H).

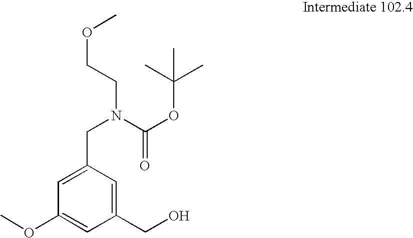

Intermediate 102.4 is synthesized by reduction of Intermediate 102.5 (366 mg, 1.04 mmol) analogously to the preparation of compound of Intermediate 1.6. Colorless oil; Rf=0.24 (AcOEt:n-Hex=1:1); ¹H NMR (400 MHz, CDCl₃); δ 1.40-1.55 (brs, 9H), 3.30 (s, 3H), 3.35-3.55 (m, 4H), 3.80 (s, 3H), 4.45 (s, 2H), 4.60 (d, 2H), 6.65-6.82 (m, 3H).

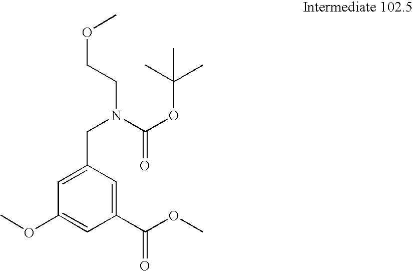

A mixture of 3-Bromomethyl-5-methoxy-benzoic acid methyl ester (300 mg, 1.16 mmol) (see e.g. Tetrahedron Lett, 1993, 31, 6313), 2-Methoxy-ethylamine (260 mg, 3.47 mmol) and K₂CO₃ (0.32 g, 2.32 mmol) in CH₃CN (5 mL) are stirred at RT, for over night. After adding H₂O (20 mL), the reaction mixture is extracted with CH₂Cl₂ (20 mL, 2×). The combined organic phases are washed with H₂O, brine and dried (Na₂SO₄), concentrated under reduced to give 3-Methoxy-5-[(2-methoxy-ethylamino)-methyl]-benzoic acid methyl ester as an oil (217 mg, 0.85 mmol; 85%; ES-MS: M+H=254; HPLC: t_{Ret}=2.29 minutes). This crude material is used without purification. To a mixture of this crude material and Et₃N (0.48 ml, 3.47 mmol), (BOC)₂O (380 mg, 1.74 mmol) in DCM (5 mL) is added at RT. After stirring for 1 h, the reaction mixture is quenched by adding H₂O, and mixture is extracted with DCM. The combined organic phases are washed with H₂O, brine and dried (MgSO₄), concentrated under reduced pressure and silica gel flash chromatography to give Intermediate 102.5 as white amorphous; Rf=0.47 (AcOEt:n-Hex=1:2) H NMR (400 MHz, CDCl₃); δ 1.40-1.55 (brs, 9H), 3.30 (s, 3H), 3.35-3.55 (m, 4H), 3.80 (s, 3H), 3.90 (s, 3H), 4.45 (brs, 2H), 6.95-7.0 (brs, 1H), 7.40 (m, 1H), 7.50 (m, 1H).

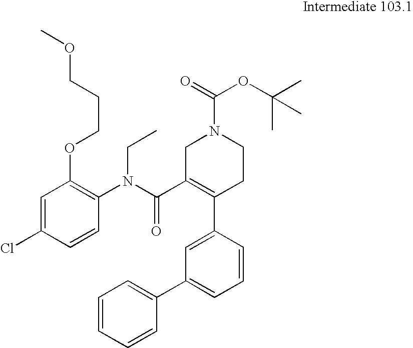

Intermediate 103.1 is synthesized by alkylation of Intermediate 103.2 (238 mg, 0.41 mmol) analogously to the preparation of Intermediate 1.1. White amorphous material; ES-MS: M+H=605; HPLC: t_{Ret}=5.82 min.

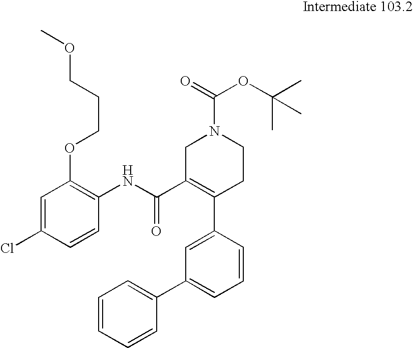

A mixture of Intermediate 1.3 (272 mg, 0.72 mmol), Intermediate 103.3 (170 mg, 0.79 mmol) and DMT-MM (239 mg, 0.86 mmol) in EtOH (5 mL) was stirred under N₂ at 60° C. for 5.5 h. After adding H₂O, the reaction mixture is extracted with EtOAc. The combined organic phases are washed with H₂O, brine and dried (MgSO₄). Concentration under reduced pressure and silica gel flash chromatography give Intermediate 103.2 as brown oil; ES-MS: M+H=577; HPLC: _{A}t_{Ret}=5.64 min.

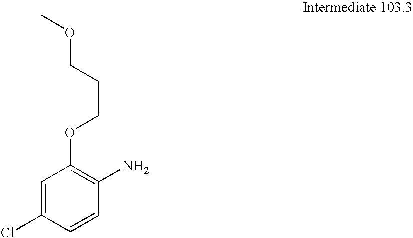

A mixture of Intermediate 103.4 (1.70 g, 6.93 mmol) and Tin(II) chloride 2-hydrate (4.69 g, 20.8 mmol) in EtOAc (30 mL) was stirred under N₂ at reflux for 8 h. After adding 8N KOH solution, the reaction mixture is extracted with EtOAc. The combined organic phases are washed with H₂O, brine and dried (MgSO₄). Concentration under reduced pressure and silica gel flash chromatography give Intermediate 103.3 as yellow oil; ES-MS: M+H=216; HPLC: _{A}t_{Ret}=2.40 min.

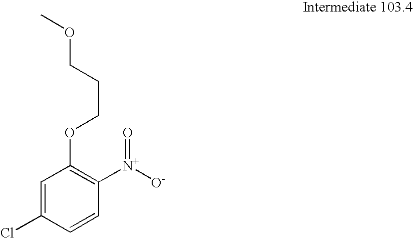

A mixture of 4-chloro-2-fluoro-nitrobenzene (1.23 g, 7.0 mmol), 3-methoxy-1-propanol (737 μL, 7.7 mmol) and TBAB (451 mg, 1.4 mmol) in 8 M KOH (10 mL) and toluene (10 mL) are stirred under N₂ at 60° C. for 3 hours. After adding H₂O to the residue, the mixture is extracted with EtOAc. The combined organic phases are washed with H₂O, brine and dried (Na₂SO₄), concentrated under reduced pressure and silica gel flash chromatography to give Intermediate 103.4 as yellow oil; Rf: 0.5 (AcOEt/Hex=1:2); ¹H NMR (400 MHz, CDCl₃) δ 2.10 (quint., 2H), 3.36 (s, 3H), 3.58 (t, 2H), 4.20 (t, 2H), 7.00 (dd, 1H), 7.10 (d, 1H), 7.82 (d, 1H).

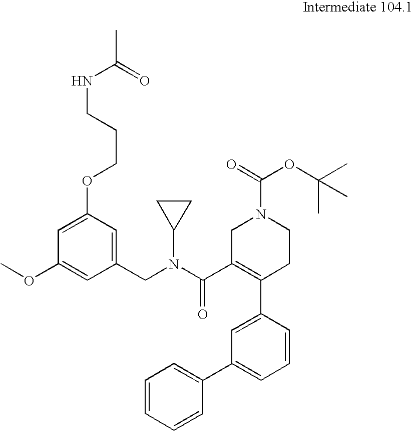

Intermediate 104.1 is synthesized by acylation of Intermediate 104.2 (150 mg, 0.25 mmol) analogously to the preparation of Intermediate 91.3. White amorphous material; ES-MS: M+H=612; HPLC: t_{Ret}=3.75 min.

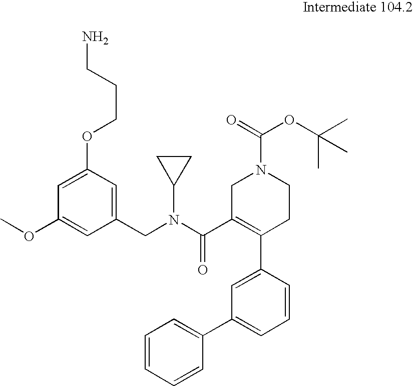

Intermediate 104.2 is synthesized by deprotection of Intermediate 104.3 (378 mg, 0.51 mmol) analogously to the preparation of Intermediate 52.1. White amorphous material; ES-MS: M+H=612; HPLC: t_{Ret}=3.75 min.

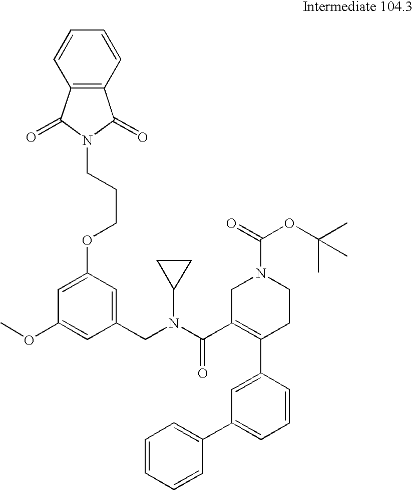

Intermediate 104.3 is synthesized by alkylation of Intermediate 79.4 (363 mg, 0.66 mmol) analogously to the preparation of Intermediate 7.3. White amorphous material; ES-MS: M+H-^{t}Bu=686; HPLC: t_{Ret}=5.55 min.

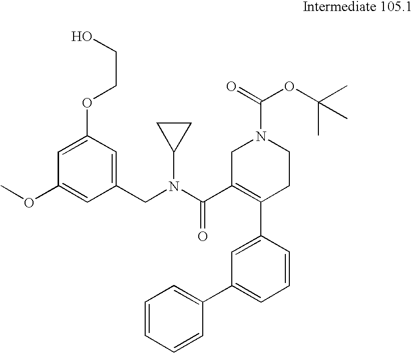

A mixture of Intermediate 1052 (101 mg, 0.15 mmol) and pTsOH—H₂O (8.4 mg, 0.044 mmol) in MeOH (10 mL) are stirred under N₂ at 60° C. for 2 hours. The MeOH is removed in vacuo. After adding saturated aqueous NaHCO₃ to the residue, the mixture is extracted with DCM. The combined organic phases are washed with H₂O, brine and dried (Na₂SO₄), concentrated under reduced pressure and silica gel flash chromatography to give Intermediate 105.1 as colorless oil; ES-MS: M+=599; HPLC: t_{Ret}=5.57 minutes.

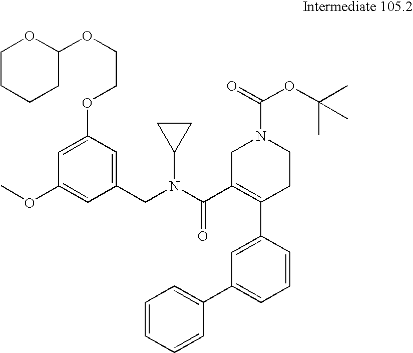

Intermediate 105.2 is synthesized by condensation of Intermediate 3.2 (101 mg, 0.15 mmol) analogously to the preparation of Intermediate 3.1. Colorless amorphous material; Rf=0.54 (EtOAc only), ¹H NMR (CDCl₃), δ: 0.25-0.72 (4H, m), 1.40-1.89 (6H, m), 1.50 (9H, s), 2.30-2.45 (1H, m), 2.68-2.90 (1H, m), 3.43-3.59 (2H, m), 3.69 (3H, s), 3.52-3.69 (3H, m), 3.70-3.79 (2H, m), 3.82-3.90 (2H, m), 3.97-4.10 (5H, m), 4.17-4.70 (3H, m), 6.17-6.38 (3H, m), 7.15 (1H, d), 7.30-7.72 (8H, m).

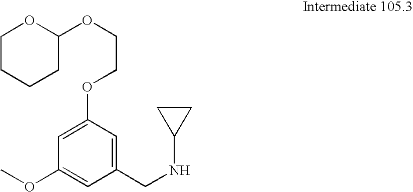

To a solution of LAH (17 mg, 0.45 mmol) in THF (5 mL), a solution of Intermediate 105.4 (100 mg, 0.30 mmol) in THF (10 mL) is added at 0° C. under N₂. After stirring at RT for 10 hours and stirring at 70° C. for 3 hours, saturated Na₂SO₄ (3 mL), is added at 0° C. The mixture is stirring for 1 h at RT, and then the suspension is filtered through a psd of Celite. The filtrate is extracted with Et₂O (20 mL, ×2). The combined organic phases are washed with H₂O, brine and dried (Na₂SO₄), concentrated under reduced pressure and silica gel flash chromatography to give Intermediate 105.3 as yellow oil; ES-MS: M+H=322; HPLC: t_{Ret}=2.77 minutes

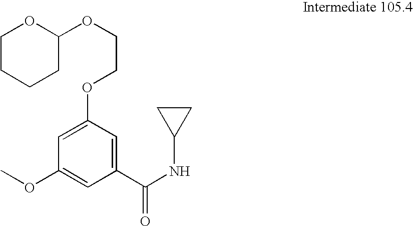

Intermediate 105.4 is synthesized by condensation of Intermediate 105.5 (1.48 g, 5.00 mmol) analogously to the preparation of compound of Intermediate 1.2. Colorless amorphous material; Rf=0.58 (EtOAc:), ¹H NMR (CDCl₃), δ: 0.49-0.65 (2H, m), 0.85-0.90 (2H, m), 1.48-1.90 (6H, m), 2.88-2.94 (1H, m), 3.49-3.58 (1H, m), 3.87 (3H, s), 3.79-3.97 (2H, m), 4.00-4.20 (3H, m), 4.69 (1H, t), 6.18 (1H, brs), 6.60 (1H, s), 6.86 (1H, d), 8.02 (1H, s).

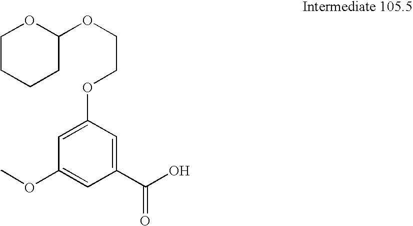

Intermediate 105.5 is synthesized by hydrolysis of Intermediate 105.6 (1.51 g, 4.87 mmol) analogously to the preparation of Intermediate 1.3. Yellow oil; Rf=0.09 (hexane/EtOAc 1:3). ¹H NMR (CDCl₃) δ 1.48-1.90 (6H, m), 3.49-3.58 (1H, m), 3.87 (3H, s), 3.79-3.97 (2H, m), 4.00-4.20 (3H, m), 4.69 (1H, t), 6.74 (1H, t), 7.21 (1H, t), 7.28 (1H, t).

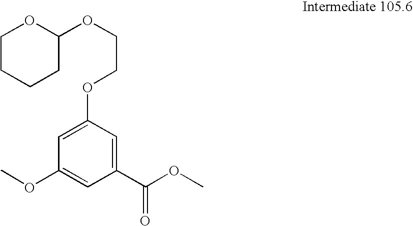

Intermediate 105.6 is synthesized by alkylation of 3-methoxy-5-hydroxybenzoic acid methylester (1.00 g, 5.45 mmol) analogously to the preparation of Intermediate 36.4. Yellow oil; Rf=0.61 (hexane/EtOAc 1:3). ¹H NMR (CDCl₃) δ 1.48-1.90 (6H, m), 3.49-3.58 (1H, m), 3.87 (3H, s), 6.91 (3H, s), 3.79-3.97 (2H, m), 4.00-4.20 (3H, m), 4.69 (1H, t), 6.69 (1H, t), 7.18 (1H, t), 7.21 (1H, t).

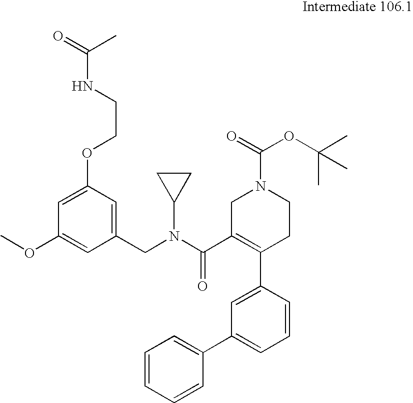

Intermediate 106.1 is synthesized by acylation of Intermediate 1062 (98 mg, 0.16 mmol) analogously to the preparation of Intermediate 91.3. Colorless amorphous material; ES-MS: M+=640; HPLC: t_{Ret}=4.60 minutes.

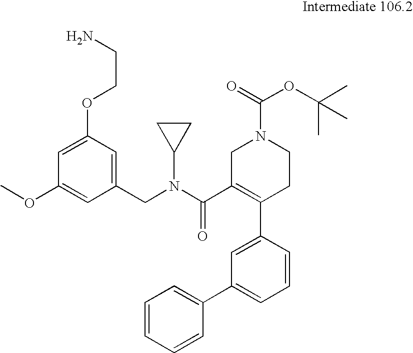

To a solution of Intermediate 106.3 (401 mg, 0.64 mmol) in THF (15 mL), 1N NaOH (1 mL) and H₂O (5 mL), PPh₃ (253 mg, 0.96 mmol) is added at 0° C. After stirring at RT for 10 hours, H₂O (10 mL), is added. The mixture is extracted with Et₂O (20 mL, ×2). The combined organic phases are washed with H₂O, brine and dried (Na₂SO₄), concentrated under reduced pressure and silica gel flash chromatography to give Intermediate 106.2 as a colorless oil; Rf=0.40 (MeOH/DCM 1:5); ¹H-NMR (CDCl₃) δ 0.41-0.70 (m, 4H), 1.51 (s, 9H), 1.53-1.67 (m, 2H), 2.29-2.43 (m, 1H), 2.68-2.75 (m, 2H), 3.00-3.12 (m, 2H), 3.55-3.76 (m, 3H), 3.71 (s, 3H), 3.82-3.98 (m, 2H), 4.02-4.61 (m, 3H), 6.05-6.35 (m, 3H), 7.12 (brs, 1H), 7.30-7.61 (m, 8H).

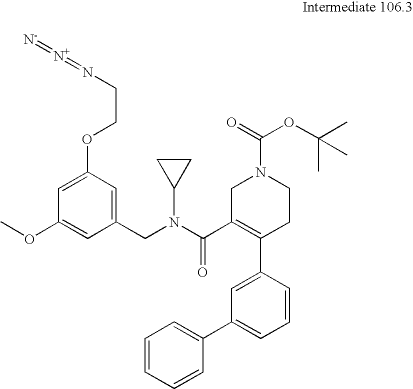

A mixture of Intermediate 106.4 (458 mg, 0.61 mmol) and NaN₃ (119 mg, 1.82 mmol) in DMF (15 mL) is stirred at 70° C. for 3.5 hours. After cooling down to 0° C., the reaction mixture is added H₂O (25 mL), the reaction mixture is extracted with Et₂O (30 mL, 2×). The combined organic phases are washed with H₂O, brine and dried (MgSO₄), concentrated under reduced pressure and silica gel flash chromatography to give Intermediate 106.3 as a colorless amorphous material; Rf=0.68 (hexane/EtOAc 1:1); ¹H-NMR (CDCl₃) δ 0.39-0.72 (m, 4H), 1.56 (s, 9H), 2.29-2.42 (m, 1H), 2.68-2.75 (m, 2H), 3.45-3.56 (m, 4H), 3.70 (s, 3H), 3.68-3.78 (m, 2H), 3.98-4.10 (m, 2H), 4.29-4.61 (m, 2H), 6.05-6.32 (m, 3H), 7.12 (d, 1H), 7.28-7.60 (m, 8H).

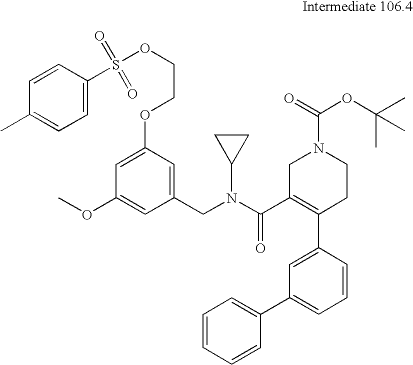

To a solution of Intermediate 105.1 (380 mg, 0.64 mmol) and NEt₃ (0.07 mL, 0.51 mmol) in DCM (15 mL), TsCl (145 mg, 0.76 mmol) is added at 0° C. After stirring at RT for 1.5 hours, H₂O (10 mL), is added. The mixture is extracted with DCM. The combined organic phases are washed with H₂O, brine and dried (Na₂SO₄), concentrated under reduced pressure and silica gel flash chromatography to give Intermediate 106.4 as colorless amorphous material; Rf=0.64 (hexane/EtOAc 1:1); ¹H-NMR (400 MHz, CDCl₃) δ 0.39-0.72 (m, 4H), 1.56 (s, 9H), 2.29-2.42 (m, 1H), 2.44 (s, 3H), 2.68-2.75 (m, 2H), 3.45-3.75 (m, 3H), 3.69 (s, 3H), 3.91-4.07 (m, 3H), 4.20-4.32 (m, 3H), 4.35-4.61 (m, 1H), 5.93-6.22 (m, 3H), 7.12 (d, 1H), 7.28-7.58 (m, 10H), 7.81 (d, 2H).

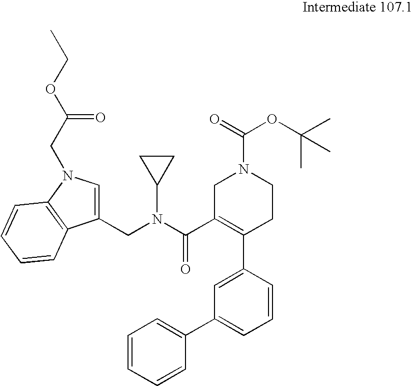

Intermediate 107.1 is synthesized by condensation of Intermediate 32 (150 mg, 0.30 mmol) and Intermediate 107.2 (120 mg, 0.44 mmol) analogously to the preparation of Intermediate 3.1. White amorphous material; ES-MS: M+H=634; HPLC: t_{Ret}=5.24 min.

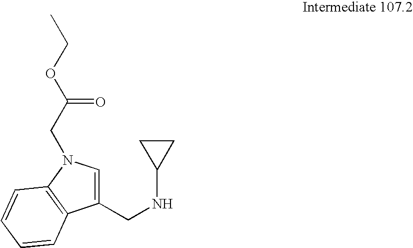

Intermediate 107.2 is synthesized by condensation of Intermediate 107.3 (500 mg, 2.16 mmol) and cyclopropylamine (232 μL, 3.24 mmol) analogously to the preparation of Intermediate 3.3. Colorless oil; ES-MS: M+H=273; HPLC: _{A}t_{Ret}=2.45 min.

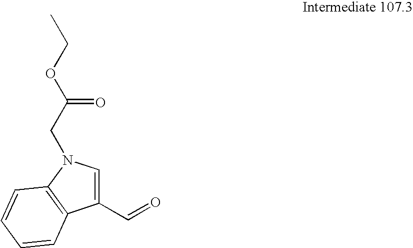

Intermediate 107.3 is synthesized by condensation of indole-3-carbaldehyde (1.00 g, 6.90 mmol) and Ethyl bromoacetate (920 μL, 8.30 mmol) analogously to the preparation of Intermediate 3.4. Colorless oil; ES-MS: M+H=232; HPLC: _{A}t_{Ret}=3.09 min.

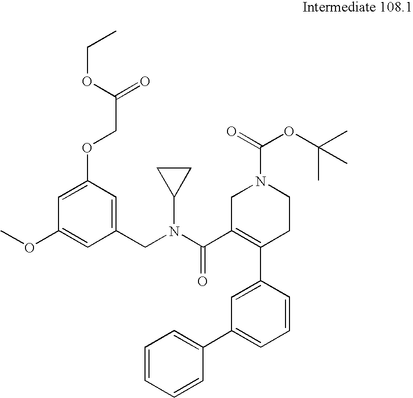

Intermediate 108.1 is synthesized by alkylation of Intermediate 79.4 (333 mg, 0.60 mmol) analogously to the preparation of Intermediate 79.3. White amorphous material; ES-MS: M+H=641; HPLC: t_{Ret}=5.05 min.

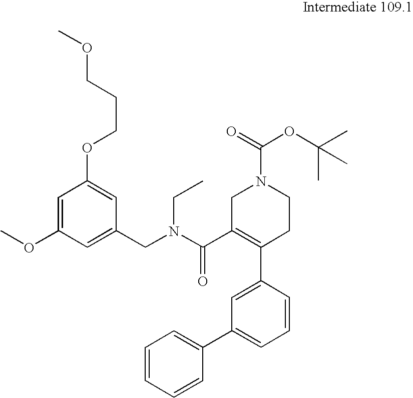

Intermediate 109.1 is synthesized by alkylation of Intermediate 109.2 (172 mg, 0.30 mmol) analogously to the preparation of Intermediate 103.1. Yellow amorphous material; ES-MS: M+H=601; HPLC: t_{Ret}=5.30 min.

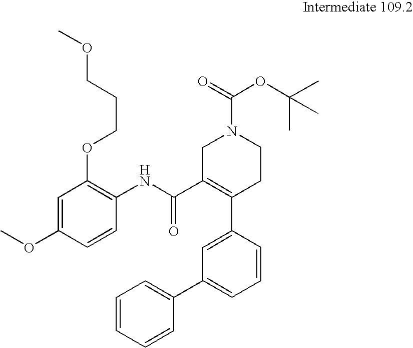

Intermediate 109.2 is synthesized by condensation of Intermediate 1.3 (253 mg, 0.67 mmol) and Intermediate 109.3 (197 mg, 0.93 mmol) analogously to the preparation of Intermediate 103.2. Yellow amorphous material; ES-MS: M+H=573; HPLC: t_{Ret}=5.00 min.

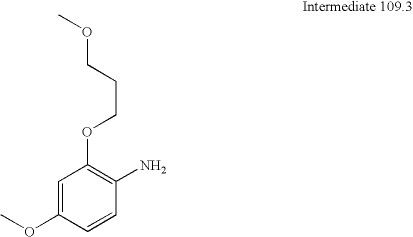

Intermediate 109.3 is synthesized by reduction of Intermediate 109.4 (1.76 g, 2.60 mmol) analogously to the preparation of Intermediate 103.3. White amorphous material; ES-MS: M+H=212; HPLC: t_{Ret}=1.93 min.

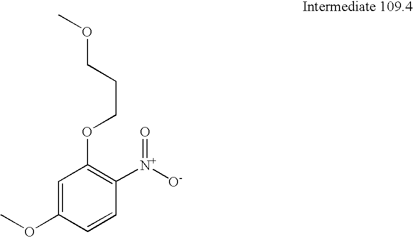

A mixture of Intermediate 109.5 (634 mg, 2.77 mmol) and TBAB (44.6 mg, 0.14 mmol) in MeOH (560 μL) and toluene (3 mL) is refluxed. After stirring for 2 hours, H₂O is added at 0° C. and the reaction mixture is extracted with Et₂O. The combined organic phases are washed with H₂O, brine and dried (Na₂SO₄), concentrated under reduced pressure and silica gel flash chromatography to give Intermediate 109.4 as yellow oil; ES-MS: M+H=242; HPLC: t_{Ret}=3.47 minutes

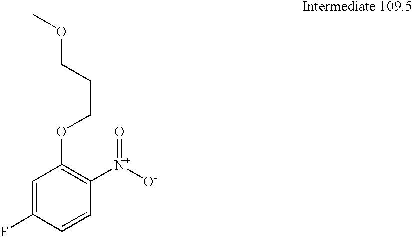

Intermediate 109.5 is synthesized by alkylation of 4-fluoro-2-hydroxy-nitrobenzene (1.57 g, 10.0 mmol) analogously to the preparation of Intermediate 3.4. Yellow oil; ES-MS: M+H=230; HPLC: t_{Ret}=3.54 min.

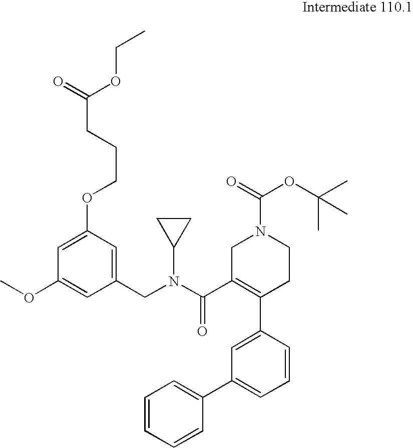

Intermediate 110.1 is synthesized by alkylation of Intermediate 79.4 (166 mg, 0.30 mmol) analogously to the preparation of Intermediate 79.3. White amorphous material; ES-MS: M+H=669; HPLC: t_{Ret}=5.35 min.

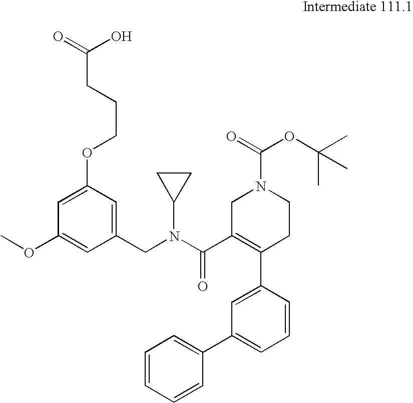

Intermediate 111.1 is synthesized by hydrolysis of Intermediate 110.1 (201 mg, 0.30 mmol) analogously to the preparation of Intermediate 1.3. White amorphous material; ES-MS: M+H=641; HPLC: t_{Ret}=4.59 min.

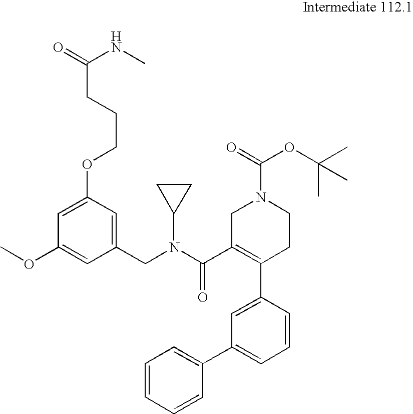

Intermediate 112.1 is synthesized by condensation of Intermediate 111.1 (112 mg, 0.17 mmol) analogously to the preparation of Intermediate 1.2. White amorphous material; ES-MS: M+H=654; HPLC: t_{Ret}=4.45 min.

### Example 113

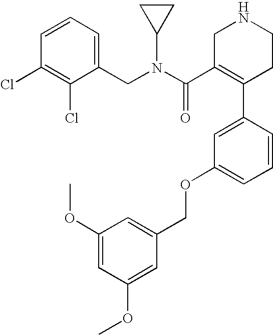

Example 113 is synthesized by deprotection of Intermediate 113.1 (205 mg, 0.3 mmol) analogously to the preparation of Example 1. White powder, ES-MS: M+H=567; HPLC: t_{Ret}=3.65 min.

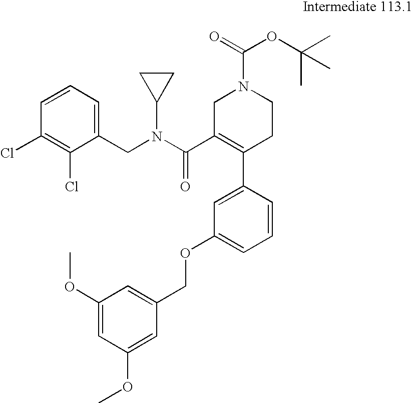

Intermediate 113.1 is synthesized by condensation of Intermediate 113.2 (400 mg, 0.79 mmol) and 1-bromomethyl-2,3-dichlorobenzene (264 mg, 0.94 mmol) analogously to the preparation of Intermediate 1.1. Colorless amorphous material; ES-MS: M+=667; HPLC: t_{Ret}=5.45 min.

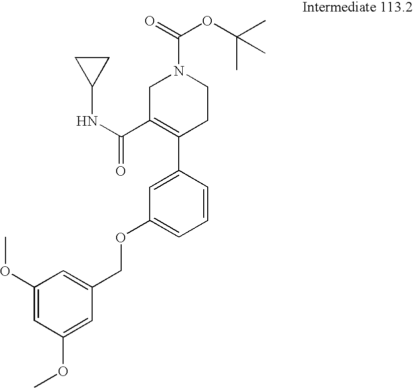

Intermediate 113.2 is synthesized by condensation of Intermediate 113.3 (1.72 g, 3.66 mmol) and an excess amount of cyclopropylamine analogously to the preparation of Intermediate 1.2. White powder; ES-MS: M+H=509; HPLC: t=4.32 min.

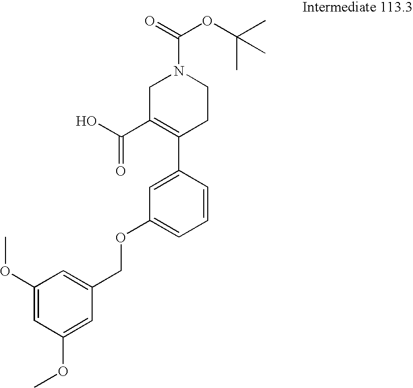

Intermediate 113.3 is synthesized by hydrolysis of Intermediate 113.4 (5.3 g, 11 mmol) analogously to the preparation of Intermediate 1.3. Colorless oil; Rf=0.20 (AcOEt); ¹H NMR (CDCl₃) δ 1.49 (s, 9H), 2.49 (brs, 2H), 3.59 (t, 2H), 3.79 (s, 6H), 4.24 (brs, 2H), 4.99 (s, 2H), 6.41 (t, 1H), 6.56 (d, 2H), 6.75-6.77 (m, 2H), 6.90-6.92 (m, 1H), 7.24 (d, 1H).

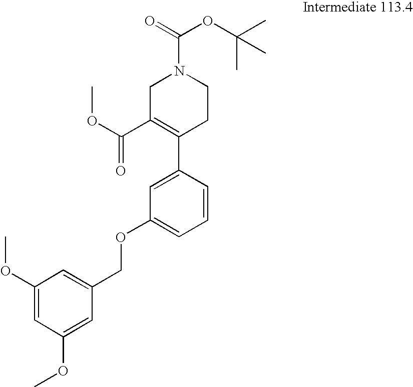

Intermediate 113.4 is synthesized by condensation of 4-trifluoromethanesulfonyloxy-5,6-dihydro-2H-pyridine-1,3-dicarboxylic acid 1-tert-butyl ester 3-methyl ester (5.84 g, 15 mmol) and 3-(3,5-dimethoxybenzyloxy)phenylboronic acid (6.5 g, 22 mmol) analogously to the preparation of Intermediate 1.4. Colorless oil; ES-MS: M-^{t}Bu=428; HPLC: t_{Ret}=4.95 min.

### Example 114

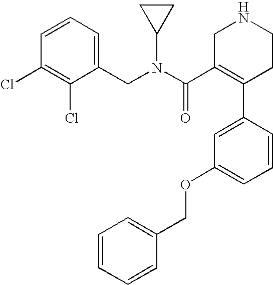

Example 114 is synthesized by deprotection of Intermediate 114.1 (115 mg, 0.19 mmol) analogously to the preparation of Example 1. Solid powder; ES-MS: M+H=507; HPLC: t_{Ret}=3.82 min.

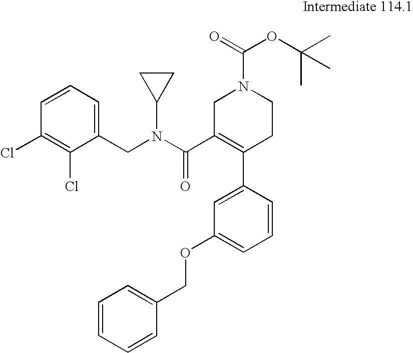

Intermediate 114.1 is synthesized by alkylation of Intermediate 114.2 (100 mg, 0.19 mmol) analogously to the preparation of Intermediate 7.3. White amorphous material; ES-MS: M+H=607; HPLC: t_{Ret}=5.84 min.

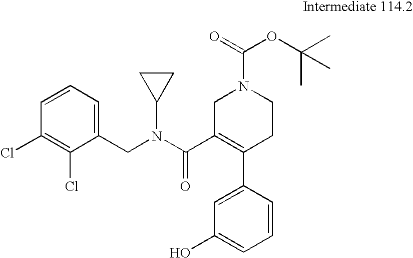

A mixture of Intermediate 114.3 (1.52 g, 2.7 mmol) and 4N dioxane solution of HCl (15 mL) is stirred under N₂ at RT. After stirring for 1 hour, the reaction mixture is concentrated under reduced pressure to give crude product. Then a mixture of crude product, Et₃N (1.12 mL, 8.1 mmol) and (Boc)₂O (707 mg, 3.2 mmol) in CH₂Cl₂ (5 mL) is stirred under N₂ at RT for 1 h. After adding aqueous KHSO₄, the reaction mixture is extracted with EtOAc. The combined organic phases are washed with H₂O, brine and dried (Na₂SO₄). Concentration under reduced pressure and silica gel flash chromatography give Intermediate 114.2 as white amorphous material; ES-MS: M+=517; HPLC: t_{Ret}=4.70 min.

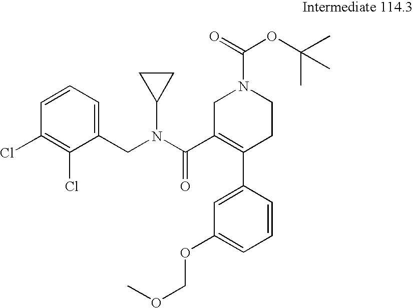

Intermediate 114.3 is synthesized by condensation of Intermediate 114.4 (10.6 g, 26.3 mmol) and 1-bromomethyl-2,3-dichlorobenzene (10.4 g, 39.5 mmol) analogously to the preparation of Intermediate 1.1. White powder; ES-MS: M+=561; HPLC: t_{Ret}=5.30 min.

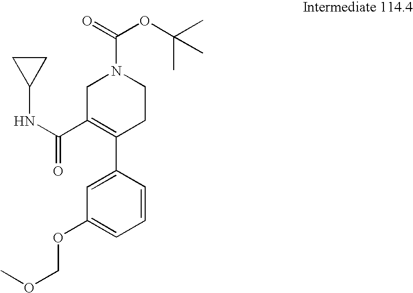

Intermediate 114.4 is synthesized by condensation of Intermediate 114.5 (2.54 g, 7.1 mmol) and cyclopropylamine (0.73 mL, 10.6 mmol) analogously to the preparation of Intermediate 1.2. Colorless oil; Rf=0.23 (EtOAc:n-Hex=1:1); ¹H NMR (CDCl₃) δ 0.5-0.6 (m, 4H), 1.52 (s, 9H), 2.48-2.53 (m, 3H), 3.51 (s, 3H), 3.62 (t, 2H), 4.27, (brs, 2H), 5.08 (brs, 1H), 5.20-5.25 (s, 2H), 6.84-6.87 (m, 2H), 7.02-7.04 (m, 1H), 7.27-7.31 (m, 1H).

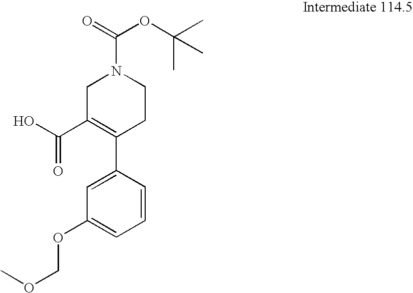

Intermediate 114.5 is synthesized by hydrolysis of Intermediate 114.6 (509 mg, 1.35 mmol) analogously to the preparation of Intermediate 1.3. Colorless oil; Rf=0.30 (EtOAc only); HPLC: t_{Ret}=3.95 min.

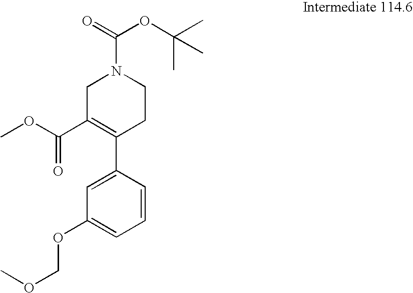

Intermediate 114.6 is synthesized by condensation of 4-trifluoromethanesulfonyloxy-5,6-dihydro-2H-pyridine-1,3-dicarboxylic acid 1-tert-butyl ester 3-methyl ester (5.35 g, 13.7 mmol) and 3-methoxylmethoxyphenylboronic acid (3.75 g, 20.6 mmol) analogously to the preparation of Intermediate 1.4. Colorless oil; ES-MS: M+H=378; HPLC: t_{Ret}=4.37 min.

The following Examples enlisted in Table 2 are synthesized analogously to the preparation of Example 113 and 114. As far as not being commercially available, the synthesis of intermediates for the preparation of compounds of Example 115-140 is described below Table 2 (an asterisk (*) indicates the end of the bond and the end thereof with which the moiety is bound to the rest of the molecule).

TABLE 2

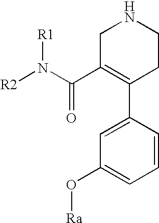

| No. | R1 | R2 | Ra | Analytical data |
|---|---|---|---|---|
| 115 | 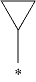 | 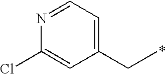 | 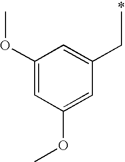 | MS: [M]⁺ = 534 HPLC t_{Ret} = 3.22 min. |
| 116 | 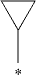 | 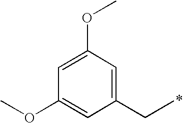 | 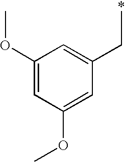 | MS: [M + 1]⁺ = 559 HPLC t_{Ret} = 3.47 min. |
| 117 | 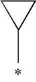 | 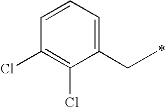 | 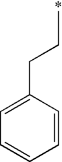 | MS: [M + 1]⁺ = 521 HPLC t_{Ret} = 3.98 min. |
| 118 | 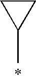 | 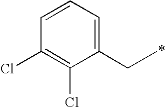 | 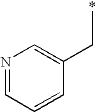 | MS: [M + 1]⁺ = 508 HPLC t_{Ret} = 2.63 min. |
| 119 | 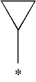 | 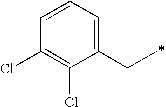 | 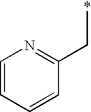 | MS: [M + 1]⁺ = 508 HPLC t_{Ret} = 2.73 min. |
| 120 | 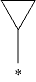 | 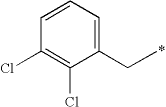 | 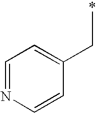 | MS: [M + 1]⁺ = 508 HPLC t_{Ret} = 2.60 min. |
| 121 | 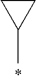 | 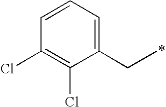 | 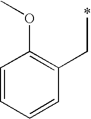 | MS: [M + 1]⁺ = 537 HPLC t_{Ret} = 3.87 min. |
| 122 | 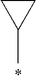 | 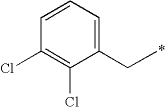 | 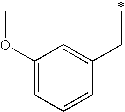 | MS: [M + 1]⁺ = 537 HPLC t_{Ret} = 3.80 min. |
| 123 | 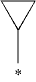 | 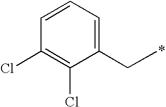 | 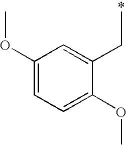 | MS: [M + 1]⁺ = 567 HPLC t_{Ret} = 3.80 min. |
| 124 | 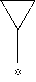 | 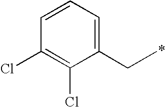 | 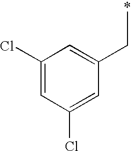 | MS: [M + 1]⁺ = 575 HPLC t_{Ret} = 4.27 min. |
| 125 | 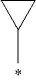 | 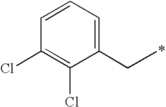 | 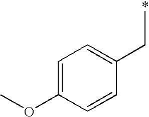 | MS: [M + 1]⁺ = 537 HPLC t_{Ret} = 3.77 min. |
| 126 | 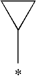 | 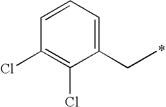 | 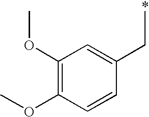 | MS: [M + 1]⁺ = 567 HPLC t_{Ret} = 3.54 min. |
| 127 | 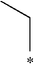 | 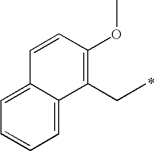 | 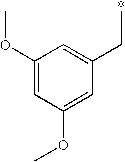 | MS: [M + 1]⁺ = 567 HPLC t_{Ret} = 3.67 min. |
| 128 | 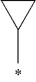 | 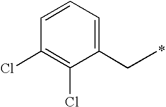 | 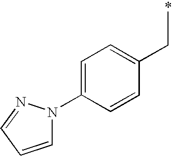 | MS: [M + 1]⁺ = 573 HPLC t_{Ret} = 3.53 min. |
| 129 | 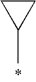 | 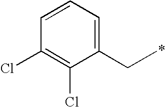 | 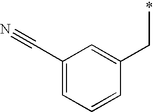 | MS: [M + 1]⁺ = 532 HPLC t_{Ret} = 3.60 min. |
| 130 | 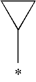 | 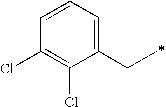 | 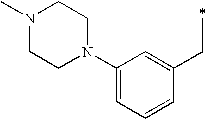 | MS: [M]⁺ = 607 HPLC t_{Ret} = 2.87 min. |
| 131 | 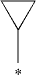 | 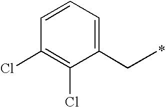 | 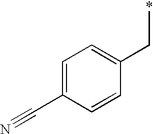 | MS: [M + 1]⁺ = 532 HPLC t_{Ret} = 3.59 min. |
| 132 | 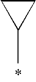 | 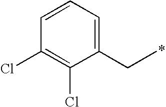 | 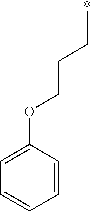 | MS: [M + 1]⁺ = 551 HPLC t_{Ret} = 3.98 min. |
| 133 | 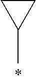 | 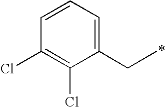 | 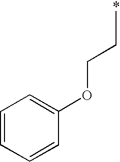 | MS: [M + 1]⁺ = 537 HPLC t_{Ret} = 3.82 min. |
| 134 | 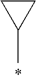 | 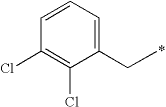 | 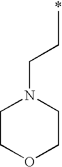 | MS: [M + 1]⁺ = 530 HPLC t_{Ret} = 2.57 min. |
| 135 | 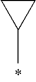 | 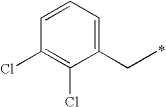 | 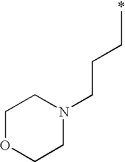 | MS: [M + 1]⁺ = 544 HPLC t_{Ret} = 2.65 min. |
| 136 | 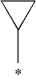 | 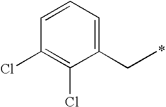 | 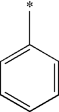 | MS: [M + 1]⁺ = 493 HPLC t_{Ret} = 3.90 min. |
| 137 | 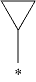 | 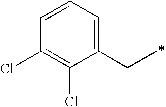 | 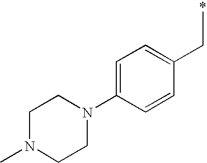 | MS: [M + 1]⁺ = 605 HPLC t_{Ret} = 2.84 min. |
| 138 | 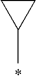 | 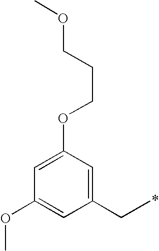 | 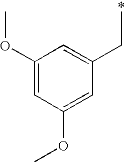 | MS: [M + 1]⁺ = 617 HPLC t_{Ret} = 3.46 min. |
| 139 | 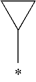 | 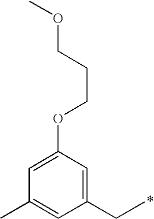 | 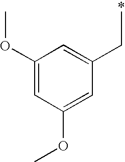 | MS: [M + 1]⁺ = 601 HPLC t_{Ret} = 3.60 min. |
| 140 | 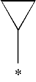 | 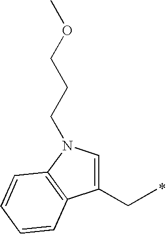 | 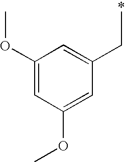 | MS: [M + 1]⁺ = 610 HPLC t_{Ret} = 3.77 min. |

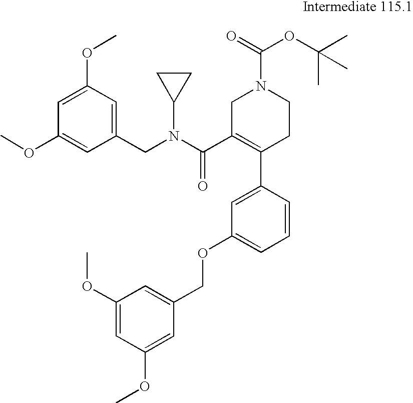

Intermediate 115.1 is synthesized by condensation of Intermediate 115.2 (166 mg, 0.35 mmol) analogously to the preparation of Intermediate 1.1. Colorless amorphous material; ES-MS: M-Boc=559; Rf=0.44 (EtOAc:n-Hex=1:1)

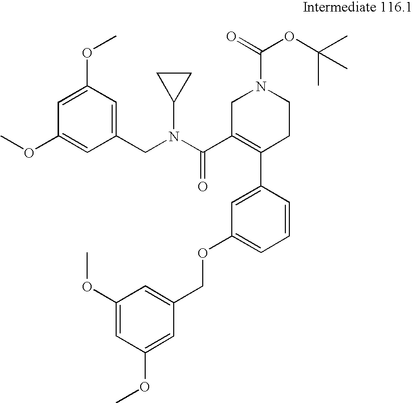

Intermediate 116.1 is synthesized by condensation of Intermediate 116.2 (166 mg, 0.35 mmol) analogously to the preparation of Intermediate 1.1. Colorless amorphous material; ES-MS: M-Boc=559; Rf=0.44 (EtOAc:n-Hex=1:1)

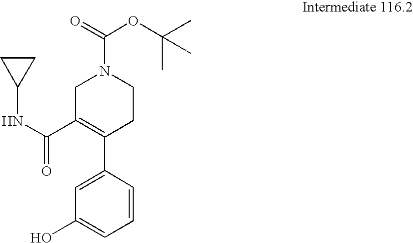

Intermediate 116.2 is synthesized by condensation of Intermediate 116.3 (220 mg, 0.69 mmol) analogously to the preparation of Intermediate 1.2. Colorless oil; ES-MS: M+H=359; HPLC: t_{Ret}=3.10 min.

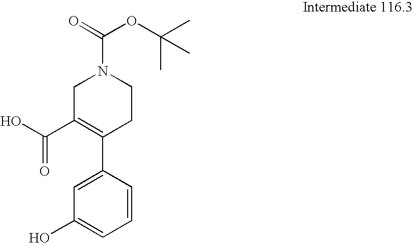

Intermediate 116.3 is synthesized by hydrolysis of Intermediate 116.4 (340 mg, 1.02 mmol) analogously to the preparation of Intermediate 1.3. Colorless oil; ES-MS: M+H=320; HPLC: t_{Ret}=3.14 min.

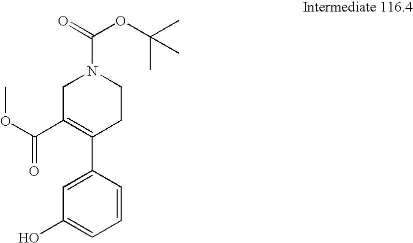

Intermediate 116.4 is synthesized by condensation of 4-trifluoromethanesulfonyloxy-5,6-dihydro-2H-pyridine-1,3-dicarboxylic add 1-tert-butyl ester 3-methyl ester (416 mg, 1.07 mmol) and 3-hydroxyphenylboronic acid (306 mg, 1.39 mmol) analogously to the preparation of Intermediate 1.4. Colorless oil; Rf=0.27 (EtOAc:n-Hex=1:2); HPLC: t_{Ret}=3.63 min.

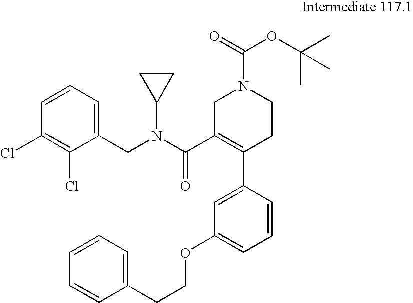

Intermediate 117.1 is synthesized by alkylation of Intermediate 115.2 (100 mg, 0.19 mmol) analogously to the preparation of Intermediate 7.3. White amorphous material; Rf=0.32 (EtOAc Only)

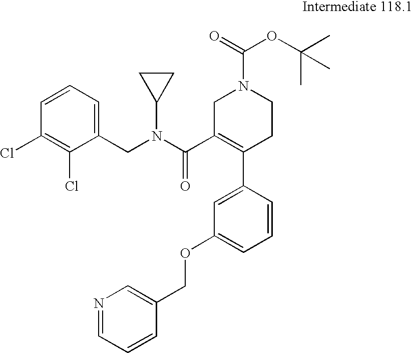

Intermediate 118.1 is synthesized by alkylation of Intermediate 114.2 (100 mg, 0.19 mmol) analogously to the preparation of Intermediate 7.3. White amorphous material; Rf=0.50 (EtOAc)

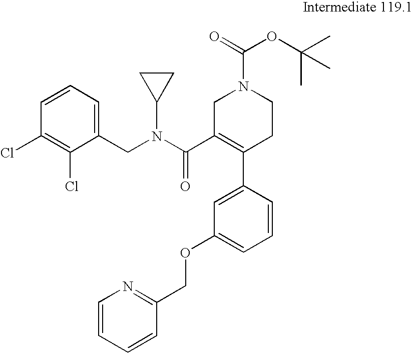

Intermediate 119.1 is synthesized by alkylation of Intermediate 114.2 (100 mg, 0.19 mmol) analogously to the preparation of Intermediate 7.3. White amorphous material; Rf=0.50 (EtOAc)

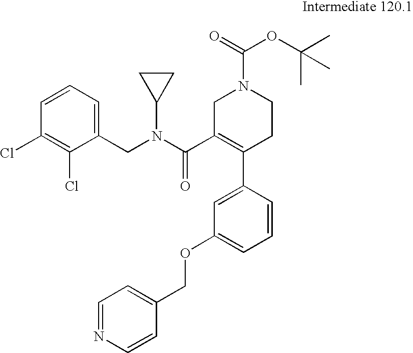

Intermediate 120.1 is synthesized by alkylation of Intermediate 114.2 (100 mg, 0.19 mmol) analogously to the preparation of Intermediate 7.3. While amorphous material; Rf=0.50 (EtOAc)

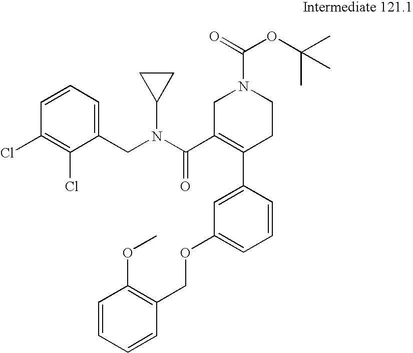

Intermediate 121.1 is synthesized by alkylation of Intermediate 114.2 (100 mg, 0.19 mmol) analogously to the preparation of Intermediate 7.3. White amorphous material; Rf=0.40 (EtOAc)

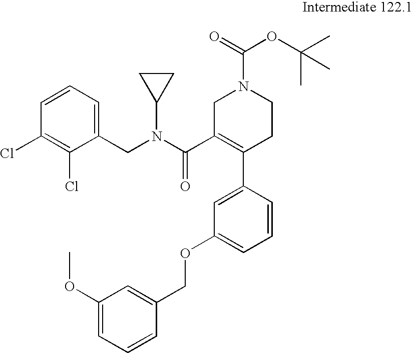

Intermediate 122.1 is synthesized by alkylation of Intermediate 114.2 (100 mg, 0.19 mmol) analogously to the preparation of Intermediate 7.3. White amorphous material; Rf=0.40 (EtOAc)

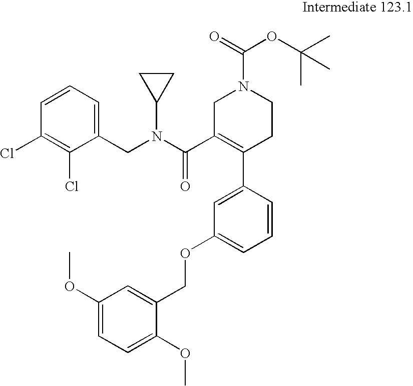

Intermediate 123.1 is synthesized by alkylation of Intermediate 114.2 (100 mg, 0.19 mmol) analogously to the preparation of Intermediate 7.3. White amorphous material; ES-MS: M+H=667, HPLC: t_{Ret}=5.82 min.

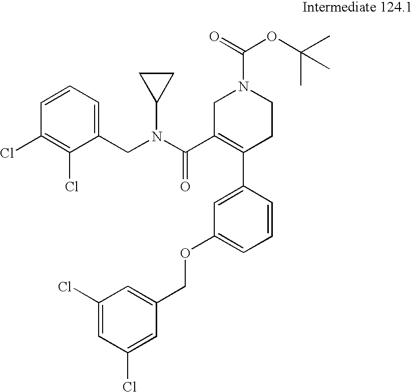

Intermediate 124.1 is synthesized by alkylation of Intermediate 114.2 (100 mg, 0.19 mmol) analogously to the preparation of Intermediate 7.3. White amorphous material; ES-MS: M+H=677; HPLC: t_{Ret}=6.24 min.

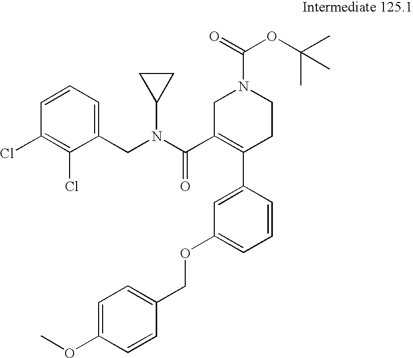

Intermediate 125.1 is synthesized by alkylation of Intermediate 114.2 (100 mg, 0.19 mmol) analogously to the preparation of Intermediate 7.3. White amorphous material; ES-MS: M+H=637; HPLC: t_{Ret}=5.72 min.

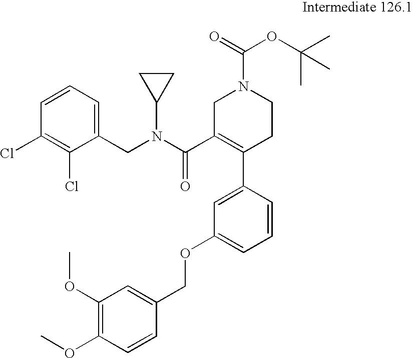

Intermediate 126.1 is synthesized by alkylation of Intermediate 114.2 (100 mg, 0.19 mmol) analogously to the preparation of Intermediate 7.3. White amorphous material; ES-MS: M+H=667; HPLC: t_{Ret}=5.49 min.

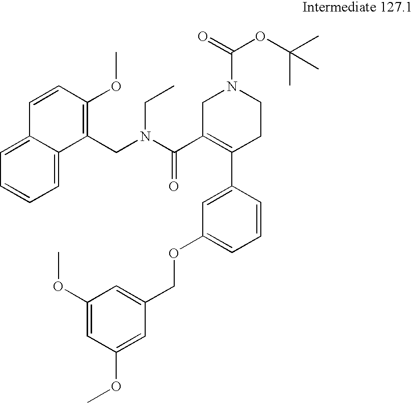

Intermediate 127.1 is synthesized by alkylation of Intermediate 127.2 (114.3 mg, 0.18 mmol) analogously to the preparation of Intermediate 7.3. White amorphous material; ES-MS: M+H=667; HPLC: t_{Ret}=5.52 min.

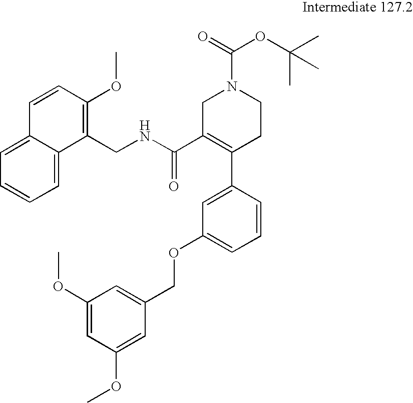

Intermediate 127.2 is synthesized by alkylation of Intermediate 113.3 (304.6 mg, 0.65 mmol) analogously to the preparation of Intermediate 1.2. White amorphous material; ES-MS: M+H=639; HPLC: t_{Ret}=5.22 min.

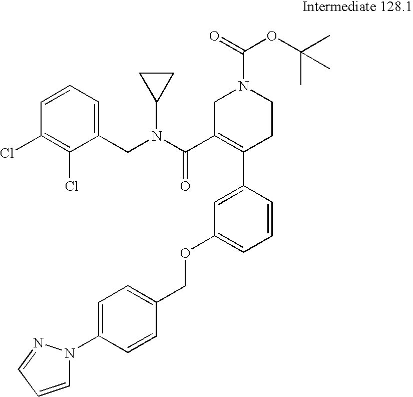

Intermediate 128.1 is synthesized by alkylation of Intermediate 114.2 (100 mg, 0.19 mmol) analogously to the preparation of Intermediate 7.3. White amorphous material; Rf=0.20 (EtOAc:n-Hex=1:2)

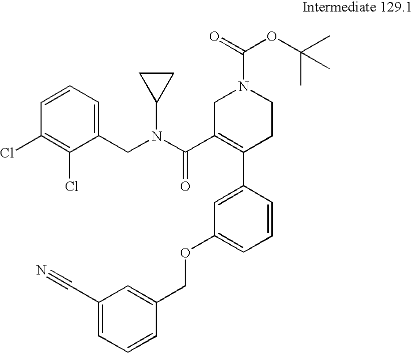

Intermediate 129.1 is synthesized by alkylation of Intermediate 1142 (100 mg, 0.19 mmol) analogously to the preparation of Intermediate 7.3. White amorphous material; Rf=0.24 (EtOAc:n-Hex=1:2)

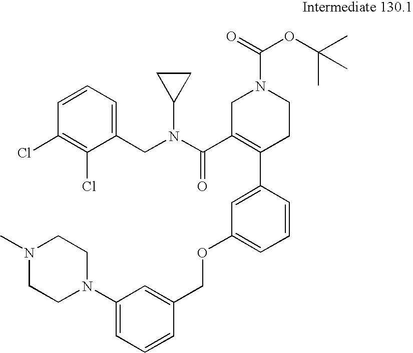

Intermediate 130.1 is synthesized by alkylation of Intermediate 114.2 (258 mg, 0.38 mmol) analogously to the preparation of Intermediate 7.3. White amorphous material; ES-MS: M+=705; HPLC: t_{Ret}=4.17 min.

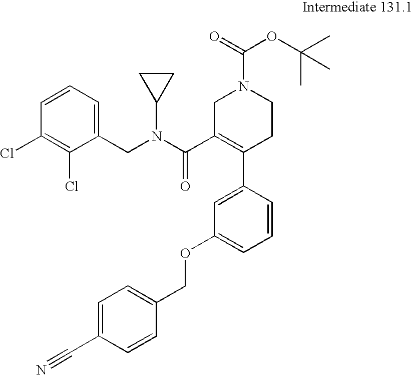

Intermediate 131.1 is synthesized by alkylation of Intermediate 114.2 (100 mg, 0.19 mmol) analogously to the preparation Intermediate 7.3. White amorphous material; Rf=0.24 (EtOAc:n-Hex=1:2)

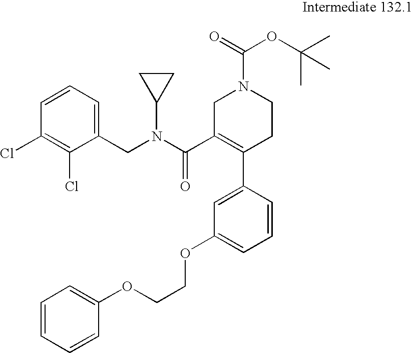

Intermediate 132.1 is synthesized by alkylation of Intermediate 114.2 (100 mg, 0.19 mmol) analogously to the preparation of Intermediate 7.3. White amorphous material; ES-MS: M+H-^{t}Bu=581; Rf=0.29 (n-Hex/EtOAc=3/1)

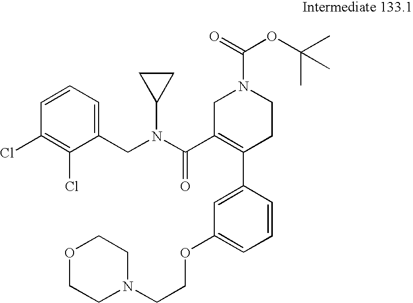

Intermediate 133.1 is synthesized by alkylation of Intermediate 114.2 (100 mg, 0.19 mmol) analogously to the preparation of Intermediate 7.3. White amorphous material; ES-MS: M+H=630; HPLC: t_{Ret}=3.77 min.

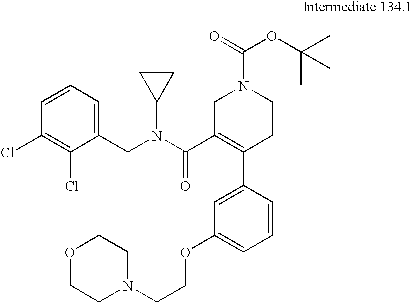

Intermediate 134.1 is synthesized by alkylation of Intermediate 114.2 (100 mg, 0.19 mmol) analogously to the preparation of Intermediate 7.3. White amorphous material; ES-MS: M+H=630; HPLC: t_{Ret}=3.77 min.

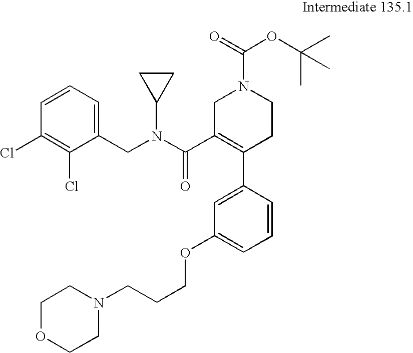

Intermediate 135.1 is synthesized by alkylation of Intermediate 114.2 (100 mg, 0.19 mmol) analogously to the preparation of Intermediate 7.3. White amorphous material; ES-MS: M+H=644; HPLC: t_{Ret}=3.87 min.

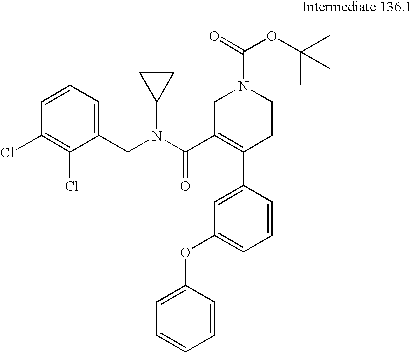

Intermediate 136.1 is synthesized by coupling of Intermediate 114.2 (517 mg, 1.0 mmol) analogously to the preparation of Intermediate 7.3. White amorphous material; ES-MS: M+H=593; HPLC: t_{Ret}=5.82 min.

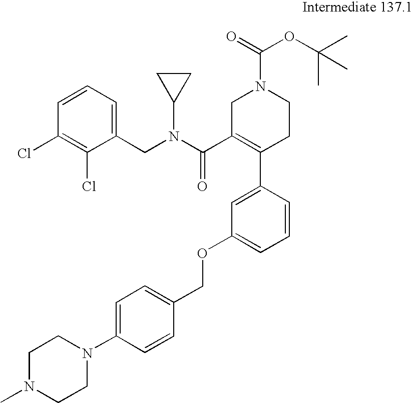

Intermediate 137.1 is synthesized by alkylation of Intermediate 114.2 (400 mg, 0.58 mmol) analogously to the preparation of Intermediate 7.3. White amorphous material; ES-MS: M+=705; HPLC: t_{Ret}=4.12 min.

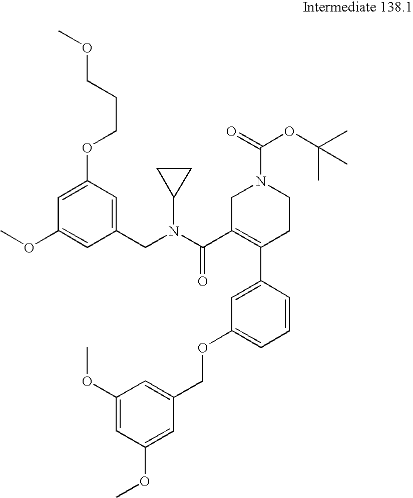

Intermediate 138.1 is synthesized by condensation of Intermediate 113.2 (210 mg, 0.41 mmol) analogously to the preparation of Intermediate 7.3. White amorphous material; ES-MS: M+H=718; HPLC: t_{Ret}=5.25 min.

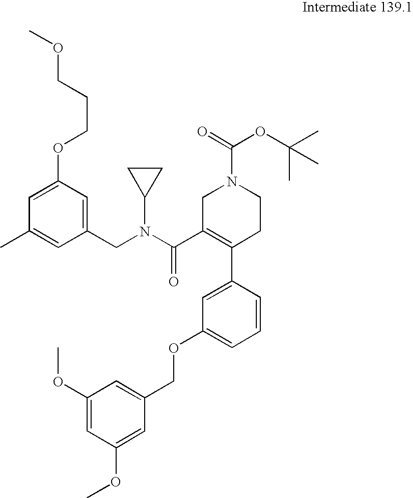

Intermediate 139.1 is synthesized by condensation of Intermediate 113.2 (280 mg, 0.55 mmol) analogously to the preparation of Intermediate 7.3. White amorphous material; Rf=0.20 (n-Hex:EtOAc=4:1)

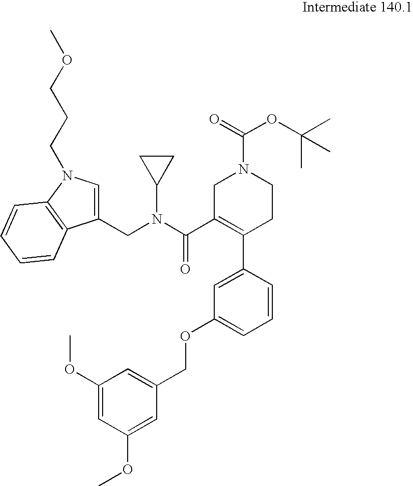

Intermediate 140.1 is synthesized by coupling of Intermediate 113.3 (200 mg, 0.42 mmol) analogously to the preparation of Intermediate 3.1. White amorphous material; ES-MS: M+H=710; HPLC: t_{Ret}=5.47 min.

### Example 141

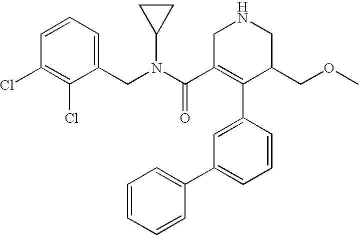

A mixture of Intermediate 141.1 (84 mg, 0.137 mmol) and 1-chloroethyl chloroformate (0.21 mL) in 1,2-dichloroethane (1.5 mL) is stirred under N₂ at 90° C. for 2 h. After MeOH (32 mL) is added, the reaction mixture is refluxed for 2 h. After adding H₂O, the reaction mixture is extracted with EtOAc. The combined organic phases are washed with H₂O, brine and dried (Na₂SO₄). Concentration under reduced pressure and silica gel flash chromatography give Example 141 as amorphous material; ES-MS: M+=521; HPLC: t_{Ret}=3.77 min.

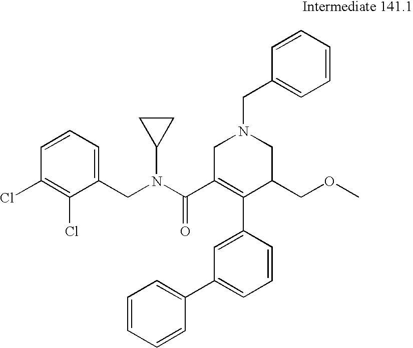

Intermediate 141.1 is synthesized by condensation of Intermediate 141.2 (102 mg, 0.23 mmol) and 1-bromomethyl-2,3-dichlorobenzene (67 mg, 0.27 mmol) analogously to the preparation of Intermediate 1.1. Colorless oil; ES-MS: M+=611; HPLC: t_{Ret}=4.55 min.

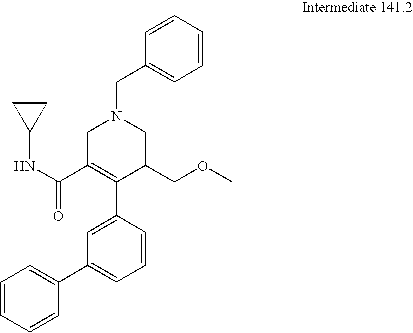

Intermediate 141.2 is synthesized by condensation of Intermediate 141.3 (190 mg, 0.46 mmol) and cyclopropylamine (0.11 mL, 1.65 mmol) analogously to the preparation of Intermediate 1.2. Colorless oil; ES-MS: M+H=453; HPLC: t_{Ret}=3.22 min.

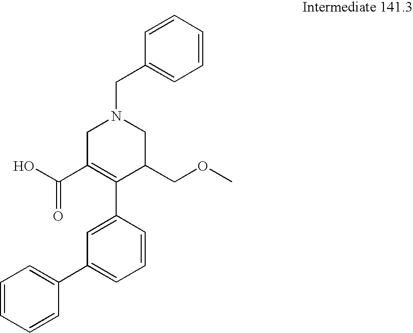

Intermediate 141.3 is synthesized by hydrolysis of Intermediate 141.4 (235 mg, 0.55 mmol) analogously to the preparation of Intermediate 1.3. Amorphous material; ES-MS: M+H 414; HPLC: t_{Ret}=3.55 min.

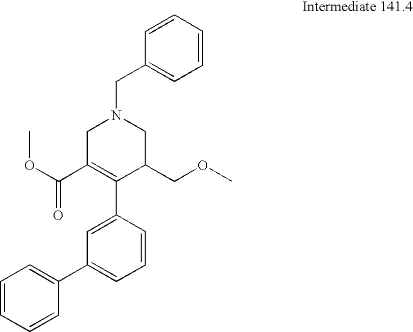

Intermediate 141.4 is synthesized by condensation of Intermediate 141.5 (330 mg, 0.78 mmol) and 3-biphenylboronic acid (232 mg, 1.17 mmol) analogously to the preparation of Intermediate 1.4. Amorphous material; ES-MS: M+H=428; HPLC: t_{Ret}=3.87 min.

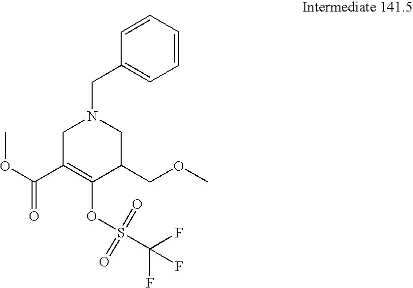

A mixture of methyl 1-benzyl-4-oxo-3-piperidine-carboxylate hydrochloride (6.0 g, 21.1 mmol) and 2M THF solution of LDA (42.2 ml, 84.4 mmol) in THF (40 mL) is stirred under N₂ at 0° C. After stirring at 0° C. for 40 min, MOMCl (1.72 mL, 29.6 mmol) is added, and the reaction mixture is stirred at RT for 1 h. After adding H₂O, the reaction mixture is extracted with EtOAc. The combined organic phases are washed with H₂O, brine and dried (Na₂SO₄). Concentration under reduced pressure gives crude product. This crude product is used without purification. To a mixture of this crude and DIEA (3.5 ml, 25.3 mmol) in DCM (40 mL), Tf₂O (1.38 mL, 8.44 mmol) is added at −78° C. After stirring at RT for 1 h, the reaction mixture is quenched by slowly adding H₂O, and the mixture is extracted with EtOAc. The combined organic phases are washed with H₂O, brine and dried (Na₂SO₄). Concentration under reduced pressure and silica gel flash chromatography give Intermediate 141.5 as white amorphous material; ES-MS: M+H=424; HPLC: t_{Ret}=2.97 min.

### Example 142

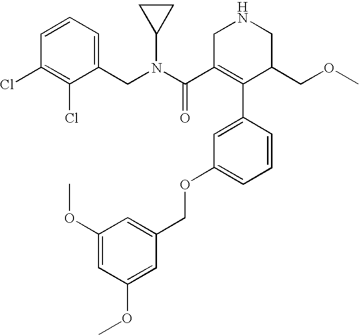

Example 142 is synthesized by deprotection of Intermediate 142.1 (160 mg, 0.23 mmol) analogously to the preparation of Example 1. Amorphous material; ES-MS: M+=611; HPLC: t_{Ret}=3.80 min.

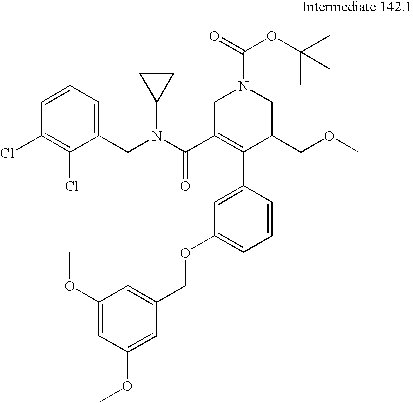

Intermediate 142.1 is synthesized by condensation of Intermediate 142.2 (194 mg, 0.35 mmol) and 1-bromomethyl-2,3-dichlorobenzene (140 mg, 0.53 mmol) analogously to the preparation of Intermediate 1.1. Colorless oil; ES-MS: M+=711; HPLC: t_{Ret}=5.45 min.

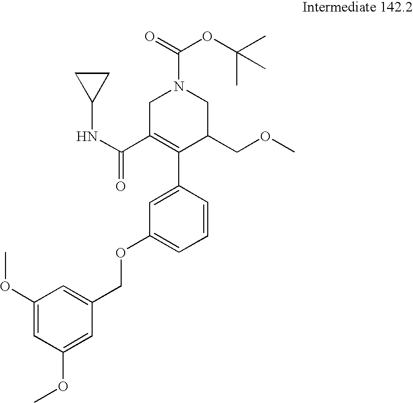

Intermediate 142.2 is synthesized by condensation of Intermediate 142.3 (328 mg, 0.64 mmol) and cyclopropylamine (0.15 mL, 2.2 mmol) analogously to the preparation of Intermediate 1.2. Amorphous material; ES-MS: M+H=553; HPLC: t_{Ret}=4.12 min.

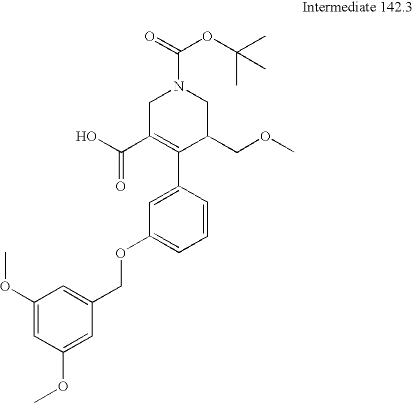

Intermediate 142.3 is synthesized by hydrolysis of Intermediate 142.4 (390 mg, 0.74 mmol) analogously to the preparation of Intermediate 1.3. Amorphous material; Rf=0.20 (EtOAc); ¹H NMR (CDCl₃) δ 1.50 (s, 9H), 2.68-2.70 (m, 1H), 3.11-3.21 (m, 6H), 3.78 (s, 9H), 4.97-5.03, (m, 2H), 5.08 (brs, 1H), 5.20-5.25 (s, 2H), 6.08-7.29 (m, 6H).

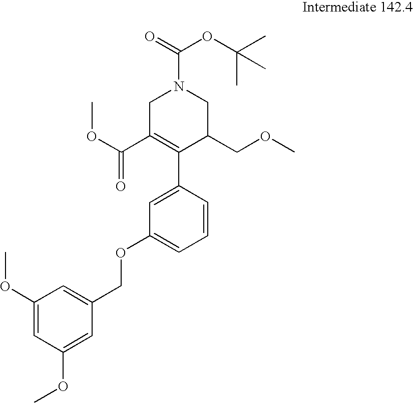

Intermediate 142.4 is synthesized by condensation of Intermediate 142.5 (410 mg, 0.95 mmol) and 3-(3,5-dimethoxybenzyloxy)phenylboronic acid (684 mg, 2.37 mmol) analogously to the preparation of Intermediate 1.4. Amorphous material; ES-MS: M+=528; HPLC: t_{Ret}=4.75 min.

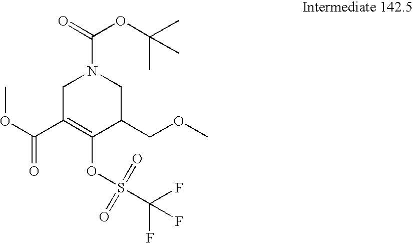

Intermediate 142.5 is synthesized by alkylation of 4-trifluoromethanesulfonyloxy-5,6-dihydro-2H-pyridine-1,3-dicarboxylic acid 1-tert-butyl ester 3-methyl ester (3.0 g, 11.7 mmol) analogously to the preparation of Intermediate 141.5. Amorphous material; ES-MS: M+H 434; HPLC: t_{Ret}=4.32 min.

### Example 143

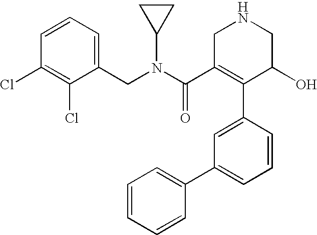

Example 143 is synthesized by deprotection of Intermediate 143.1 (88 mg, 0.14 mmol) analogously to the preparation of Example 1. Colorless amorphous material; ES-MS: M+=493; HPLC: t_{Ret}=3.52 min.

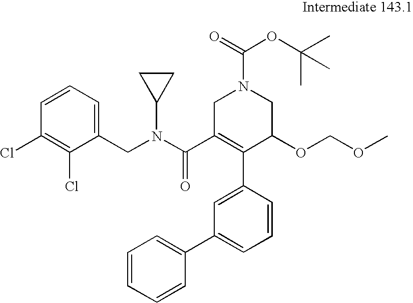

Intermediate 143.1 is synthesized by condensation of Intermediate 1432 (88 mg, 0.18 mmol) and 1-bromomethyl-2,3-dichlorobenzene (53 mg, 0.22 mmol) analogously to the preparation of Intermediate 1.1. Yellow oil; ES-MS: M+=637.4; HPLC: t_{Ret}=5.64 min.

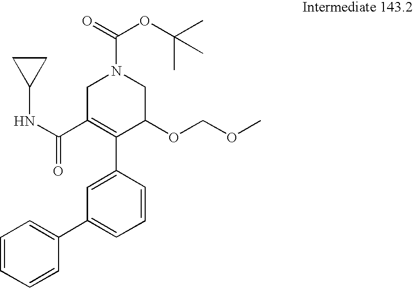

Intermediate 143.2 is synthesized by condensation of Intermediate 143.3 (100 mg, 0.23 mmol) and cyclopropylamine (0.02 mL, 0.27 mmol) analogously to the preparation of Intermediate 1.2 White amorphous material; ¹H-NMR (CDCl₃) δ 0.48-0.52 (m, 4H), 1.50 (s, 9H), 2.42-2.50 (m, 1H), 3.04 (s, 3H), 3.31 (dd, 1H), 4.00 (d, 1H), 4.10-4.22 (m, 1H), 4.32-4.49 (m, 1H), 4.56 (d, 1H), 4.60-4.75 (m, 1H), 4.72 (d, 1H), 5.13 (brs, 1H), 7.22 (d, 1H), 7.32-7.40 (m, 1H), 7.41-7.48 (m, 3H), 7.51 (s, 1H), 7.56-7.60 (m, 3H). Rf=0.57 (hexane/EtOAc 1:3).

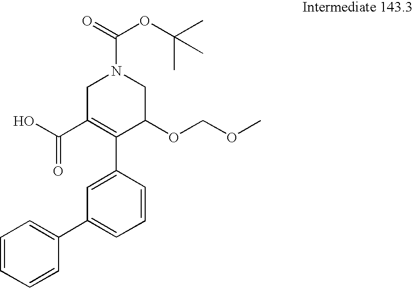

Intermediate 143.3 is synthesized by hydrolysis of Intermediate 143.4 (408 mg, 0.90 mmol) analogously to the preparation of Intermediate 1.3. White amorphous material; ¹H-NMR (CDCl₃) δ 1.50 (s, 9H), 2.91 (s, 3H), 3.28-3.35 (m, 1H), 3.89-4.02 (m, 1H), 4.13-4.22 (m, 1H), 4.34 (d, J=12.0 Hz, 1H), 4.39-4.52 (m, 1H), 4.69 (d, 1H), 4.57-4.85 (m, 1H), 7.22 (d, 1H), 7.31-7.50 (m, 5H), 7.52-7.60 (m, 3H). Rf=0.48 (hexane/EtOAc 1:3).

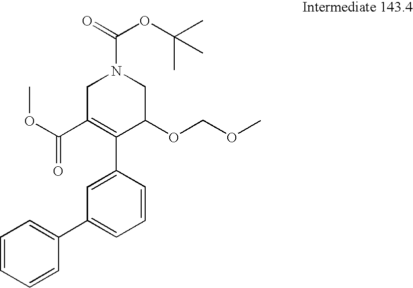

To a solution of Intermediate 143.5 (514 mg, 1.26 mmol) in DIEA (5 mL) and DCM (10 mL), MOMCl (0.14 mL, 1.88 mmol) is added at 0° C. After stirring at RT for 10 h and adding H₂O (15 mL), the reaction mixture is extracted with EtOAc (30 mL, 2×). The combined organic phases are washed with H₂O, brine and dried (MgSO₄). Concentration under reduced pressure and silica gel flash chromatography give Intermediate 143.4 as a yellow amorphous material; ¹H-NMR (400 MHz, CDCl₃) δ 1.50 (s, 9H), 2.29 (s, 3H), 3.28-3.32 (m, 1H), 3.50 (s, 3H), 3.86-4.00 (m, 1H), 4.12-4.25 (m, 1H), 4.37 (d, 1H), 4.40-4.55 (m, 1H), 4.69 (d, 1H), 4.59-4.86 (m, 1H), 7.19 (d, 1H), 7.31-7.48 (m, 5H), 7.50-7.59 (m, 3H). Rf=0.22 (hexane/EtOAc 3:1).

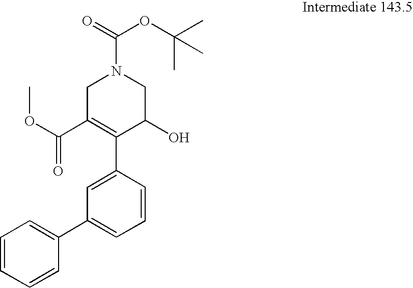

A mixture of Intermediate 143.6 (128 mg, 0.31 mmol) and NaOMe (25 mg, 0.47 mmol) in MeOH (15 mL) is refluxed at 95° C. for 2 h. After cooling down to RT, the reaction mixture is concentrated under reduced pressure. After adding saturated NaHCO₃ solution (15 mL), the reaction mixture is extracted with DCM (30 mL, 2×). The combined organic phases are washed with H₂O, brine and dried (MgSO₄). Concentration under reduced pressure and silica gel flash chromatography give Intermediate 143.5 as a colorless amorphous material; ¹H-NMR (CDCl₃) δ 1.52 (s, 9H), 1.90 (brs, 1H), 3.56 (s, 3H), 3.54-3.61 (m, 1H), 3.39-3.99 (m, 1H), 4.02-4.12 (m, 1H), 4.43-4.59 (m, 2H), 7.20 (d, 1H), 7.35 (t, 1H), 7.40-7.49 (m, 4H), 7.54-7.62 (m, 3H) Rf=0.19 (hexane/EtOAc 3:1).

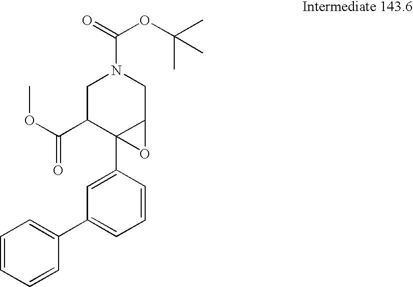

To a solution of Intermediate 143.7 (155 mg, 0.39 mmol) in DCM (10 mL), m-CPBA (243 mg, 0.99 mmol) is added at 0° C. After stirring at RT for 10 h and adding saturated NaHCO₃ solution (15 mL) and Na₂S₂O₃ solution (15 mL) at 0° C., the reaction mixture is extracted with DCM (30 mL, 2×). The combined organic phases are washed with H₂O, brine and dried (MgSO₄). Concentration under reduced pressure and silica gel flash chromatography give Intermediate 143.6 as a colorless amorphous material; ¹H-NMR (CDCl₃) δ 1.43-1.52 (m, 9H), 2.52-2.60 (m, 0.7H), 3.15-3.20 (m, 0.3H), 3.35-3.79 (m, 3H), 3.50 (s, 1H), 3.58 (s, 2H), 4.02-4.38 (m, 2H), 7.32-7.61 (m, 9H). Rf=0.33 (hexane/EtOAc 3:1).

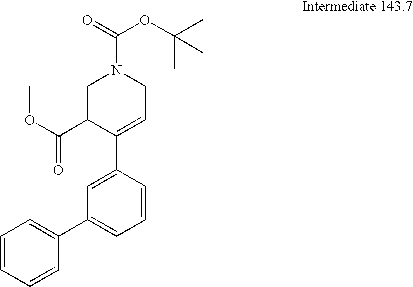

To a mixture of 2M THF solution of LDA (0.26 mL, 0.52 mmol) and HMPA (0.01 mL, 0.52 mmol) in THF (3 mL), a solution of Intermediate 1.4 (185 mg, 0.47 mmol) in THF (5 mL) is added under N₂ at −78° C. under N₂ for 5 min. The reaction mixture is stirred at −78° C. for 1 h, and then added to saturated NH₄Cl solution (15 mL) at 0° C. for 10 min. After adding H₂O, the reaction mixture is extracted with Et₂O (30 mL, 2×). The combined organic phases are washed with H₂O, brine and dried (MgSO₄). Concentration under reduced pressure and silica gel flash chromatography give Intermediate 143.7 as a colorless oil; ¹H-NMR (400 MHz, CDCl₃) δ 1.48 (s, 9H), 3.33-3.46 (m, 1H), 3.58 (s, 3H), 3.69-3.78 (m, 1H), 3.82-3.99 (m, 1H), 4.32-4.56 (m, 2H), 6.20-6.30 (m, 1H), 7.29-7.60 (m, 9H). Rf=0.33 (hexane/EtOAc 3:1).

### Example 144

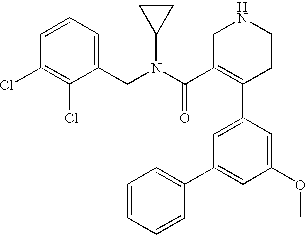

Example 144 is synthesized by deprotection of Intermediate 144.1 (165 mg, 0.3 mmol) analogously to the preparation of Example 1. Amorphous material; ES-MS: M+H=507; HPLC: t_{Ret}=3.73 min.

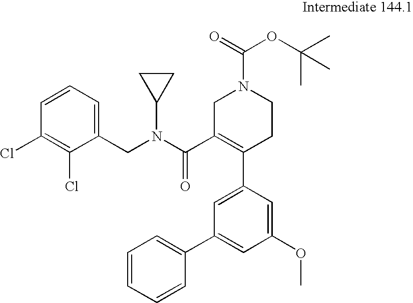

Intermediate 144.1 is synthesized by condensation of Intermediate 1442 (205 mg, 0.5 mmol) and 2,3-dichlorobenzylbromide (132 mg, 0.55 mmol) analogously to the preparation of Intermediate 1.1. White amorphous material; ES-MS: M+H-Boc 507; HPLC: t_{Ret}=3.70 min.

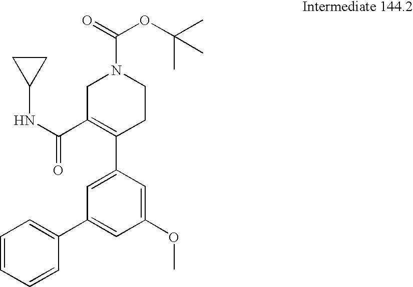

Intermediate 144.2 is synthesized by condensation of Intermediate 144.3 (204.7 mg, 0.5 mmol) and cyclopropylamine (41.3 mL, 0.6 mmol) analogously to the preparation of Intermediate 1.2. White amorphous material; ES-MS: M+H=607; HPLC: t_{Ret}=5.60 min.

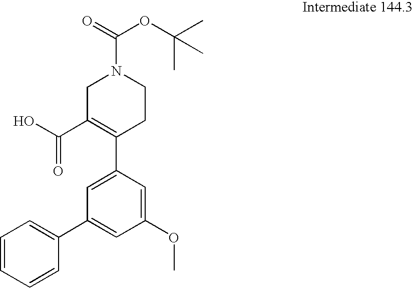

Intermediate 144.3 is synthesized by hydrolysis of Intermediate 144.4 (390.0 mg, 0.9 mmol) analogously to the preparation of Intermediate 1.3. Colorless oil; ES-MS: M-^{t}BuO=336; HPLC: t_{Ret}=4.43 min.

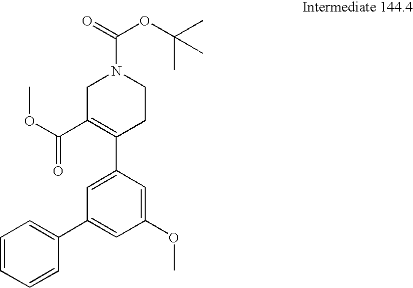

Intermediate 144.4 is synthesized by condensation of 4-trifluoromethanesulfonyloxy-5,6-dihydro-2H-pyridine-1,3-dicarboxylic acid 1-tert-butyl ester 3-methyl ester (1.15 g, 2.89 mmol) and boronate (980 mg, 3.16 mmol) made from Intermediate 144.5 analogously to the preparation of Intermediate 1.4. Colorless oil; ES-MS: M-87=336; HPLC: t_{Ret}=4.43 min.

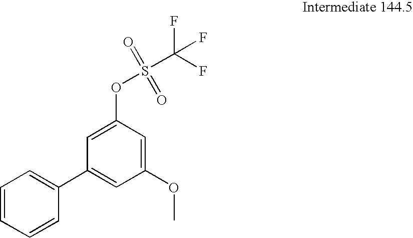

A mixture of 3-methoxy-5-phenyl-phenol (848 mg, 4.23 mmol) (see e.g. Tetrahedron Letters (1991), 32(29), 3441-3444), Tf₂O (0.76 mL, 4.65 mmol) and DIEA (0.87 mL, 5.08 mmol) in DCM (20 mL) is stirred at 0° C. for 3.5 h. After adding saturated NaHCO₃ solution, the reaction mixture is extracted with DCM. The combined organic phases are washed with H₂O, brine and dried (MgSO₄). Concentration under reduced pressure and silica gel flash chromatography give Intermediate 144.5 as a colorless amorphous material; ES-MS: M+H=333; HPLC: t_{Ret}=5.12 min.

### Example 145

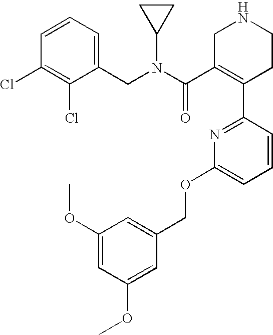

Example 145 is synthesized by deprotection of Intermediate 145.1 (52 mg, 0.078 mmol) analogously to the preparation of Example 1. Solid powder; ES-MS: M=568; HPLC: t_{Ret}=3.75 min.

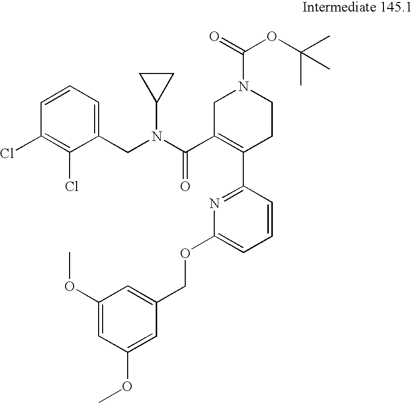

Intermediate 145.1 is synthesized by condensation of Intermediate 145.2 and 1-bromomethyl-2,3-dichlorobenzene (182 mg, 0.76 mmol) analogously to the preparation of Intermediate 1.1. Colorless oil; ES-MS: M+=668; HPLC: t_{Ret}=5.75 min.

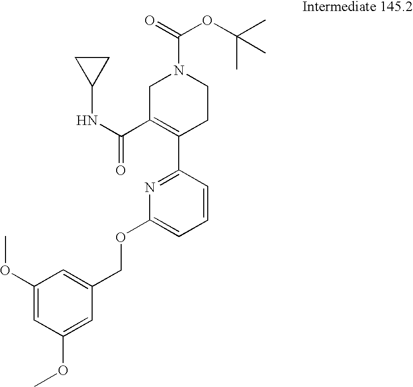

Intermediate 145.2 is synthesized by condensation of Intermediate 145.3 (180.3 mg, 0.38 mmol) and cyclopropylamine (0.057 mL, 0.77 mmol) analogously to the preparation of Intermediate 1.2. White amorphous material; ES-MS: M+H=510; HPLC: t_{Ret}=4.25 min.

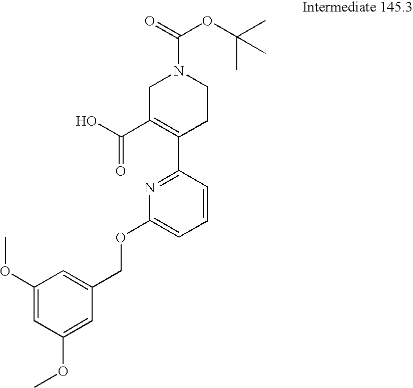

Intermediate 145.3 is synthesized by hydrolysis of Intermediate 145.4 (201 mg, 0.41 mmol) analogously to the preparation of Intermediate 1.3. Colorless oil; ES-MS: M+H=471; HPLC: t_{Ret}=4.18 min.

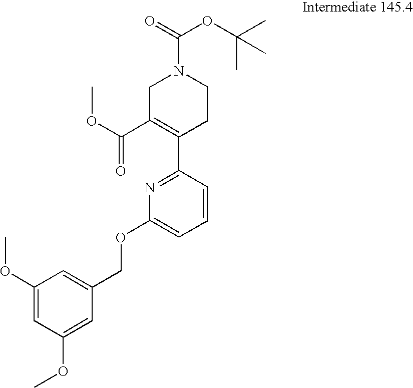

Intermediate 145.4 is synthesized by condensation of 4-trifluoromethanesulfonyloxy-5,6-dihydro-2H-pyridine-1,3-dicarboxylic acid 1-tert-butyl ester 3-methyl ester (238 mg, 0.61 mmol) and Intermediate 145.5 (177 mg, 0.61 mmol) analogously to the preparation of Intermediate 1.4. Colorless oil; ES-MS: M+H=485; HPLC: t_{Ret}=4.85 min.

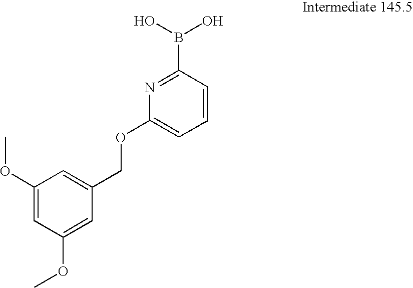

A mixture of Intermediate 145.6 (1.04 g, 3.2 mmol) and 1.6M hexane solution of nBuLi (2.4 mL, 3.85 mmol) in THF (16 mL) is stirred under N₂ at −78° C. After stirring at −78° C. for 1 h, (iPrO)₃B (0.9 mL, 3.85 mmol) is added, and the reaction mixture is stirred at RT for 3 h. The reaction mixture is adjusted to weakly acidic pH by slowly adding 2N HCl, and the mixture is extracted with EtOAc. The combined organic phases are washed with H₂O, brine and dried (Na₂SO₄). Concentration under reduced pressure and silica gel flash chromatography give Intermediate 145.5 as white amorphous material; ES-MS: M+H=290; HPLC: t_{Ret}=2.75 min.

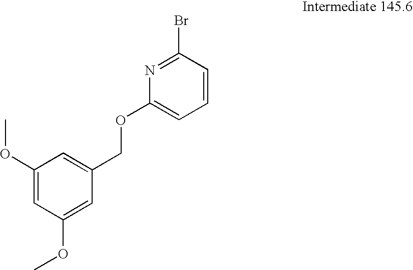

A mixture of 2,6-dibromopyridine (2.06 g, 8.7 mmol), 3,5-dimethoxybenzyl alcohol (1.39 g, 8.26 mmol) and NaH (383 mg, 9.57 mmol) in DMF (35 mL) is stirred under N₂ at 0° C. for 2.5 h. After adding H₂O, the reaction mixture is extracted with EtOAc. The combined organic phases are washed with H₂O, brine and dried (Na₂SO₄). Concentration under reduced pressure and silica gel flash chromatography give Intermediate 145.6 as amorphous material; ES-MS: M+H=326; HPLC: t_{Ret}=4.65 min.

### Example 146

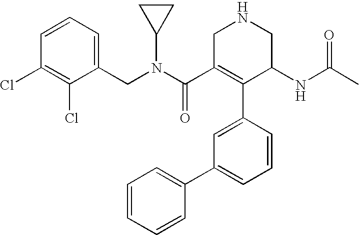

Example 146 is synthesized by deprotection of Intermediate 146.1 (54 mg, 0.09 mmol) analogously to the preparation of Example 1. Colorless amorphous material; ES-MS: M+=534.4; HPLC: t_{Ret}=3.67 min.

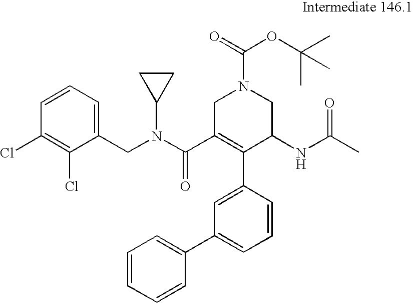

To a mixture of Intermediate 146.2 (80 mg, 0.14 mmol) and NEt₃ (0.03 mL, 0.20 mmol) in DCM (10 mL), AcCl (0.01 mL, 0.16 mmol) in DCM (3 mL) is added at 0° C. After stirring at RT for 4 h, H₂O (10 mL) is added. The mixture is extracted with DCM (20 mL, ×2). The combined organic phases are washed with H₂O, brine and dried (MgSO₄). Concentration under reduced pressure and silica gel flash chromatography give Intermediate 146.1 as a colorless oil; ¹H-NMR (CDCl₃) δ 0.32-0.58 (m, 2H), 0.62-0.73 (m, 2H), 1.51 (s, 9H), 1.58 (brs, 2H), 2.19-2.20 (m, 1H), 3.27-4.97 (m, 7H), 6.30-6.40 (m, 1H), 6.78 (t, 1H), 6.41 (d, 1H), 6.84 (t, 1H), 7.20-7.61 (m, 10H). Rf=0.35 (hexane/EtOAc 1:1).

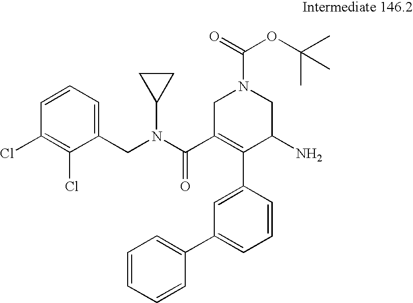

To a solution of Intermediate 146.3 (89 mg, 0.14 mmol) in THF (10 mL) and H₂O (3 mL), PPh₃ (57 mg, 0.22 mmol) is added at 0° C. After stirring at RT for 10 h, H₂O (10 mL) is added. The mixture is extracted with Et₂O (20 mL, ×2). The combined organic phases are washed with H₂O, brine and dried (Na₂SO₄). Concentration under reduced pressure and silica gel flash chromatography give Intermediate 146.2 as a colorless oil; ¹H-NMR (CDCl₃) δ 0.40-0.55 (m, 2H), 0.62-0.73 (m, 2H), 1.51 (s, 9H), 2.01-2.12 (m, 1H), 2.05 (s, 3H), 3.26-3.53 (m, 1H), 3.75-4.40 (m, 3H), 4.54-4.85 (m, 2H), 5.04-5.29 (m, 1H), 5.53 (d, 2H) 6.30-6.40 (m, 1H), 6.78 (t, 1H), 7.17-7.71 (m, 10H). Rf=0.01 (EtOAc).

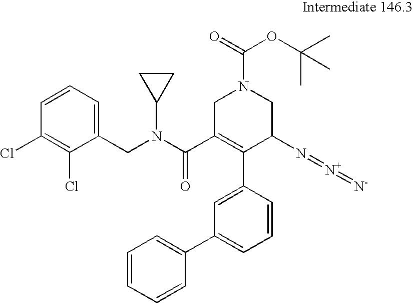

A mixture of Intermediate 146.4 (180 mg, 0.27 mmol) and NaN₃ (53 mg, 0.80 mmol) in DMF (15 mL) is stirred at 95° C. for 10 h. After cooling down to 0° C., to the reaction mixture H₂O is added (25 mL), and the reaction mixture is extracted with Et₂O (30 mL, 2×). The combined organic phases are washed with H₂O, brine and dried (MgSO₄). Concentration under reduced pressure and silica gel flash chromatography give Intermediate 146.3 as a brown amorphous material; ¹H-NMR (CDCl₃) δ 0.49-0.80 (m, 4H), 1.51 (s, 9H), 2.10-2.48 (m, 1H), 3.27-4.97 (m, 7H), 6.22-6.45 (m, 1H), 6.72-6.85 (m, 1H), 7.20-7.79 (m, 10H). Rf=0.61 (hexane/EtOAc 1:1).

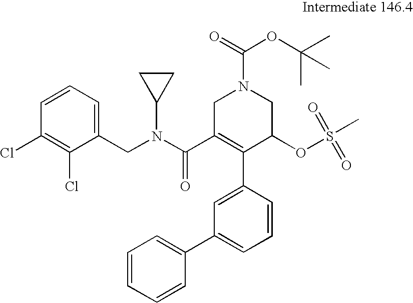

To a solution of Intermediate 146.5 (150 mg, 0.25 mmol) and NEt₃ (0.07 mL, 0.51 mmol) in DCM (15 mL), MsCl (0.03 mL, 0.38 mmol) is added at 0° C. After stirring at RT for 10 h, H₂O (10 mL) is added. The mixture is extracted with DCM. The combined organic phases are washed with H₂O, brine and dried (Na₂SO₄). Concentration under reduced pressure and silica gel flash chromatography give Intermediate 146.4 as yellow solid; ¹H-NMR (400 MHz, CDCl₃) δ 0.49-0.92 (m, 4H), 1.53 (s, 9H), 2.10-2.21 (m, 1H), 3.13 (s, 3H), 3.44-5.00 (m, 7H), 6.28-6.43 (m, 1H), 6.78 (t, 1H), 7.19-7.65 (m, 10H). Rf=0.80 (hexane/EtOAc 1:1).

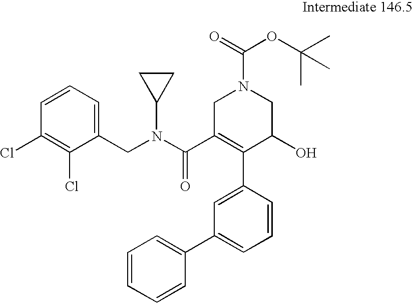

To a mixture of Example 143 (200 mg, 0.33 mmol) in dioxane (10 mL) and 1N NaOH solution, Boc₂O (0.3 mL, 0.91 mmol) is added at 0° C. After stirring at RT for 3 h, H₂O is added. The mixture is extracted with Et₂O (30 mL, ×2). The combined organic phases are washed with H₂O, brine and dried (MgSO₄). Concentration under reduced pressure and silica gel flash chromatography give Intermediate 146.5 as a yellow oil; ¹H-NMR (CDCl₃) δ 0.42-0.59 (m, 2H), 0.62-0.81 (m, 2H), 1.53 (s, 9H), 1.76 (brs, 1H), 2.08-2.15 (m, 1H), 2.80-3.90 (m, 3H), 4.00-5.00 (m, 4H), 6.38-6.47 (m, 1H), 6.78-6.88 (m, 1H), 7.20-7.62 (m, 10H). Rf=0.79 (EtOAc).

### Example 147

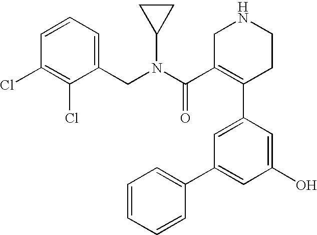

Example 147 is synthesized by deprotection of Intermediate 147.1 (121 mg, 0.19 mmol) analogously to the preparation of Example 1. Solid powder; ES-MS: M+H 493; HPLC: t_{Ret}=3.55 min.

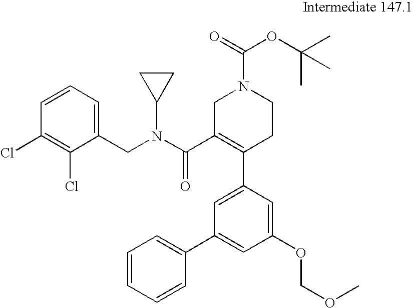

Intermediate 147.1 is synthesized by condensation of Intermediate 147.2 and cyclopropyl-(2,3-dichloro-benzyl)-amine (139 mg, 0.6 mmol) analogously to the preparation of Intermediate 1.1. Colorless oil; ES-MS: M+H=636; HPLC: t_{Ret}=5.75 min.

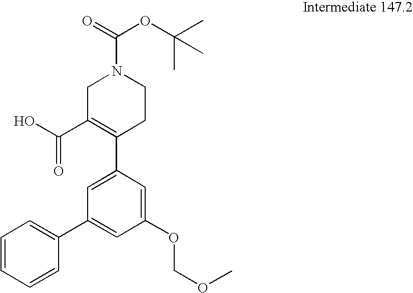

Intermediate 147.2 is synthesized by hydrolysis of Intermediate 147.3 (1.51 mg, 3.3 mmol) analogously to the preparation of Intermediate 1.3. Colorless oil; ES-MS: M-^{t}BuO=366; HPLC: t_{Ret}=4.47 min.

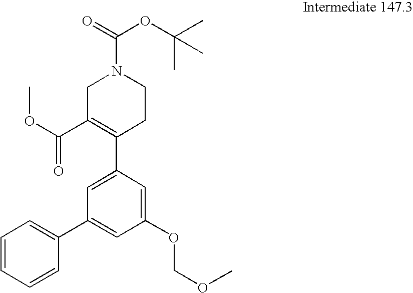

Intermediate 147.3 is synthesized by condensation of 4-trifluoromethanesulfonyloxy-5,6-dihydro-2H-pyridine-1,3-dicarboxylic acid 1-tert-butyl ester 3-methyl ester (1.62 g, 4.2 mmol) and Intermediate 147.4 (1.69 g, 5.0 mmol) analogously to the preparation of Intermediate 1.4. Colorless oil; ¹H-NMR (CDCl₃) δ 1.51 (s, 9H), 2.55 (br s, 2H), 3.50 (s, 3H), 3.52 (s, 3H), 3.62 (t, 2H), 4.26 (br s, 2H), 5.21 (s, 2H), 6.82 (m, 1H), 7.02 (m, 1H), 7.19-7.20 (m, 1H), 7.32-7.36 (m, 1H), 7.42 (t, 2H), 7.55-7.57 (m, 2H). Rf=0.16 (EtOAc:n-Hex=1:5).

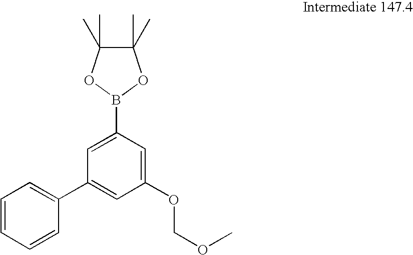

A mixture of 5-phenylresorcinol (3.57 g, 19.1 mmol) (see e.g. J. Chem. Soc., Chemical Communications (1978), (3), 118), MOMCl (1.22 mL, 21.1 mmol) and DIEA (3.61 mL, 21.1 mmol) in DCM (100 mL) is stirred at 0° C. for 30 min. After adding saturated NaHCO₃ solution, the reaction mixture is extracted with DCM. The combined organic phases are washed with H₂O, brine and dried (MgSO₄). Concentration under reduced pressure and silica gel flash chromatography give mono-MOM ether as a yellow oil. A mixture of the mono-ether (1.73 g, 7.5 mmol), Tf₂O (1.35 mL, 8.25 mmol) and DIEA (1.67 mL, 9.75 mmol) in DCM (30 mL) is stirred at 0° C. for 30 min. After adding saturated NaHCO₃ solution, the reaction mixture is extracted with EtOAc The combined organic phases are washed with H₂O, brine and dried (MgSO₄). Concentration under reduced pressure gives crude mono-triflate as a yellow oil. This crude product is used without purification. A mixture of this crude, bis(pinacolato)diboron (2.87 g, 11.3 mmol), KOAc (2.94 g, 30 mmol) and Pd(PPh₃)₄ (866 mg, 0.75 mmol) in DMF (30 mL) is stirred under N₂ at 110° C. After stirring for 8 h, the reaction mixture is quenched by slowly adding H₂O, and the mixture is extracted with EtOAc. The combined organic phases are washed with H₂O, brine and dried (Na₂SO₄). Concentration under reduced pressure and silica gel flash chromatography give Intermediate 147.4 as yellow oil; ES-MS: M+H=341; HPLC: t_{Ret}=4.09 min.

The following Examples enlisted in Table 3 are synthesized analogously to the preparation of Examples 141-147. As far as not being commercially available, the synthesis of intermediates for the preparation of compounds of Example 148-159 is described below Table 3 (an asterisk (*) indicates the end of the bond and the end thereof with which the moiety is bound to the rest of the molecule).

TABLE 3

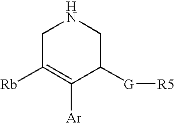

| No. | Rb | Ar | G—R5 | Analytical data |
|---|---|---|---|---|
| 148 | 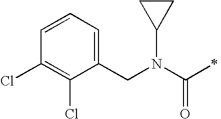 | 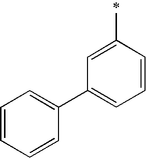 | 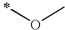 | MS: [M]⁺ = 507 HPLC t_{Ret} = 3.75 min. |
| 149 | 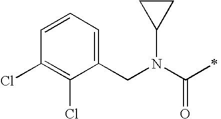 | 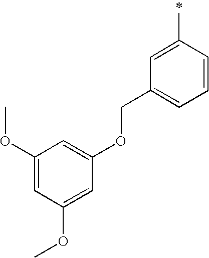 | H | MS: [M ]⁺ = 567 HPLC t_{Ret} = 3.75 min. |
| 150 | 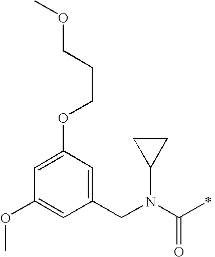 | 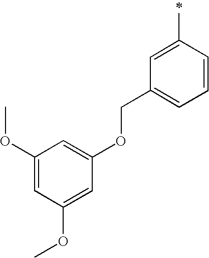 | H | MS: [M + H]⁺ = 617 HPLC t_{Ret} = 3.43 min. |
| 151 | 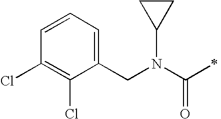 | 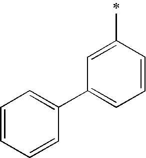 | 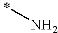 | MS: [M]⁺ = 492 HPLC t_{Ret} = 3.23 min. |
| 152 | 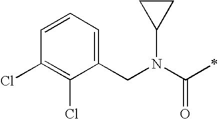 | 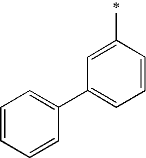 | 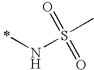 | MS: [M]⁺ = 570 HPLC t_{Ret} = 3.77 min. |
| 153 | 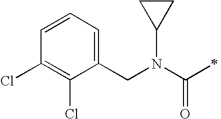 | 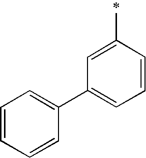 | 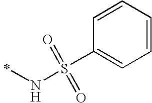 | MS: [M]⁺ = 632 HPLC t_{Ret} = 4.05 min. |
| 154 | 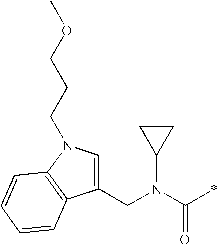 | 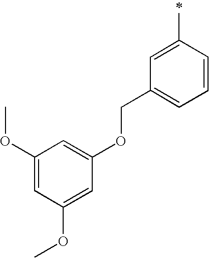 | H | MS: [M]⁺ = 610 HPLC t_{Ret} = 3.97 min. |
| 155 | 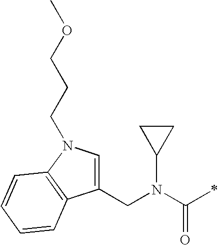 | 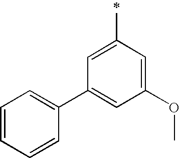 | H | MS: [M + 1]⁺ = 550 HPLC t_{Ret} = 3.68 min |
| 156 | 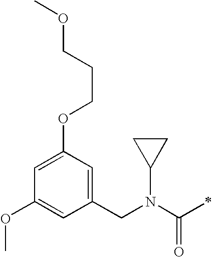 | 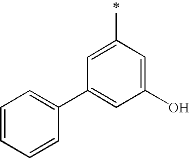 | H | MS: [M + 1]⁺ = 543 HPLC t_{Ret} = 3.29 min |
| 157 | 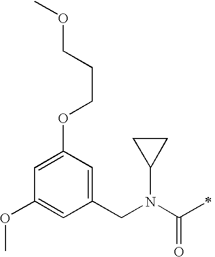 | 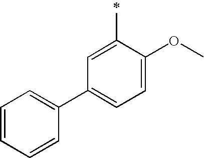 | H | MS: [M + H]⁺ = 557 HPLC t_{Ret} = 3.63 min. |
| 158 | 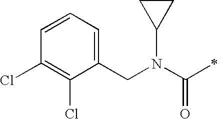 | 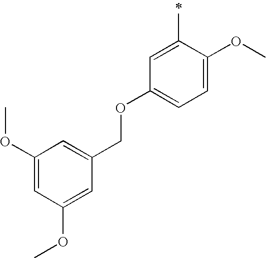 | H | MS: [M]⁺ = 597 HPLC t_{Ret} = 3.72 min. |
| 159 | 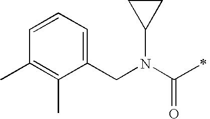 | 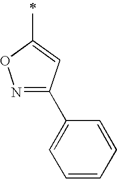 | H | MS: [M]⁺ = 428 HPLC t_{Ret} = 3.37 min. |

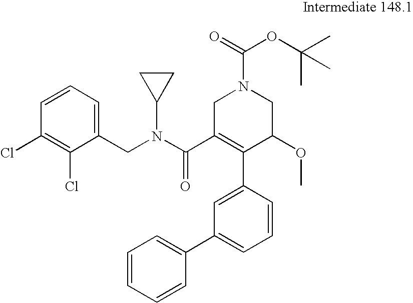

Intermediate 148.1 is synthesized by condensation of Intermediate 148.2 (81 mg, 0.18 mmol) analogously to the preparation of Intermediate 1.1. Colorless amorphous material; ¹H-NMR (CDCl₃) δ 0.37-0.57 (m, 2H), 0.65-0.90 (m, 2H), 1.54 (s, 9H), 2.02-2.20 (m, 1H), 2.99-3.19 (m, 1H), 3.51 (brs, 3H), 3.66-4.72 (m, 6H), 6.30-6.45 (m, 1H), 6.70-6.85 (m, 1H), 7.29-7.49 (m, 6H), 7.51-7.82 (m, 4H) Rf=0.60 (hexane/EtOAc 3:2).

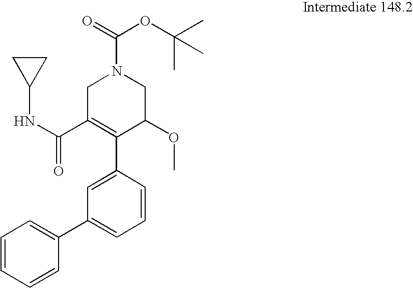

Intermediate 148.2 is synthesized by condensation of Intermediate 148.3 (89 mg, 0.22 mmol) analogously to the preparation of Intermediate 1.2. Colorless solid; ¹H-NMR (CDCl₃) δ −0.15-−0.05 (m, 2H), 0.45-0.55 (m, 2H), 1.50 (s, 9H), 2.42-2.50 (m, 1H), 2.99-3.19 (m, 1H), 3.41 (s, 3H), 3.95-4.12 (m, 2H), 4.33 (dd, J=2.8, 14 Hz, 1H), 4.48-4.72 (m, 1H), 5.09-5.20 (m, 1H), 7.29-7.60 (m, 9H) Rf=0.50 (hexane/EtOAc 1:1).

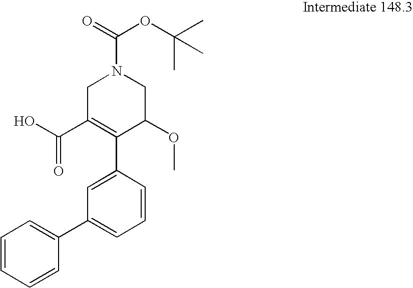

Intermediate 148.3 is synthesized by hydrolysis of Intermediate 148.4 (121 mg, 0.29 mmol) analogously to the preparation of Intermediate 1.3. Colorless amorphous material; ¹H-NMR (CDCl₃) δ 1.50 (s, 9H), 3.15-3.31 (m, 1H), 3.30 (s, 3H), 3.81-4.05 (m, 2H), 4.20-4.30 (m, 1H), 4.41-4.80 (m, 1H), 7.19-7.60 (m, 9H) Rf=0.27 (hexane/EtOAc 1:1).

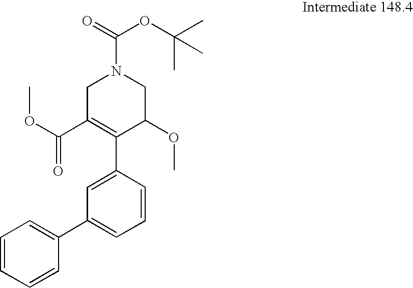

To a solution of Intermediate 143.5 (130 mg, 0.32 mmol) in THF (10 mL), NaH (16 mg, 0.38 mmol) is added at 0° C. After stirring at RT for 30 min and adding MeI (0.04 mL, 0.63 mmol) at 0° C., the reaction mixture is stirred for 10 h at RT. After adding saturated NaHCO₃ solution (15 mL), the reaction mixture is extracted with EtOAc. The combined organic phases are washed with H₂O, brine and dried (MgSO₄). Concentration under reduced pressure and silica gel flash chromatography give Intermediate 148.4 as a colorless oil; ¹H-NMR (CDCl₃) δ 1.47 (s, 9H), 2.99-3.07 (m, 1H), 3.28 (s, 3H), 3.48 (s, 3/2H), 3.64-3.70 (m, 1H), 3.68 (s, 3/2H), 4.19-4.22 (m, 1H), 4.29 (dd, J=4.8, 8 Hz, 1H), 4.83-4.95 (m, 1H), 7.12-7.64 (m, 9H) Rf=0.63 (hexane/EtOAc 3:2).

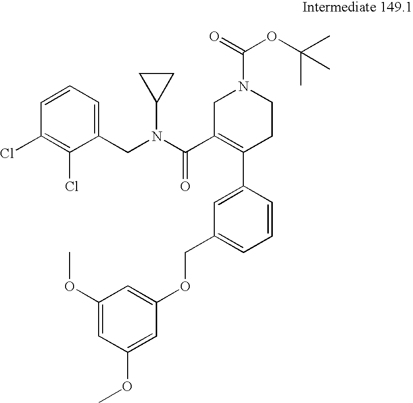

Intermediate 149.1 is synthesized by condensation of Intermediate 149.2 (190 mg, 0.37 mmol) analogously to the preparation of Intermediate 1.1. Colorless oil; ES-MS: M+=667; HPLC: t_{Ret}=5.65 min.

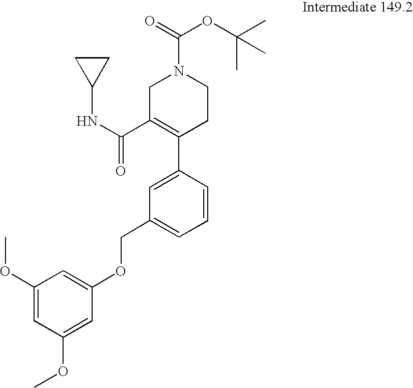

Intermediate 149.2 is synthesized by condensation of Intermediate 149.3 (2.0 g, 4.3 mmol) analogously to the preparation of Intermediate 1.2. Colorless oil; ES-MS: M+H=509; HPLC: t_{Ret}=4.28 min.

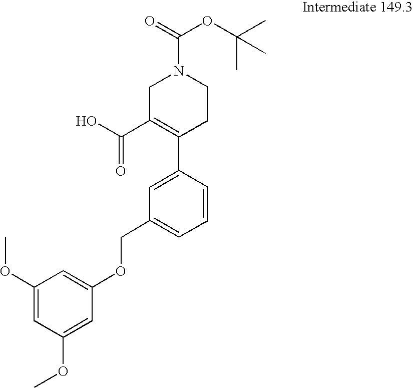

Intermediate 149.3 is synthesized by hydrolysis of Intermediate 149.4 (4.0 g, 8.27 mmol) analogously to the preparation of Intermediate 1.3. Colorless oil; ES-MS: M+H=470; HPLC: t_{Ret}=4.35 min.

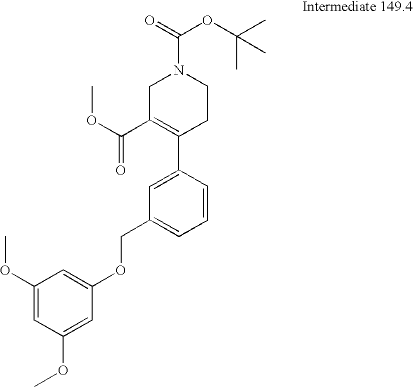

Intermediate 149.4 is synthesized by coupling of 4-trifluoromethanesulfonyloxy-5,6-dihydro-2H-pyridine-1,3-dicarboxylic acid 1-tert-butyl ester 3-methyl ester (3.89 g, 10 mmol) analogously to the preparation of Intermediate 1.4. Colorless oil; Rf=0.30 (AcOEt:n-Hex=1:4); ¹H NMR (CDCl₃) δ 1.52 (s, 9H), 2.53 (brs, 2H), 3.49 (s, 2H), 3.61-3.64 (m, 2H), 3.78 (s, 6H), 4.27 (brs, 2H), 5.02 (s, 2H), 6.12 (t, 1H), 6.18 (d, 2H), 7.10-7.12 (m, 1H), 7.22 (brs, 1H), 7.37 (d, 2H).

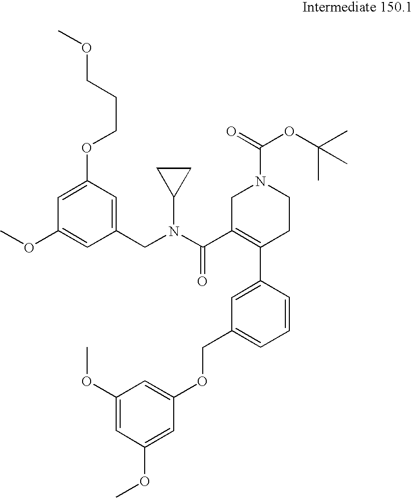

Intermediate 150.1 is synthesized by condensation of Intermediate 149.2 (215 mg, 0.43 mmol) analogously to the preparation of Intermediate 1.1. Colorless oil; ES-MS: M+H=718; HPLC: t_{Ret}=5.24 min.

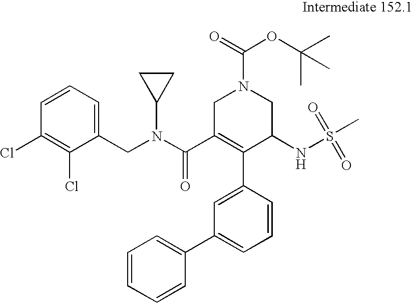

Intermediate 152.1 is synthesized by condensation of Intermediate 1462 (80 mg, 0.14 mmol) analogously to the preparation of Intermediate 146.4. Colorless amorphous material; ¹H-NMR (CDCl₃) δ 0.47-0.61 (m, 2H), 0.65-0.69 (m, 2H), 1.53 (s, 9H), 2.12-2.25 (m, 1H), 2.30-2.68 (brs, 3H), 3.42-3.50 (m, 1H), 3.75-3.90 (m, 1H), 4.18-4.89 (m, 6H), 6.39 (d, J=7.5 Hz, 1H), 6.80 (t, J=7.5 Hz, 1H), 7.30-7.65 (m, 10H) Rf=0.68 (hexane/EtOAc 1:1).

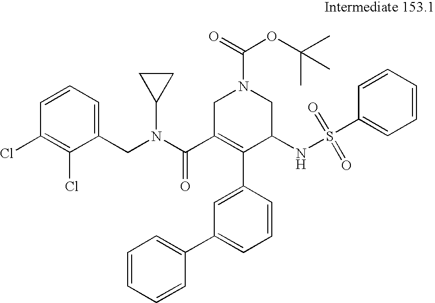

Intermediate 153.1 is synthesized by condensation of Intermediate 146.2 (40 mg, 0.08 mmol) analogously to the preparation of Intermediate 146.4. Colorless amorphous material; ¹H-NMR (CDCl₃) δ 0.37-0.55 (m, 2H), 0.60-0.75 (m, 2H), 1.50 (s, 6H), 1.55 (s, 3H), 1.90-2.45 (m, 1H), 3.08-3.51 (m, 1H), 3.77-3.90 (m, 1H), 4.18-4.85 (m, 6H), 6.29 (d, J=7.5 Hz, 1H), 6.75 (d, J=7.5 Hz, 1H), 6.80 (t, J=7.5 Hz, H), 6.91-8.02 (m, 15H) Rf=0.26 (EtOAc).

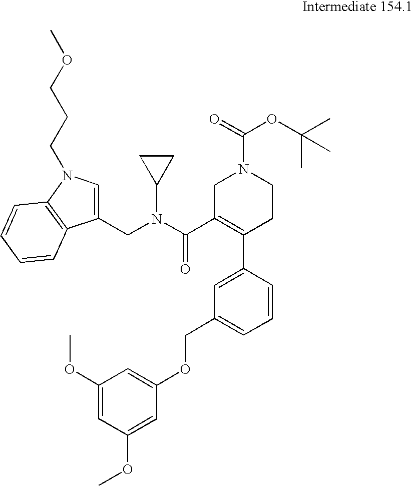

Intermediate 154.1 is synthesized by condensation of Intermediate 149.3 (200 mg, 0.42 mmol) analogously to the preparation of Intermediate 3.1. Colorless oil; ES-MS: M+H=711; HPLC: t_{Ret}=5.45 min.

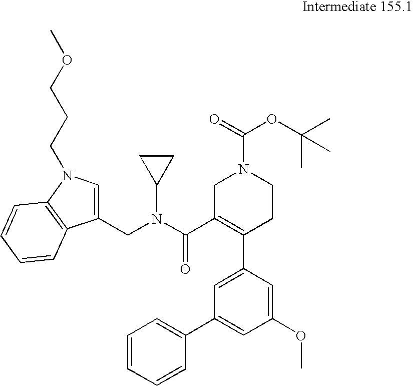

Intermediate 155.1 is synthesized by condensation of Intermediate 144.3 (191 mg, 0.73 mmol) analogously to the preparation of Intermediate 3.1. Colorless oil; ES-MS: M+H 650; HPLC: t_{Ret}=5.43 min.

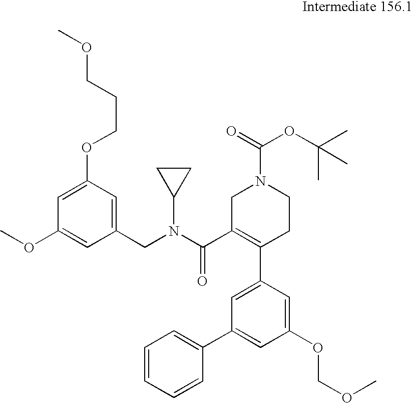

Intermediate 156.1 is synthesized by condensation of Intermediate 1472 (181 mg, 0.41 mmol) analogously to the preparation of Intermediate 3.1. Colorless oil; ES-MS: M+H=687; HPLC: t_{Ret}=5.25 min.

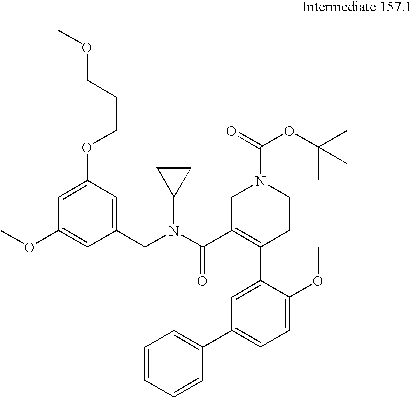

Intermediate 157.1 is synthesized by condensation of Intermediate 157.2 (82 mg, 0.20 mmol) analogously to the preparation of Intermediate 3.1. Colorless oil; ES-MS: M+H=657; HPLC: t_{Ret}=5.39 min.

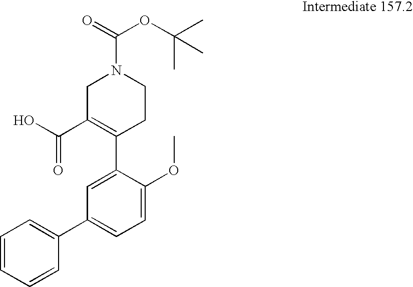

Intermediate 1572 is synthesized by hydrolysis of Intermediate 157.3 (170 mg, 0.4 mmol) analogously to the preparation of Intermediate 1.3. Colorless oil; Rf=0.08 (EtOAc:n-Hex=1:1)

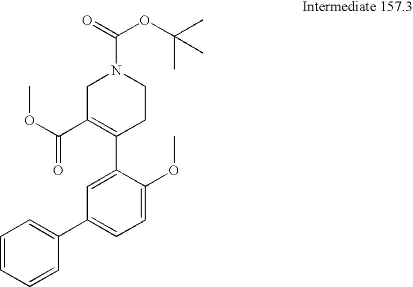

Intermediate 157.3 is synthesized by coupling of 4-trifluoromethanesulfonyloxy-5,6-dihydro-2H-pyridine-1,3-dicarboxylic acid 1-tert-butyl ester 3-methyl ester (594 mg, 1.2 mmol) analogously to the preparation of Intermediate 1.4. Colorless oil; Rf=0.56 (EtOAc:n-Hex=1:2), ¹H NMR (CDCl₃), δ: 1.51 (9H, s), 2.48-2.57 (2H, m), 3.48 (3H, s), 3.56-3.67 (2H, m), 3.83 (3H, s), 4.23-4.36 (2H, m), 6.96 (1H, d), 7.22-7.31 (2H, m), 7.38-7.42 (2H, m), 7.48-7.54 (3H, m).

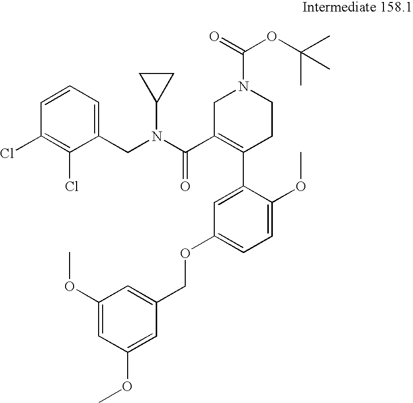

Intermediate 158.1 is synthesized by condensation of Intermediate 158.2 (200 mg, 0.40 mmol) analogously to the preparation of Intermediate 3.1. Colorless oil; ES-MS: M+H=697; HPLC: t_{Ret}=5.74 min.

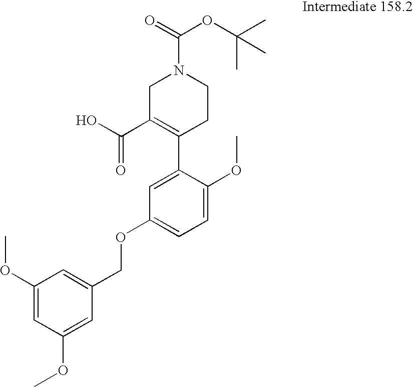

Intermediate 158.2 is synthesized by hydrolysis of Intermediate 158.3 (250 mg, 0.47 mmol) analogously to the preparation of Intermediate 1.3. Colorless oil; ES-MS: M+H=500; HPLC: t_{Ret}=4.42 min.

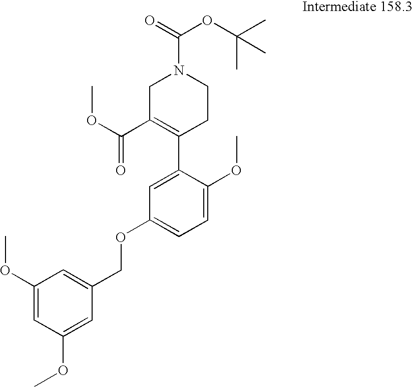

Intermediate 158.3 is synthesized by coupling of 4-trifluoromethanesulfonyloxy-5,6-dihydro-2H-pyridine-1,3-dicarboxylic acid 1-tert-butyl ester 3-methyl ester (436 mg, 1.2 mmol) analogously to the preparation of Intermediate 1.4. Colorless oil; Rf=0.40 (EtOAc:n-Hex=1:2), ¹H NMR (CDCl₃), δ: 1.50 (9H, s), 2.42-2.50 (2H, m), 3.48 (3H, s), 3.52-3.63 (2H, m), 3.74 (3H, s), 3.79 (6H, s), 4.21-4.29 (2H, m), 4.93 (2H, s), 6.40 (1H, t), 6.57 (2H, s), 6.64 (1H, d), 6.78-6.84 (2H, m).

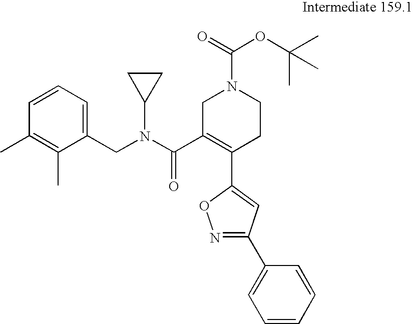

Intermediate 159.1 is synthesized by condensation of Intermediate 1592 (61 mg, 0.12 mmol) analogously to the preparation of compound of Intermediate 3.1. Yellow oil; ES-MS: M+H=528; ¹H NMR (CDCl₃), δ: 0.53-1.12 (4H, m), 1.52 (9H, s), 2.08 (1H, d), 220 (1H, s), 2.23 (3H, s), 2.24 (3H, s), 2.31-2.91 (2H, m), 3.32-5.02 (5H, m), 6.54 (1H, s), 6.88-7.03 (3H, m), 7.40-7.50 (3H, m), 7.69-7.80 (2H, m).

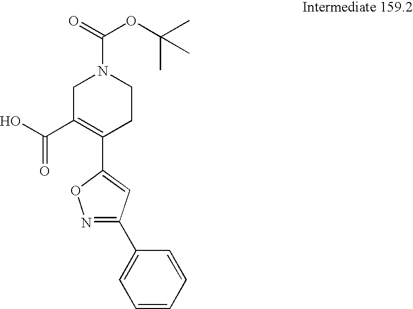

Intermediate 159.2 is synthesized by hydrolysis of Intermediate 159.3 (98 mg, 0.26 mmol) analogously to the preparation of Intermediate 1.3. Colorless amorphous; ES-MS: M+H=371; ¹H NMR (CDCl₃), δ: 1.50 (9H, s), 2.60-2.67 (2H, m), 3.64 (2H, t), 4.31 (2H, brs), 6.69 (1H, s), 7.40-7.47 (3H, m), 7.75-7.80 (2H, m).

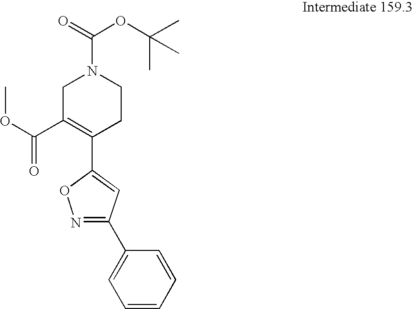

A mixture of Intermediate 159.4 (300 mg, 1.13 mmol), phenylcarboximidoyl chloride (211 mg, 1.36 mmol) and NEt₃ (0.24 mL, 1.70 mmol) in dichloromethane (15 mL) are stirred under N₂ at RT for 10 hours. After adding H₂O, the reaction mixture is extracted with DCM. The combined organic phases are washed with H₂O, brine and dried (MgSO₄), concentrated under reduced pressure and silica gel flash chromatography to give Intermediate 159.3 as yellow solid; ES-MS: M+H=385; HPLC: t_{Ret}=4.67 minutes.

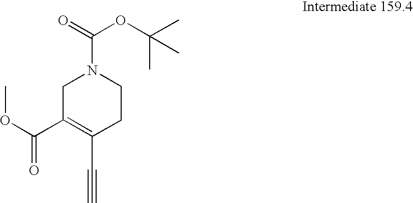

A mixture of Intermediate 159.5 (400 mg, 1.19 mmol) and CsF (432 mg, 2.84 mmol) in MeOH (10 mL)-H₂O (2 mL) are stirred under N₂ at RT for 10 hours. After evaporating, the residue is added H₂O and DCM. The mixture is extracted with DCM. The combined organic phases are washed with H₂O, brine and dried (MgSO₄), concentrated under reduced pressure and silica gel flash chromatography to give Intermediate 159.4 as white solid (278 mg, 1.04 mmol; 88%); ES-MS: M+H-^{t}Bu=210; HPLC: t_{Ret}=4.00 minutes.

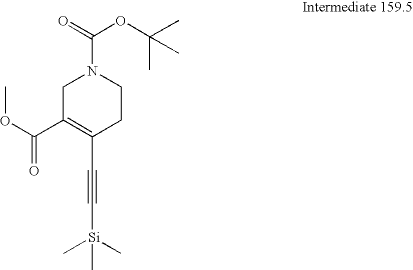

A mixture of 4-trifluoromethanesulfonyloxy-5,6-dihydro-2H-pyridine-1,3-dicarboxylic acid 1-tert-butyl ester 3-methyl ester (600 mg, 1.54 mmol), (Trimethylsilyl)acethylene (0.66 mL, 4.62 mmol), CuI (30.0 mg, 0.15 mmol), NEt₃ (1.08 mL, 7.72 mmol) and Pd(PPh₃)₄ (54.0 mg, 0.08 mmol) in DMF (10 mL) are stirred under N₂ at 60° C. for 2.5 hours. After adding H₂O, the reaction mixture is extracted with Et₂O. The combined organic phases are washed with H₂O, brine and dried (MgSO₄), concentrated under reduced pressure and silica gel flash chromatography to give Intermediate 159.5 as white amorphous material; Rf=0.65 (EtOAc:n-Hex=1:4), ¹H NMR (CDCl₃), δ: 0.22 (9H, s), 1.03 (9H, s), 1.96-2.01 (2H, m), 3.02 (2H, t), 3.34 (3H, s), 3.78 (2H, brs).

### Example 160

### Soft Capsules

5000 soft gelatin capsules, each comprising as active ingredient 0.05 g of any one of the compounds of formula I mentioned in any one of the preceding Examples, are prepared as follows:

Composition

| | |
|---|---|
| Active ingredient | 250 g |
| Lauroglycol | 2 liters |

Preparation process: The pulverized active ingredient is suspended in Lauroglykol® (propylene glycol laurate, Gattefossé S.A., Saint Priest, France) and ground in a wet pulverizer to produce a particle size of about 1 to 3 μm. 0.419 g portions of the mixture are then introduced into soft gelatin capsules using a capsule-filling machine.

### Example 161

### Tablets Comprising Compounds of the Formula I

Tablets, comprising, as active ingredient, 100 mg of any one of the compounds of formula I in any one of the preceding Examples are prepared with the following composition, following standard procedures:

Composition

| | | |
|---|---|---|
| Active Ingredient | 100 | mg |
| crystalline lactose | 240 | mg |
| Avicel | 80 | mg |
| PVPPXL | 20 | mg |
| Aerosil | 2 | mg |
| magnesium stearate | 5 | mg |
| | 447 | mg |

Manufacture: The active ingredient is mixed with the carrier materials and compressed by means of a tabletting machine (Korsch EKO, stamp diameter 10 mm).

Avicel® is microcrystalline cellulose (FMC, Philadelphia, USA). PVPPXL is polyvinylpolypyrrolidone, cross-linked (BASF, Germany). Aerosil® is silicon dioxide (Degussa, Germany).

1. A compound of the formula I 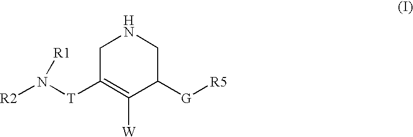
wherein
R1 is unsubstituted or substituted alkyl, unsubstituted or substituted alkenyl, unsubstituted or substituted alkynyl, unsubstituted or substituted aryl, unsubstituted or substituted heterocyclyl or unsubstituted or substituted cycloalkyl;
R2 is hydrogen, unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, or acyl;
W is a moiety selected from those of the formulae IA, IB and IC, 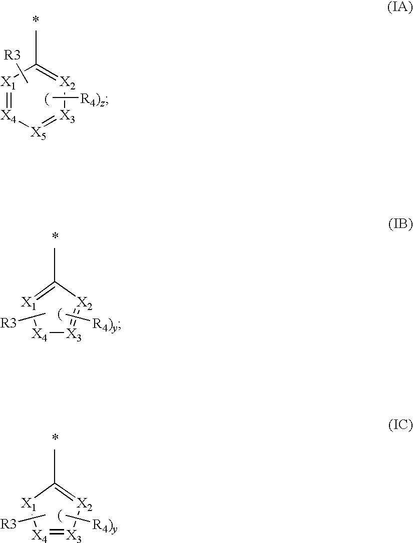
wherein the asterisk (*) denotes the position where the moiety W is bound to the 4-carbon in the piperidine ring in formula I, and wherein
X₁, X₂, X₃, X₄ and X₅ are independently selected from carbon and nitrogen, where X₄ in formula IB and X₁ in formula IC may have one of these meanings or further be selected from S and O, where carbon and nitrogen ring atoms can carry the required number of hydrogen or substituents R₃ or—if present—R₄ to complete the number of bonds emerging from a ring carbon to four, from a ring nitrogen to three; with the proviso that in formula IA at least 2 of X₁ to X₅ are carbon and in formulae IB and IC at least one of X₁ to X₄ is carbon;
y is 0, 1, 2 or 3;
z is 0, 1, 2, 3 or 4
R3 which can only be bound to any one of X₁, X₂, X₃ and X₄ is hydrogen or preferably unsubstituted or substituted C₁-C₇-alkyl, unsubstituted or substituted C₂-C₇-alkenyl, unsubstituted or substituted C₂-C₇-alkynyl, unsubstituted or substituted aryl, unsubstituted or substituted heterocyclyl, unsubstituted or substituted cycloalkyl, halo, hydroxy, etherified or esterified hydroxy, unsubstituted or substituted mercapto, unsubstituted or substituted sulfinyl, unsubstituted or substituted sulfonyl, amino, mono- or di-substituted amino, carboxy, esterified or amidated carboxy, unsubstituted or substituted sulfamoyl, nitro or cyano, with the proviso that if R3 is hydrogen then y and z are 0;
R4 is—if y or z is 2 or more, independently—selected from a group of substituents consisting of unsubstituted or substituted C₁-C₇-alkyl, unsubstituted or substituted C₂-C₇-alkenyl, unsubstituted or substituted C₂-C₇-alkynyl, halo, hydroxy, etherified or esterified hydroxy, unsubstituted or substituted mercapto, unsubstituted or substituted sulfinyl, unsubstituted or substituted sulfonyl, amino, mono- or di-substituted amino, carboxy, esterified or amidated carboxy, unsubstituted or substituted sulfamoyl, nitro and cyano;
T is carbonyl; and
G is methylene, oxy, thio, imino or substituted imino —NR6- wherein R6 is unsubstituted or substituted alkyl; and R5 is hydrogen, unsubstituted or substituted alkyl, unsubstituted or substituted alkyloxy or acyl;
or -G-R5 is hydrogen;
or a salt thereof.

2. A compound of the formula I according to claim 1, wherein
R1 is unsubstituted or substituted alkyl, unsubstituted or substituted alkenyl, unsubstituted or substituted alkynyl, unsubstituted or substituted aryl, unsubstituted or substituted heterocyclyl or unsubstituted or substituted cycloalkyl;
R2 is hydrogen, unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, or acyl;
W is a moiety selected from those of the formulae IA, IB and IC, 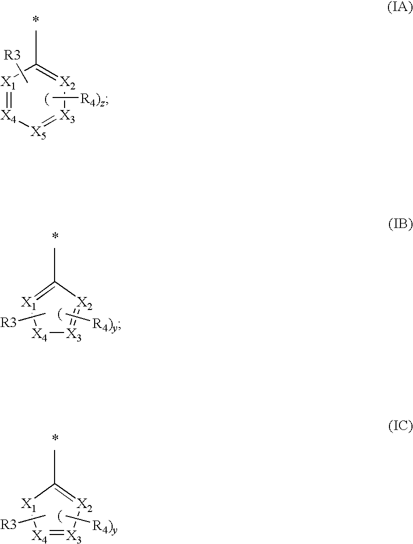
wherein the asterisk (*) denotes the position where the moiety W is bound to the 4-carbon in the piperidine ring in formula I, and wherein
X₁, X₂, X₃, X₄ and X₅ are independently selected from carbon and nitrogen, where X₄ in formula IB and X₁ in formula IC may have one of these meanings or further be selected from S and O, where carbon and nitrogen ring atoms can carry the required number of hydrogen or substituents R₃ or—if present—R₄ to complete the number of bonds emerging from a ring carbon to four, from a ring nitrogen to three; with the proviso that in formula IA at least 2, preferably at least 3 of X₁ to X₅ are carbon and in formulae IB and IC at least one of X₁ to X₄ is carbon, preferably two of X₁ to X₄ are carbon;
y is 0, 1, 2 or 3;
z is 0, 1, 2, 3 or 4
R3 which can only be bound to any one of X₁, X₂, X₃ and X₄ is hydrogen or preferably unsubstituted or substituted C₁-C₇-alkyl, unsubstituted or substituted C₂-C₇-alkenyl, unsubstituted or substituted C₂-C₇-alkynyl, unsubstituted or substituted aryl, unsubstituted or substituted heterocyclyl, unsubstituted or substituted cycloalkyl, halo, hydroxy, etherified or esterified hydroxy, unsubstituted or substituted mercapto, unsubstituted or substituted sulfinyl, unsubstituted or substituted sulfonyl, amino, mono- or di-substituted amino, carboxy, esterified or amidated carboxy, unsubstituted or substituted sulfamoyl, nitro or cyano, with the proviso that if R3 is hydrogen then y and z are 0;
R4 is—if y or z is 2 or more, independently—selected from a group of substituents consisting of unsubstituted or substituted C₁-C₇-alkyl, unsubstituted or substituted C₂-C₇-alkenyl, unsubstituted or substituted C₂-C₇-alkynyl, halo, hydroxy, etherified or esterified hydroxy, unsubstituted or substituted mercapto, unsubstituted or substituted sulfinyl (—S(═O)—), unsubstituted or substituted sulfonyl (—S(═O)₂—), amino, mono- or di-substituted amino, carboxy, esterified or amidated carboxy, unsubstituted or substituted sulfamoyl, nitro and cyano;
T is carbonyl; and
G is methylene, oxy (—O—), thio (—S—), imino (—NH—) or substituted imino (—NR6-) wherein R6 is unsubstituted or substituted alkyl; and
R5 is hydrogen, unsubstituted or substituted alkyl, unsubstituted or substituted alkyloxy or acyl;
or -G-R5 is hydrogen;
where in each case of occurrence in this claim
unsubstituted or substituted alkyl is C₁-C₂₀-alkyl, more preferably C₁-C₇-alkyl, that is straight-chained or branched one or, if desired and possible, more times, and which is unsubstituted or substituted by one or more, e.g. up to three moieties selected from unsubstituted or substituted aryl as described below, especially phenyl or naphthyl each of which is unsubstituted or substituted as described below for unsubstituted or substituted aryl, unsubstituted or substituted heterocyclyl as described below, especially pyrrolyl, furanyl, thienyl, pyrazolyl, triazolyl, tetrazolyl, oxetidinyl, 3-(C₁-C₇-alkyl)-oxetidinyl, pyridyl, pyrimidinyl, morpholino, thiomorpholino, piperidinyl, piperazinyl, pyrrolidinyl, tetrahydrofuran-onyl, tetrahydro-pyranyl, indolyl, 1H-indazanyl, benzofuranyl, benzothiophenyl, quinolinyl, isoquinolinyl, 1,2,3,4-tetrahydro-1,4-benzoxazinyl, 2H-1,4-benzoxazin-3(4H)-onyl, 2H,3H-1,4-benzodioxinyl or benzo[1,2,5]oxadiazolyl each of which is unsubstituted or substituted as described below for unsubstituted or substituted heterocyclyl, unsubstituted or substituted cycloalkyl as described below, especially cyclopropyl, cyclobutyl, cyclopentyl or cyclohexyl each of which is unsubstituted or substituted as described below for unsubstituted or substituted cycloalkyl, halo, hydroxy, C₁-C₇-alkoxy, halo-C₁-C₇-alkoxy, such as trifluoromethoxy, hydroxy-C₁-C₇-alkoxy, C₁-C₇-alkoxy-C₁-C₇-alkoxy, phenyl- or naphthyloxy, phenyl- or naphthyl-C₁-C₇-alkyloxy, C₁-C₇-alkanoyloxy, benzoyl- or naphthoyloxy, C₁-C₇-alkylthio, halo-C₁-C₇-alkylthio, such as trifluoromethylthio, C₁-C₇-alkoxy-C₁-C₇-alkylthio, phenyl- or naphthylthio, phenyl- or naphthyl-C₁-C₇-alkylthio, C₁-C₇-alkanoylthio, benzoyl- or naphthoylthio, nitro, amino, mono- or di-(C₁-C₇-alkyl and/or C₁-C₇-alkoxy-C₁-C₇-alkyl)-amino, mono- or di-(naphthyl- or phenyl-C₁-C₇-alkyl)-amino, C₁-C₇-alkanoylamino, benzoyl- or naphthoylamino, C₁-C₇-alkylsulfonylamino, phenyl- or naphthylsulfonylamino wherein phenyl or naphthyl is unsubstituted or substituted by one or more, especially one to three, C₁-C₇-alkyl moieties, phenyl- or naphthyl-C₁-C₇-alkylsulfonylamino, carboxyl, C₁-C₇-alkyl-carbonyl, C₁-C₇-alkoxy-carbonyl, phenyl- or naphthyloxycarbonyl, phenyl- or naphthyl-C₁-C₇-alkoxycarbonyl, carbamoyl, N— mono- or N,N-di-(C₁-C₇-alkyl)-aminocarbonyl, N-mono- or N,N-di-(naphthyl- or phenyl-C₁-C₇-alkyl)-aminocarbonyl, cyano, C₁-C₇-alkenylene or -alkynylene, C₁-C₇-alkylenedioxy, sulfenyl, sulfinyl, C₁-C₇-alkylsulfinyl, phenyl- or naphthylsulfinyl wherein phenyl or naphthyl is unsubstituted or substituted by one or more, especially one to three, C₁-C₇-alkyl moieties, phenyl- or naphthyl-C₁-C₇-alkylsulfinyl, sulfonyl, C₁-C₇-alkylsulfonyl, phenyl- or naphthylsulfonyl wherein phenyl or naphthyl is unsubstituted or substituted by one or more, especially one to three, C₁-C₇-alkyl moieties, phenyl- or naphthyl-C₁-C₇-alkylsulfonyl, sulfamoyl and N-mono or N,N-di-(C₁-C₇-alkyl, phenyl, naphthyl, phenyl-C₁-C₇-alkyl or naphthyl-C₁-C₇-alkyl)-aminosulfonyl;
unsubstituted or substituted alkenyl has 2 to 20 carbon atoms and includes one or more double bonds, and is more preferably C₂-C₇-alkenyl that is unsubstituted or substituted as described above for unsubstituted or substituted alkyl;
unsubstituted or substituted alkynyl has 2 to 20 carbon atoms and includes one or more triple bonds, and is more preferably C₂-C₇-alkynyl that is unsubstituted or substituted as described above for unsubstituted or substituted alkyl;
unsubstituted or substituted aryl is a mono- or polycyclic, especially monocyclic, bicyclic or tricyclic aryl moiety with 6 to 22 carbon atoms, especially phenyl, naphthyl, indenyl, fluorenyl, acenapthylenyl, phenylenyl or phenanthryl, and is unsubstituted or substituted by one or more, especially one to three, moieties, preferably independently selected from the group consisting of a substituent of the formula —(C₀-C₇-alkylene)-(X)ᵣ—(C₁-C₇-alkylene)-(Y)ₛ—(C₀-C₇-alkylene)-H where C₀-alkylene means that a bond is present instead of bound alkylene, r and s, each independently of the other, are 0 or 1 and each of X and Y, if present and independently of the others, is —O—, —NV—, —S—, —C(═O)—, —C(═S), —O—CO—, —CO—O—, —NV—CO—; —CO—NV—; —NV—SO₂—, —SO₂—NV; —NV—CO—NV—, —NV—CO—O—, —O—CO—NV—, —NV—SO₂—NV— wherein V is hydrogen or unsubstituted or substituted alkyl as defined below, especially selected from C₁-C₇-alkyl, phenyl, naphthyl, phenyl- or naphthyl-C₁-C₇-alkyl and halo-C₁-C₇-alkyl; e.g. C₁-C₇-alkyl, such as methyl, ethyl, n-propyl, isopropyl, n-butyl, isobutyl, sec-butyl or tert-butyl, hydroxy-C₁-C₇-alkyl, C₁-C₇-alkoxy-C₁-C₇-alkyl, such as 3-methoxypropyl or 2-methoxyethyl, C₁-C₇-alkoxy-C₁-C₇-alkoxy-C₁-C₇-alkyl, C₁-C₇-alkanoyloxy-C₁-C₇-alkyl, C₁-C₇-alkyloxycarbonyl-C₁-C₇-alkyl, amino-C₁-C₇-alkyl, such as aminomethyl, (N—) mono- or (N,N-) di-(C₁-C₇-alkyl)-amino-C₁-C₇-alkyl, C₁-C₇-alkoxy-C₁-C₇-alkylamino-C₁-C₇-alkyl, mono-(naphthyl- or phenyl)-amino-C₁-C₇-alkyl, mono-(naphthyl- or phenyl-C₁-C₇-alkyl)-amino-C₁-C₇-alkyl, C₁-C₇-alkanoylamino-C₁-C₇-alkyl, C₁-C₇-alkyl-O—CO—NH—C₁-C₇-alkyl, C₁-C₇-alkylsulfonylamino-C₁-C₇-alkyl, C₁-C₇-alkyl-NH—CO—NH—C₁-C₇-alkyl, C₁-C₇-alkyl-NH—SO₂—NH—C₁-C₇-alkyl, C₁-C₇-alkoxy, hydroxy-C₁-C₇-alkoxy, C₁-C₇-alkoxy-C₁-C₇-alkoxy, C₁-C₇-alkanoylamino-C₁-C₇-alkyloxy, carboxy-C₁-C₇-alkyloxy, C₁-C₇-alkyloxycarbonyl-C₁-C₇-alkoxy, mono- or di-(C₁-C₇-alkyl)-aminocarbonyl-C₁-C₇-alkyloxy, C₁-C₇-alkanoyloxy, mono- or di-(C₁-C₇-alkyl)-amino, mono-di-(naphthyl- or phenyl-C₁-C₇-alkyl)-amino, N-mono-C₁-C₇-alkoxy-C₁-C₇-alkylamino, C₁-C₇-alkanoylamino, C₁-C₇-alkylsulfonylamino, C₁-C₇-alkyl-carbonyl, halo-C₁-C₇-alkylcarbonyl, hydroxy-C₁-C₇-alkylcarbonyl, C₁-C₇-alkoxy-C₁-C₇-alkylcarbonyl, amino-C₁-C₇-alkylcarbonyl, (N—) mono- or (N,N-) di-(C₁-C₇-alkyl)-amino-C₁-C₇-alkylcarbonyl, C₁-C₇-alkanoylamino-C₁-C₇-alkylcarbonyl, C₁-C₇-alkoxy-carbonyl, hydroxy-C₁-C₇-alkoxycarbonyl, C₁-C₇-alkoxy-C₁-C₇-alkoxycarbonyl, amino-C₁-C₇-alkoxycarbonyl, (N-) mono-(C₁-C₇-alkyl)-amino-C₁-C₇-alkoxycarbonyl, C₁-C₇-alkanoylamino-C₁-C₇-alkoxycarbonyl, N-mono- or N,N-di-(C₁-C₇-alkyl)-aminocarbonyl, N—C₁-C₇-alkoxy-C₁-C₇-alkylcarbamoyl or N-mono- or N,N-di-(C₁-C₇-alkyl)-aminosulfonyl;
from C₂-C₇-alkenyl, C₂-C₇-alkynyl, phenyl, naphtyl, heterocyclyl, especially as defined below for heterocyclyl, preferably selected from pyrrolyl, furanyl, thienyl, pyrimidinyl, pyrazolyl, pyrazolidinonyl, N—(C₁-C₇-alkyl, phenyl, naphthyl, phenyl-C₁-C₇-alkyl or naphthyl-C₁-C₇-alkyl)-pyrazolidinonyl, triazolyl, tetrazolyl, oxetidinyl, 3-C₁-C₇-alkyl-oxetidinyl, pyridyl, pyrimidinyl, morpholino, piperidinyl, piperazinyl, pyrrolidinyl, tetrahydrofuran-onyl, tetrahydro-pyranyl, indolyl, indazolyl, 1H-indazolyl, benzofuranyl, benzothiophenyl, quinolinyl, isoquinolinyl, 1,2,3,4-tetrahydro-1,4-benzoxazinyl, 2H-1,4-benzoxazin-3(4H)-onyl, benzo[1,2,5]oxadiazolyl or 2H,3H-1,4-benzodioxinyl, phenyl- or naphthyl- or heterocyclyl-C₁-C₇-alkyl or —C₁-C₇-alkyloxy wherein heterocyclyl is as defined below, preferably selected from pyrrolyl, furanyl, thienyl, pyrimidinyl, pyrazolyl, pyrazolidinonyl, N—(C₁-C₇-alkyl, phenyl, naphthyl, phenyl-C₁-C₇-alkyl or naphthyl-C₁-C₇-alkyl)-pyrazolidinonyl, triazolyl, tetrazolyl, oxetidinyl, pyridyl, pyrimidinyl, morpholino, piperidinyl, piperazinyl, tetrahydrofuran-onyl, indolyl, indazolyl, 1H-indazanyl, benzofuranyl, benzothiophenyl, quinolinyl, isoquinolinyl, 1,2,3,4-tetrahydro-1,4-benzoxazinyl, 2H-1,4-benzoxazin-3(4H)-only or benzo[1,2,5]oxadiazolyl; such as benzyl or naphthylmethyl, halo-C₁-C₇-alkyl, such as trifluoromethyl, phenyloxy- or naphthyloxy-C₁-C₇-alkyl, phenyl-C₁-C₇-alkoxy- or naphthyl-C₁-C₇-alkoxy-C₁-C₇-alkyl, di-(naphthyl- or phenyl)-amino-C₁-C₇-alkyl, di-(naphthyl- or phenyl-C₁-C₇-alkyl)-amino-C₁-C₇-alkyl, benzoyl- or naphthoylamino-C₁-C₇-alkyl, phenyl- or naphthylsulfonylamino-C₁-C₇-alkyl wherein phenyl or naphthyl is unsubstituted or substituted by one or more, especially one to three, C₁-C₇-alkyl moieties, phenyl- or naphthyl-C₁-C₇-alkylsulfonylamino-C₁-C₇-alkyl, carboxy-C₁-C₇-alkyl, halo, especially fluoro or chloro, hydroxy, phenyl-C₁-C₇-alkoxy wherein phenyl is unsubstituted or substituted by C₁-C₇-alkoxy and/or halo, halo-C₁-C₇-alkoxy, such as trifluoromethoxy, phenyl- or naphthyloxy, phenyl- or naphthyl-C₁-C₇-alkyloxy, phenyl- or naphthyl-oxy-C₁-C₇-alkyloxy, benzoyl- or naphthoyloxy, halo-C₁-C₇-alkylthio, such as trifluoromethylthio, phenyl- or naphthylthio, phenyl- or naphthyl-C₁-C₇-alkylthio, benzoyl- or naphthoylthio, nitro, amino, di-(naphthyl- or phenyl-C₁-C₇-alkyl)-amino, benzoyl- or naphthoylamino, phenyl- or naphthylsulfonylamino wherein phenyl or naphthyl is unsubstituted or substituted by one or more, especially one to three, C₁-C₇-alkoxy-C₁-C₇-alkyl or C₁-C₇-alkyl moieties, phenyl- or naphthyl-C₁-C₇-alkylsulfonylamino, carboxyl, (N,N-) di-(C₁-C₇-alkyl)-amino-C₁-C₇-alkoxycarbonyl, halo-C₁-C₇-alkoxycarbonyl, phenyl- or naphthyloxycarbonyl, phenyl- or naphthyl-C₁-C₇-alkoxycarbonyl, (N,N-) di-(C₁-C₇-alkyl)-amino-C₁-C₇-alkoxycarbonyl, carbamoyl, N-mono or N,N-di-(naphthyl-, phenyl-, C₁-C₇-alkyloxyphenyl and/or C₁-C₇-alkyloxynapthtyl)aminocarbonyl, N-mono- or N,N-di-(naphthyl- or phenyl-C₁-C₇-alkyl)-aminocarbonyl, cyano, C₁-C₇-alkylene which is unsubstituted or substituted by up to four C₁-C₇-alkyl substituents and bound to two adjacent ring atoms of the aryl moiety, C₂-C₇-alkenylene or -alkynylene which are bound to two adjacent ring atoms of the aryl moiety, sulfenyl, sulfinyl, C₁-C₇-alkylsulfinyl, phenyl- or naphthylsulfinyl wherein phenyl or naphthyl is unsubstituted or substituted by one or more, especially one to three, C₁-C₇-alkoxy-C₁-C₇-alkyl or C₁-C₇-alkyl moieties, phenyl- or naphthyl-C₁-C₇-alkylsulfinyl, sulfonyl, C₁-C₇-alkylsulfonyl, halo-C₁-C₇-alkylsulfonyl, hydroxy-C₁-C₇-alkylsulfonyl, C₁-C₇-alkoxy-C₁-C₇-alkylsulfonyl, amino-C₁-C₇-alkylsulfonyl, (N,N-) di-(C₁-C₇-alkyl)-amino-C₁-C₇-alkylsulfonyl, C₁-C₇-alkanoylamino-C₁-C₇-alkylsulfonyl, phenyl- or naphthylsulfonyl wherein phenyl or naphthyl is unsubstituted or substituted by one or more, especially one to three, C₁-C₇-alkoxy-C₁-C₇-alkyl or C₁-C₇-alkyl moieties, phenyl- or naphthyl-C₁-C₇-alkylsulfonyl, sulfamoyl and N-mono or N,N-di-(C₁-C₇-alkyl, phenyl-, naphthyl, phenyl-C₁-C₇-alkyl and/or naphthyl-C₁-C₇-alkyl)-aminosulfonyl; especially preferably aryl is phenyl or naphthyl, each of which is unsubstituted or substituted by one or more, e.g. up to three, substituents independently selected from the group consisting of C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, amino-C₁-C₇-alkyl, C₁-C₇-alkoxy-C₁-C₇-alkylamino-C₁-C₇-alkyl, carboxy-C₁-C₇-alkyl, C₁-C₇-alkoxycarbonyl-C₁-C₇-alkyl, halo, especially fluoro, chloro or bromo, hydroxy, C₁-C₇-alkoxy, hydroxy-C₁-C₇-alkoxy, C₁-C₇-alkoxy-C₁-C₇-alkoxy, amino-C₁-C₇-alkoxy, N—C₁-C₇-alkanoylamino-C₁-C₇-alkoxy, carboxyl-C₁-C₇-alkyloxy, C₁-C₇-alkoxycarbonyl-C₁-C₇-alkyloxy, carbamoyl-C₁-C₇-alkoxy, N-mono- or N,N-di-(C₁-C₇-alkyl)-carbamoyl-C₁-C₇-alkoxy, morpholino-C₁-C₇-alkoxy, pyridyl-C₁-C₇-alkoxy, amino, C₁-C₇-alkanoylamino, C₁-C₇-alkanoyl, C₁-C₇-alkoxy-C₁-C₇-alkanoyl, carboxy, carbamoyl, N—(C₁-C₇-alkoxy-C₁-C₇-alkyl)-carbamoyl, pyrazolyl, pyrazolyl-C₁-C₇-alkoxy, 4-C₁-C₇-alkylpiperidin-1-yl, nitro and cyano;
unsubstituted or substituted heterocyclyl is a mono- or polycyclic, preferably a mono-, bi- or tricyclic-, unsaturated, partially saturated or saturated ring system with preferably 3 to 22 (more preferably 3 to 14) ring atoms and with one or more, preferably one to four, heteroatoms independently selected from nitrogen, oxygen, sulfur, S(═O)— or S—(═O)₂, and is unsubstituted or substituted by one or more, e.g. up to three, substitutents preferably independently selected from the substitutents mentioned above for aryl and from oxo; where preferably, heterocyclyl which is unsubstituted or substituted as just mentioned is selected from the following moieties wherein the asterisk marks the point of binding to the rest of the molecule of formula I: 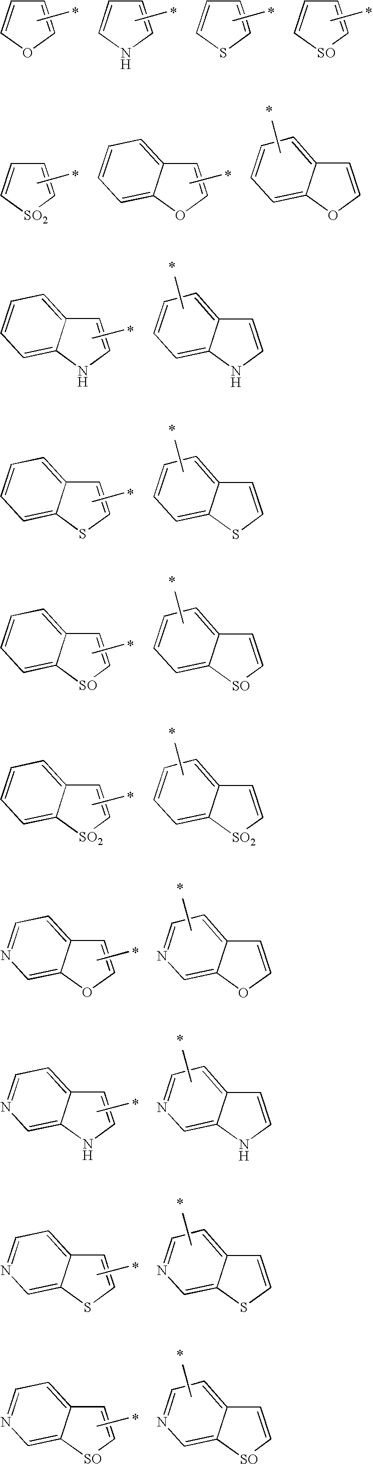 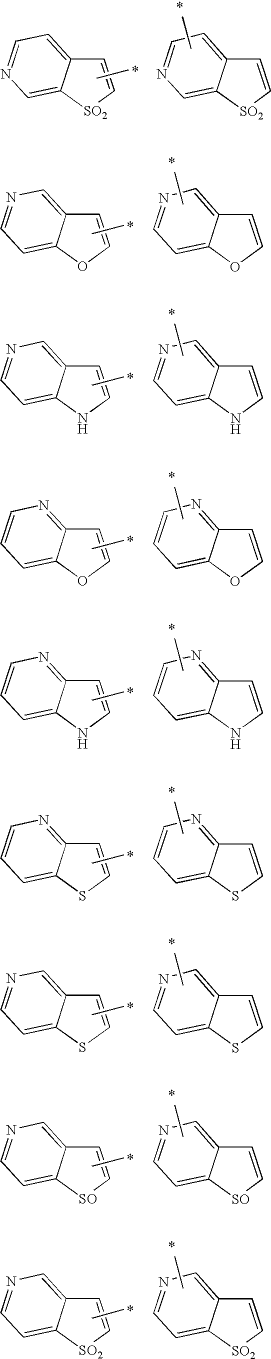 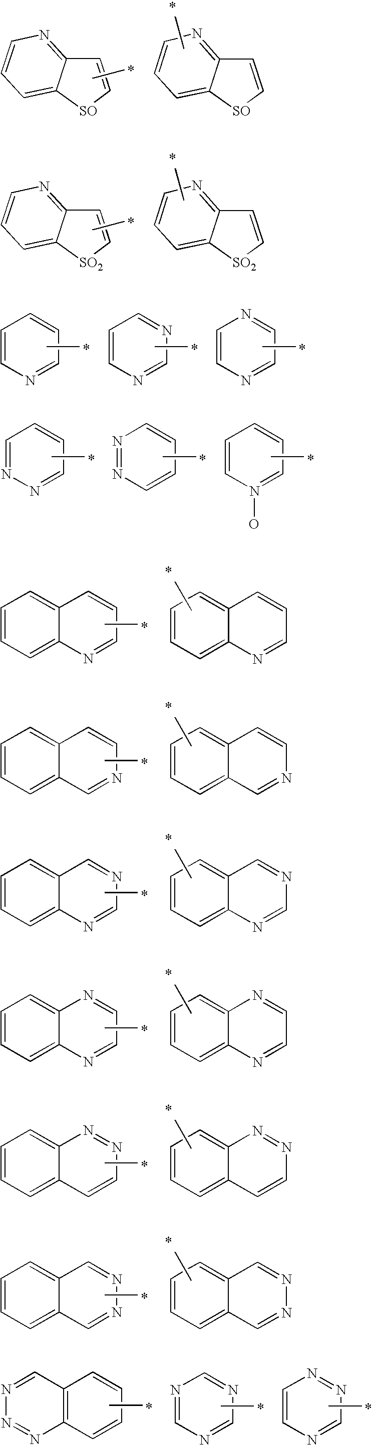 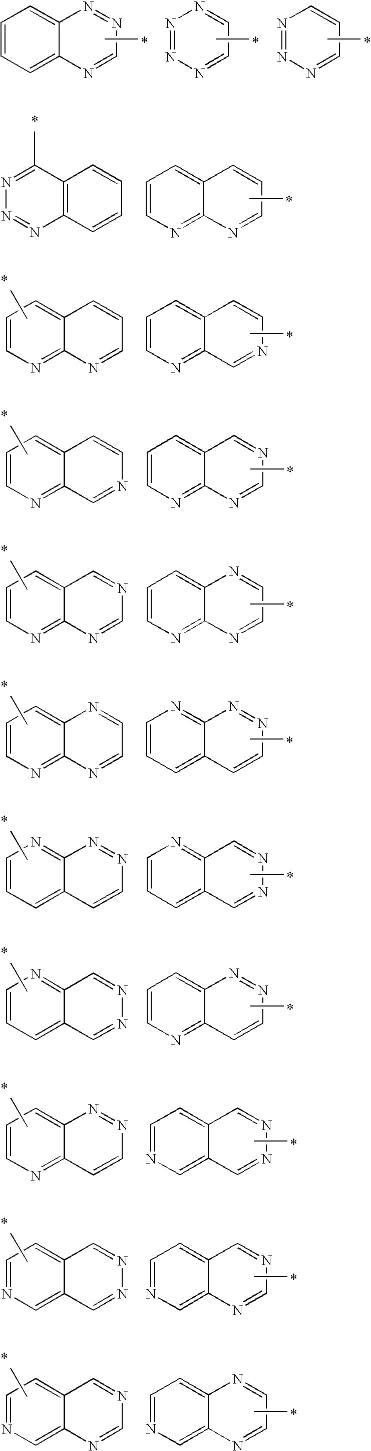 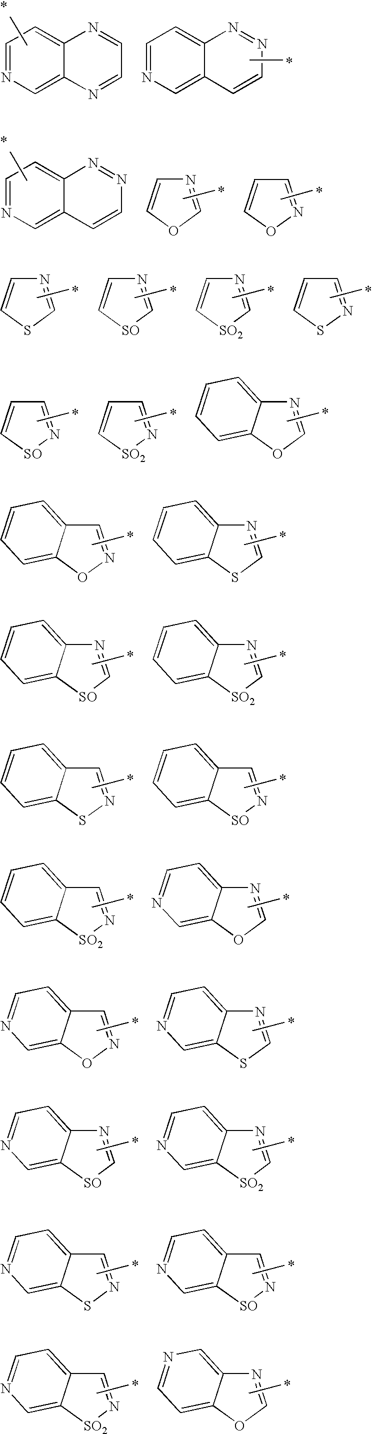 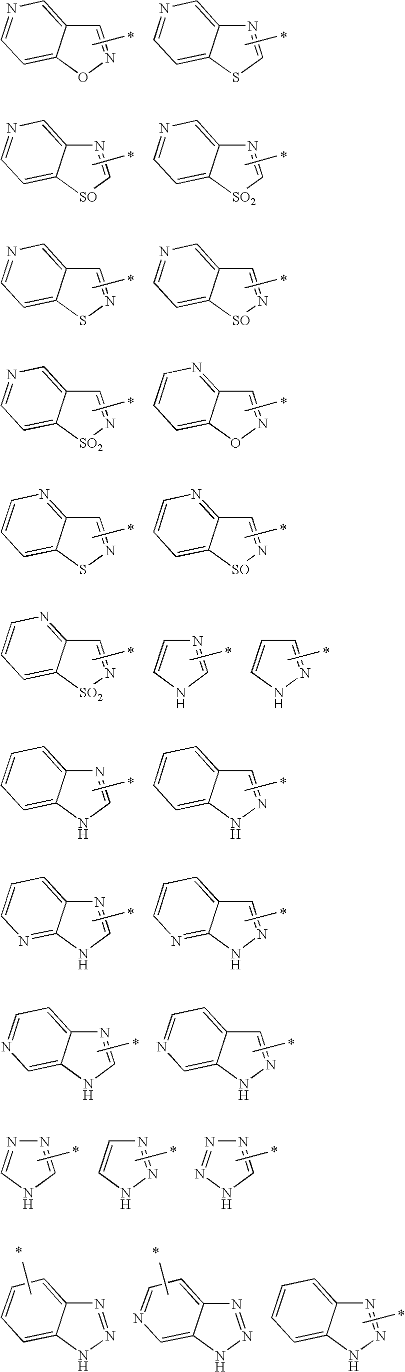 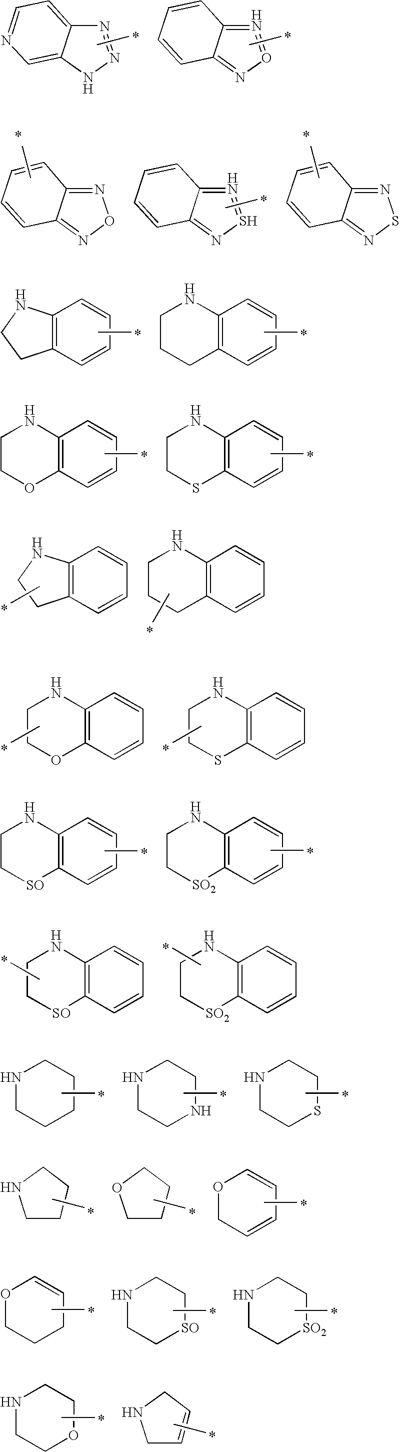 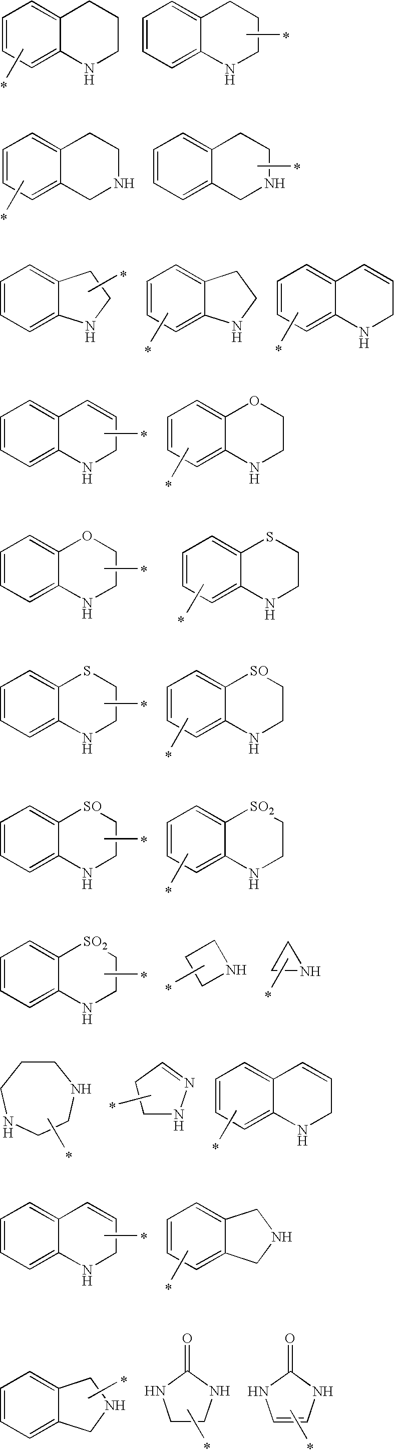 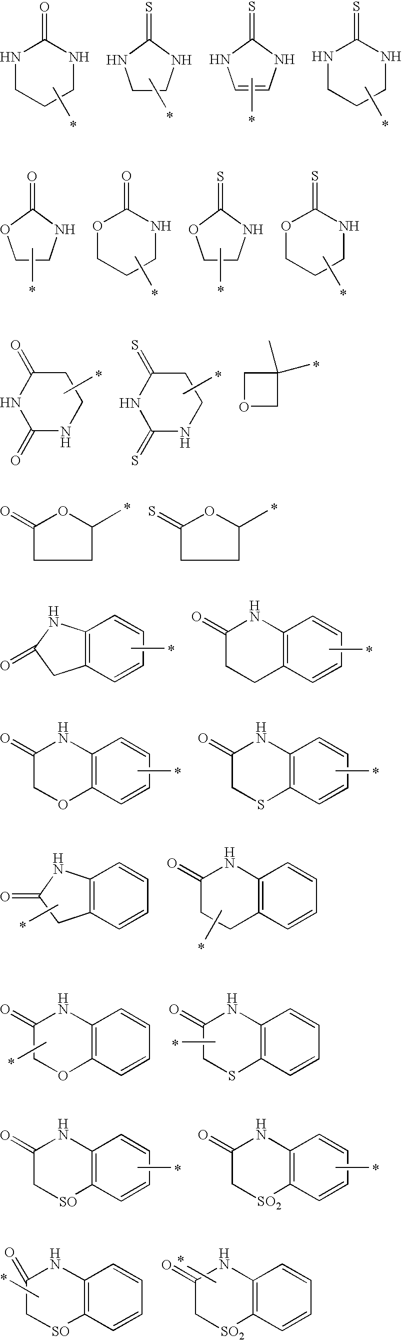 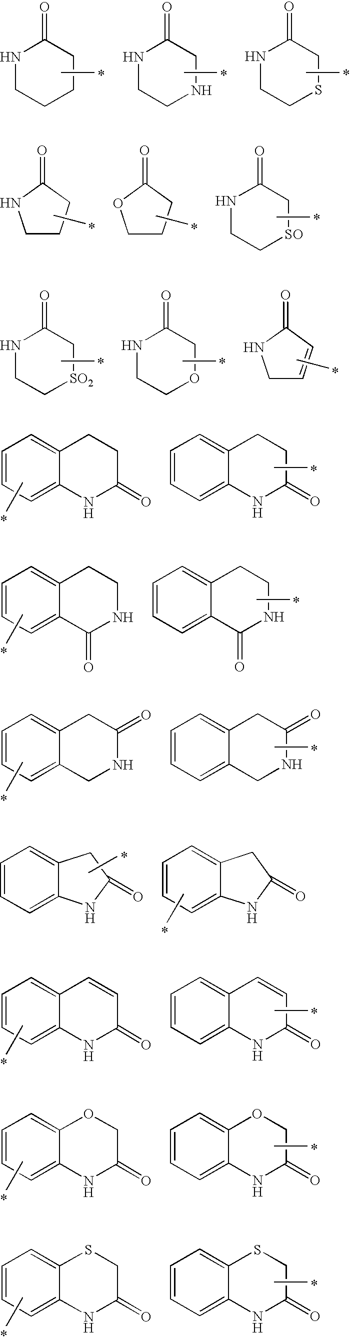 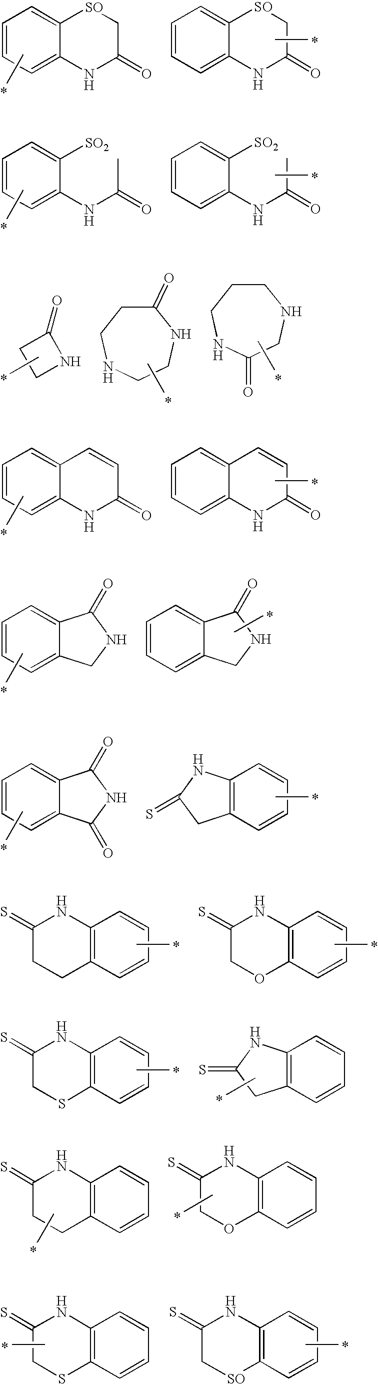 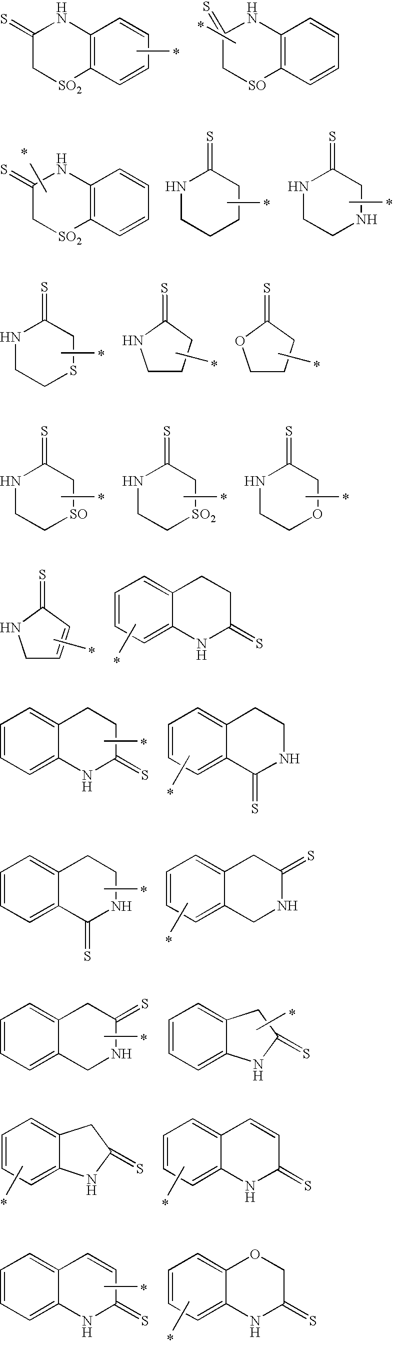 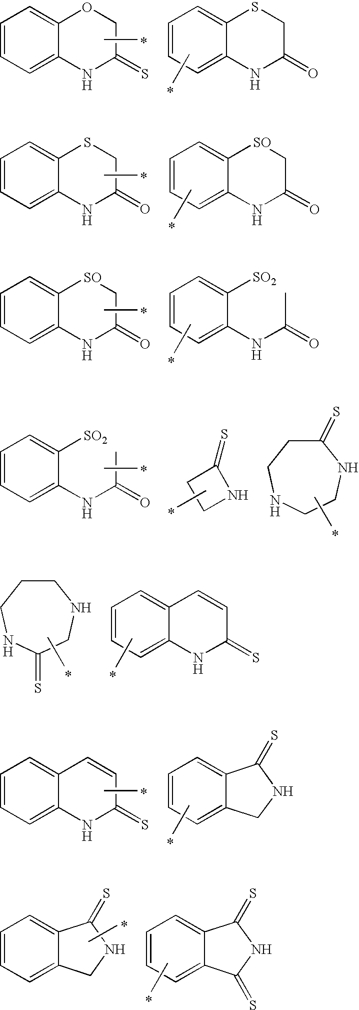
where in each case where an NH is present the bond with the asterisk connecting the respective heterocyclyl moiety to the rest of the molecule the H may be replaced with said bond and/or the H may be replaced by a substituent, preferably as defined above; especially preferred as heterocyclyl is pyrrolyl, furanyl, thienyl, pyrimidinyl, pyrazolyl, pyrazolidinonyl (=oxo-pyrazolidinyl), triazolyl, tetrazolyl, oxetidinyl, pyridyl, pyrimidinyl, morpholino, piperidinyl, piperazinyl, pyrrolidinyl, tetrahydrofuran-onyl (=oxo-tetrahydrofuranyl), tetrahydro-pyranyl, indolyl, indazolyl, 1H-indazanyl, benzofuranyl, benzothiophenyl, quinolinyl, isoquinolinyl, 1,2,3,4-tetrahydro-1,4-benzoxazinyl, 2H-1,4-benzoxazin-3(4H)-onyl, 2H,3H-1,4-benzodioxinyl, benzo[1,2,5]oxadiazolyl or thiophenyl, each of which is unsubstituted or substituted by one or more, e.g. up to three, substituents as mentioned above for substituted aryl, preferably independently selected from the group consisting of C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, amino-C₁-C₇-alkyl, C₁-C₇-alkoxy-C₁-C₇-alkylamino-C₁-C₇-alkyl, carboxy-C₁-C₇-alkyl, C₁-C₇-alkoxy-C₁-C₇-alkyl, halo, hydroxy, C₁-C₇-alkoxy, C₁-C₇-alkoxy-C₁-C₇-alkoxy, amino-C₁-C₇-alkoxy, N—C₁-C₇-alkanoylamino-C₁-C₇-alkoxy, carbamoyl-C₁-C₇-alkoxy, N—C₁-C₇-alkylcarbamoyl-C₁-C₇-alkoxy, C₁-C₇-alkanoyl, C₁-C₇-alkoxy-C₁-C₇-alkanoyl, carboxy, carbamoyl and N—C₁-C₇-alkoxy-C₁-C₇-alkylcarbamoyl. In the case of heterocycles including an NH ring member, the substitutents, as far as bound via a carbon or oxygen atom, are preferably bound at the nitrogen instead of the H;
unsubstituted or substituted cycloalkyl is mono- or polycyclic, more preferably monocyclic, C₃-C₁₀-cycloalkyl which may include one or more double and/or triple bonds, and is unsubstituted or substituted by one or more, e.g. one to three substitutents preferably independently selected from those mentioned above as substituents for aryl; where cyclopropyl, cyclobutyl, cyclopentyl, cyclohexyl or cycloheptyl is preferred;
acyl is unsubstituted or substituted aryl-carbonyl or -sulfonyl, unsubstituted or substituted heterocyclylcarbonyl or -sulfonyl, unsubstituted or substituted cycloalkylcarbonyl or -sulfonyl, formyl, unsubstituted or substituted alkylcarbonyl or -sulfonyl, substituted aryl-oxycarbonyl or -oxysulfonyl, unsubstituted or substituted heterocyclyloxycarbonyl or -oxysulfonyl, unsubstituted or substituted cycloalkyloxycarbonyl or -oxysulfonyl, unsubstituted or substituted alkyloxycarbonyl or -oxysulfonyl or N-mono- or N,N-di-(substituted aryl-, unsubstituted or substituted heterocyclyl, unsubstituted or substituted cycloalkyl or unsubstituted or substituted alkyl)-aminocarbonyl; wherein unsubstituted or substituted aryl, unsubstituted or substituted heterocyclyl, unsubstituted or substituted cycloalkyl and unsubstituted or substituted alkyl are preferably as described above; where C₁-C₇-alkanoyl, unsubstituted or mono-, di- or tri-(halo)-substituted benzoyl or naphthoyl, unsubstituted or phenyl-substituted pyrrolidinylcarbonyl, especially phenyl-pyrrolidinocarbonyl, C₁-C₇-alkylsulfonyl or (unsubstituted or C₁-C₇-alkyl-substituted) phenylsulfonyl are preferred;
etherified or esterified hydroxy is hydroxy that is esterified with acyl as defined above, especially in C₁-C₇-alkanoyloxy; or preferably etherified with alkyl, alkenyl, alkynyl, aryl, heterocyclyl or cycloalkyl each of which is unsubstituted or substituted and is preferably as described above for the corresponding unsubstituted or substituted moieties, where unsubstituted or especially substituted C₁-C₇-alkyloxy is especially preferred, especially with a substituent selected from C₁-C₇-alkoxy; phenyl, tetrazolyl, tetrahydrofuran-onyl, oxetidinyl, 3-(C₁-C₇-alkyl)-oxetidinyl, pyridyl or 2H,3H-1,4-benzodioxinyl, each of which is unsubstituted or substituted by one or more, preferably up to three, e.g. 1 or two substituents independently selected from C₁-C₇-alkyl, hydroxy, C₁-C₇-alkoxy, phenyloxy wherein phenyl is unsubstituted or substituted by C₁-C₇-alkoxy and/or halo, phenyl-C₁-C₇-alkoxy wherein phenyl is unsubstituted or substituted by C₁-C₇-alkoxy and/or halo; halo, amino, N-mono- or N,N-di(C₁-C₇-alkyl, phenyl, naphthyl, phenyl-C₁-C₇-alkyl or naphthyl-C₁-C₇-alkyl)amino, C₁-C₇-alkanoylamino, carboxy, N-mono- or N,N-di(C₁-C₇-alkyl, phenyl, naphthyl, phenyl-C₁-C₇-alkyl or naphthyl-C₁-C₇-alkyl)-aminocarbonyl, morpholino, morpholino-C₁-C₇-alkoxy, pyridyl-C₁-C₇-alkoxy, pyrazolyl, 4-C₁-C₇-alkylpiperidin-1-yl and cyano;
or selected from morpholino; or unsubstituted or substituted aryloxy with unsubstituted or substituted aryl as described above, especially phenyloxy with phenyl that is unsubstituted or substituted as just described; or
unsubstituted or substituted heterocyclyloxy with unsubstituted or substituted heterocyclyl as described above, preferably tetrahydropyranyloxy;
substituted mercapto is mercapto that is thioesterified with acyl as defined above, especially with lower alkanoyloxy; or preferably thioetherified with alkyl, alkenyl, alkynyl, aryl, heterocyclyl or cycloalkyl each of which is unsubstituted or substituted and is preferably as described above for the corresponding unsubstituted or substituted moieties, where unsubstituted or especially substituted C₁-C₇-alkylthio or unsubstituted or substituted arylthio with unsubstituted or substituted C₁-C₇-alkyl or aryl as just described for the corresponding moieties under etherified hydroxy are especially preferred;
substituted sulfinyl or sulfonyl is sulfonyl substituted with alkyl, alkenyl, alkynyl, aryl, heterocyclyl or cycloalkyl each of which is unsubstituted or substituted and is preferably as described above for the corresponding unsubstituted or substituted moieties, where unsubstituted or especially substituted C₁-C₇-alkylsulfinyl or -sulfonyl or unsubstituted or substituted arylsulfinyl or -sulfonyl with unsubstituted or substituted C₁-C₇-alkyl or aryl as just described for the corresponding moieties under etherified hydroxy are especially preferred;
in mono- or di-substituted amino, amino is substituted by one or more substituents selected from one acyl, especially C₁-C₇-alkanoyl, phenylcarbonyl (=benzoyl), C₁-C₇-alkylsulfonyl or phenylsulfonyl wherein phenyl is unsubstituted or substituted by one to 3 C₁-C₇-alkyl groups, and one or two moieties selected from alkyl, alkenyl, alkynyl, aryl, heterocyclyl and cycloalkyl each of which is unsubstituted or substituted and is preferably as described above for the corresponding unsubstituted or substituted moieties; where C₁-C₇-alkanoylamino, mono- or di-(phenyl, naphthyl, C₁-C₇-alkoxy-phenyl, C₁-C₇-alkoxynaphthyl, naphthyl-C₁-C₇-alkyl or phenyl-C₁-C₇-alkyl)-carbonylamino (e.g. 4-methoxybenzoylamino), mono- or di-(C₁-C₇-alkyl and/or C₁-C₇-alkoxy-C₁-C₇-alkyl)-amino or mono- or di-(phenyl, naphthyl, C₁-C₇-alkoxy-phenyl, C₁-C₇-alkoxynaphthyl, phenyl-C₁-C₇-alkyl, naphthyl-C₁-C₇-alkyl, C₁-C₇-alkoxy-naphthyl-C₁-C₇-alkyl or C₁-C₇-alkoxy-phenyl-C₁-C₇-alkyl)-amino is especially preferred;
esterified carboxy is alkyloxycarbonyl, aryloxycarbonyl, heterocyclyloxycarbonyl or cycloalkyloxycarbonyl, wherein alkyl, aryl, heterocyclyl and cycloalkyl are unsubstituted or substituted and the corresponding moieties and their substituents are preferably as described above, where C₁-C₇-alkoxycarbonyl, phenyl-C₁-C₇-alkyloxycarbonyl, phenoxycarbonyl or naphthoxycarbonyl is especially preferred;
in amidated carboxy, the amino part bound to the carbonyl in the amido function D₂N—C(═O)— wherein each D is independently of the other hydrogen or an amino substituent is unsubstituted or substituted as described for substituted amino, where mono- or di-(C₁-C₇-alkyl and/or C₁-C₇-alkoxy-C₁-C₇-alkyl)-aminocarbonyl or mono- or di-(C₁-C₇-alkyloxyphenyl, C₁-C₇-alkyloxynaphthyl, naphthyl-C₁-C₇-alkyl or phenyl-C₁-C₇-alkyl)-aminocarbonyl is especially preferred;
in substituted sulfamoyl, the amino part bound to the sulfonyl in the sulfamoyl function D₂N—S(═O)₂— wherein each D is independently of the other hydrogen or an amino substituent is unsubstituted or substituted as described for substituted amino, where mono- or di-(C₁-C₇-alkyl and/or C₁-C₇-alkoxy-C₁-C₇-alkyl)-aminosulfonyl or mono- or di-(C₁-C₇-alkyloxyphenyl, C₁-C₇-alkyloxynaphthyl, naphthyl-C₁-C₇-alkyl or phenyl-C₁-C₇-alkyl)-aminosulfonyl is especially preferred.
unsubstituted or substituted C₁-C₇-alkyl, unsubstituted or substituted C₂-C₇-alkenyl and un-substituted or substituted C₂-C₇-alkynyl and their substituents are defined as above under the corresponding (un)substituted alkyl, (un)substituted alkynyl and (un)substituted alkynyl moieties but with the given number of carbon atoms in the alkyl, alkenyl or alkynyl moieties;
or a salt thereof.

3. A compound of the formula I according to claim 1, wherein
R1 is C₁-C₇-alkyl, halo-C₁-C₇-alkyl, di-(phenyl)-C₁-C₇-alkyl, C₃-C₈-cyclopropyl, (unsubstituted or C₁-C₇-alkoxy-substituted naphthyl)-C₁-C₇-alkyl, (halo-phenyl)-C₁-C₇-alkyl or phenyl substituted by C₁-C₇-alkyl, halo, C₁-C₇-alkyloxy and/or C₁-C₇-alkoxy-C₁-C₇-alkyloxy,
R2 is hydrogen, phenyl-C₁-C₇-alkyl, di-(phenyl)-C₁-C₇-alkyl, naphthyl-C₁-C₇-alkyl, phenyl, naphthyl, pyridyl-C₁-C₇-alkyl, indolyl-C₁-C₇-alkyl, 1H-indazolyl-C₁-C₇-alkyl, quinolyl-C₁-C₇-alkyl, isoquinolyl-C₁-C₇-alkyl, 1,2,3,4-tetrahydro-1,4-benzoxazinyl-C₁-C₇-alkyl, 2H-1,4-benzoxazin-3(4H)-onyl-C₁-C₇-alkyl, 1-benzothiophenyl-C₁-C₇-alkyl, pyridyl, indolyl, 1H-indazolyl, quinolyl, isoquinolyl, 1,2,3,4-tetrahydro-1,4-benzoxazinyl, 2H-1,4-benzoxazin-3(4H onyl, 1-benzothiophenyl, phenylcarbonyl (benzoyl) or naphthylcarbonyl (naphthoyl), where each phenyl, naphthyl, pyridyl, indolyl, 1H-indazolyl, quinolyl, isoquinolyl, 1,2,3,4-tetrahydro-1,4-benzoxazinyl, 2H-1,4-benzoxazin-3(4H)-onyl or 1-benzothiophenyl is unsubstituted or substituted by one or more, e.g. up to three, substituents independently selected from the group consisting of C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkanoyloxy-C₁-C₇-alkyl, amino-C₁-C₇-alkyl, C₁-C₇-alkoxy-C₁-C₇-alkylamino-C₁-C₇-alkyl, C₁-C₇-alkanoylamino-C₁-C₇-alkyl, C₁-C₇-alkylsulfonylamino-C₁-C₇-alkyl, carboxy-C₁-C₇-alkyl, C₁-C₇-alkoxycarbonyl-C₁-C₇-alkyl, halo, hydroxy, C₁-C₇-alkoxy, hydroxy-C₁-C₇-alkyloxy, C₁-C₇-alkoxy-C₁-C₇-alkoxy, amino-C₁-C₇-alkoxy, N—C₁-C₇-alkanoylamino-C₁-C₇-alkoxy, carboxy-C₁-C₇-alkyloxy, C₁-C₇-alkyloxycarbonyl-C₁-C₇-alkoxy, carbamoyl-C₁-C₇-alkoxy, N-mono- or N,N-di-(C₁-C₇-alkyl)-carbamoyl-C₁-C₇-alkoxy, morpholino-C₁-C₇-alkoxy, pyridyl-C₁-C₇-alkoxy, amino, C₁-C₇-alkanoylamino, C₁-C₇-alkanoyl, C₁-C₇-alkyloxy-C₁-C₇-alkanoyl, C₁-C₇-alkoxy-C₁-C₇-alkanoyl, carboxyl, carbamoyl, N—C₁-C₇-alkoxy-C₁-C₇-alkylcarbamoyl, pyrazolyl, pyrazolyl-C₁-C₇-alkoxy, 4-C₁-C₇-alkylpiperidin-1-yl, nitro and cyano;
W is a moiety of the formula IA, 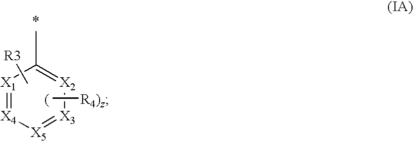
wherein the asterisk (*) denotes the position where the moiety W is bound to the 4-carbon in the piperidine ring in formula I, and wherein one of X₁ and X₂ is nitrogen or CH, while the other and X₃, X₄ and X₅ are CH; preferably with the proviso that R3 is bound to X₁ or X₂ or preferably to X₃ or X₄; or a moiety of the formula IB, 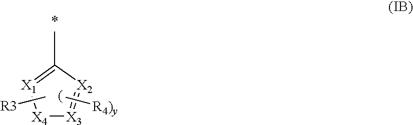
wherein the asterisk (*) denotes the position where the moiety W is bound to the 4-carbon in the piperidine ring in formula I, and wherein X₄ is CH₂, NH, S or O and one of X₁, X₂ and (preferably if X₄ is CH₂ or N) X₃, more preferably X₂, is N, while the others are each CH, with the proviso that at least one ring nitrogen (N or in the case or X₄ NH) is present and that R3 is then preferably bound to X₃; preferably, X₁ is CH or N, X₂ is CH or N, X₃ is CH or N and X₄ is NH, O or S, with the proviso that not more than one of X₁, X₂ and X₃ is N; and preferably with the proviso that R3 is bound to X₁ or X₂ or preferably to X₃ or X₄;
or a moiety of the formula IC, 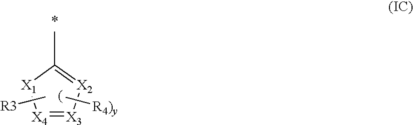
wherein the asterisk (*) denotes the position where the moiety W is bound to the 4-carbon in the piperidine ring in formula I, and wherein
X₁ is CH₂, NH, S or O and one of X₂, X₃ and X₄ is N, while the others are CH, with the proviso that at least one ring nitrogen (N or in the case or X₁ NH) is present; preferably, X₁ is S or O, X₂ is CH or N, X₃ is CH or N, and X₄ is CH or N, with the proviso that not more than one of X₂, X₃ and X₄ is N; and preferably with the proviso that R3 is bound to X₂ or preferably to X₃ or X₄;
where in each case where R3 is bond to a moiety of the formula IA, IB or IC, instead of a hydrogen atom at a ring member NH, CH₂ or CH mentioned so far where R3 is bound a moiety R3 is present;
y is 0 or 1, preferably 0, and z is 0, 1 or 2, preferably 0 or 1; R3 is hydrogen or preferably C₁-C₇-alkyloxy-C₁-C₇-alkyloxy, phenyloxy-C₁-C₇-alkyl, phenyl, phenyl-C₁-C₇-alkoxy, naphthyl, naphthyl-C₁-C₇-alkoxy, pyridyl, pyridyl-C₁-C₇-alkoxy, phenyloxy, napthyloxy, phenyloxy-C₁-C₇-alkoxy, morpholino-C₁-C₇-alkoxy, tetrahydropyranyloxy, 2H,3H-1,4-benzodioxinyl-C₁-C₇-alkoxy, phenylaminocarbonyl or phenylcarbonylamino,
wherein in each case where present under R3 phenyl, naphthyl or pyridyl is unsubstituted or substituted by one or more, preferably up to three, moieties independently selected from the group consisting of C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, amino-C₁-C₇-alkyl, C₁-C₇-alkoxy-C₁-C₇-alkylamino-C₁-C₇-alkyl, carboxy-C₁-C₇-alkyl, halo, especially fluoro, chloro or bromo, hydroxy, C₁-C₇-alkoxy, C₁-C₇-alkoxy-C₁-C₇-alkoxy, amino-C₁-C₇-alkoxy, N—C₁-C₇-alkanoylamino-C₁-C₇-alkoxy, carbamoyl-C₁-C₇-alkoxy, N-mono- or N,N-di-(C₁-C₇-alkyl)-carbamoyl-C₁-C₇-alkoxy, morpholino-C₁-C₇-alkoxy, pyridyl-C₁-C₇-alkoxy, amino, C₁-C₇-alkanoylamino, C₁-C₇-alkanoyl, C₁-C₇-alkoxy-C₁-C₇-alkanoyl, carboxy, carbamoyl, N—(C₁-C₇-alkoxy-C₁-C₇-alkyl)-carbamoyl, pyrazolyl, pyrazolyl-C₁-C₇-alkoxy, 4-C₁-C₇-alkylpiperidin-1-yl, nitro and cyano
R4 if present (which is the case if y or z is other than zero) is hydroxy, halo or C₁-C₇-alkoxy;
T is carbonyl; and
G is methylene, oxy or imino; and R5 is hydrogen, C₁-C₇-alkyl, C₁-C₇-alkoxy-C₁-C₇-alkyl, C₁-C₇-alkoxy, C₁-C₇-alkanoyl, C₁-C₇-alkylsulfonyl or (unsubstituted or C₁-C₇-alkyl-substituted phenyl)-sulfonyl or
-G-R5 is hydrogen;
or a pharmaceutically acceptable salt thereof.

4. A compound of the formula I according to claim 1, wherein
R1 is C₁-C₇-alkyl, halo-C₁-C₇-alkyl, di-(phenyl)-C₁-C₇-alkyl, C₃-C₈-cyclopropyl, (unsubstituted or C₁-C₇-alkoxy-substituted naphthyl)-C₁-C₇-alkyl, (halo-phenyl)-C₁-C₇-alkyl or phenyl substituted by C₁-C₇-alkyl, halo, C₁-C₇-alkyloxy and/or C₁-C₇-alkoxy-C₁-C₇-alkyloxy,
R2 is hydrogen, phenyl-C₁-C₇-alkyl, di-(phenyl)-C₁-C₇-alkyl, naphthyl-C₁-C₇-alkyl, phenyl, naphthyl, pyridyl-C₁-C₇-alkyl, indolyl-C₁-C₇-alkyl, 1H-indazolyl-C₁-C₇-alkyl, quinolyl-C₁-C₇-alkyl, isoquinolyl-C₁-C₇-alkyl, 1-benzothiophenyl-C₁-C₇-alkyl or phenylcarbonyl (benzoyl), where each phenyl, naphthyl, pyridyl, indolyl, 1H-indazolyl, quinolyl, isoquinolyl or 1-benzothiophenyl is unsubstituted or substituted by one or more, e.g. up to three, substituents independently selected from the group consisting of C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, amino-C₁-C₇-alkyl, C₁-C₇-alkoxy-C₁-C₇-alkylamino-C₁-C₇-alkyl, C₁-C₇-alkanoylamino-C₁-C₇-alkyl, C₁-C₇-alkoxycarbonyl-C₁-C₇-alkyl, halo, C₁-C₇-alkoxy, hydroxy-C₁-C₇-alkyloxy, C₁-C₇-alkoxy-C₁-C₇-alkoxy, amino-C₁-C₇-alkoxy, N—C₁-C₇-alkanoylamino-C₁-C₇-alkoxy, carboxy-C₁-C₇-alkyloxy, C₁-C₇-alkyloxycarbonyl-C₁-C₇-alkoxy, carbamoyl-C₁-C₇-alkoxy, N-mono- or N,N-di-(C₁-C₇-alkyl)-carbamoyl-C₁-C₇-alkoxy, C₁-C₇-alkanoyl, C₁-C₇-alkyloxy-C₁-C₇-alkanoyl, carbamoyl and N—C₁-C₇-alkoxy-C₁-C₇-alkylcarbamoyl;
W is a moiety of the formula IA, 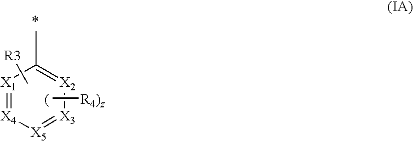
wherein the asterisk (*) denotes the position where the moiety W is bound to the 4-carbon in the piperidine ring in formula I, and wherein
X₁ is N or CH and each of X₂, X₃, X₄ and X₅ is CH;
or a moiety of the formula IC, 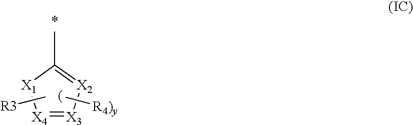
wherein the asterisk (*) denotes the position where the moiety W is bound to the 4-carbon in the piperidine ring in formula I, and wherein X₁ is CH₂ or O, X₄ is N and X₂ and X₃ each are CH, with the proviso that R3 is bound to X₃ instead of the hydrogen;
z is 0 or 1; y is 0;
R3 is phenyl, phenyl-C₁-C₇-alkoxy, pyridyl, pyridyl-C₁-C₇-alkoxy, phenyloxy, phenyloxy-C₁-C₇-alkoxy or morpholino-C₁-C₇-alkoxy, wherein in each case where present under R3 phenyl or pyridyl is unsubstituted or substituted by one or more, preferably up to three, moieties independently selected from the group consisting of halo, especially fluoro, chloro or bromo, hydroxy, C₁-C₇-alkoxy, morpholino-C₁-C₇-alkoxy, C₁-C₇-alkanoylamino, pyrazolyl, 4-C₁-C₇-alkylpiperidin-1-yl and cyano;
R4 (present if z is 1) is a moiety independently selected from hydroxy and C₁-C₇-alkoxy;
T is carbonyl; and
G-R5 is hydrogen, hydroxy, C₁-C₇-alkyloxy, C₁-C₇-alkoxy-C₁-C₇-alkyloxy, amino, C₁-C₇-alkanoylamino, C₁-C₇-alkylsulfonylamino or (unsubstituted or C₁-C₇-alkyl-substituted phenyl)-sulfonylamino;
or a pharmaceutically acceptable salt thereof.

5. A compound of the formula I according to claim 1 of the formula A, 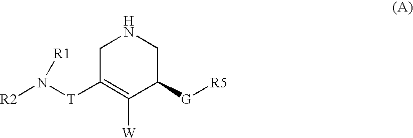
wherein R1, R2, R5, T, G and W are as defined for a compound of the formula I in claim 1, or a pharmaceutically acceptable salt thereof.

6. A compound of the formula I according to claim 1 of the formula B, 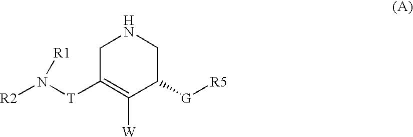
wherein R1, R2, R5, T, G and W are as defined for a compound of the formula I in claim 1, or a pharmaceutically acceptable salt thereof.

7. A compound of the formula I according to claim 1, selected from the group of compounds represented by any one of the following formulae: 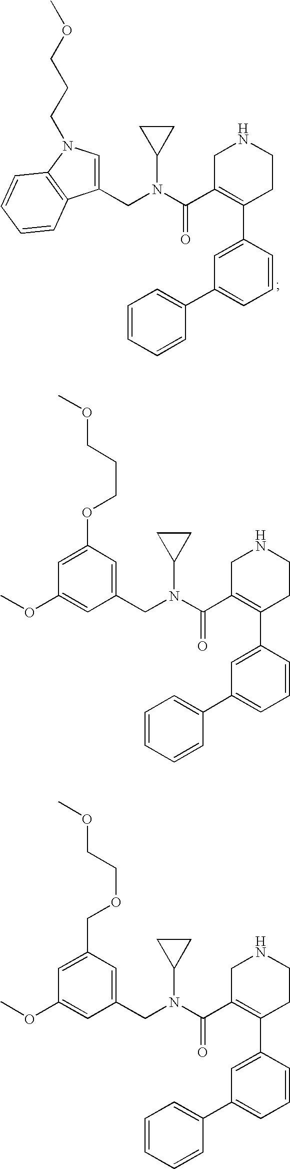 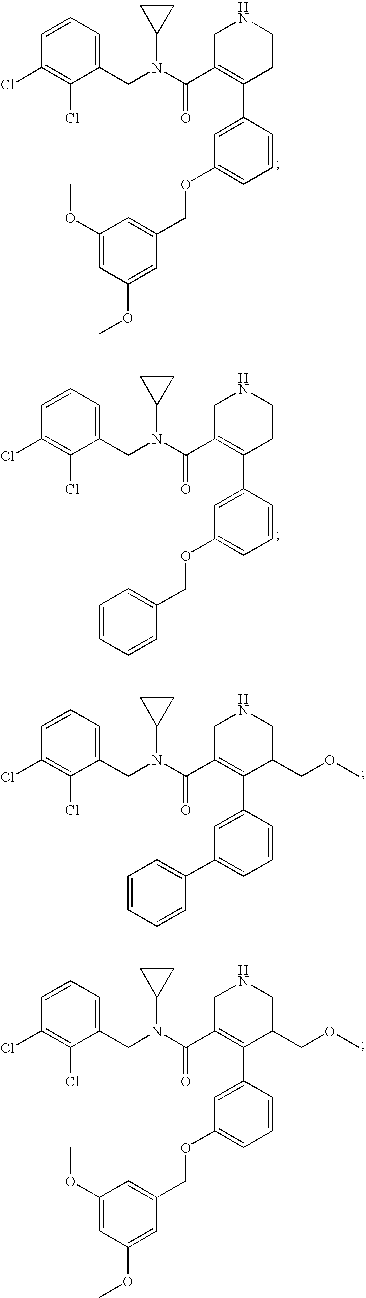 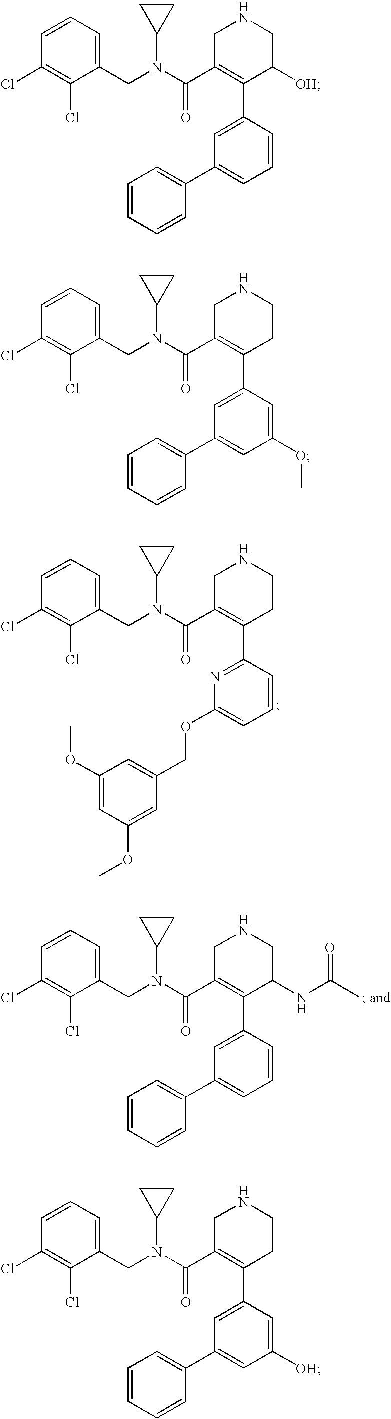
or a pharmaceutically acceptable salt thereof.

8. A compound of the formula I according to claim 1, selected from the group of compounds represented by the formula 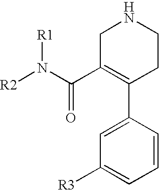
as represented in the following table:

| Compound No. | R1 | R2 | R3 |
|---|---|---|---|
| 1 | 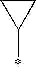 | 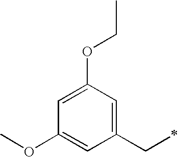 | 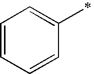 |
| 2 | 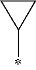 | 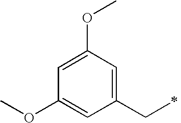 | 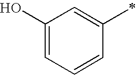 |
| 3 | 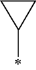 | 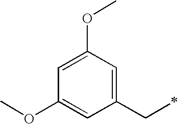 | 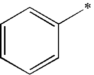 |
| 4 | 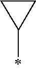 | 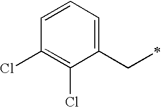 | 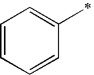 |
| 5 | 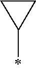 | 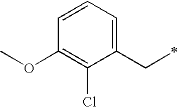 | 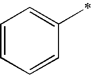 |
| 6 | 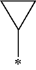 | 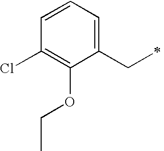 | 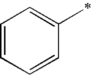 |
| 7 | 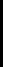 | 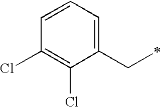 | 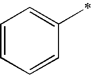 |
| 8 | 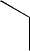 | 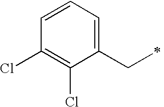 | 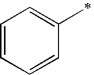 |
| 9 | 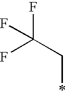 | 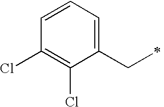 | 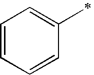 |
| 10 | 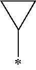 | 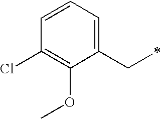 | 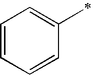 |
| 11 | 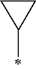 | 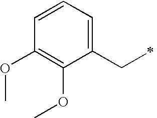 | 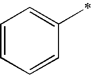 |
| 12 | 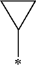 | 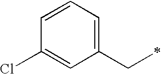 | 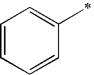 |
| 13 | H | 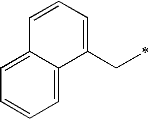 | 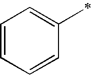 |
| 14 | 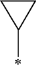 | 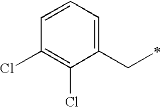 | 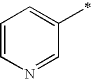 |
| 15 | 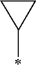 | 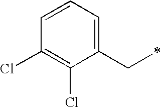 | 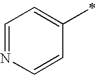 |
| 16 | 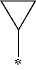 | 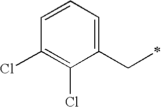 | 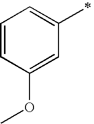 |
| 17 | 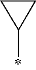 | 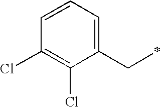 | 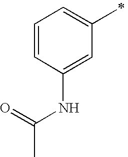 |
| 18 | 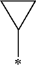 | 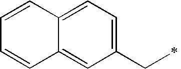 | 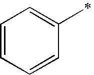 |
| 19 | 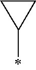 | 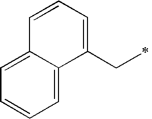 | 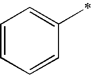 |
| 20 | 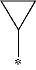 | 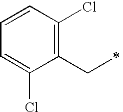 | 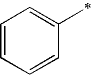 |
| 21 | 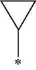 | 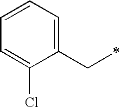 | 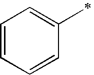 |
| 22 | 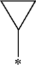 | 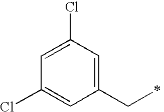 | 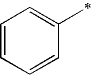 |
| 23 | 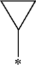 | 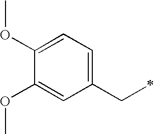 | 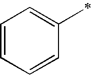 |
| 24 | 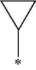 | 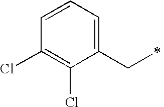 | 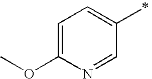 |
| 25 | 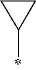 | 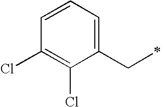 | 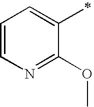 |
| 26 | 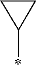 | 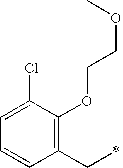 | 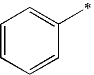 |
| 27 | 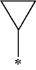 | 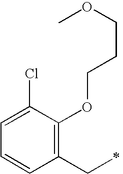 | 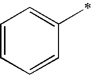 |
| 28 | 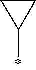 | 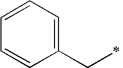 | 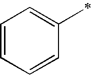 |
| 29 | 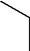 | 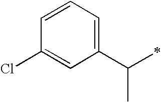 | 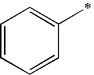 |
| 30 | H | 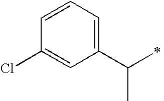 | 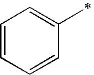 |
| 31 | 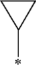 | 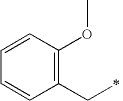 | 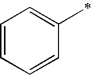 |
| 32 | 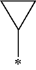 | 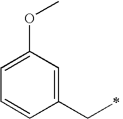 | 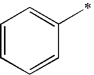 |
| 33 | 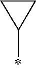 | 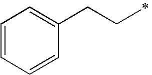 | 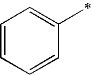 |
| 34 | 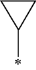 | 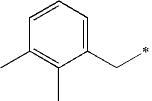 | 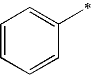 |
| 35 | 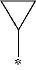 | 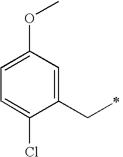 | 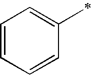 |
| 36 | 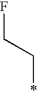 | 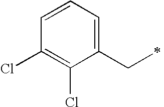 | 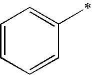 |
| 37 | 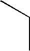 | 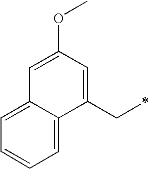 | 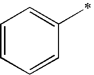 |
| 38 | 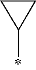 | 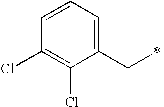 | 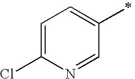 |
| 39 | 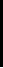 | 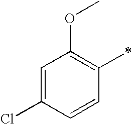 | 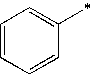 |
| 40 | 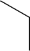 | 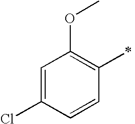 | 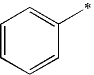 |
| 41 | 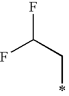 | 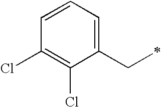 | 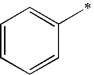 |
| 42 | 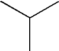 | 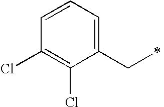 | 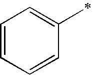 |
| 43 | 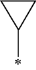 | 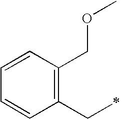 | 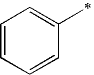 |
| 44 | 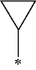 | 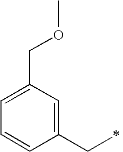 | 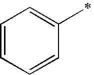 |
| 45 | 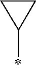 | 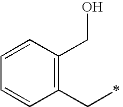 | 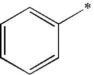 |
| 46 | 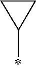 | 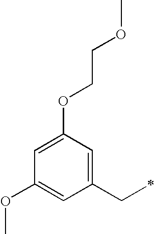 | 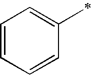 |
| 47 | 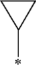 | 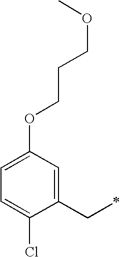 | 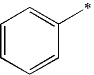 |
| 48 | 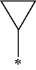 | 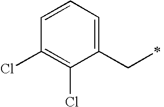 | 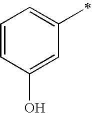 |
| 49 | 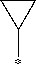 | 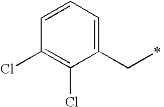 | 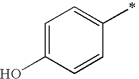 |
| 50 | 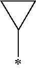 | 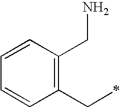 | 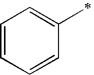 |
| 51 | 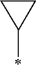 | 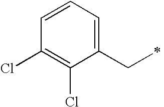 | 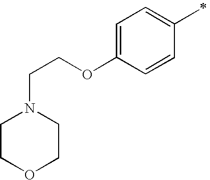 |
| 52 | 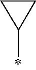 | 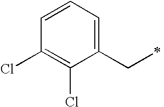 | 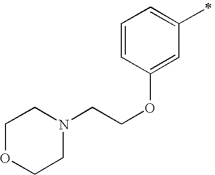 |
| 53 | 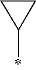 | 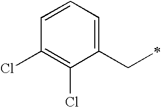 | 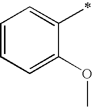 |
| 54 | 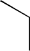 | 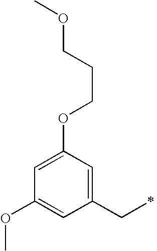 | 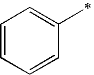 |
| 55 | 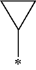 | 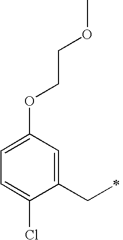 | 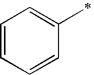 |
| 56 | 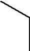 | 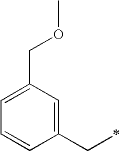 | 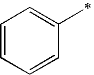 |
| 57 | 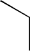 | 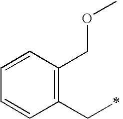 | 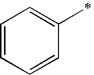 |
| 58 | 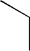 | 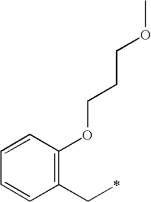 | 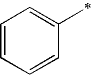 |
| 59 | 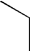 | 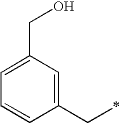 | 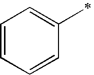 |
| 60 | 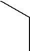 | 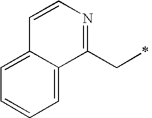 | 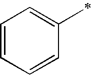 |
| 61 | 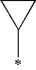 | 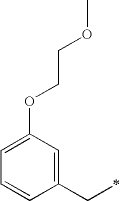 | 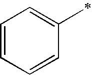 |
| 62 | 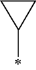 | 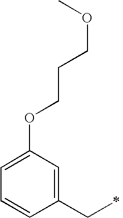 | 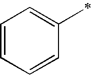 |
| 63 | 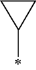 | 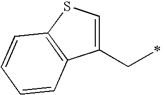 | 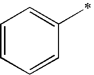 |
| 64 | 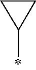 | 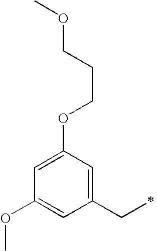 | 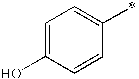 |
| 65 | 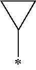 | 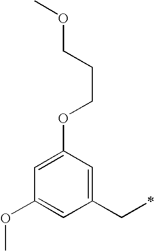 | 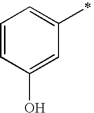 |
| 66 | 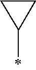 | 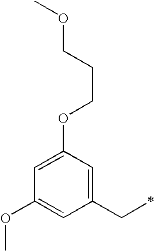 | 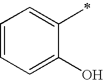 |
| 67 | 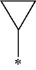 | 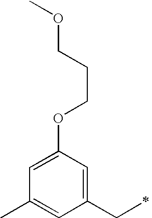 | 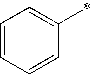 |
| 68 | 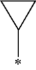 | 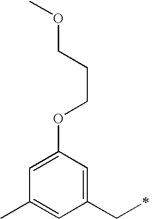 | 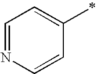 |
| 69 | 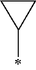 | 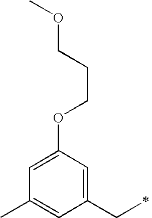 | 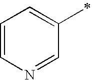 |
| 70 | 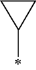 | 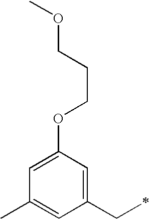 | 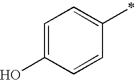 |
| 71 | 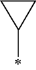 | 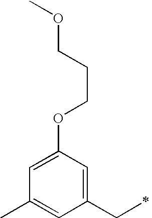 | 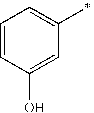 |
| 72 | 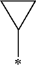 | 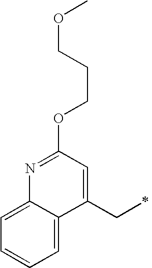 | 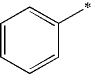 |
| 73 | 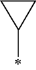 | 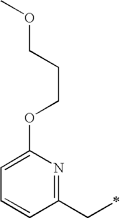 | 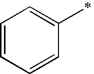 |
| 74 | 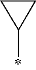 | 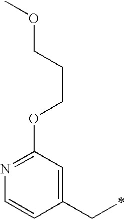 | 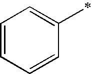 |
| 75 | 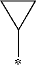 | 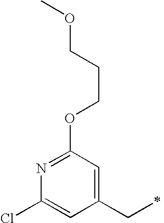 | 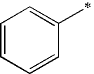 |
| 76 | 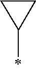 | 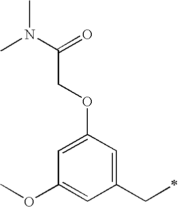 | 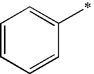 |
| 77 | 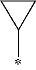 | 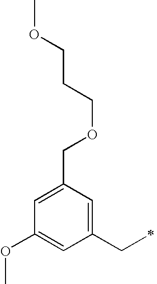 | 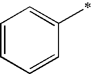 |
| 78 | 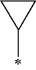 | 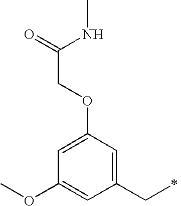 | 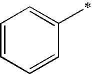 |
| 79 | 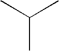 | 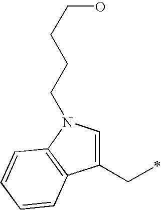 | 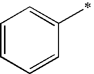 |
| 80 | 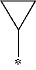 | 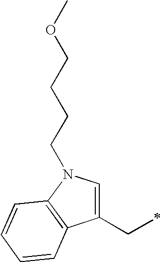 | 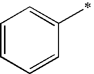 |
| 81 | 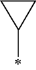 | 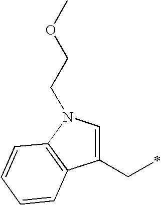 | 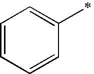 |
| 82 | 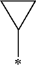 | 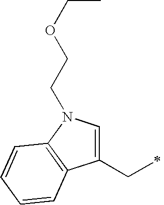 | 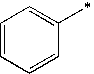 |
| 83 | 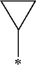 | 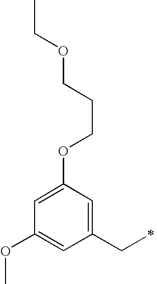 | 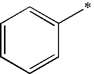 |
| 84 | 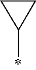 | 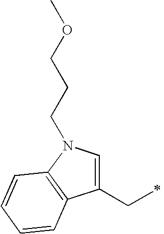 | 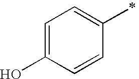 |
| 85 | 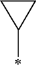 | 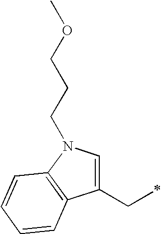 | 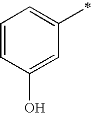 |
| 86 | 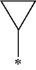 | 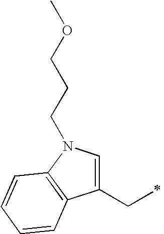 | 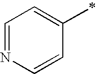 |
| 87 | 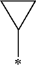 | 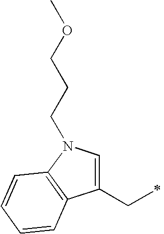 | 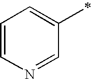 |
| 88 | 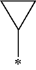 | 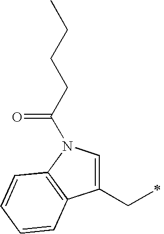 | 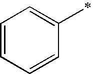 |
| 89 | 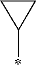 | 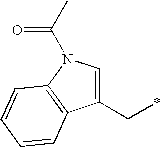 | 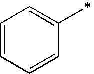 |
| 90 | 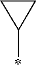 | 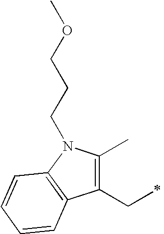 | 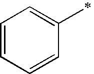 |
| 91 | 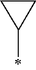 | 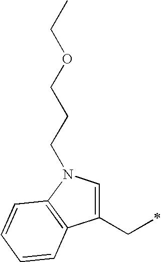 | 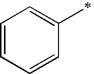 |
| 92 | 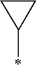 | 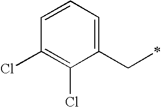 | 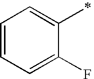 |
| 93 | 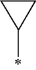 | 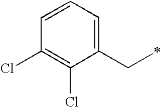 | 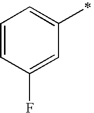 |
| 94 | 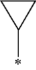 | 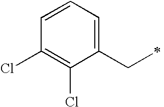 | 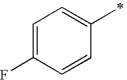 |
| 95 | 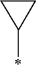 | 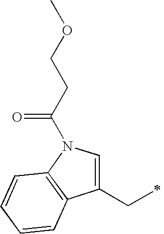 | 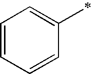 |
| 96 | 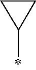 | 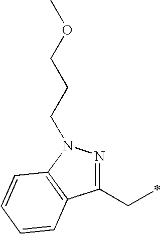 | 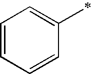 |
| 97 | 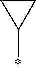 | 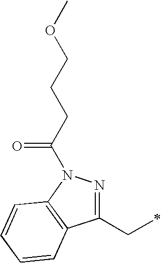 | 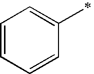 |
| 98 | 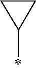 | 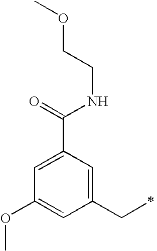 | 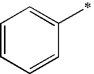 |
| 99 | 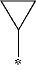 | 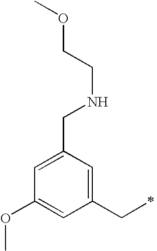 | 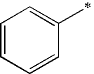 |
| 100 | 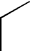 | 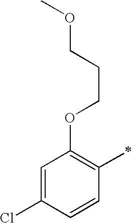 | 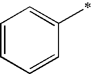 |
| 101 | 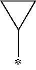 | 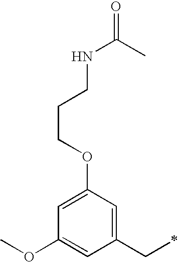 | 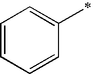 |
| 102 | 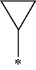 | 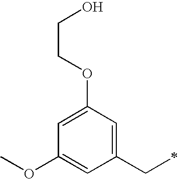 | 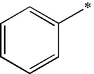 |
| 103 | 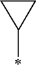 | 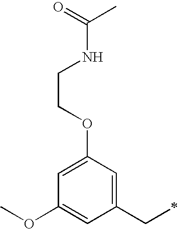 | 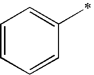 |
| 104 | 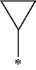 | 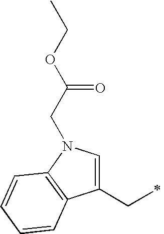 | 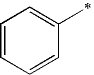 |
| 105 | 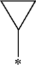 | 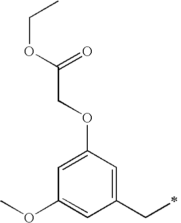 | 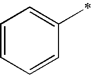 |
| 106 | 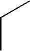 | 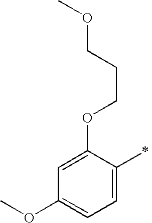 | 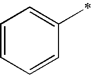 |
| 107 | 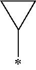 | 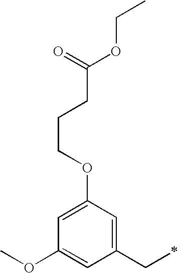 | 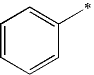 |
| 108 | 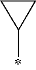 | 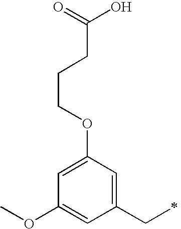 | 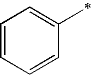 |
| 109 | 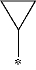 | 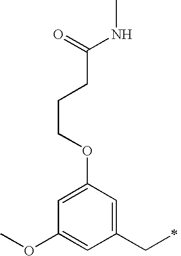 | 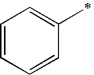 |

or a pharmaceutically acceptable salt thereof.

9. A compound of the formula I according to claim 1, selected from the group of compounds represented by the formula 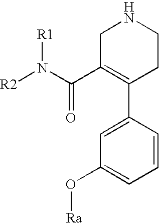
as represented in the following table:

| Compound No. | R1 | R2 | Ra |
|---|---|---|---|
| 110 | 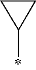 | 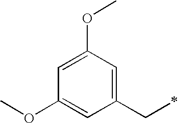 | 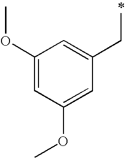 |
| 111 | 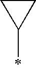 | 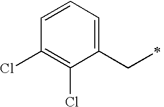 | 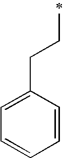 |
| 112 | 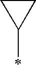 | 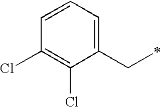 | 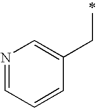 |
| 113 | 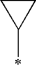 | 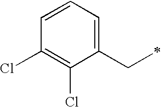 | 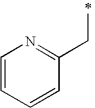 |
| 114 | 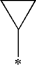 | 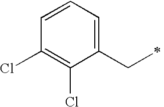 | 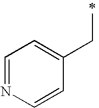 |
| 115 | 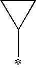 | 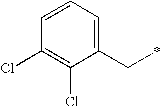 | 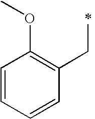 |
| 116 | 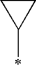 | 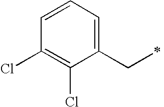 | 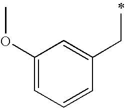 |
| 117 | 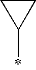 | 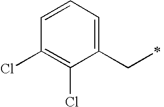 | 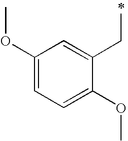 |
| 118 | 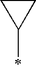 | 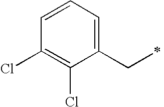 | 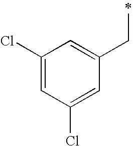 |
| 119 | 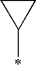 | 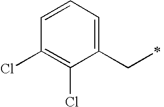 | 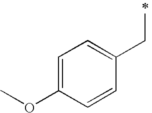 |
| 120 | 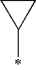 | 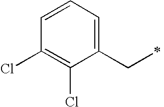 | 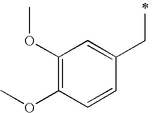 |
| 121 | 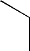 | 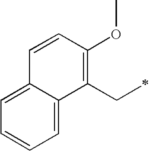 | 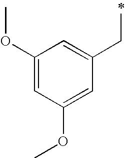 |
| 122 | 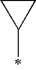 | 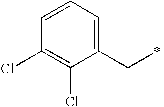 | 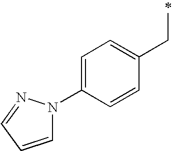 |
| 123 | 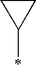 | 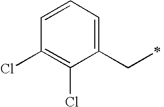 | 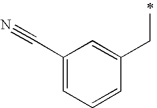 |
| 124 | 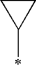 | 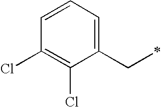 | 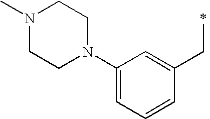 |
| 125 | 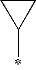 | 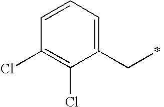 | 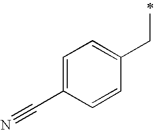 |
| 126 | 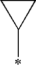 | 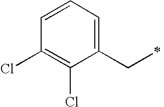 | 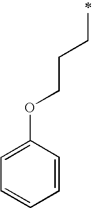 |
| 127 | 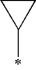 | 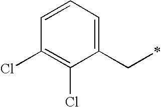 | 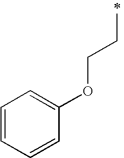 |
| 128 | 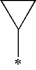 | 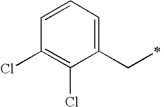 | 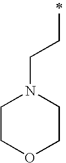 |
| 129 | 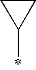 | 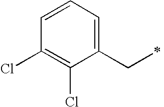 | 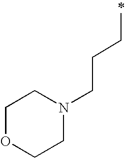 |
| 130 | 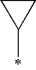 | 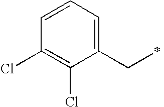 | 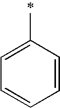 |
| 131 | 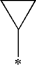 | 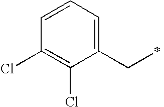 | 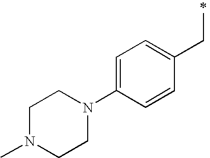 |
| 132 | 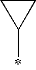 | 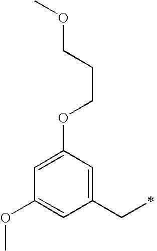 | 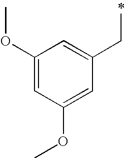 |
| 133 | 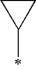 | 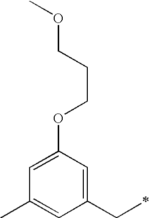 | 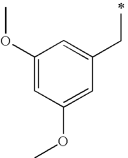 |
| 134 | 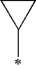 | 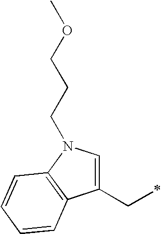 | 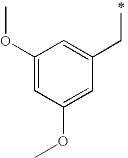 |
| 135 | 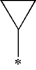 | 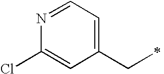 | 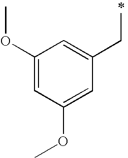 |

or a pharmaceutically acceptable salt thereof.

10. A compound of the formula I according to claim 1, selected from the group of compounds represented by the formula 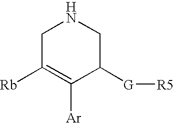
as represented in the following table:

| Compound No. | Rb | Ar | G—R5 |
|---|---|---|---|
| 136 | 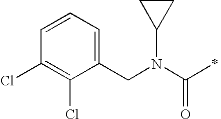 | 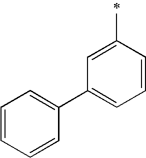 | 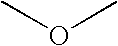 |
| 137 | 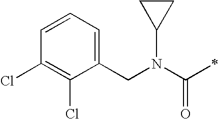 | 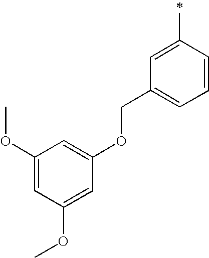 | H |
| 138 | 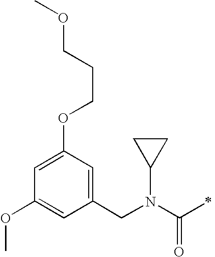 | 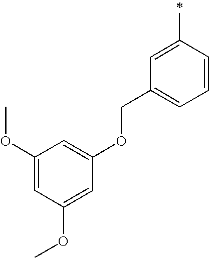 | H |
| 139 | 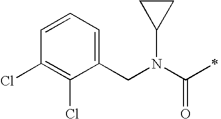 | 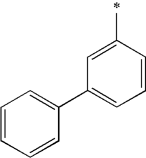 | 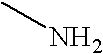 |
| 140 | 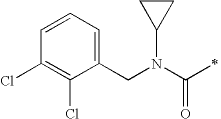 | 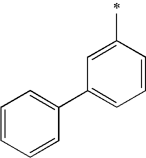 | 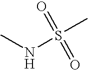 |
| 141 | 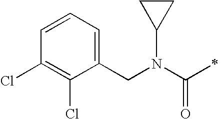 | 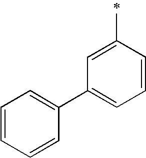 | 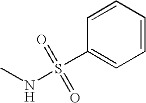 |
| 142 | 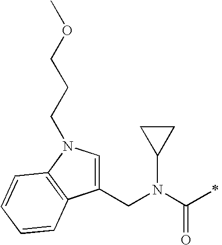 | 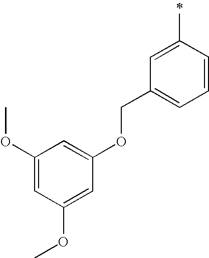 | H |
| 143 | 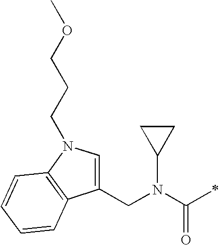 | 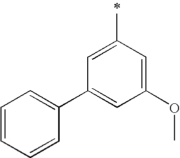 | H |
| 144 | 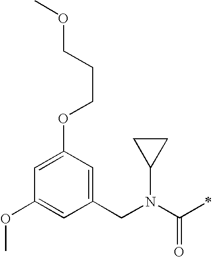 | 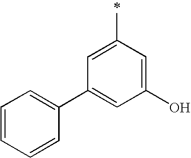 | H |
| 145 | 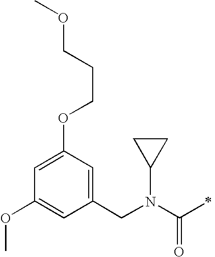 | 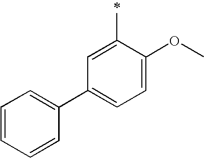 | H |
| 146 | 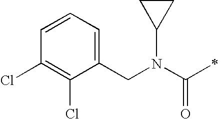 | 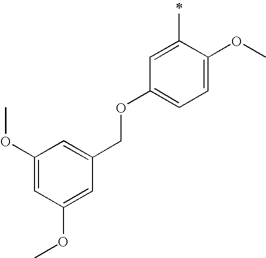 | H |
| 147 | 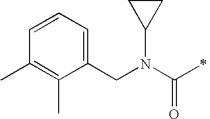 | 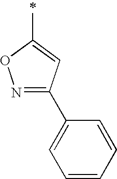 | H |

or a pharmaceutically acceptable salt thereof.

11. A compound of the formula I, or a pharmaceutically acceptable salt thereof, according to claim 1 for use in the diagnostic or therapeutic treatment of a warm-blooded animal.

12. A compound of the formula I, or a pharmaceutically acceptable salt thereof, according to claim 1 for use according to claim 11 in the treatment of a disease that depends on activity of renin.

13. The use of a compound of the formula I, or a pharmaceutically acceptable salt thereof, according to claim 1 for the manufacture of a pharmaceutical composition for the treatment of a disease that depends on activity of renin.

14. The use of a compound of the formula I, or a pharmaceutically acceptable salt thereof, according to claim 1 for the treatment of a disease that depends on activity of renin.

15. A pharmaceutical formulation, comprising a compound of the formula I, or a pharmaceutically acceptable salt thereof, according to claim 1 and at least one pharmaceutically acceptable carrier material.

16. A method of treatment a disease that depends on activity of renin, comprising administering to a warm-blooded animal, especially a human, in need of such treatment a pharmaceutically effective amount of a compound of the formula I, or a pharmaceutically acceptable salt thereof, according to claim 1

17. A process for the manufacture of a compound of the formula I, or a pharmaceutically acceptable salt thereof, according to claim 1, comprising
(a) for the synthesis of a compound of the formula I wherein the moieties are as defined for a compound of the formula I, reacting a carbonic acid compound of the formula II 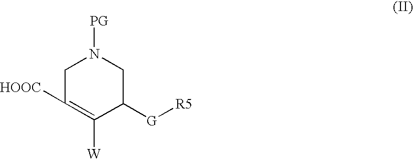
wherein W, G and R5 or -G- are as defined for a compound of the formula I and PG is a protecting group, or an active derivative thereof, with an amine of the formula III, 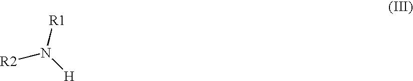
wherein R1 and R2 are as defined for a compound of the formula I, and removing protecting groups to give the corresponding compound of the formula I, or
(b) for the preparation of a compound of the formula I wherein R₃ is unsubstituted or substituted aryl or unsubstituted or substituted alkyoxy and W is a moiety of the formula IA given above, by reacting a compound of the formula IV, 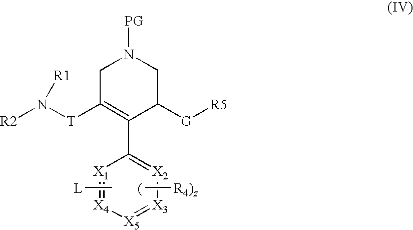
wherein R1, R2, T, G, R5, X₁, X₂, X₃, X₄, X₅, z and R₄ are as defined for a compound of the formula I, PG is a protecting group and L is a leaving group or hydroxy, with a compound of the formula V,
R3-Q (V)
wherein R3 is as just defined and Q is —B(OH) or a leaving group, and removing protecting groups to give the corresponding compound of the formula I, or
and, if desired, subsequent to any one or more of the processes mentioned above converting an obtainable compound of the formula I or a protected form thereof into a different compound of the formula I, converting a salt of an obtainable compound of formula I into the free compound or a different salt, converting an obtainable free compound of formula I into a salt thereof, and/or separating an obtainable mixture of isomers of a compound of formula I into individual isomers;
where in any of the starting materials, in addition to specific protecting groups mentioned, further protecting groups may be present, and any protecting groups are removed at an appropriate stage in order to obtain a corresponding compound of the formula I, or a salt thereof.
